(19) United States
(12) Patent Application Publication
(10) Pub. No.: US 2023/0242558 A1
(43) Pub. Date: Aug. 3, 2023

(54) NICOTINATE AND NICOTINAMIDE RIBOSIDE-BASED COMPOUNDS AND DERIVATIVES THEREOF

(71) Applicant: New Frontier Bio, Inc., Beverly, MA (US)

(72) Inventors: Bradley L. Pentelute, Revere, MA (US); Dinara S. Gunasekera, Cambridge, MA (US); Kevin Kong, Irvine, CA (US); Sabrina Johnson, Beverly, MA (US)

(21) Appl. No.: 18/104,051

(22) Filed: Jan. 31, 2023

Related U.S. Application Data
(60) Provisional application No. 63/304,904, filed on Jan. 31, 2022.

(51) Int. Cl. C07F 9/54; C07D 405/04; C07D 401/14; C07D 213/56; C07D 213/55

(52) U.S. Cl. CPC C07F 9/5435; C07D 405/04; C07D 401/14; C07D 213/56; C07D 213/55

(57) ABSTRACT

Disclosed herein are compounds related to nicotinate and nicotinamide riboside and methods of making and using said compounds. Also disclosed herein are methods of making nicotinic acid mononucleoside (NAMN).

### RELATED APPLICATIONS

This application claims the benefit of priority to U.S. Provisional Patent Application No. 63/304,904, filed Jan. 31, 2022.

### BACKGROUND

Nicotinamide adenine dinucleotide (NAD) and its derivative compounds are known as essential coenzymes in cellular redox reactions in all living organisms. Several lines of evidence have also shown that NAD participates in a number of important signaling pathways in mammalian cells, including poly-ADP-ribosylation in DNA repair (Menissier de Murcia et al., EMBO J., 22:2255-2263 (2003)), mono-ADP-ribosylation in the immune response and G-protein coupled signaling (Corda and Di Girolamo, EMBO J., 22:1953-8 (2003)), and the synthesis of ADP-cyclic ribose and nicotinate adenine dinucleotide phosphate (NAADP) in intracellular calcium signaling (Lee, Annu. Rev. Pharmacol. Toxicol., 41:317-345(2001)). Recently, NAD and its derivatives have also been shown to play an important role in transcriptional regulation (Lin and Guarente, Curr. Opin. Cell. Biol., 15:241-246 (2003)). In particular, the discovery of Sir2 NAD-dependent deacetylase activity (e.g., Imai et al., Nature, 403:795-800 (2000); Landry et al., Biochem. Biophys. Res. Commun., 278:685-690 (2000); Smith et al., Proc. Natl. Acad. Sci. USA, 97:6658-6663 (2000)) drew attention to this new role for NAD.

NAD+ is thought to be related to the aging process. This is demonstrated in the replicative life span of S. cerevisiae, which is typically defined as the number of buds or “daughter cells” produced by an individual “mother cell” (Barton, A., J. Gen. Microbiol., 4:84-86(1950)). Mother cells undergo age-dependent changes including an increase in size, a slowing of the cell cycle, enlargement of the nucleolus, an increase in steady-state NAD⁺ levels, increased gluconeogenesis and energy storage, and sterility resulting from the loss of silencing at telomeres and mating-type loci (Sinclair et al., Science, 277(5330): 1313-6 (1997); Mortimer et al., Nature, 183: 1751-1752 (1959); Kennedy et al., J. Cell Biol., 127(6): 1985-93 (1994); Kim et al., Biochem. Biophys. Res. Commun., 219(2): 370-6 (1996); Ashrafi et al., Genes Dev., 14(15): 1872-85 (2000); Lin et al., J. Biol. Chem., (2001)).

A key regulator of aging in yeast is the Sir2 silencing protein (Kaeberlein et al., Genes Dev., 13(19): 2570-80 (1999)), a nicotinamide adenine dinucleotide (NAD+)-dependent deacetylase (Tanner et al., Proc. Natl. Acad. Sci. USA, 97(26) 14178-82 (2000); Imai et al., Nature, 403(6771): 795-800 (2000); Smith et al., Proc. Natl. Acad. Sci. USA, 97(12): 6658-63 (2000); Landry et al., Proc. Natl. Acad. Sci. USA, 97(11): 5807-11(2000)). Sir2 is a component of the heterotrimeric Stir2/3/4 complex that catalyzes the formation of silent heterochromatin at telomeres and the two silent mating-type loci (Laurenson et al., Microbiol. Rev., 56(4): 543-60 (1992)). Sir2 is also a component of the RENT complex that is required for silencing at the rDNA locus and exit from telophase (Straight et al., Cell, 97(2): 245-56 (1999); Shou et al., Cell, 97(2): 233-44 (1999)). This complex has also recently been shown to directly stimulate transcription of rRNA by Pol I and to be involved in regulation of nucleolar structure (Shou et al., Mol. Cell., 8(1): 45-55 (2001)).

Biochemical studies have shown that Sir2 can readily deacetylate the amino-terminal tails of histones H3 and H4, resulting in the formation of 1-O-acetyl-ADP-ribose and nicotinamide (Tanner et al., Proc. Natl. Acad. Sci. USA, 97(26) 14178-82 (2000); Imai et al., Nature, 403(6771): 795-800 (2000); Smith et al., Proc. Natl. Acad. Sci. USA, 97(12): 6658-63 (2000); Tanny et al., Proc. Natl. Acad. Sci. USA, 98(2): 415-20 (2001)). Strains with additional copies of SIR2 display increased rDNA silencing (Smith et al., Mol. Cell Biol., 19(4): 3184-97 (1999)) and a 30% longer life span (Kaeberlein et al., Genes Dev 13(19): 2570-80 (1999)). It has recently been shown that additional copies of the C. elegans SIR2 homolog, sir-2.1, greatly extend life span in that organism (Tissenbaum et al., Nature, 410(6825): 227-30 (2001)). This implies that the SIR2-dependent regulatory pathway for aging arose early in evolution and has been well conserved (Kenyon, C., Cell, 105: 165-168 (2001)).

In most organisms, there are two pathways of NAD+ biosynthesis. NAD+ may be synthesized de novo from tryptophan or recycled in four steps from nicotinamide via the NAD+ salvage pathway. The first step in the bacterial NAD⁺ salvage pathway, the hydrolysis of nicotinamide to nicotinic acid and ammonia, is catalyzed by the pncA gene product (Foster et al., J Bacteriol, 137(3): 1165-75 (1979)). An S. cerevisiae gene with homology to pncA, YGLO37, was recently assigned the name PNC1 (SGD) (Ghislain et al., Yeast, 19(3): 215-224 (2002)). A nicotinate phosphoribosyltransferase, encoded by the NPT1 gene in S. cerevisiae, converts the nicotinic acid from this reaction to nicotinic acid mononucleotide (NaMN) (Wubbolts et al., J. Biol. Chem., 265(29): 17665-72 (1990); Vinitsky et al., J. Bacteriol., 173(2): 536-40 (1991); Imsande, J. Biochim. Biophys. Acta., 85, 255-273 (1964); Grubmeyer et al., Methods Enrymol., 308: 28-48 (1999)). At this point, the NAD⁺ salvage pathway and the de novo NAD⁺ pathway converge and NaMN is converted to desamido-NAD⁺ (NaAD) by a nicotinate mononucleotide adenylyltransferase (NaMNAT). In S. cerevisiae, there are two putative open reading frames (ORFs) with homology to bacterial NaMNAT genes, YLR328 (Emanuelli et al., FEBS Lett., 455(1-2): 13-7 (1999)) and an uncharacterized ORF, YGR010 (Smith et al., Proc. Natl. Acad. Sci. USA, 97(12): 6658-63 (2000); Emanuelli et al., FEBS Lett., 455(1-2): 13-7 (1999)). In Salmonella, the final step in the regeneration of NAD+ is catalyzed by an NAD synthetase (Hughes et al., J. Bacteriol., 170(5): 2113-20 (1988)).

Sir2 is a limiting component of yeast longevity. A single extra copy of the SIR2 gene extends the yeast life span by 40% (Kaeberlein et al., Genes Dev., 13(19): 2570-80 (1999); Lin et al., Science, 289(5487): 2126-8 (2000); Anderson et al., J. Biol. Chem., 277(21): 18881-90 (2002)). Recently, it has been shown that increased dosage of the Sir2 homologue sir2.1 extends the life span of the nematode C. elegans (Tissenbaum et al., Nature, 410(6825): 227-30 (2001)). The nearest human homologue SIRT1, has been shown to inhibit apoptosis through deacetylation of p53 (Vaziri et al., Cell, 107(2):149-59 (2001); Luo et al., Cell, 107(2): 137-48 (2001)). These findings suggest that Sir2 and its homologues have a conserved role in the regulation of survival at the cellular and organismal level.

Recently, a great deal of insight has been gained into the biochemistry of Sir2-like deacetylases (reviewed by Moazed, D., Curr Opin Cell Biol, 13(2): 232-8 (2001)). In vitro, Sir2 has specificity for lysine 16 of histone H4 and lysines 9 and 14 of histone H3 (Imai et al., Nature, 403:795-800 (2000); Landry et al., Biochem. Biophys. Res. Commun., 278:685-690 (2000); Smith et al., Proc. Natl. Acad. Sci. USA, 97:6658-6663 (2000)). The Sir2 reaction requires NAD+ as a cofactor, allowing regulation of Sir2 activity through changes in availability of this co-substrate (Imai et al., Nature, 403:795-800 (2000); Landry et al., Biochem. Biophys. Res. Commun., 278:685-690 (2000); Smith et al., Proc. Natl. Acad. Sci. USA, 97:6658-6663 (2000); Tanner et al., Proc. Natl. Acad. Sci. USA, 97(26) 14178-82 (2000)). Sir2 deacetylation is coupled to cleavage of the high-energy glycosidic bond that joins the ADP-ribose moiety of NAD+ to nicotinamide. Upon cleavage, Sir2 catalyzes the transfer of an acetyl group to ADP-ribose (Smith et al., Proc. Natl. Acad. Sci. USA, 97:6658-6663 (2000); Tanner et al., Proc. Natl. Acad. Sci. USA, 97(26) 14178-82 (2000); Tanny et al., Proc. Natl. Acad. Sci. USA, 98(2): 415-20 (2001); Sauve et al., Biochemistry, 40(51): 15456-63 (2001)). The product of this transfer reaction is O-acetyl-ADP-ribose, a novel metabolite, which has recently been shown to cause a delay/block in the cell cycle and oocyte maturation of embryos (Borra et al., J Biol Chem, 277(15): 12632-41 (2002)).

The other product of deacetylation is nicotinamide, a precursor of nicotinic acid and a form of vitamin B3 (Dietrich, L. S., Amer. J. Clin. Nut., 24: 800-804 (1971)). High doses of nicotinamide and nicotinic acid are often used interchangeably to self-treat a range of conditions including anxiety, osteoarthritis, psychosis, and nicotinamide is currently in clinical trials as a therapy for cancer and type I diabetes (Kaanders et al., Int. J. Radiat. Oncol. Biol. Phys., 52(3): 769-78(2002)). Despite the important biological role of NAD+ and its association with the aging process, there still exists a need for methods of forming NAD+ precursors in a simple and cost-effective manner.

### SUMMARY OF THE INVENTION

In one aspect, the present disclosure provides compounds having a structure represented by formula (VI) or a pharmaceutically acceptable salt thereof:

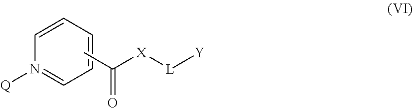

wherein:
Q is absent,

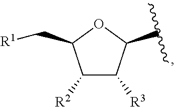

### or H;

R¹ is HPO₄, H₂PO₄, —OH, -OAcyl, or —OC(O)R⁴;
R² and R³ are independently —OH, —C(O)R⁴, —C(O)OR⁴, —C(O)NHR⁴ or halogen;
R⁴ is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
L is a bond, C₁₋₂₀ alkyl, aryl, heteroaryl, arylalkylaryl, arylalkyl, alkoxy, or —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R¹, —CO₂R¹, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
Y is —C(O)NH₂, —C(O)OH, —R⁵, —P(R⁷)₃, —NH₂, —NHR⁵,

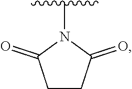 [SMILES: *N1C(=O)CCC1=O]

### —SH, or —OH;

R⁵ is —C(O)R⁴,

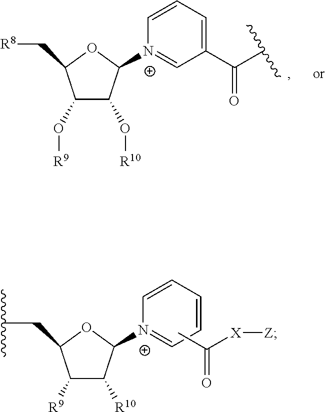

R⁷ is individually selected at each occurrence from the group consisting of substituted or unsubstituted alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —H, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z is H, or C₁₋₂₀ alkyl; or Z and R¹ are optionally taken together as a bond, forming a macrocycle,
   with the proviso that if X is O, NH, or NR⁷, and L is C₁₋₂₀ alkyl, aryl, heteroaryl, or alkoxy, then Y is not —C(O)NH₂, —C(O)OH, —R⁵, —NH₂, —NHR⁵, —SH, or —OH.

In further aspects, the present disclosure provides methods of making and using the compounds disclosed erien.

In another aspect, the present disclosure provides methods of making nicotinic acid mononucleoside (NAMN).

### BRIEF DESCRIPTION OF THE DRAWINGS

FIG. 1 is a graphic depiction of the average total NAD concentration from the NAD Assay comparing nicotinic acid mononucleotide (NaMN, Sample 1) to nicotinamide mononucleotide (NMN).

FIG. 2 is a graphic depiction of the average total NAD concentration from the NAD Assay Comparing 1-((2R,3R,4S,5R)-3,4-dihydroxy-5-((phosphonooxy)-methyl)tetrahydrofuran-2-yl)-3-((6-(triphenylphosphonio)hexyl)carbamoyl)-pyridin-1-ium (7, Sample 2) to nicotinamide mononucleotide (NMN).

FIG. 3 is a graphic depiction of the average total NAD concentration from the NAD Assay comparing 6-(nicotinamido)hexyl)triphenylphosphonium (10, Sample 5) to nicotinamide mononucleotide (NMN).

FIG. 4A is a schematic depiction of exemplary flow chemistry setups for synthesizing compounds disclosed herein.

FIG. 4B is a schematic depiction of exemplary flow chemistry setups for synthesizing compounds disclosed herein.

### DETAILED DESCRIPTION OF THE INVENTION

### Compounds of Formula (VI) and Related Formulas

In one aspect, the present disclosure relates to compounds having a structure represented by formula (VI) or a pharmaceutically acceptable salt thereof:

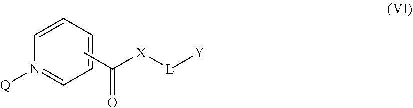

wherein:
Q is absent,

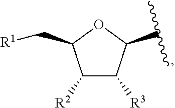

### or H;

R¹ is HPO₄, H₂PO₄, —OH, -OAcyl, or —OC(O)R⁴;
R² and R³ are independently —OH, —C(O)R⁴, —C(O)OR⁴, —C(O)NHR⁴ or halogen;
R⁴ is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
L is a bond, C₁₋₂₀ alkyl, aryl, heteroaryl, arylalkylaryl, arylalkyl, alkoxy, or —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
   Y is —C(O)NH₂, —C(O)OH, —R¹, —P(R⁷)₃, —NH₂, —NHR⁵,

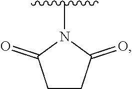 [SMILES: *N1C(=O)CCC1=O]

### —SH, or —OH;

R⁵ is —C(O)R⁴,

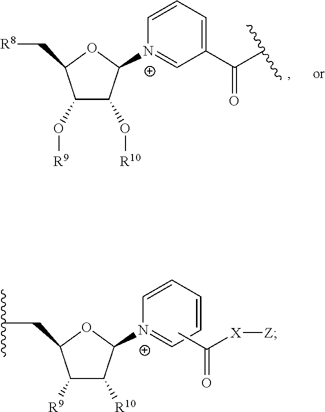

R⁷ is individually selected at each occurrence from the group consisting of substituted or unsubstituted alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —H, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z is H, or C₁₋₂₀ alkyl; or Z and R¹ are optionally taken together as a bond, forming a macrocycle,
with the proviso that if X is O, NH, or NR⁷, and L is C₁₋₂₀ alkyl, aryl, heteroaryl, or alkoxy, then Y is not —C(O)NH₂, —C(O)OH, —R⁵, —NH₂, —NHR⁵, —SH, or —OH.

In certain embodiments, the compound has a structure represented by formula VIa:

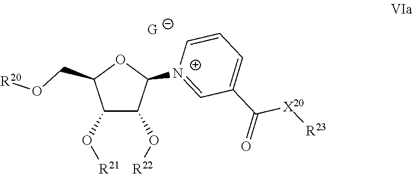

wherein,

R²⁰ is H, P(O)₂OH, P(O)(OH)₂, or acyl;

R²¹ and R²² are each independently H or acyl;

R²³ is H, alkyl, cycloalkyl, aralkyl, or aryl;

R²⁴ is H or alkyl;

X²⁰ is O, N(R²⁴), or S; and

G is an anion.

In certain embodiments, R² is H. In certain embodiments, R² is P(O)(OH)₂. In certain embodiments, R² is acyl (e.g., alkylacyl or heteroarylacyl).

In certain embodiments, R²¹ is H. In certain embodiments, R²¹ is acyl (e.g., alkylacyl or heteroarylacyl).

In certain embodiments, R²² is H. In certain embodiments, R²² is acyl (e.g., alkylacyl or heteroarylacyl).

In certain embodiments, X²⁰ is O. In certain embodiments, X²⁰ is NH. In certain embodiments, X²⁰ is S.

In certain embodiments, R²³ is H. In certain embodiments, R²³ is alkyl. In certain embodiments, R²³ is alkylaminoalkyl. In certain embodiments, R²³ is alkylamidoalkyl. In certain embodiments, R²³ is aralkyl (e.g., benzyl). In certain embodiments, R²³ is aryl (e.g., phenyl). In certain embodiments, R²³ is cycloalkyl (e.g., cyclohexyl).

In certain embodiments, R²³ is substituted with triarylphosphonium (e.g., P⁺(Ph)₃). In certain embodiments, R²³ is substituted with vinyl (e.g., phenylvinyl, such as dihydroxyphenylvinyl or diacetylphenylvinyl). In certain embodiments, R²³ is substituted with amido. In certain embodiments, R²³ is substituted with

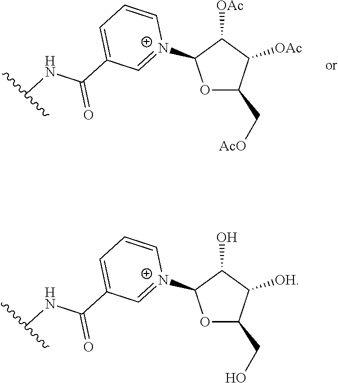 [SMILES: *NC(=O)c1ccc[n+]([C@@H]2O[C@H](COC(C)=O)[C@@H](OC(C)=O)[C@H]2OC(C)=O)c1 ; *NC(=O)c1ccc[n+]([C@@H]2O[C@H](CO)[C@@H](O)[C@H]2O)c1]

In certain embodiments, R²³ is substituted with ester. In certain embodiments, R²³ is substituted with

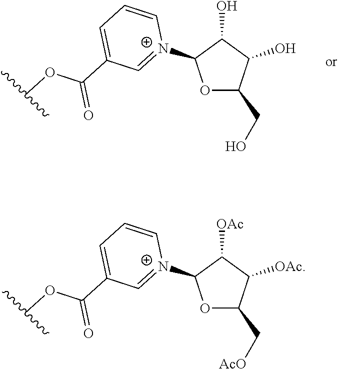 [SMILES: *OC(=O)c1ccc[n+]([C@@H]2O[C@H](CO)[C@@H](O)[C@H]2O)c1 ; *OC(=O)c1ccc[n+]([C@@H]2O[C@H](COC(C)=O)[C@@H](OC(C)=O)[C@H]2OC(C)=O)c1]

In certain embodiments, R²³ is substituted with halo (e.g., bromo). In certain embodiments, R²³ is substituted with alkyl.

In certain embodiments, G is a pharmaceutically acceptable anion.

In certain embodiments, the compound is selected from the group consisting of:

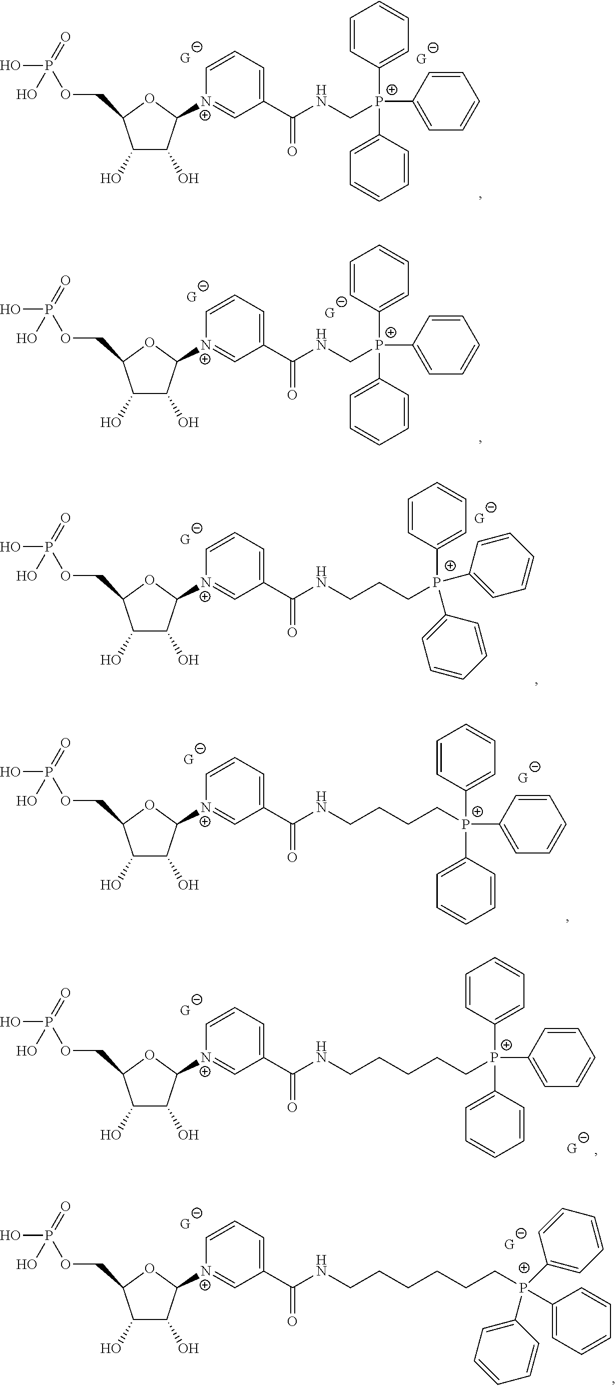

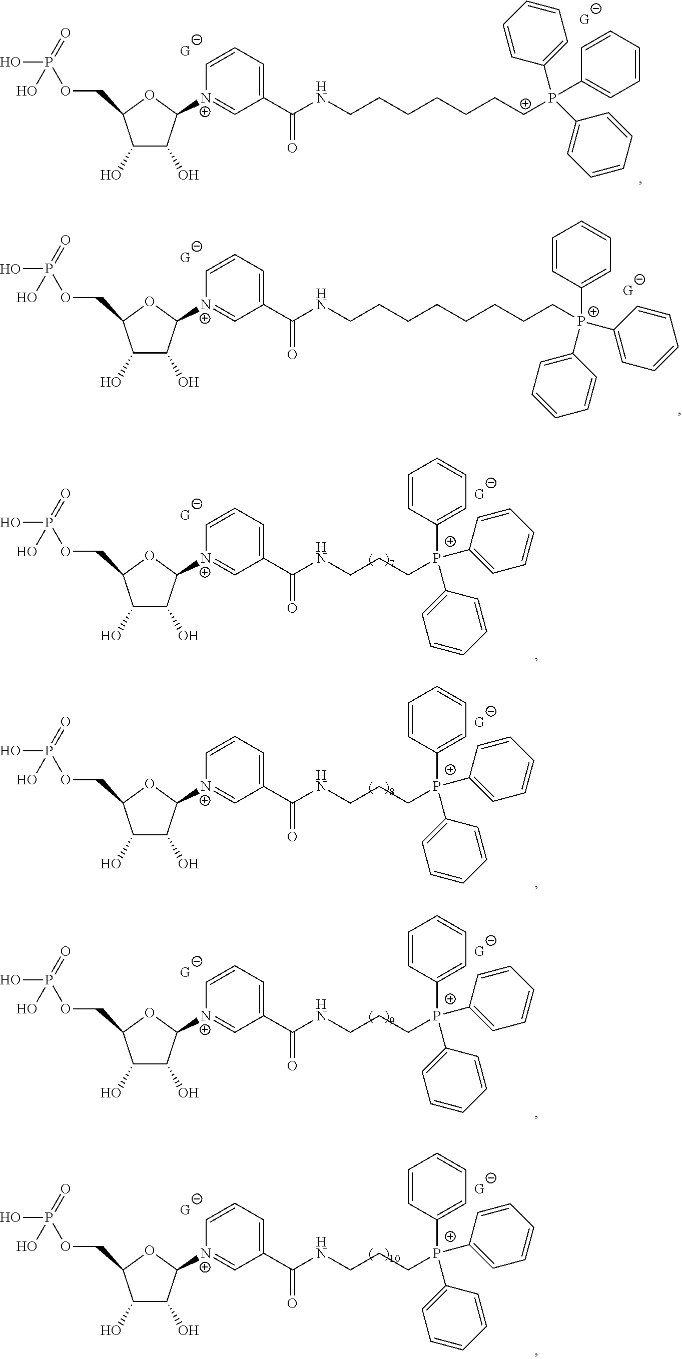

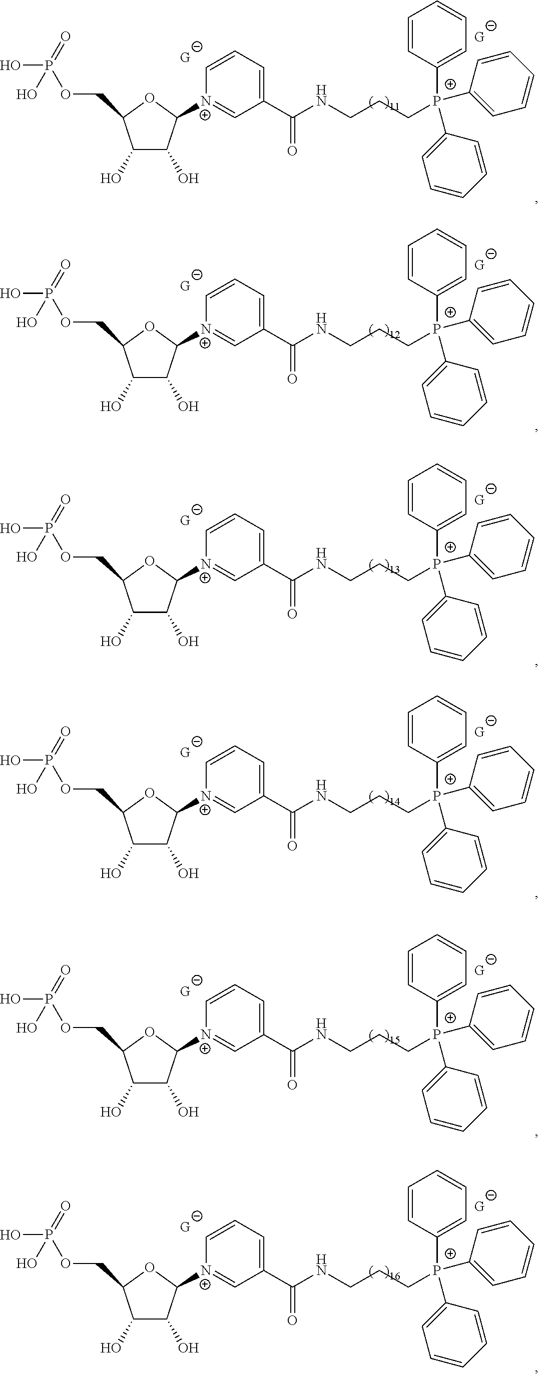

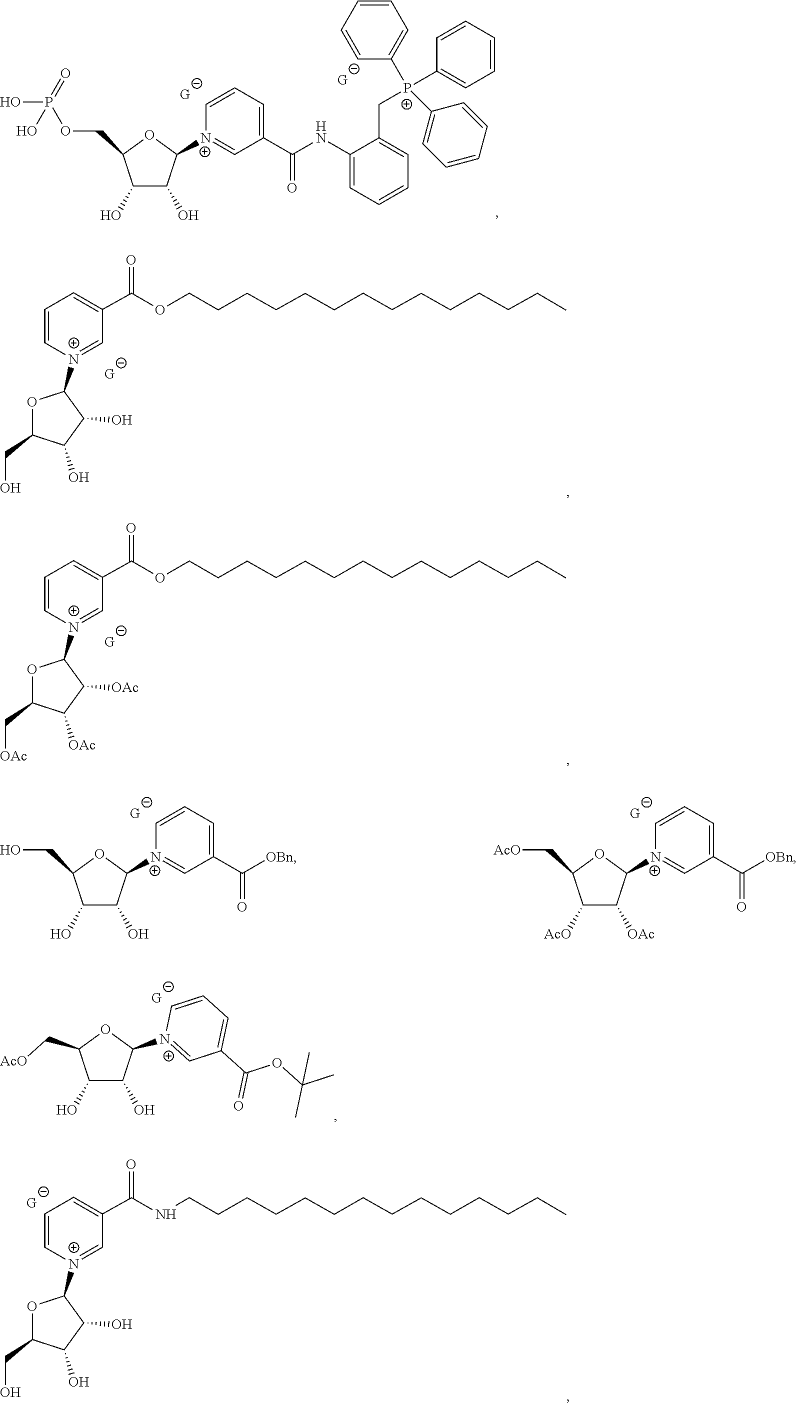

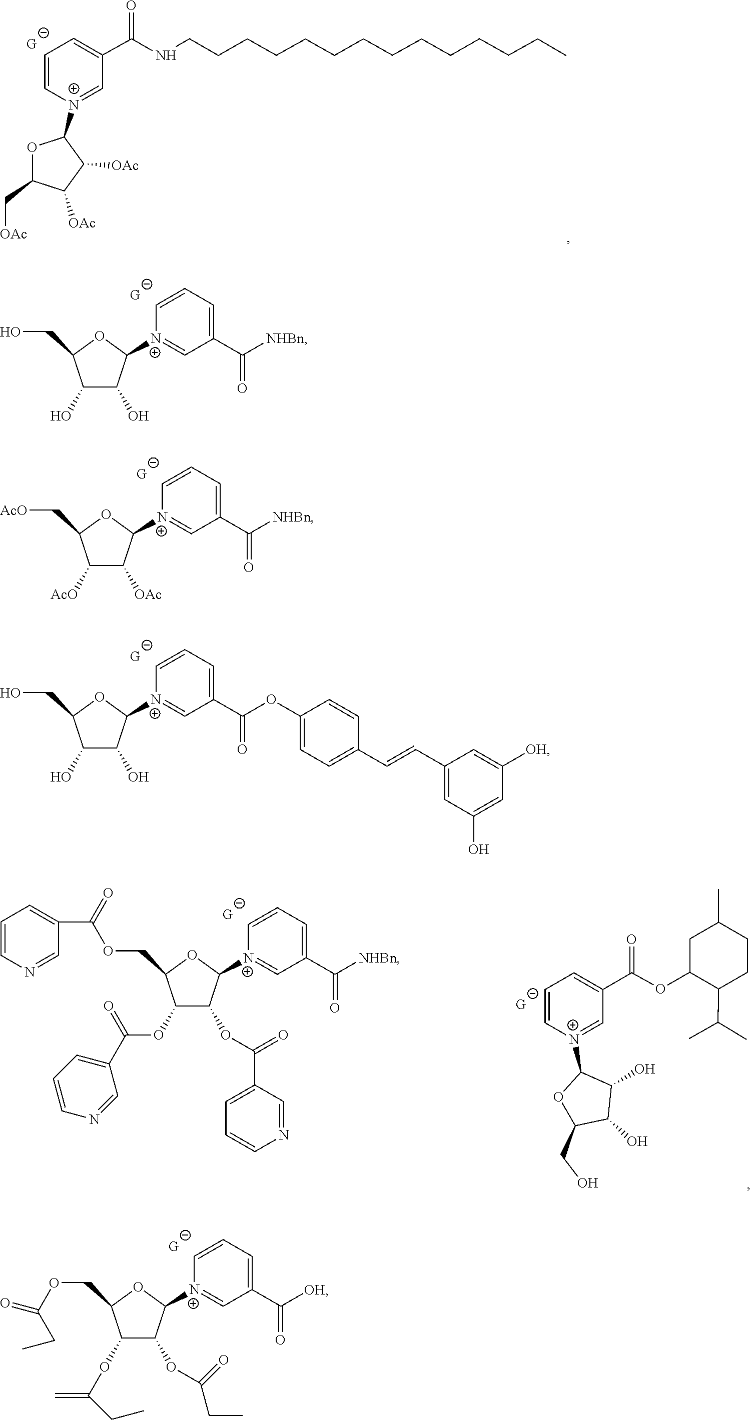

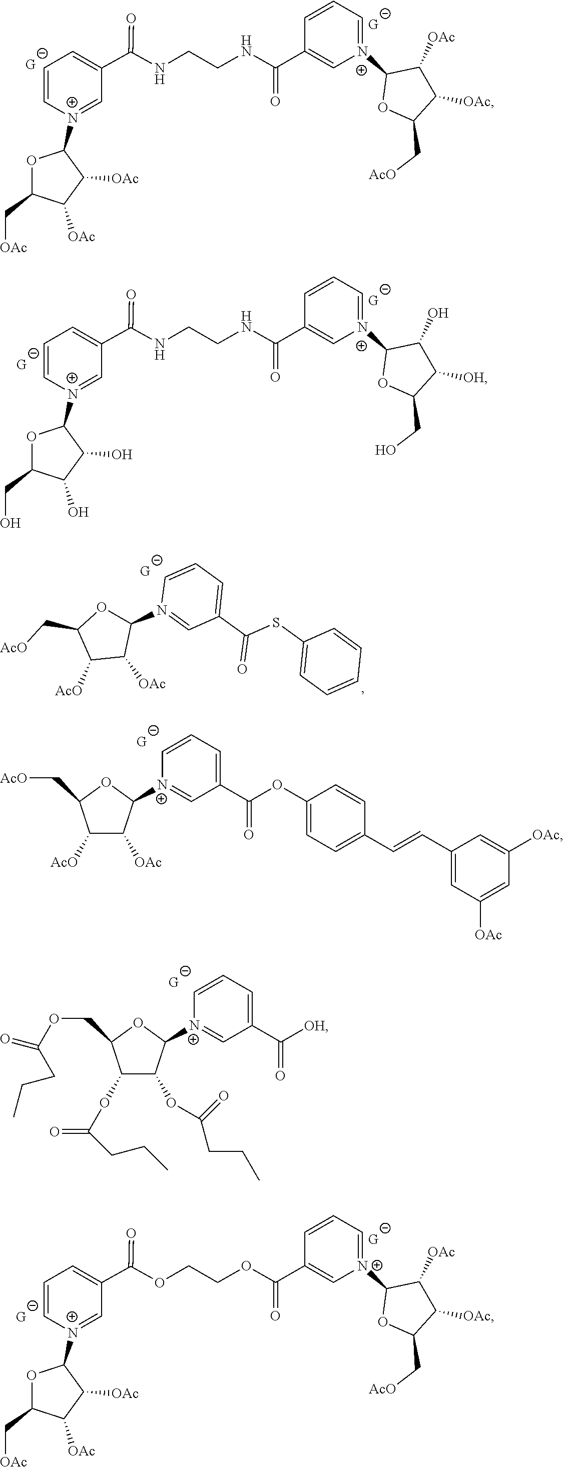

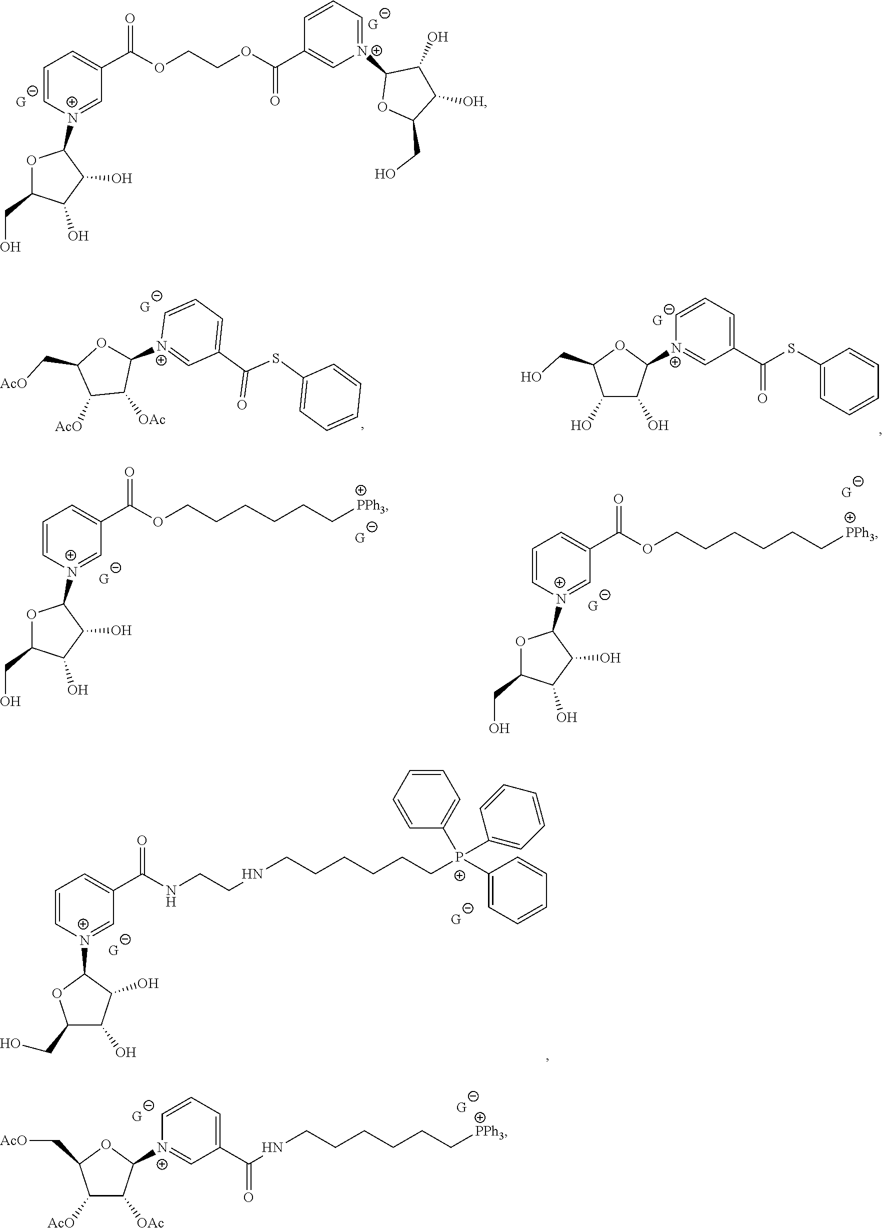

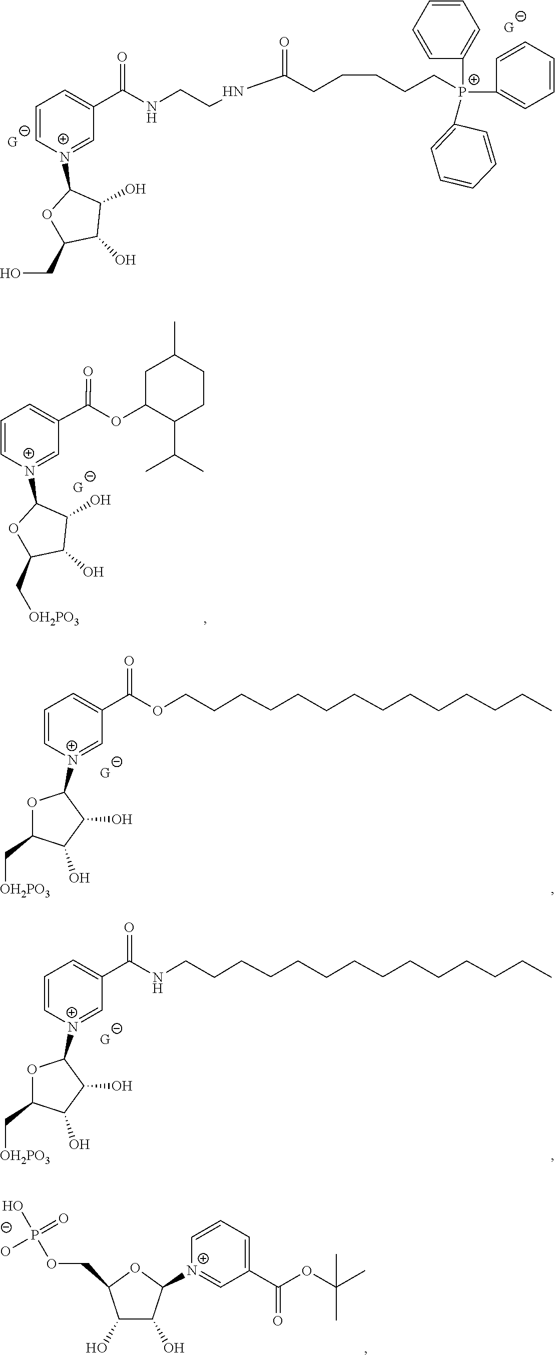

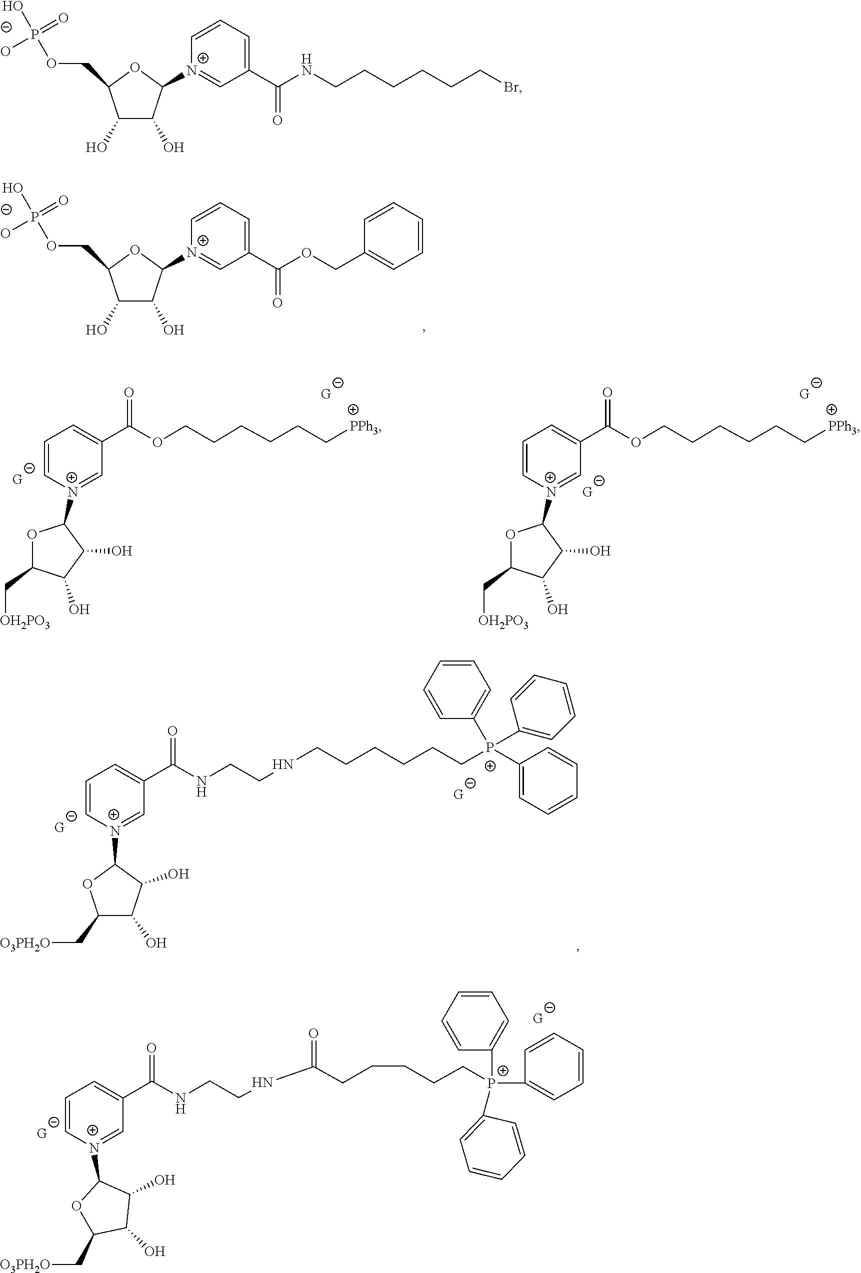

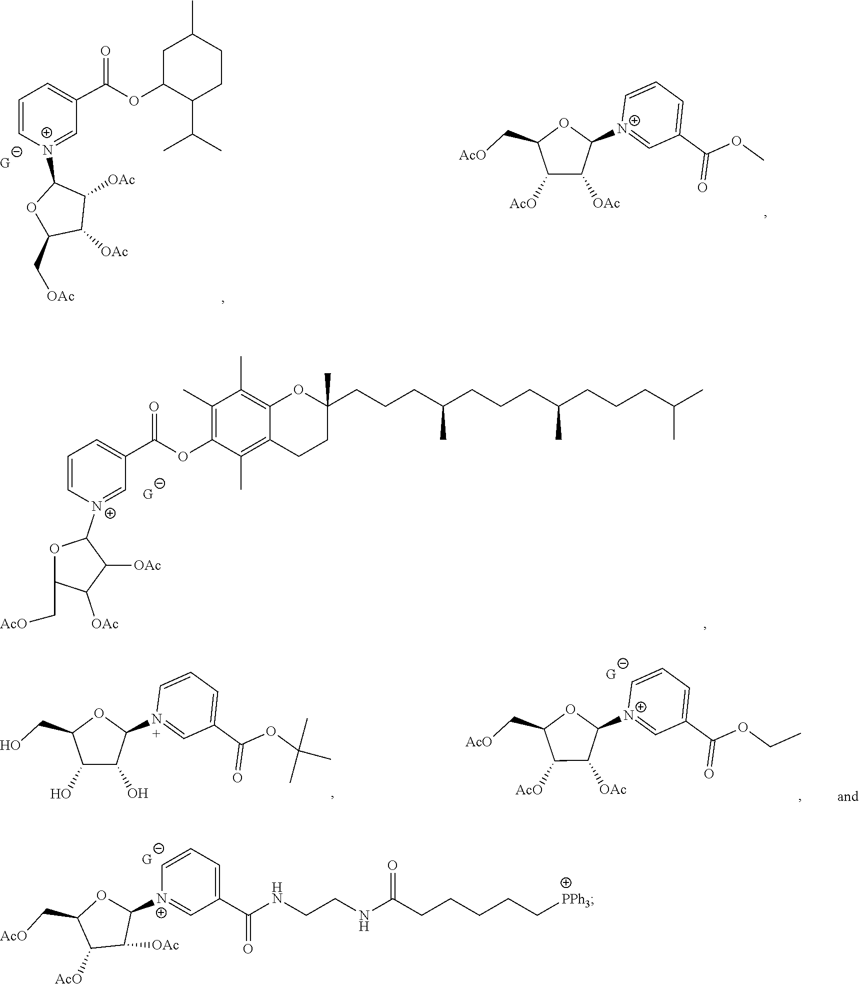

and
wherein G is a pharmaceutically acceptable anion.

In certain embodiments, the compound has a structure represented by formula VIb:

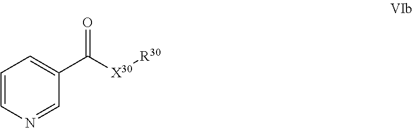

wherein,

R³⁰ is alkyl, aryl, heteroaryl, or cycloalkyl;

X³⁰ is O, N(R³⁴), or S; and

R³⁴ is H or alkyl.

In certain embodiments, X³⁰ is NH. In certain embodiments, X³⁰ is O.

In certain embodiments, R³⁰ is alkyl. In certain embodiments, R³⁰ is cycloalkyl. In certain embodiments, R³⁰ is aryl. In certain embodiments, R³⁰ is heteroaryl.

In certain embodiments, R³⁰ is substituted with triarylphosphonium (e.g., P⁺(Ph)₃). In certain embodiments, R³⁰ is substituted with alkyl. In certain embodiments, R³⁰ is substituted with hydroxyl. In certain embodiments, R³⁰ is substituted with amido (e.g., alkylamido, esteralkylamido, alkylarylalkylamido, arylaminoaralkylamido, retionylamido, or triarylphosphoniumalkylamido). In certain embodiments, R³⁰ is substituted with amino (e.g., triarylphosphoniumalkylamino). In certain embodiments, R³⁰ is substituted with alkoxy (e.g., triarylphosphoniumalkoxy). In certain embodiments, R³⁰ is substituted with alkenyl (e.g., arylvinyl). In certain embodiments, R³⁰ is substituted with ester (e.g., alkylarylester, arylaminoaralkylester, retionylester, or triarylphosphoniumalkylester).

In certain embodiments, the compound is selected from the group consisting of:

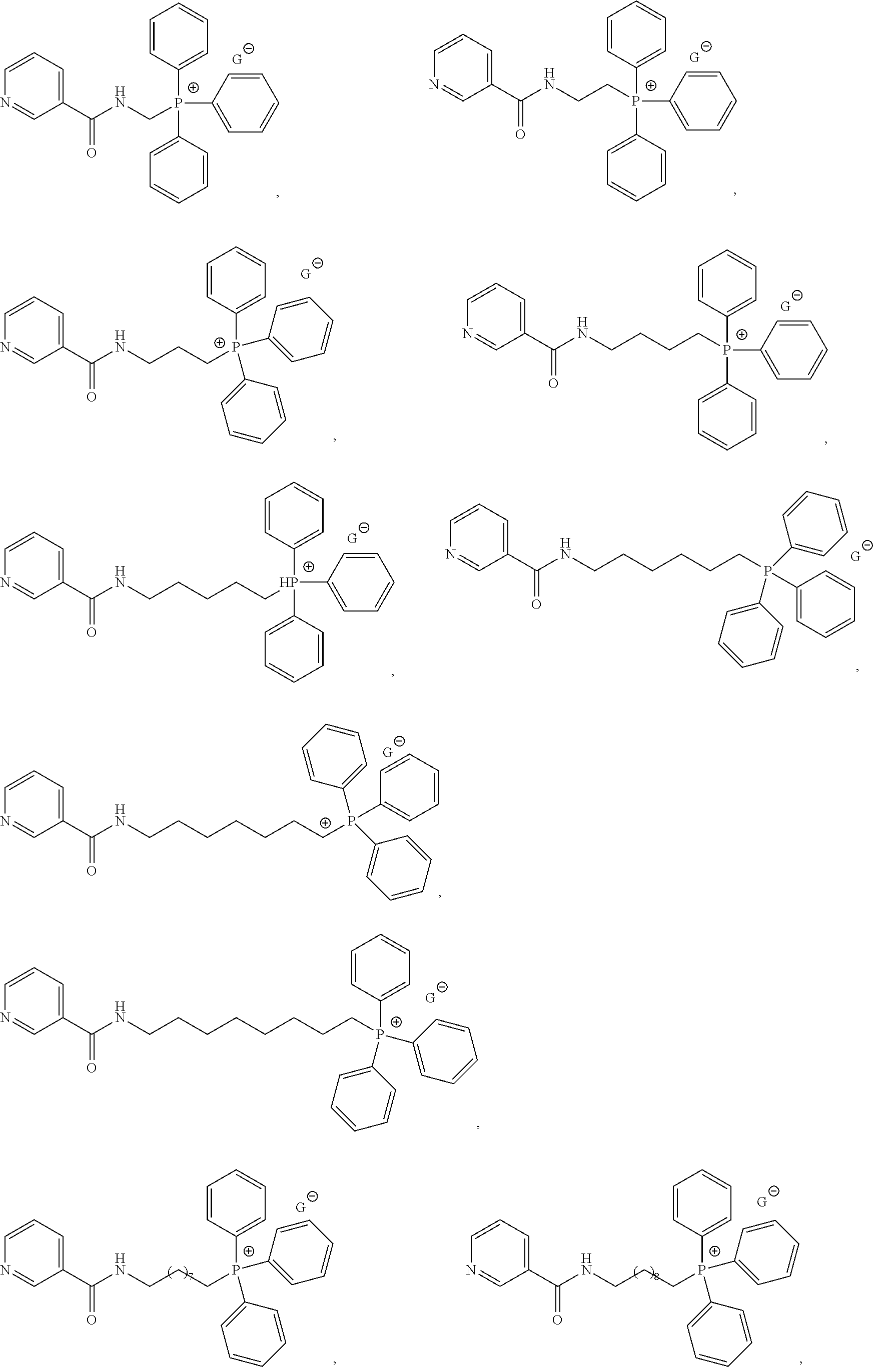

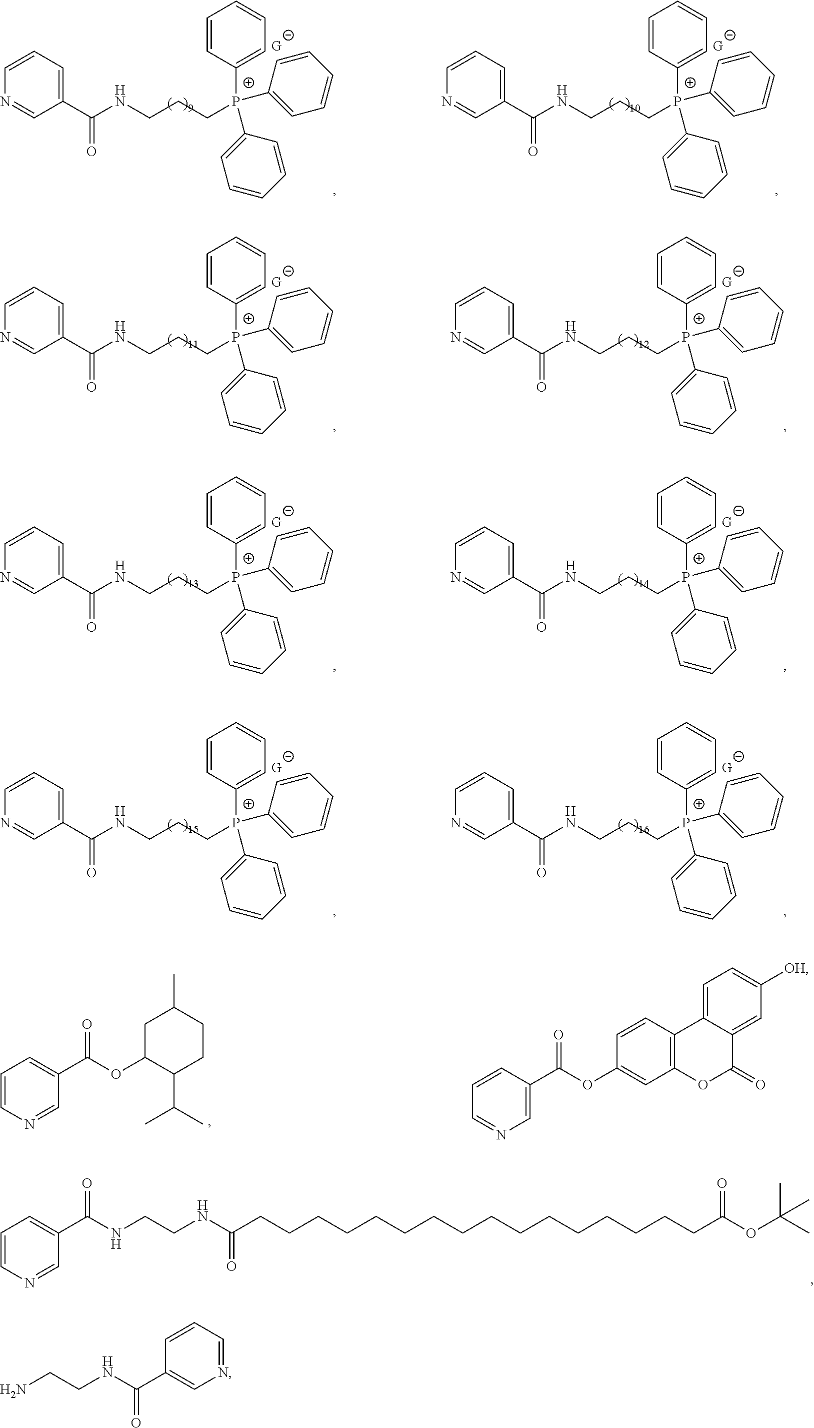

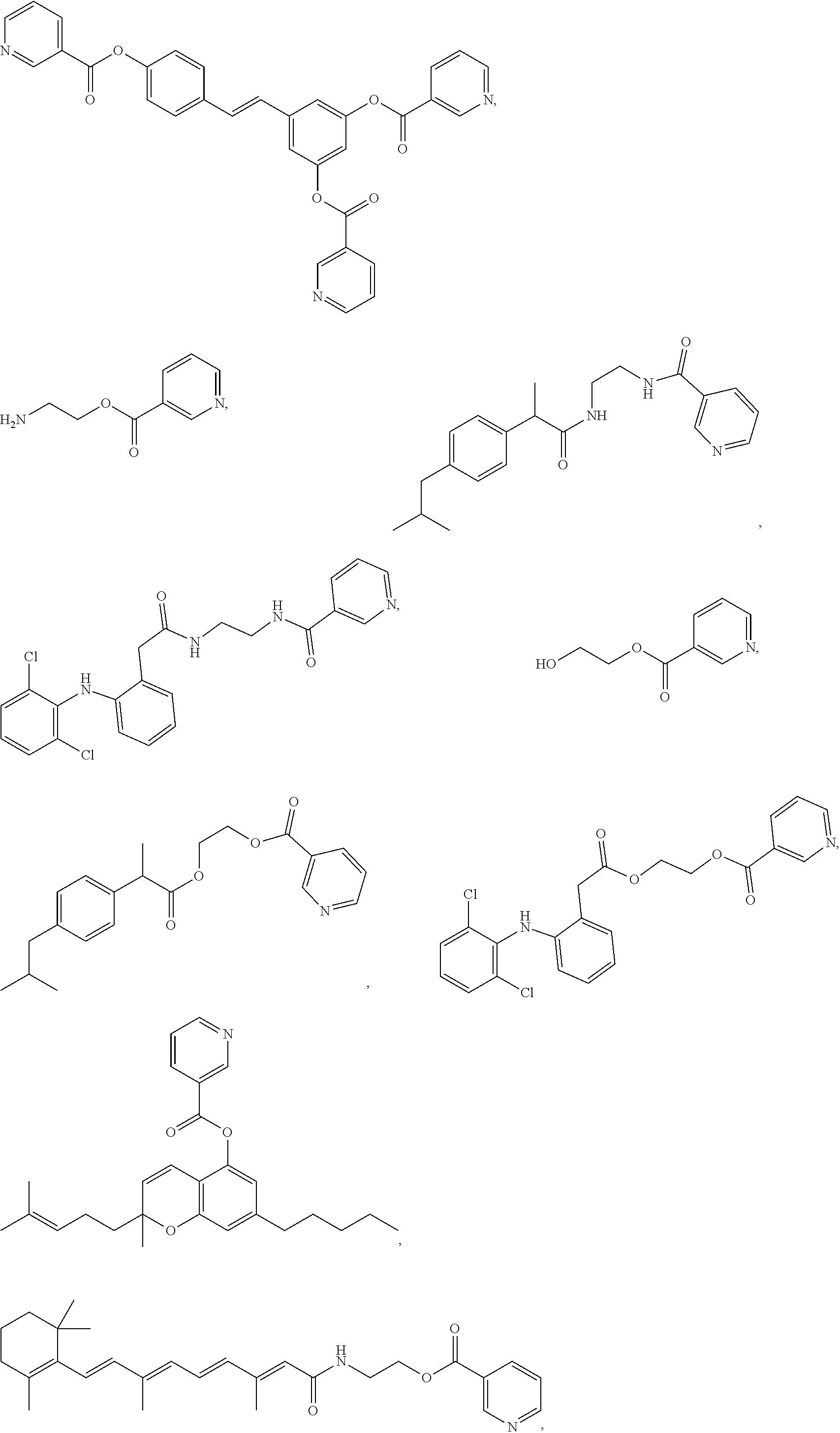

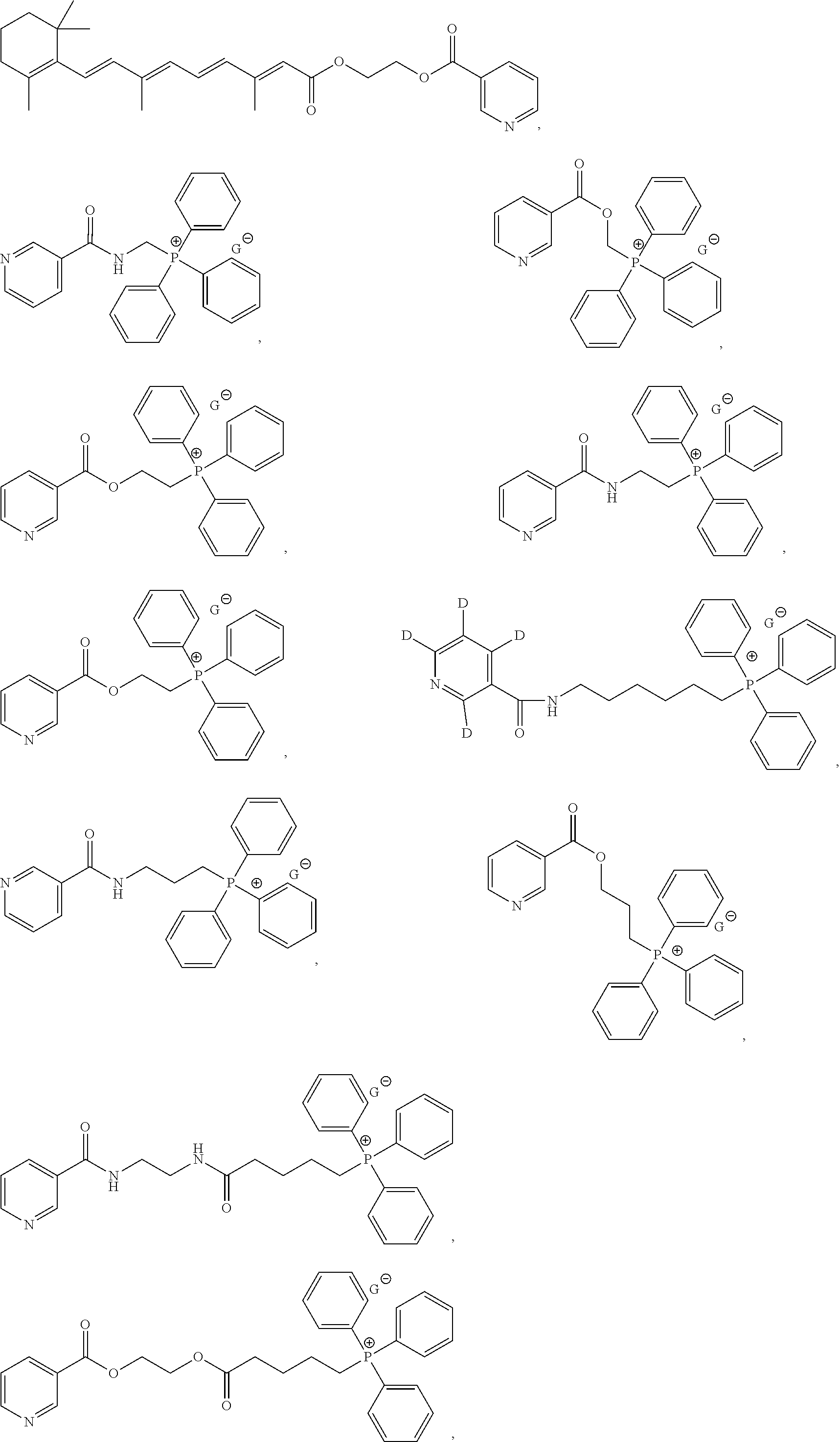

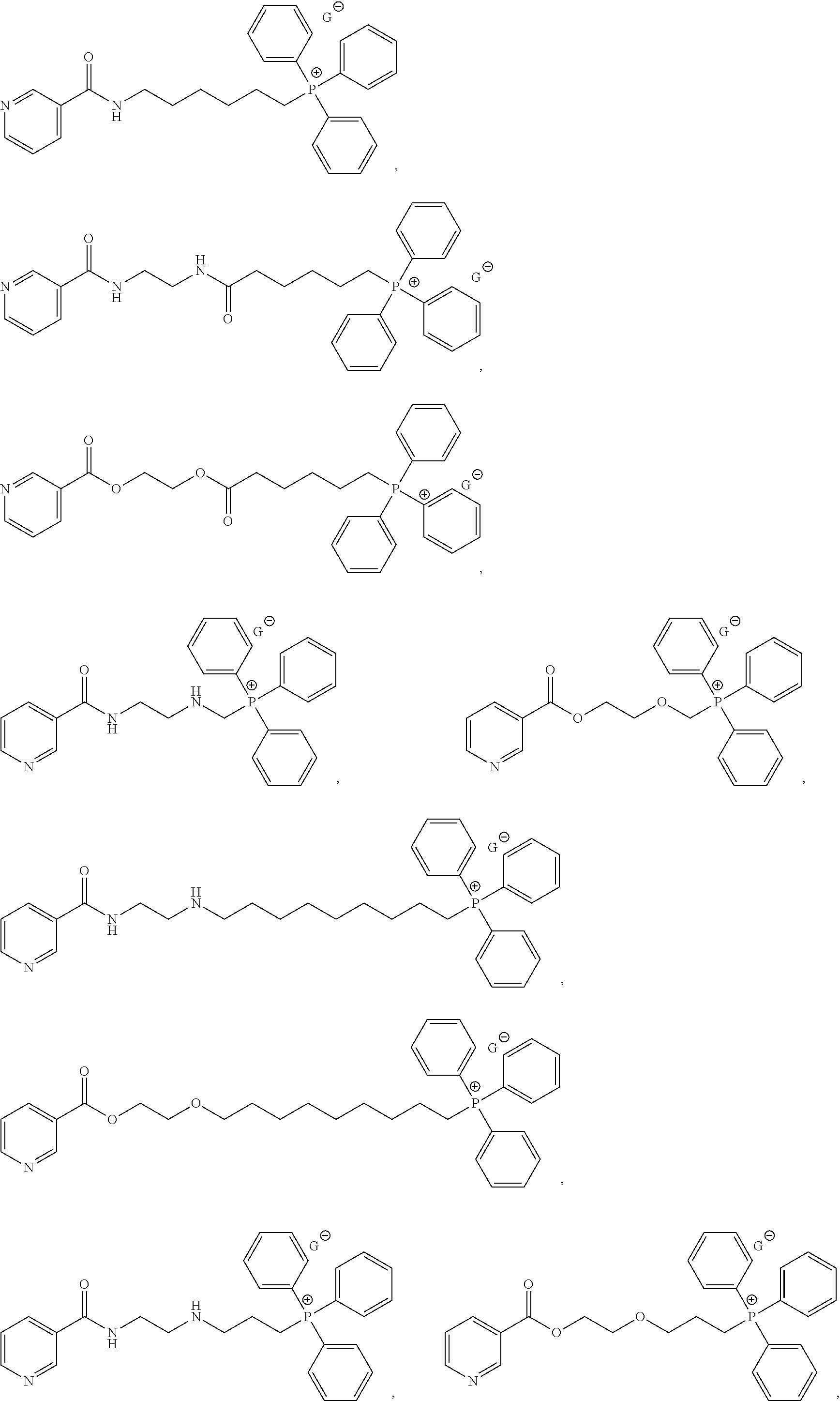

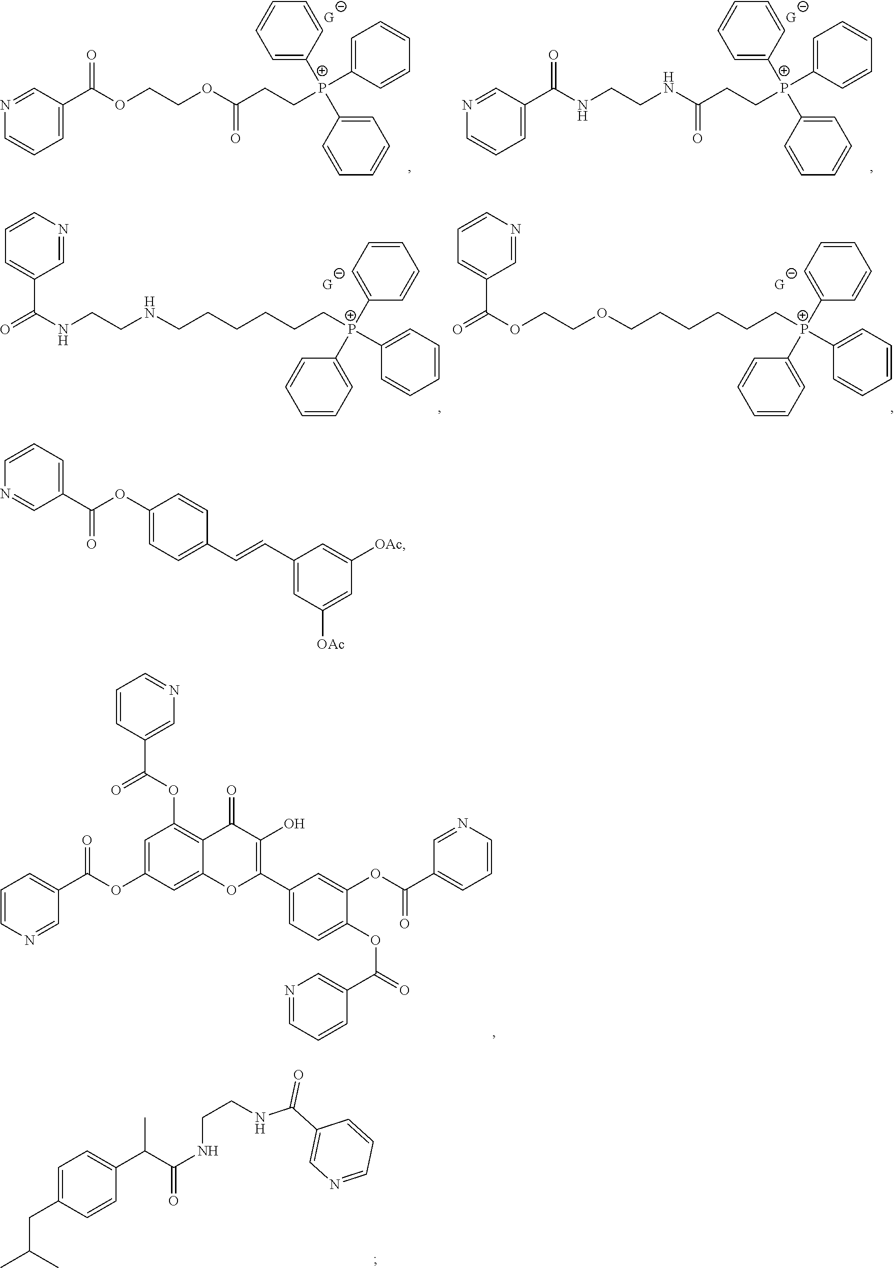

and
wherein G is a pharmaceutically acceptable anion.

A further aspect of the present invention relates to the nicotinate/nicotinamide riboside compound or derivative of formula (V), or a salt, hydrate, or solvate thereof:

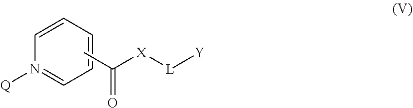

wherein:
Q is absent,

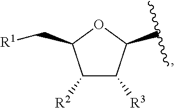

### or —H;

R¹ is HPO₄, H₂PO₄, —OH, or —OC(O)R⁴;
   R² and R³ are independently —OH, —C(O)R⁴, —C(O)OR⁴, —C(O)NHR⁴ or halogen;
R⁴ is —H, C₁₋₂₀ alkyl, C₃₋₁₀cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
   L is a bond, C₁₋₂₀ alkyl, aryl, heteroaryl, arylalkylaryl, arylalkyl, alkoxy, —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
   Y is —C(O)NH₂, —C(O)OH, —R⁵, —P(R⁷)₃, —NH₂, —NHR⁵,

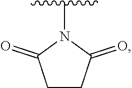 [SMILES: *N1C(=O)CCC1=O]

### —SH, or —OH;

R⁵ is —C(O)R⁴,

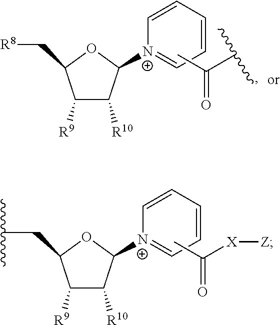

R⁷ is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —OH, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O— heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z is H, or C₁₋₂₀ alkyl; or Z and R¹ are optionally taken together as a bond, forming a macrocycle, with the proviso that if X is O, NH, or NR⁷, and L is C₁₋₂₀ alkyl, aryl, heteroaryl, or alkoxy, then Y is not —C(O)NH₂, —C(O)OH, —R⁵, —NH₂, —NHR⁵, —SH, or —OH.

In some embodiments, the compound or derivative of formula (V) is a compound of formula (Va), or a salt, hydrate, or solvate thereof,

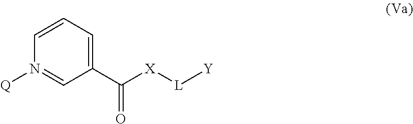

wherein Q, X, L and Y are as defined in the compound of formula (V).

In some embodiments of the compound of formula (V),

### Q is

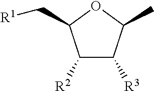

R¹ is H₂PO₄;
R² is —OH;
R³ is —OH;

### X is NH; and

Y is —P(R⁷)₃.

Compounds of this embodiment include without limitation

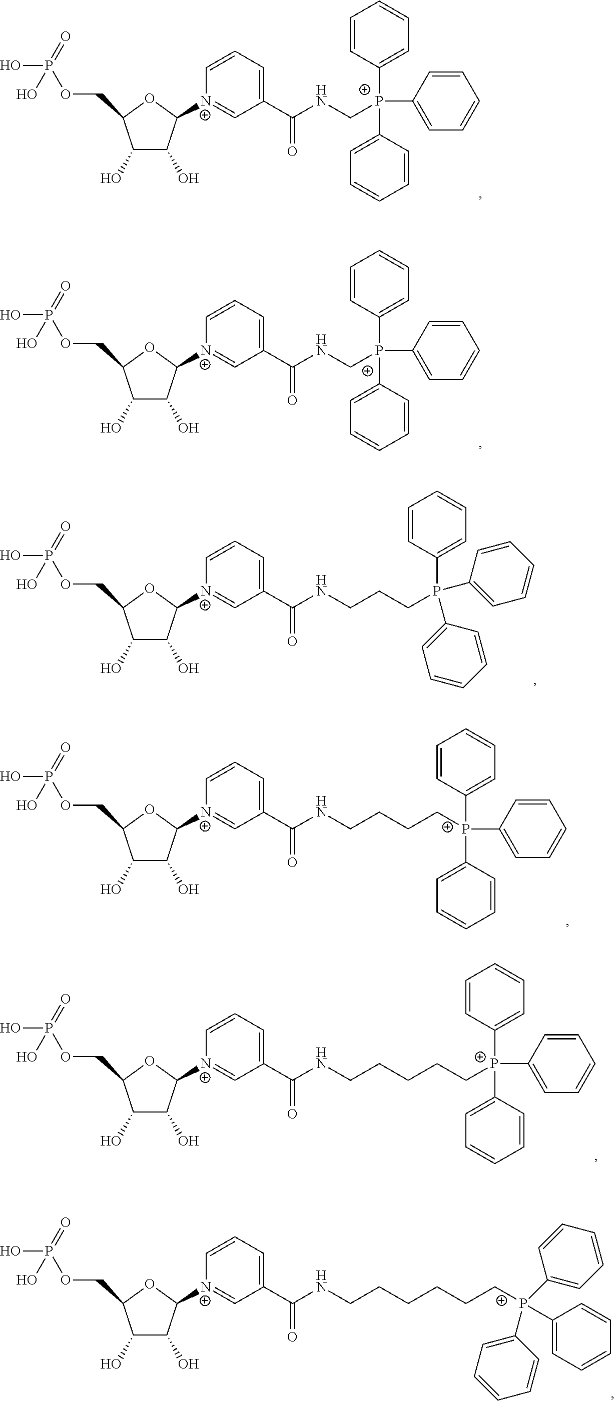

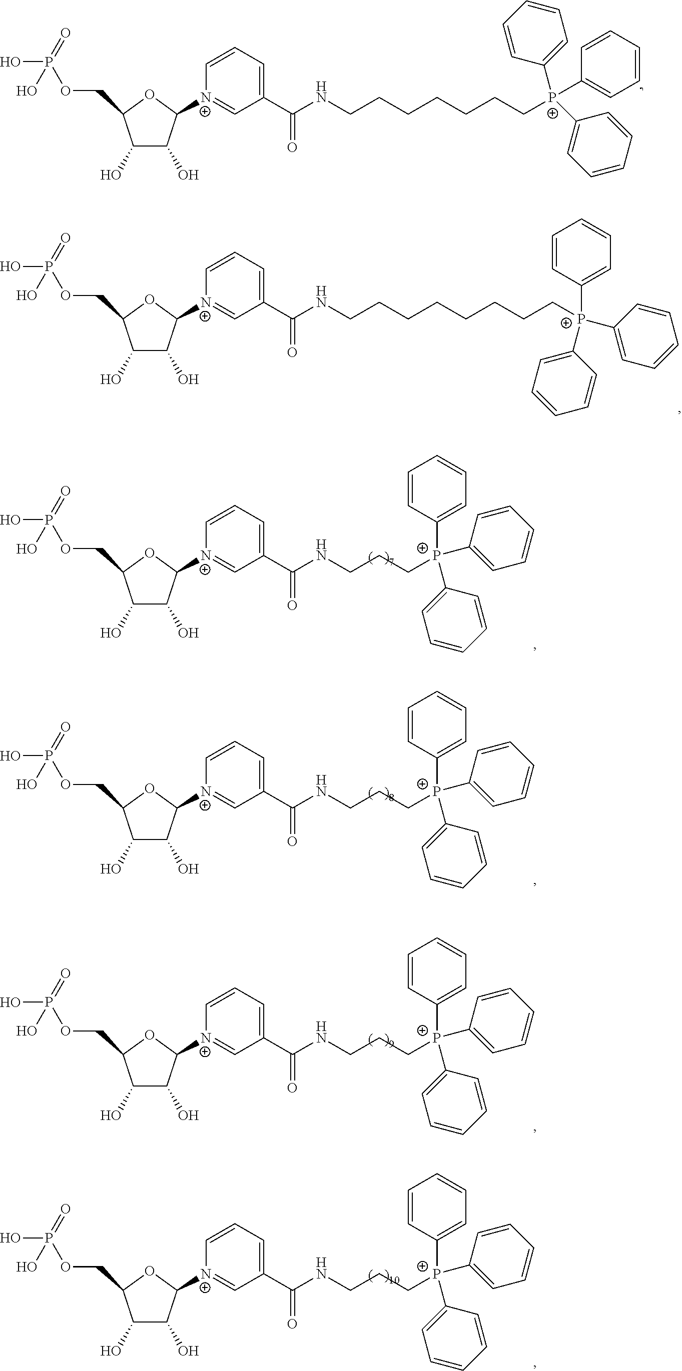

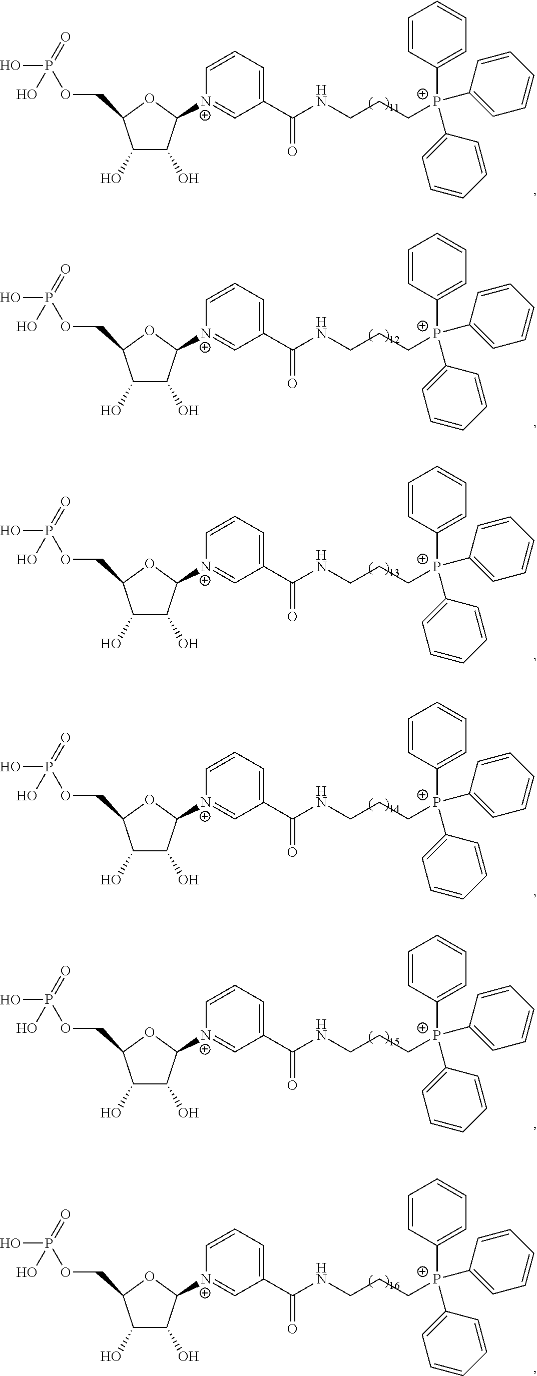

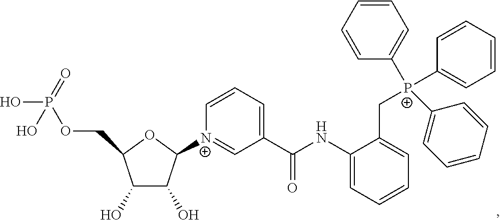 [SMILES: O=C(Nc1ccccc1C[P+](c1ccccc1)(c1ccccc1)c1ccccc1)c1ccc[n+]([C@@H]2O[C@H](COP(=O)(O)O)[C@@H](O)[C@H]2O)c1]

and combinations thereof.

In some embodiments of the compound of formula (V),

Q is absent
R² is —OH;
R³ is —OH;

### X is NH; and

Y is —P(R⁷)₃.

Compounds of this embodiment include, without limitation,

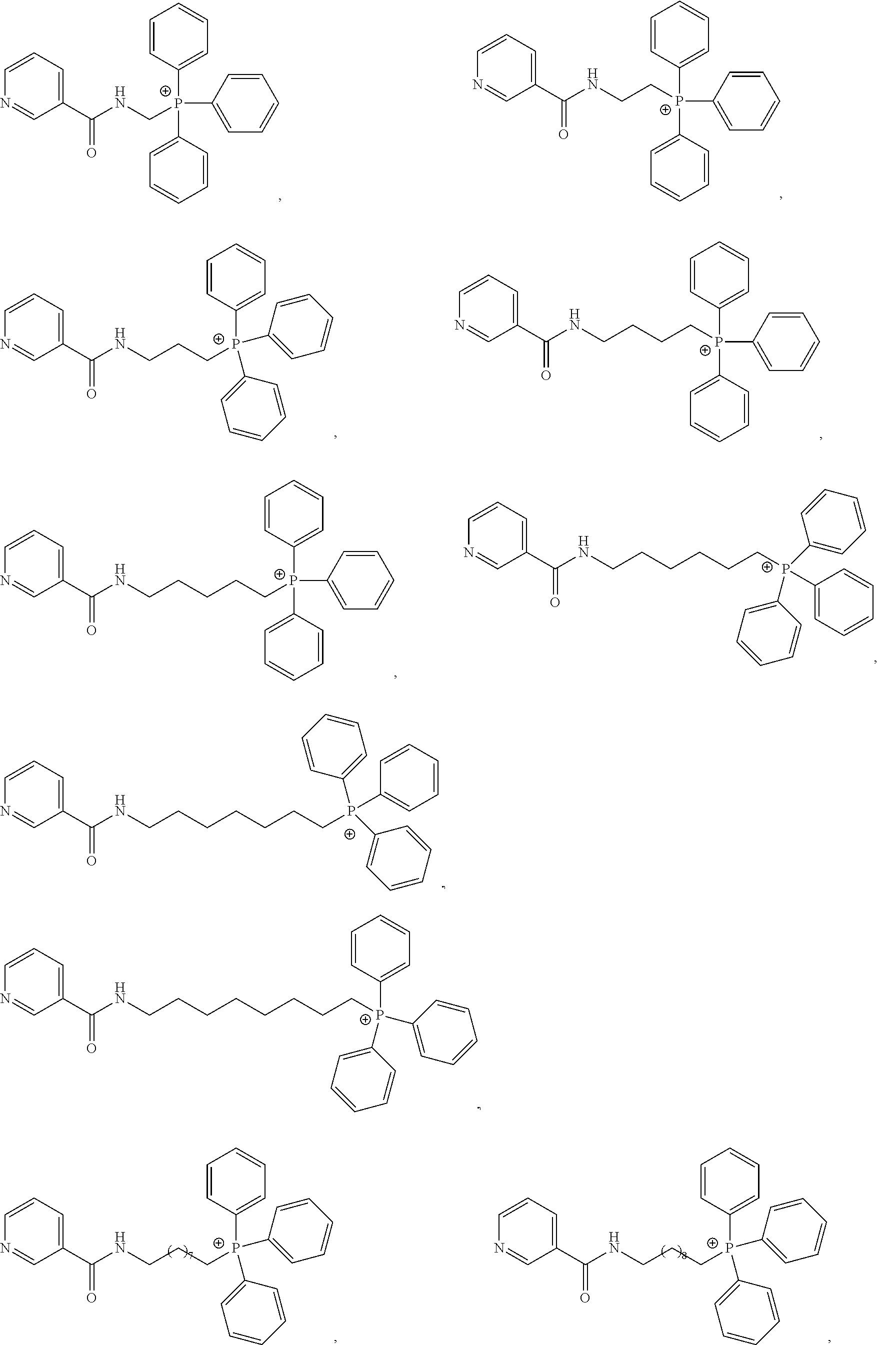

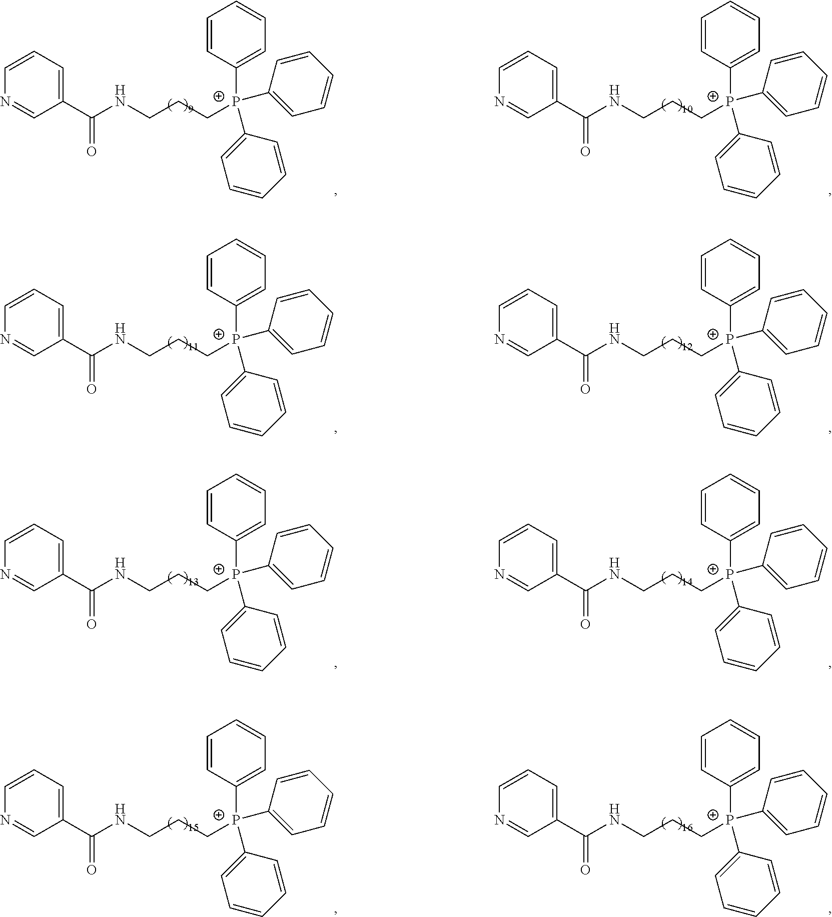

and combinations thereof.

A further aspect of the present invention relates to the nicotinate/nicotinamide riboside compound or derivative of formula (IV), or a salt, hydrate, or solvate thereof:

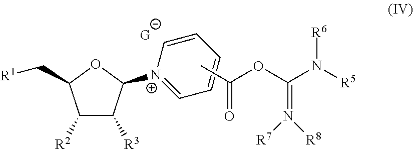

wherein:

R¹ is HPO₄, H₂PO₄, —OH, or —OC(O)R⁴;

R² and R³ are independently —OH, —C(O)R⁴, —C(O)OR⁴, —C(O)NHR⁴ or a halogen;

R⁴ is —H, C₁₋₂₀ alkyl, C₃₋₁₀ cycloalkyl, heterocyclyl, aryl, or heteroaryl, wherein the C₁₋₂₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;

R⁵, R⁶, R⁷, R⁸ are independently a lone pair, H, a C₁₋₁₀ alkyl, C₃₋₁₀ cycloalkyl, heterocyclyl, aryl, or heteroaryl, wherein the C₁₋₁₀ alkyl and C₃₋₁₀ cycloalkyl are optionally substituted with -alkyl, —O-alkyl, —N(R⁹)₂; and

R⁹ is —H, or a C₁₋₁₀ alkyl.

In some embodiments of the compound of formula (IV),

R¹ is H₂PO₄;

R² is —OH;

R³ is —OH; and

G is a pharmaceutically acceptable anion.

Compounds of this embodiment include, without limitation,

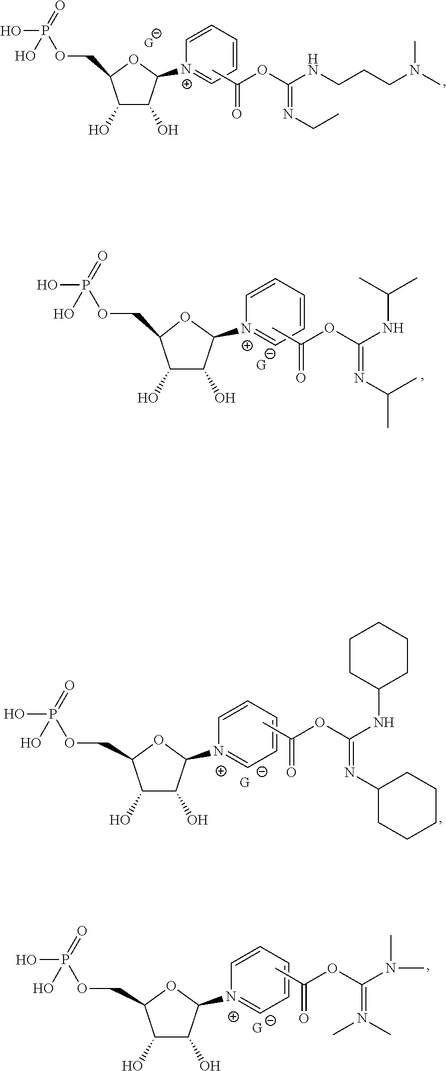

and combinations thereof; wherein G is a pharmaceutically acceptable anion.

In some embodiments, the compound or derivative of formula (IV) is a compound of formula (IVa), or a salt, hydrate, or solvate thereof,

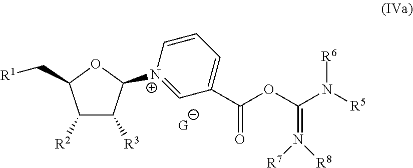

wherein R¹, R², R³, R⁵, R⁶, R⁷ and R⁸ are as defined in the compound of formula (IV).

In some embodiments of the compound of formula (IVa),

R¹ is H₂PO₄; R² is —OH;

R³ is —OH; and

G is a pharmaceutically acceptable anion.

Compounds of this embodiment include, without limitation,

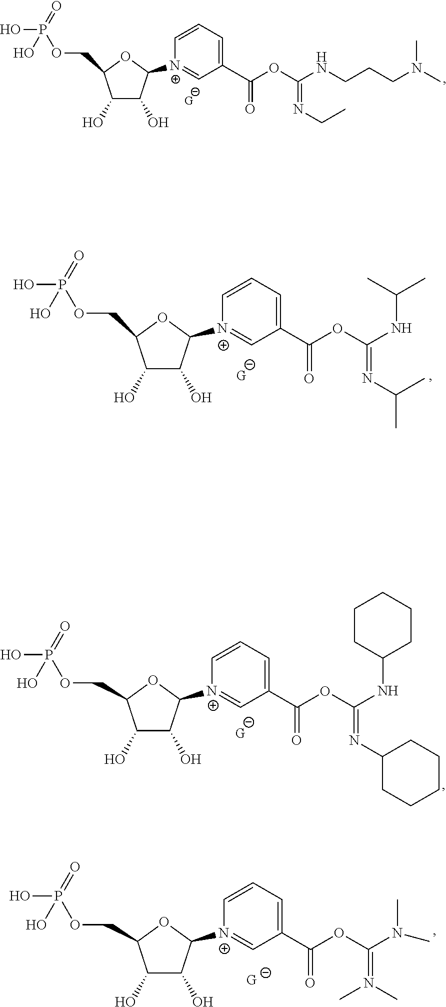

and combinations thereof; wherein G is a pharmaceutically acceptable anion.

A further aspect of the present invention relates to the nicotinate/nicotinamide riboside compound or derivative of formula (IV), or a salt, hydrate, or solvate thereof:

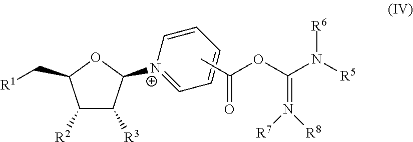

wherein:

R¹ is HPO₄, H₂PO₄, —OH, or —OC(O)R⁴;

R² and R³ are independently —OH, —C(O)R⁴, —C(O)OR⁴, —C(O)NHR⁴ or a halogen;

R⁴ is —H, C₁₋₂₀ alkyl, C₃₋₁₀ cycloalkyl, heterocyclyl, aryl, or heteroaryl, wherein the C₁₋₂₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;

R⁵, R⁶, R⁷, R⁸ are independently a lone pair, H, a C₁₋₁₀ alkyl, C₃₋₁₀ cycloalkyl, heterocyclyl, aryl, or heteroaryl, wherein the C₁₋₁₀ alkyl and C₃₋₁₀ cycloalkyl are optionally substituted with -alkyl, —O-alkyl, —N(R⁹)₂; and

R⁹ is —H, or a C₁₋₁₀ alkyl.

In some embodiments of the compound of formula (IV),

R¹ is H₂PO₄;

R² is —OH; and

R³ is —OH.

Compounds of this embodiment include, without limitation,

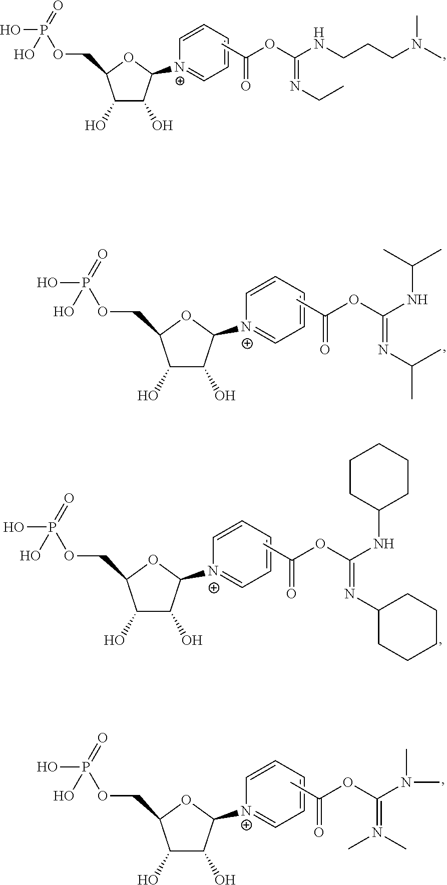

and combinations thereof.

In some embodiments, the compound or derivative of formula (IV) is a compound of formula (IVa), or a salt, hydrate, or solvate thereof,

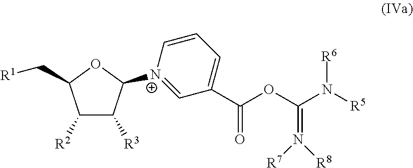

wherein R¹, R², R³, R⁵, R⁶, R⁷ and R⁸ are as defined in the compound of formula (IV).

In some embodiments of the compound of formula (IVa),

R¹ is H₂PO₄; R² is —OH; and

R³ is —OH.

Compounds of this embodiment include, without limitation,

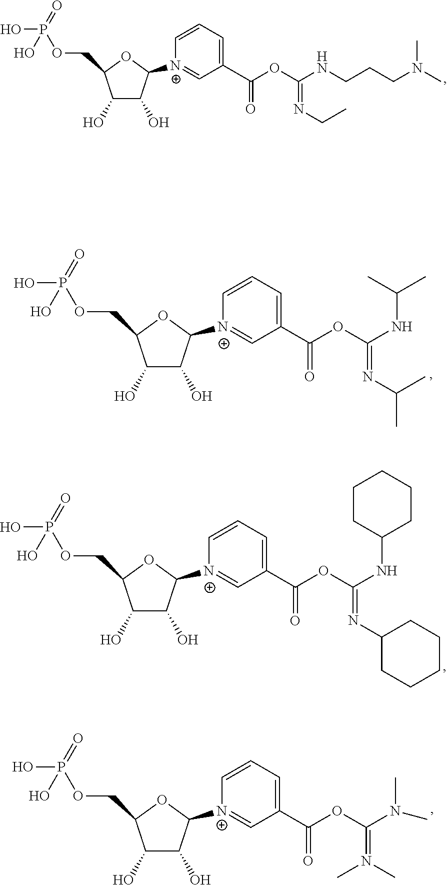

and combinations thereof.

In another aspect, the present disclosure provides pharmaceutical compositions comprising a compound disclosed herein and a pharmaceutically acceptable excipient.

In some embodiments, the compounds or derivatives of formula (V) and/or of formula (Va) are formed into a composition with a carrier. These compositions may be useful for pharmaceutical and/or cosmetic applications. In some embodiments, the carrier is a pharmaceutically acceptable carrier. In some embodiments, the carrier is a cosmetically acceptable carrier.

This disclosure also includes all suitable isotopic variations of a compound of the disclosure. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Such variants may also have advantageous optical properties arising, for example, from changes to vibrational modes due to the heavier isotope. Isotopic variations of a compound of the invention can generally be prepared by conventional procedures known by a person skilled in the art such as by the illustrative methods or by the preparations described in the examples hereafter using appropriate isotopic variations of suitable reagents.

Methods of Treatment using Compounds of Formula (VI)

In one aspect, the present disclosure provides methods of treating a disease or disorder in a subject in need thereof comprising administering a therapeutically amount of a compound of the disclosure or a pharmaceutically acceptable salt thereof to the subject. In certain embodiments, the disease or disorder is selected from the group consisting of Parkinson's disease; Alzheimer's disease; multiple sclerosis; amyotropic lateral sclerosis; muscular dystrophy; AIDS; fulminant hepatitis; Creutzfeld-Jakob disease; retinitis pigmentosa; cerebellar degeneration; myelodysplasis; aplastic anemia; ischemic diseases; myocardial infarction; stroke; hepatic diseases; alcoholic hepatitis; hepatitis B; hepatitis C; osteoarthritis; atherosclerosis; alopecia; damage to the skin due to UV light; lichen planus; atrophy of the skin; cataract; graft rejections; and cell death caused by surgery, drug therapy, chemical exposure or radiation exposure.

In one aspect, the present disclosure provides methods of treating a skin condition in a subject in need thereof comprising administering a therapeutically amount of a compound of the disclosure or a pharmaceutically acceptable salt thereof to the subject. In certain embodiments, the skin condition is selected from the group consisting of contact dermatitis, irritant contact dermatitis, allergic contact dermatitis, atopic dermatitis, actinic keratosis, keratinization disorders, eczema, epidermolysis bullosa diseases, exfoliative dermatitis, seborrheic dermatitis, erythema multiformed, erythema nodosum, damage caused by the sun or other light sources, discoid lupus erythematosus, dermatomyositis, psoriasis, skin cancer and the effects of natural aging

In one aspect, the present disclosure provides methods of treating a disease or disorder in a subject in need thereof comprising administering a therapeutically amount of a compound of the disclosure or a pharmaceutically acceptable salt thereof to the subject.

The compounds and compositions described herein may be used in a method of increasing the level of NAD+ in a cell. This method includes contacting a cell with a compound described herein under conditions effective to increase the level of NAD+ in the cell.

In some embodiments, the cell may be a skill cell. Skin cells may be contacted with a pharmaceutical or cosmetic composition comprising a compound disclosed herein.

Another aspect of the present invention relates to a method of treating a skin affliction or skin condition including administering to a subject in need thereof, a therapeutically effective amount of a composition disclosed herein. The skin affliction or skin condition may be disorders or diseases associated with or caused by inflammation, sun damage or natural aging. For example, the composition may be used to treat contact dermatitis (including irritant contact dermatitis and allergic contact dermatitis), atopic dermatitis (also known as allergic eczema), actinic keratosis, keratosis disorder (including eczema), epidermolysis bullous diseases (including pemphigus), exfoliative dermatitis, seborrheic dermatitis, erythema (including polymorphic and erythema nodosum), injuries caused by the sun or other light sources. The compositions may be utilized in the prevention or treatment of the effects of discoid lupus erythematosus, dermatomyositis, psoriasis, skin cancer and natural aging. In another embodiment, the compositions described herein may be used to treat wounds and/or bums (e.g., to promote healing), including thermal burns, chemical burns, or electrical burns. The formulations may be applied to the skin or mucosal tissue, within the context of an effective dosage regimen to produce the desired result. The compositions may be administered as ointments, lotions, creams, microemulsions, gels, or as a solution.

Another aspect of the present invention relates to a method of increasing intercellular NAD+ in a subject including administering to a subject a compound disclosed herein in an amount effective to increase the intercellular NAD+ in the subject. In some embodiments, the subject is a human subject.

The compositions of the present invention can also be used as a prophylactic, e.g., as chemopreventive composition. When used in chemoprophylaxis, susceptible skin is treated prior to visible pathology in certain individuals. For example, the compounds described herein may be administered to subjects who have recently received or are likely to receive a dose of radiation. The dose of radiation may be initiation is received as part of a work-related or medical procedure, e.g., working in a nuclear power plant, flying an airplane, an X-ray, CAT scan, or the administration of a radioactive dye for medical imaging; wherein the agent may be administered as a prophylactic measure. The radiation exposure may be received unintentionally, e.g., as a result of an industrial accident, terrorist act, or act of war involving radioactive material. In such a case, the agent may be administered as soon as possible after the exposure to inhibit apoptosis and the subsequent development of acute radiation syndrome. The compounds described herein could also be used to protect non-cancerous cells from the effects of chemotherapy, such as to protect neurons in the case of preventing neuropathies, hematoxicity, renal toxicity, and gastrointestinal toxicity due to chemotherapy.

Administration of the compounds described herein may be followed by measuring a factor in the subject, such as measuring level of NAD+, NADH or nicotinamide. A cell may be obtained from a subject following administration of a compound described herein to the subject, such as by obtaining a biopsy, and the factor is determined in the biopsy. Alternatively, biomarkers, such as plasma biomarkers may be followed. The cell may be any cell of the subject, but in cases in which an agent is administered locally, the cell is preferably a cell that is located in the vicinity of the site of administration.

Other factors that may be monitored include a symptom of aging, weight, body mass, blood glucose sugar levels, blood lipid levels and any other factor that may be measured for monitoring diseases or conditions described herein.

Another aspect of the present invention relates to a method of treating a disease or disorder associate with cell death, or to protect cells from cell death, the method comprising administering to a subject in need thereof, the composition described herein. For example, compounds herein may be administered to a subject for the treatment of a chronic disease.

Exemplary diseases include those associated with neuronal death, neuronal dysfunction, or muscular cell death or dysfunction, such as Parkinson's disease; Alzheimer's disease; multiple sclerosis; amyotropic lateral sclerosis; muscular dystrophy; AIDS; fulminant hepatitis; Creutzfeld-Jakob disease; retinitis pigmentosa; cerebellar degeneration; myelodysplasis; aplastic anemia; ischemic diseases; myocardial infarction; stroke; hepatic diseases; alcoholic hepatitis; hepatitis B; hepatitis C; osteoarthritis; atherosclerosis; alopecia; damage to the skin due to UV light; lichen planus; atrophy of the skin; cataract; graft rejections; and cell death caused by surgery, drug therapy, chemical exposure or radiation exposure.

In another embodiment, the compositions described herein are used to reduce the rate of aging in a subject. The compounds described herein may be delivered to a tissue or organ within a subject, such as by injection, to extend the life span of the cells or protect the cells against certain stresses, to prevent or treat diseases of aging, the process of aging itself, diseases or afflictions associate with cell death, infection and toxic agents. For example, an agent can be taken by subjects as food supplements. The compounds described herein may be a component of a multi-vitamin complex. In some embodiments, the skin can be protected from aging (e.g., wrinkle development, loss of elasticity, etc.) by treating the skin or epithelial cells with a compound described herein.

The contents of all figures and all references, Genbank sequences, journal publications, patents, and published patent applications cited throughout this application are expressly incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

The following examples are merely illustrative and should not be construed as limiting the scope of this disclosure in any way as many variations and equivalents will become apparent to those skilled in the art upon reading the present disclosure

### Methods of Making Compounds of Formula (VI)

Provided herein is a method of making nicotinate/nicotinamide riboside-based compounds and derivatives. Ribosylated nicotinamide possesses a relatively labile glycosidic bond, rendering its synthesis and its manipulation challenging (Makarov and Migaud, Beilstein J. Org. Chem. 15:401-430 (2019), which is hereby incorporated by reference in its entirety).

Many existing coupling reactions can cleave this labile glycosidic bond, resulting in nicotinic acid and ribonucleotide. Moreover, coupling protocols known in the art may also lead to intermolecular polymerization through the carboxylic acid group of NaMN with an accessible OH of another NaMN molecule. Previous methods of forming nicotinamide riboside-based compounds commonly relied on the reaction between nicotinamide and a peracylated (halo)-D-ribofuranose resulting in an acylated intermediate which is then converted into nicotinamide riboside. Id.

The method of the present invention is able to form the nicotinate/nicotinamide riboside-based compounds and derivatives starting from NaMN and NaR without cleavage of the glycosidic bond. It was discovered that upon activation of the carboxylic acid on NaMN in the presence of a nucleophile containing ester- and/or amide-forming moieties, small molecule novel NaR and NaMN variants resulted. Surprisingly, off-pathway chemistries such as self-dimerization, polymerization, and degradation did not occur. Unlike the previous methods of forming nicotinate/nicotinamide riboside-based compounds and derivatives, protecting groups (e.g., acetyl groups) are not necessary to prevent polymerization of the NaMN molecule, thus reducing the total number of synthetic steps to obtain the product. The present method proceeds in yields ranging from 30 to 80%. This method is an efficient route for the late-stage diversification of NaMN and NaR produced from total synthesis through fermentation. The method of the present invention is able to form novel NAD precursors.

In one aspect, the present disclosure provides methods of making a compound having a structure represented by Formula (VI) comprising:
providing a nicotinate/nicotinamide riboside compound or derivative of formula (II), or a salt, hydrate, or solvate thereof

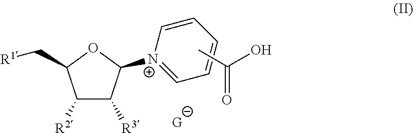

wherein:
R^{1′} is HPO₄, H₂PO₄, —OH, or —OC(O)R^{4′};
R^{2′} and R^{3′} are independently —OH, —C(O)R^{4′}, —C(O)OR^{4′}, —C(O)NHR^{4′} or halogen; and R^{4′} is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
contacting the compound or derivative of formula (II), or a salt, hydrate, or solvate thereof, with a coupling agent and a compound of formula (III)

H—X′-L′-Y′ (III)

wherein:
X′ is O, NH, NR^{7′} or S;
L′ is a bond, C₁₋₂₀ alkyl, aryl, heteroaryl, arylalkylaryl, arylalkyl, alkoxy, —R^{11′}—S—S—R^{11′}—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
R^{4″} is a C₁₋₂₀ alkyl optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a′}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
Y′ is C₁₋₂₀ alkyl; perfluoroalkyl, —C(O)NH₂, —C(O)OH, —R^{5′}, —C(R^{6′})₃, —P(R^{7′})₃, —NH₂, —NHR^{5′},

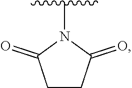 [SMILES: *N1C(=O)CCC1=O]

### —SH, —OH;

R^{5′} is —C(O)R^{4″},

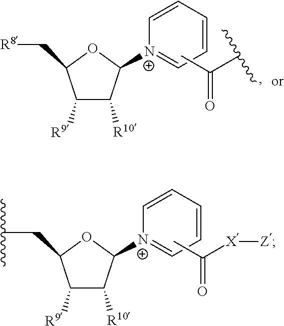

R^{6′} is individually selected at each occurrence from the group consisting of C₁₋₆ alkyl, cycloalkyl, heterocyclyl, heteroaryl, aryl, —H, -halogen, —OH, and —NH₂;
R^{7′} is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R^{8′} is HPO₄, H₂PO₄, —OH or —OC(O)R^{4″};
R^{9′} and R^{10′} are independently —OH, —C(O)R^{4″}, —C(O)OR^{4″}, —C(O)NHR^{4″} or halogen;
R^{11′} is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
Z′ is H, or C₁₋₂₀ alkyl; and
G is an anion

One aspect of the present invention relates to a method of making a nicotinate/nicotinamide riboside compound or derivative of formula (I), or a salt, hydrate, or solvate thereof:

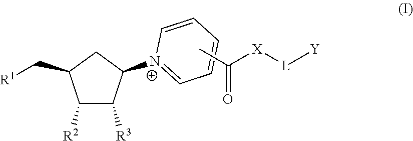

wherein:

R¹ is HPO₄, H₂PO₄, —OH, or —OC(O)R⁴;

R² and R³ are independently —OH, —C(O)R⁴, —C(O)OR⁴, —C(O)NHR⁴ or a halogen;

R⁴ is —H, C₁₋₂₀ alkyl, C₃₋₁₀ cycloalkyl, heterocyclyl, aryl, or heteroaryl, wherein the C₁₋₂₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;

X is O, NH, NR⁷, or S;

L is a bond, C₁₋₂₀ alkyl, aryl, heteroaryl, arylalkylaryl, arylalkyl, alkoxy, —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;

Y is C₁₋₂₀ alkyl, perfluoroalkyl, —C(O)NH₂, —C(O)OH, —R⁵, —C(R⁶)₃, —P(R⁷)₃, —NH₂, —NHR⁵,

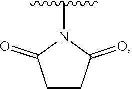 [SMILES: *N1C(=O)CCC1=O]

### —SH, or —OH;

R⁵ is —C(O)R⁴,

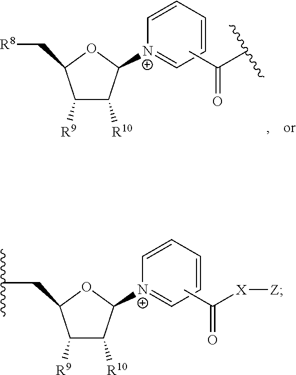

R⁶ is individually selected at each occurrence from the group consisting of C₁₋₆ alkyl, cycloalkyl, heterocyclyl, heteroaryl, aryl, —H, -halogen, —OH, and —NH₂;

R⁷ is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;

R⁸ is HPO₄, H₂PO₄, —OH or —OC(O)R⁴;

R⁹ and R¹⁰ are independently —H, —C(O)R⁴, —C(O)OR⁴, —C(O)NHR⁴ or halogen;

R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and

Z is H, or C₁₋₂₀ alkyl; or Z and R¹ are optionally taken together as a bond, forming a macrocycle;

comprising the steps of:

providing a nicotinate/nicotinamide riboside compound or derivative of formula (II), or a salt, hydrate, or solvate thereof

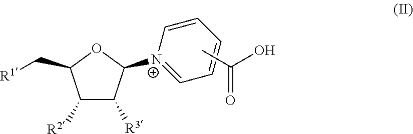

wherein:

R^{1′} is HPO₄, H₂PO₄, —OH, or —OC(O)R^{4′};

R^{2′} and R^{3′} are independently —OH, —C(O)R^{4′}, —C(O)OR^{4′}, —C(O)NHR^{4′} or halogen; and

R^{4′} is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;

contacting the compound or derivative of formula (II), or a salt, hydrate, or solvate thereof, with a coupling agent and a compound of formula (III)

H—X′-L′-Y′ (III)

wherein:

X′ is O, NH, NR^{7′} or S;

L′ is a bond, C₁₋₂₀ alkyl, aryl, heteroaryl, arylalkylaryl, arylalkyl, alkoxy, —R^{11′}—S—S—R^{11′}—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;

R^{4″} is a C₁₋₂₀ alkyl optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a′}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;

Y′ is C₁₋₂₀ alkyl; perfluoroalkyl, —C(O)NH₂, —C(O)OH, —R^{5′}, —C(R^{6′})₃, —P(R^{7′})₃, —NH₂, —NHR^{5′},

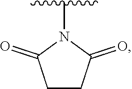 [SMILES: *N1C(=O)CCC1=O]

### —SH, —OH;

R^{5′} is —C(O)R^{4″},

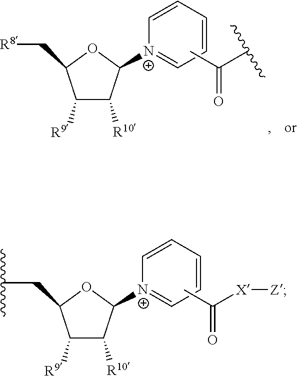

R^{6′} is individually selected at each occurrence from the group consisting of C₁₋₆ alkyl, cycloalkyl, heterocyclyl, heteroaryl, aryl, —H, -halogen, —OH, and —NH₂;

R^{7′} is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;

R^{8′} is HPO₄, H₂PO₄, —OH or —OC(O)R^{4″};

R^{9′} and R^{10′} are independently —OH, —C(O)R^{4″}, —C(O)OR^{4″}, —C(O)NHR^{4″} or halogen;

R^{11′} is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and

Z′ is H, or C₁₋₂₀ alkyl;

under conditions to produce the compound or derivative of formula (I), or salt, hydrate, or solvate thereof; and

isolating the compound or derivative of formula (I), or salt, hydrate, or solvate thereof.

In some embodiments, the method yields at least about 30% of the compound or derivative of formula (I), or salt, hydrate, or solvate thereof. In some embodiments, the method yields at least about 40%, about 50%, about 60%, about 70%, about 80% or more. In some embodiments, the compound or derivative of formula (I), or salt, hydrate, or solvate thereof is formed in a yield ranging from about 30% to about 60%, from about 40% to about 60%, from about 50% to about 60%, from about 30% to about 70%, from about 40% to about 70%, from about 50% to about 70%, from about 60% to about 70%, from about 30% to about 80%, from about 40% to about 80%, from about 50% to about 80%, from about 60% to about 80%, from about 70% to about 80%, or from about 75% to about 80%. In some embodiments, the compound or derivative of formula (I), or salt, hydrate, or solvate thereof is formed in a yield ranging from about 30% to about 80%. In some embodiments the method is optimized and the compound or derivative of formula (I), or salt, hydrate, or solvate thereof is formed in a yield of at least about 80%, 81%, 82%, 83%, 84%, 85%, 86%, 87%, 88%, 89%, 90%, 91%, 92%, 93%, 94%, 95%, 96%, 97%, 98%, 99%, or 99.5% yield.

In some embodiments of the method of the present invention, the nicotinate/nicotinamide riboside compound or derivative of formula (I) is a compound of formula (Ia) or a salt, hydrate, or solvate thereof:

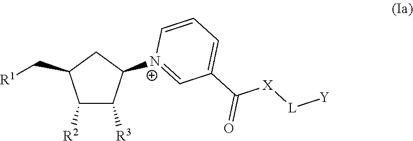

wherein:

R¹, R², R³ X, L, and Y are as defined in formula (I).

In some embodiments of the compound of formula (Ia)

R¹ is H₂PO₄;

R² and R³ are —OH;

R⁴ is a C₁₋₂₀ alkyl;

L is a bond, C₁₋₂₀ alkyl, arylalkylaryl, —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;

Y is C₁₋₂₀ alkyl, —C(O)NH₂, —C(O)OH, —R⁵, —C(R⁶)₃, —P(R⁷)₃, —NH₂, —NHR⁵,

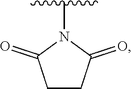 [SMILES: *N1C(=O)CCC1=O]

### —SH, or —OH;

R⁵ is —C(O)R⁴ or

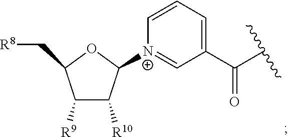

R⁶ is individually selected at each occurrence from the group consisting of C₁₋₆ alkyl, cycloalkyl, heterocyclyl, heteroaryl, aryl, —H, -halogen, —OH, and —NH₂;

R⁷ is aryl;

R⁸ is H₂PO₄;

R⁹ and R¹⁰ are —OH; and

R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl.

Exemplary compounds of this embodiment include, but are not limited to,

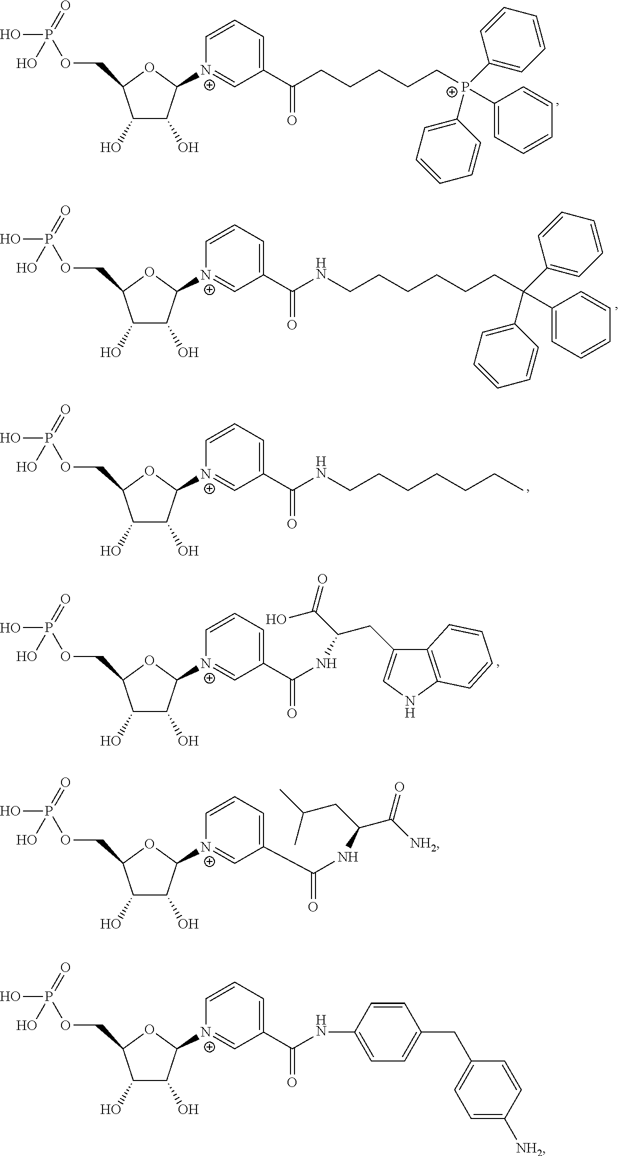

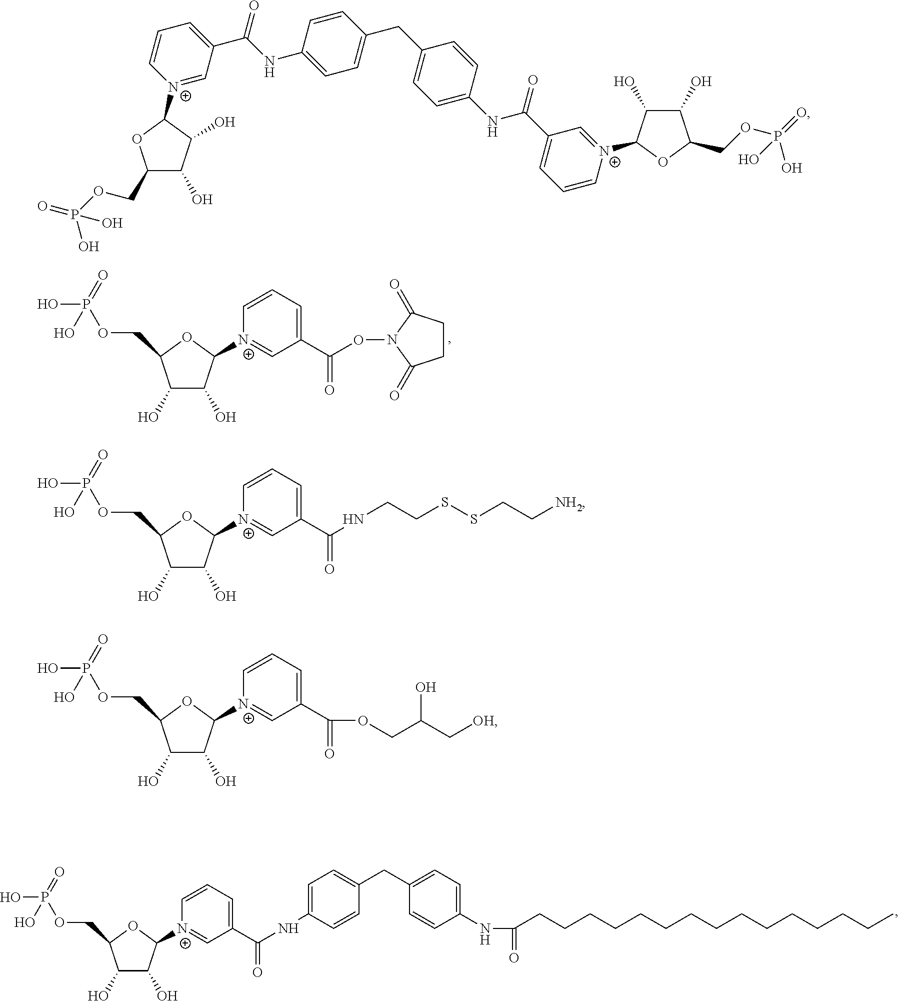 [SMILES: O=C(Nc1ccc(Cc2ccc(NC(=O)c3ccc[n+]([C@@H]4O[C@H](COP(=O)(O)O)[C@@H](O)[C@H]4O)c3)cc2)cc1)c1ccc[n+]([C@@H]2O[C@H](COP(=O)(O)O)[C@@H](O)[C@H]2O)c1 ; O=C(ON1C(=O)CCC1=O)c1ccc[n+]([C@@H]2O[C@H](COP(=O)(O)O)[C@@H](O)[C@H]2O)c1 ; NCCSSCCNC(=O)c1ccc[n+]([C@@H]2O[C@H](COP(=O)(O)O)[C@@H](O)[C@H]2O)c1 ; O=C(OCC(O)CO)c1ccc[n+]([C@@H]2O[C@H](COP(=O)(O)O)[C@@H](O)[C@H]2O)c1 ; CCCCCCCCCCCCCCCC(=O)Nc1ccc(Cc2ccc(NC(=O)c3ccc[n+]([C@@H]4O[C@H](COP(=O)(O)O)[C@@H](O)[C@H]4O)c3)cc2)cc1]

and combinations thereof.

In some embodiments of the compound of formula (Ia)

L is a bond, or C₁₋₂₀ alkyl;

X is NH;

Y is C₁₋₂₀ alkyl —C(R⁶)₃, —P(R⁷)₃;

R⁶ is aryl; and

R⁷ is aryl.

Exemplary compounds of this embodiment include, but are not limited to,

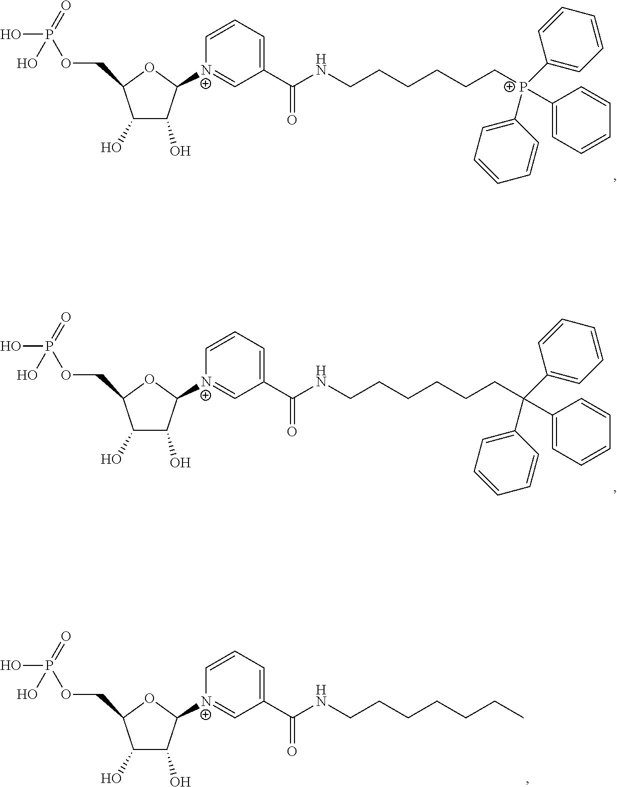 [SMILES: O=C(NCCCCCC[P+](c1ccccc1)(c1ccccc1)c1ccccc1)c1ccc[n+]([C@@H]2O[C@H](COP(=O)(O)O)[C@@H](O)[C@H]2O)c1 ; O=C(NCCCCCCC(c1ccccc1)(c1ccccc1)c1ccccc1)c1ccc[n+]([C@@H]2O[C@H](COP(=O)(O)O)[C@@H](O)[C@H]2O)c1 ; CCCCCCCNC(=O)c1ccc[n+]([C@@H]2O[C@H](COP(=O)(O)O)[C@@H](O)[C@H]2O)c1]

and combinations thereof.

In some embodiments of the compound of formula (Ia)

R² and R³ are —OH;

X is O, or NH;

L is a bond, C₁₋₂₀ alkyl, or arylalkylaryl, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;

Y is C₁₋₂₀ alkyl; perfluoroalkyl, —P(R⁷)₃, —NH₂, or —NHR⁵;

R⁵ is

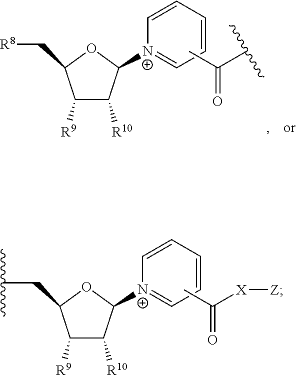

R⁷ is aryl;

R⁸ is —OH;

R⁹ is —OH;

R¹⁰ is —OH; and

Z is —H, or C₁₋₂₀ alkyl; or Z and R¹ are optionally taken together as a bond, forming a macrocycle.

Exemplary compounds of this embodiment include, but are not limited to,

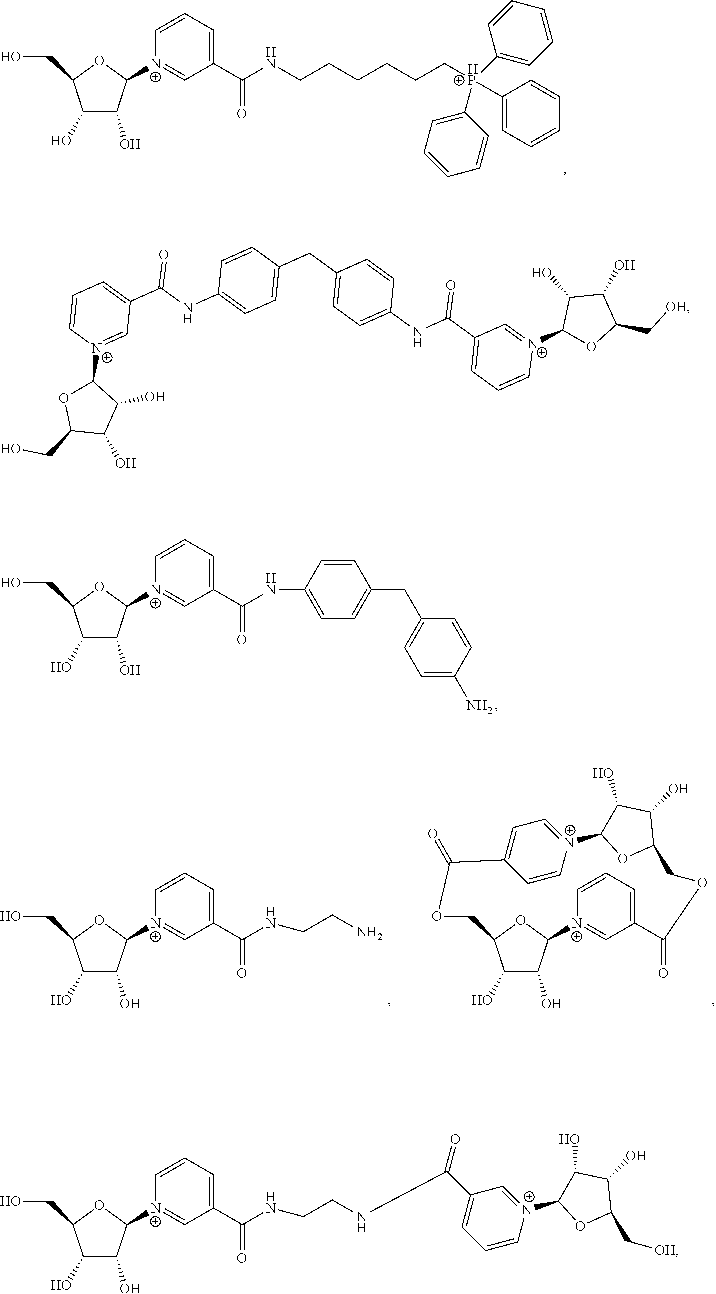

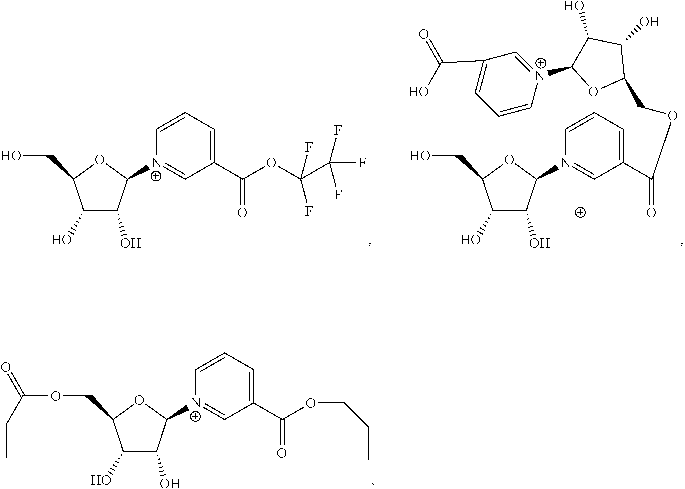

and combinations thereof.

In some embodiments of the method of the present invention, the conditions to produce the compound or derivative of formula (I) comprise reacting the compound of formula (III) with the compound of formula (II) in the presence of base and a coupling agent.

In some embodiments of the method of the present invention, the coupling agent is selected from the group consisting of 1-ethyl-3-(3-dimethylaminopropyl)carbodiimide (EDC); dicyclohexylcarbodiimide (DCC); diisopropylcarbodiimide (DIC); (Benzotriazol-1-yloxy)tris(dimethylamino)phosphonium hexafluorophosphate (BOP); (Benzotriazol-1-yloxy)tripyrrolidinophosphonium hexafluorophosphate (PyBOP); (7-Azabenzotriazol-1-yloxy)tripyrrolidinophosphonium hexafluorophosphate (PyAOP); Bromotripyrrolidinophosphonium hexafluorophosphate (PyBrOP); O-(Benzotriazol-1-yl)-N,N,N′,N′-tetramethyluronium tetrafluoroborate (TBTU); O-(6-Chlorobenzotriazol-1-yl)-N,N,N′,N′-tetramethyluronium tetrafluoroborate (TCTU); O—(N-succinimidyl)-1,1,3,3-tetramethyl-uronium tetrafluoroborate (TSTU); O-(5-Norbornene-2,3-dicarboximido)-N,N,N′,N′-tetramethyluronium tetrafluoroborate (TNTU); (2-(1H-benzotriazol-1-yl)-1,1,3,3-tetramethyluronium hexafluorophosphate) (HBTU); O-(7-Azabenzotriazol-1-yl)-N,N,N′,N′-tetramethyl uronium hexafluorophosphate (HATU); O-(6-Chlorobenzotriazol-1-yl)-N,N,N′,N′-tetramethyluronium hexafluorophosphate (HCTU); 3-(Diethylphosphoryloxy)-1,2,3-benzotriazin-4(3H)-one (DEPBT); 1,1′-carbonyldiimidazole (CDI), and combinations thereof. In some embodiments, the coupling agent is a carbodiimide based coupling agent (e.g., 1-ethyl-3-(3-dimethylaminopropyl)carbodiimide (EDC)).

In some embodiments, the coupling agent is present in a molar equivalent to the compound of formula (II) and/or the compound of formula (III). In some embodiments, the coupling agent is present in a molar excess to the compound of formula (II) and/or the compound of formula (III). In some embodiments, the coupling agent is present in about 1, about 1.025, about 1.05, about 1.1, about 1.2, about 1.3, about 1.4, about 1.5, about 1.6, about 1.7, about 1.8, about 1.9, about 2, about 2.5, or about 3 molar equivalents of the compound of formula (II) and/or the compound of formula (III). The coupling agent may be present in about 1, about 1.025, about 1.05, about 1.1, about 1.2, about 1.3, about 1.4, about 1.5, about 1.6, about 1.7, about 1.8, about 1.9, about 2, or about 2.5 molar equivalents up to about 1.025, about 1.05, about 1.1, about 1.2, about 1.3, about 1.4, about 1.5, about 1.6, about 1.7, about 1.8, about 1.9, about 2, about 2.5, or about 3 molar equivalents of the compound of formula (II) and/or the compound of formula (III). Alternatively, the coupling agent may be present in a molar amount less than the compound of formula (II) and/or the compound of formula (III). In some embodiments, the coupling agent is present in about 0.025, about 0.05, about 0.1, about 0.2, about 0.3, about 0.4, about 0.5, about 0.6, about 0.7, about 0.8, or about 0.9 molar equivalents of the compound of formula (II) and/or the compound of formula (III). The coupling agent may be present in about 0.025, about 0.05, about 0.1, about 0.2, about 0.3, about 0.4, about 0.5, about 0.6, about 0.7, or about 0.8 molar equivalents up to about 0.05, about 0.1, about 0.2, about 0.3, about 0.4, about 0.5, about 0.6, about 0.7, about 0.8, or about 0.9 molar equivalents of the compound of formula (II) and/or the compound of formula (III).

In some embodiments of the method of the present invention, the base is an amine base. The amine base may be a sterically hindered base that is unable to partake in addition and/or substitution reactions. In some embodiments, the base is selected from the group consisting of triethylamine; diisopropylethylamine; tributylamine; N-methylmorpholine; pyridine; 2,6-lutidine; N-methylimidazole, and combinations thereof. In some embodiments, the base is diisopropylethylamine.

In some embodiments of the method of the present invention, the base may be added in excess to the other reagents in the reaction mixture (i.e., the compound of formula (II), the coupling agent, and/or the compound of formula (III)). In some embodiments, the base is present in about 1.1 molar equivalents or greater of the coupling agent, the compound of formula (II) and/or the compound of formula (III). The base may be present in about 1.05, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 7, about 8, about 9, or about 10 molar equivalents of the coupling agent, the compound of formula (II) and/or the compound of formula (III). In some embodiments, the base is present in about 1.05, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 7, about 8, or about 9 molar equivalents up to about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 7, about 8, about 9, or about 10 molar equivalents of the coupling agent, the compound of formula (II) and/or the compound of formula (III).

In some embodiments, compound of formula (II) may be the limiting reagent (i.e., present in lower molar equivalence to the base, the coupling reagent, the and/or the compound of formula (III)). In some embodiments, the compound of formula (II) is present in about 0.025, about 0.05, about 0.1, about 0.2, about 0.3, about 0.4, about 0.5, about 0.6, about 0.7, about 0.8, or about 0.9 molar equivalents of the compound of formula (III) and/or the coupling reagent. The compound of formula (II) may be present in about 0.025, about 0.05, about 0.1, about 0.2, about 0.3, about 0.4, about 0.5, about 0.6, about 0.7, or about 0.8 molar equivalents up to about 0.05, about 0.1, about 0.2, about 0.3, about 0.4, about 0.5, about 0.6, about 0.7, about 0.8, or about 0.9 molar equivalents of the compound of formula (III) and/or the coupling reagent. In some embodiments, the compound of formula (II) is present in a molar equivalent to the compound of formula (III) and/or the coupling reagent. Alternatively, the compound of formula (II) may be present in a molar excess to the coupling agent and/or the compound of formula (III). In some embodiments, the compound of formula (II) is present in about 1, about 1.025, about 1.05, about 1.1, about 1.2, about 1.3, about 1.4, about 1.5, about 1.6, about 1.7, about 1.8, about 1.9, about 2, about 2.5, or about 3 molar equivalents of the compound of formula (III) and/or the coupling reagent. The compound of formula (II) may be present in about 1, about 1.025, about 1.05, about 1.1, about 1.2, about 1.3, about 1.4, about 1.5, about 1.6, about 1.7, about 1.8, about 1.9, about 2, or about 2.5 molar equivalents up to about 1.025, about 1.05, about 1.1, about 1.2, about 1.3, about 1.4, about 1.5, about 1.6, about 1.7, about 1.8, about 1.9, about 2, about 2.5, or about 3 molar equivalents of the compound of formula (III) and/or the coupling reagent.

In some embodiments, the nicotinate/nicotinamide riboside compound or derivative of formula (II) is a compound of formula (IIa), or a salt, hydrate, or solvate thereof;

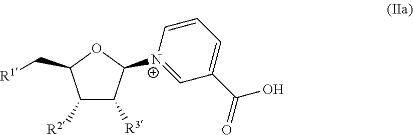

wherein:

R^{1′} is HPO₄, H₂PO₄, —OH, or —OC(O)R^{4′};

R^{2′} and R^{3′} are independently —OH, —C(O)R^{4′}, —C(O)OR^{4′}, —C(O)NHR^{4′} or halogen; and

R^{4′} is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl.

In some embodiments of the compound of formula (IIa),

R^{1′} is H₂PO₄ or —OC(O)R^{4′};

R^{2′} and R^{3′} are independently —OH, —C(O)R^{4′}, —C(O)OR^{4′}; and

R^{4′} is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl.

In some embodiments of the present invention, the reacting comprises: (i) dissolving the compound of formula (II) in a solvent, or solvent mixture, to form a first solution; (ii) adding the base and coupling agent to the first solution to form a basic solution; (iii) adding the compound of formula (III) to the basic solution; and (iv) isolating the compound or derivative of formula (I), or salt, hydrate, or solvate thereof.

The base and/or coupling agent may be dissolved in a solvent forming a second solution prior to the addition of the base and/or coupling agent to the first solution. Alternatively, the base and/or coupling agent may be added neat to the first solution. The compound of formula (III) may be dissolved in a solvent forming a third solution prior to the addition of the compound of formula (III) to the basic solution. The compound of formula (II), the base, the coupling agent, and/or the compound of formula (III) may be dissolved in the same or different solvents or solvent mixtures.

In some embodiments of the method of the present invention, the solvent or solvent mixture is selected from the group consisting of water, dimethylformamide (DMF), chloroform, dichloromethane, dichloroethane, acetonitrile, dimethyl sulfoxide (DMSO), benzene, toluene, xylenes, chlorobenzene, tetrahydrofuran, methanol, ethanol, isopropanol, 1-butanol, 2-butanol, t-butyl alcohol, 2-butanone, hexane, hexane isomers, cyclohexane, ethers, diethylene glycol, acetone, ethyl acetate, butanone, 1,4-dioxane, and combinations thereof.

In some embodiments of the method of the present invention, the compound of formula (II), the base and coupling agent, and the compound of formula (III) may be dissolved in their respective solvents to form solutions ranging in concentration from about 0.05 M to about 10 M. For example, the concentration of the solutions may be about 0.05 M, about 0.1 M, about 0.2 M, about 0.3 M, about 0.4 M, about 0.5 M, about 0.6 M about 0.7 M, about 0.8 M, about 0.9 M, about 1.0 M, about 1.5 M, about 2 M, about 2.5 M, about 3 M, about 3.5 M, about 4.0 M, about 4.5 M, about 5.0 M, about 5.5 M, about 6.0 M, about 6.5 M, about 7.0 M, about 7.5 M, about 8 M, about 8.5 M, about 9.0 M, about 9.5 M, or about 10.0 M.

In some embodiments of the method of the present invention, the reacting is carried out in air. In other embodiments, the reacting is carried out under inert conditions (e.g., in a dry nitrogen or argon atmosphere). In some embodiments, the reaction reaches completion in approximately 24 to 48 hours. As will be apparent to those of skill in the art, the time required for the reaction to reach completion will vary based on a variety of factors including the reactivity of the starting materials and the temperature of the reaction. The reaction may reach completion in a period of time ranging from about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, or 49 hours, up to about 2 hours, 3 hours, 4 hours, 5 hours, 6 hours, 7 hours, 8 hours, 9 hours, 10 hours, 11 hours, 12 hours, 13 hours, 14 hours, 15 hours, 16 hours 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, or 50 hours. The reaction progress may be monitored to determine starting material consumption and/or product formation (e.g., using LCMS).

In some embodiments of the method of the present invention, the reacting is performed at a temperature of about 0° C. to about 100° C. For example, the temperature may be about 0° C., about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. up to about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C. In some embodiments, the temperature is ambient room temperature (e.g., about 25° C.).

In some embodiments, the compound or derivative of formula (I), or salt, hydrate, or solvate thereof may be isolated from the basic solution and purified by standard techniques such as filtration, liquid-liquid extraction, solid phase extraction, distillation, recrystallization, or chromatography, including flash column chromatography, preparative TLC, HPTLC, HPLC, or rp-HPLC. In one embodiment, the compound or derivative of formula (I), or salt, hydrate, or solvate thereof is isolated directly from the basic solution using flash chromatography without the need for any additional workup.

### Methods of Making NAMN

In another aspect, the present disclosure provides methods of making Compound 1 (NAMN), wherein the method is performed as depicted in Scheme I:

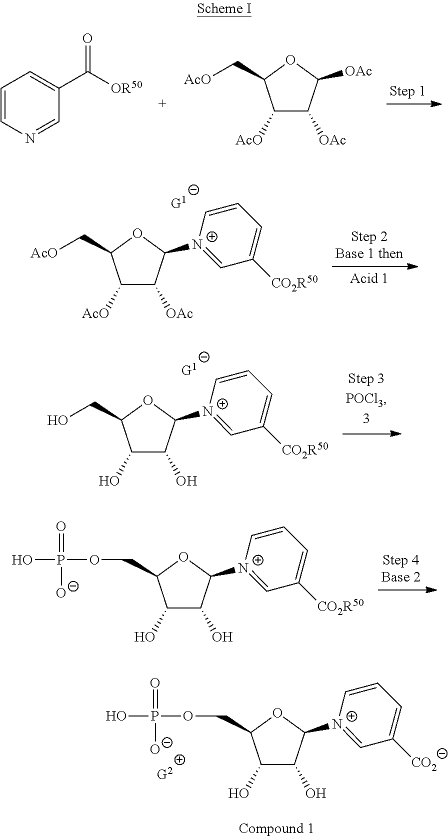

wherein
R⁵⁰ is alkyl;
G¹ is an anion; and
G² is a cation.

In certain embodiments, Step 1 is performed under flow conditions.

In certain embodiments, Step 2 is performed under flow conditions.

In certain embodiments, Step 3 is performed under flow conditions.

In certain embodiments, Step 4 is performed under flow conditions.

In certain embodiments, Base 1 is a hydroxide (e.g., sodium hydroxide).

In certain embodiments, Acid 1 is a mineral acid (e.g., sulfuric acid).

In certain embodiments, Base 2 is a hydroxide (e.g., sodium hydroxide).

In certain embodiments, the method is performed in acetonitrile.

In certain embodiments, the method is performed in a mixture of acetonitrile and ethanol.

In another aspect, the present disclosure provides methods of making Compound 1 (NAMN), wherein the method is performed as depicted in Scheme II:

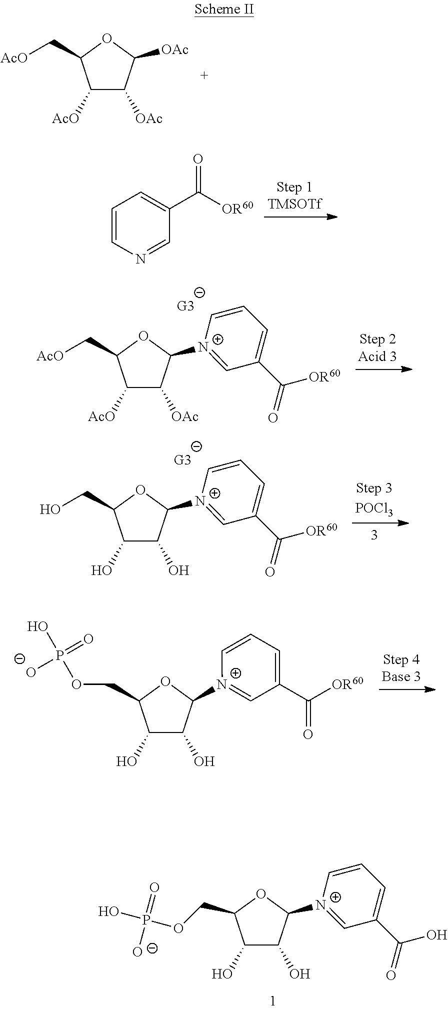

In certain embodiments, Step 1 is performed in a halogenated hydrocarbon solvent (e.g., dichloromethane).

In certain embodiments, Acid 3 is a mineral acid (e.g., aqueous hydrochloric acid). In certain embodiments, the mineral acid is the solvent.

In certain embodiments, Base 3 is a hydroxide base (e.g., aqueous lithium hydroxide).

In certain embodiments, Step 4 is performed in a mixture of an organic solvent and water (e.g., tetrahydrofuran and water).

In certain embodiments, R⁵⁰ is aralkyl (e.g., benzyl).

Pharmaceutical Compositions A pharmaceutically acceptable excipient can be a pharmaceutically acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound that adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of a reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl-or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations/compositions provided herein. Suitable preservatives for use in the compositions of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995)

Pharmaceutical compositions be formulated in a conventional manner using one or more physiologically acceptable carriers or excipients. Thus, the compounds described herein may be formulated for administration by, for example, injection, inhalation or insufflation (either through the mouth or the nose) or oral, buccal, parenteral or rectal administration. The agent may be administered locally, e.g., at the site where the target cells are present, such as by the use of a patch. In some embodiments, the pharmaceutically acceptable carrier is selected from the group consisting of binders, disintegrating agents, lubricants, corrigents, solubilizing agents, suspension aids, emulsifying agents, coating agents, cyclodextrins, and/or buffers.

The compounds described herein may be formulated for a variety of loads of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the agents can be formulated in liquid solutions, for example, in physiologically compatible buffers such as Hank's solution or Ringer's solution. In addition, the agents may be formulated in solid form and redissolved or suspended immediately prior to use. Lyophilized forms are also included.

For oral administration, the compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release of the active compound.

For administration by inhalation, the compounds of the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the agent and a suitable powder base such as lactose or starch.

The compounds of the present invention may be formulated for parenteral administration by injection, e.g., by bolus injection or continuous infusion. Formulations for injection may be presented in unit dosage form, e.g., in ampoules or in multi-dose containers, with an added preservative. The compounds of the present invention may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the formulations described previously, the compounds of the present invention may also be formulated as a depot preparation. Such long acting formulations may be administered by implantation (for example subcutaneously or intramuscularly) or by intramuscular injection. Thus, for example, the compounds of the present invention may be formulated with suitable polymeric or hydrophobic materials (for example as an emulsion in an acceptable oil) or ion exchange resins, or as sparingly soluble derivatives, for example, as a sparingly soluble salt. Controlled release formula also include patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions (including cosmetic preparations) may comprise from about 0.00001 to 100% such as from 0.001 to 10% or from 0.1% to 5% by weight of one or more of the compounds described herein.

In some embodiments, the cosmetically acceptable carrier comprises at least one of the group consisting of an additive, a colorant, an emulsifier, a fragrance, a humectant, a polymerizable monomer, a stabilizer, a solvent, and a surfactant.

The compounds described herein may be incorporated into a topical formulation containing a topical carrier that is generally suited to topical drug administration or cosmetic formulation and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. The selected carrier should not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

The compounds of the present invention may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Water-soluble ointment bases may be prepared from polyethylene glycols (PEGs) of varying molecular weight; again, reference may be had to Remington's, supra, for further information.

The compounds of the present invention may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. A lotion formulation for use in conjunction with the present method may contain propylene glycol mixed with a hydrophilic petrolatum.

The compounds of the present invention may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

The compounds of the present invention may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds of the present invention may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like.

Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkanediols; miscellaneous solvents such as dimethyl fornamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark AzoneR™ from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salioylate).

In certain topical formulations, the compound of the present invention is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules.

### Kits

Also described herein are kits, e.g. kits for therapeutic purposes, including kits for modulating aging and for treating diseases, e.g., those described herein. A kit may comprise one or more compounds described herein, and optionally devices for contacting cells with the compounds of the invention. Devices include syringes, stents and other devices for introducing an agent into a subject or applying it to the skin of a subject.

Further, a kit may also contain components for measuring a factor, e.g., measuring level of NAD+, NADH or nicotinamide, e.g., in tissue samples.

The kits may include kits for diagnosing the likelihood of having or developing an aging related disease, weight gain, obesity, insulin-resistance, diabetes, cancer, precursors thereof or secondary conditions thereof. A kit may comprise an agent for measuring the activity and or expression level of NAD+, NADH, nicotinamide, and/or other intermediary compound in the NAD+ salvage pathway.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

As used herein, the singular forms “a”, “an” and “the” include plural referents unless the context clearly dictates otherwise. The use of “or” or “and” means “and/or” unless stated otherwise.

The term “about” as used herein when referring to a measurable value such as an amount, a temporal duration and the like, includes the value itself as well as encompassing variations of up to ±10% from the specified value. Unless otherwise indicated, all numbers expressing quantities of ingredients, properties such as molecular weight, reaction conditions, etc., used herein are to be understood as being modified by the term “about”.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The term “alkyl” means an aliphatic hydrocarbon group which may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxyalkyl” refers to an alkyl group substituted with an alkoxy group and may be represented by the general formula alkyl-O-alkyl.

The term “alkenyl” means an aliphatic hydrocarbon group containing a carbon-carbon double bond and which may be straight or branched having about 2 to about 6 carbon atoms in the chain. Particular alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “alkynyl” refers to an aliphatic hydrocarbon group containing a carbon-carbon triple bond and which may be straight or branched having about 2 to about 6 carbon atoms in the chain. Particular alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “alkoxy” means groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

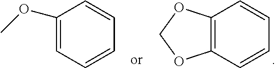

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

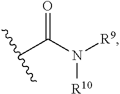

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

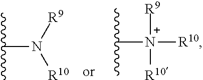

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “cycloalkyl” means a non-aromatic mono- or multicyclic ring system of about 3 to about 8 carbon atoms, preferably of about 5 to about 7 carbon atoms, and which may include at least one double bond. Exemplary cycloalkyl groups include, without limitation, cyclopropyl, cyclobutyl, cyclopentyl, cyclohexyl, cycloheptyl, cyclopropenyl, cyclobutenyl, cyclopentenyl, cyclophenyl, anti-bicyclopropane, and syn-tricyclopropane.

The term “cycloalkylalkyl” refers to a cycloalkyl-alkyl-group in which the cycloalkyl and alkyl are as defined herein. Exemplary cycloalkylalkyl groups include cyclopropylmethyl, cyclobutylmethyl, cyclopentylmethyl, cyclopropylethyl, cyclobutylethyl, and cyclopentylethyl. The alkyl radical and the cycloalkyl radical may be optionally substituted as defined herein.

The term “aryl” means an aromatic monocyclic or multi-cyclic (polycyclic) ring system of 6 to about 19 carbon atoms, or of 6 to about 10 carbon atoms, and includes arylalkyl groups.

The ring system of the aryl group may be optionally substituted. Representative aryl groups include, but are not limited to, groups such as phenyl, naphthyl, azulenyl, phenanthrenyl, anthracenyl, fluorenyl, pyrenyl, triphenylenyl, chrysenyl, and naphthacenyl.

The term “arylalkyl” means an alkyl substituted with one or more aryl groups, wherein the alkyl and aryl groups are as herein described. One particular example is an arylmethyl or arylethyl group, in which a single or a double carbon spacer unit is attached to an aryl group, where the carbon spacer and the aryl group can be optionally substituted as described herein. Representative arylalkyl groups include

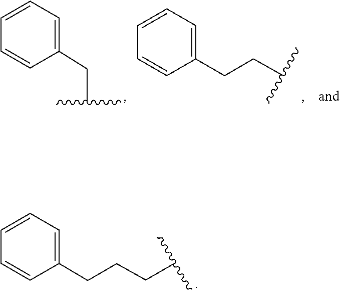 [SMILES: *Cc1ccccc1 ; *CCc1ccccc1 ; *CCCc1ccccc1]

The term “arylalkylaryl” refers to group an aryl group substituted with one or more one or more arylalkyl groups, wherein the aryl and alkyl groups are as herein described. One particular example is an arylmethyl or arylethyl group, in which the methyl or ethyl group is attached to a further aryl group. Representative arylalkylaryl groups include

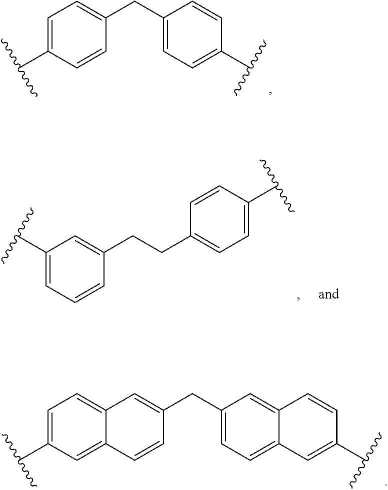 [SMILES: *c1ccc(Cc2ccc(*)cc2)cc1 ; *c1ccc(CCc2cccc(*)c2)cc1 ; *c1ccc2cc(Cc3ccc4cc(*)ccc4c3)ccc2c1]

In some embodiments, the arylalkylaryl group may be optionally substituted, in which case the alkyl group, either of the aryl groups, or any combination thereof, may be substituted as described herein.

The term “heteroaryl” refers to an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocyclyl” or “heterocycle” refers to a stable 3- to 18-membered ring (radical) which consists of carbon atoms and from one to five heteroatoms selected from the group consisting of nitrogen, oxygen, and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

In some embodiments, the heterocycle is a non-aromatic heterocycle. The term “non-aromatic heterocycle” means a non-aromatic monocyclic system containing 3 to 10 atoms, preferably 4 to about 7 carbon atoms, in which one or more of the atoms in the ring system is/are element(s) other than carbon, for example, nitrogen, oxygen, or sulfur. Representative non-aromatic heterocycle groups include pyrrolidinyl, 2-oxopyrrolidinyl, piperidinyl, 2-oxopiperidinyl, azepanyl, 2-oxoazepanyl, 2-oxooxazolidinyl, morpholino, 3-oxomorpholino, thiomorpholino, 1,1-dioxothiomorpholino, piperazinyl, tetrohydro-2H-oxazinyl, and the like.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “halo” or “halogen” means fluoro, chloro, bromo, or iodo.

The term “sulfate” is art-recognized and refers to the group —OSO₃H, or a pharmaceutically acceptable salt thereof.

The term “sulfonamido” is art-recognized and refers to the group represented by the general formulae

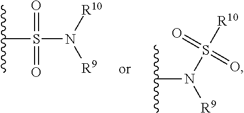

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” or “substitution” of an atom means that one or more hydrogen on the designated atom is replaced with a selection from the indicated group, provided that the designated atom's normal valency is not exceeded. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

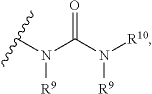

wherein R⁹ and R¹⁰ independently represent hydrogen or a hydrocarbyl.

As used herein, the term “amino acid side chain” or “side chain” refers to the characterizing substituent of the amino acid. This term refers to the substituent bound to the α-carbon of either a natural or non-natural α-amino acid. For example, the characterizing substituents of some naturally occurring amino acids are shown in Table 1.

TABLE 1
The Proteinogenic Amino Acids

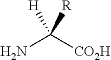

| Amino Acid | —R |
|---|---|
| Alanine | —CH₃ |
| Arginine | —(CH₂)₃NHC(═NH)NH₂ |
| Asparagine | —CH₂CONH₂ |
| Aspartic acid | —CH₂CO₂NH₂ |
| Cystine | —CH₂SH |
| Glutamine | —(CH₂)₂CONH₂ |
| Glutamic acid | —(CH₂)₂CO₂H |
| Glycine | —H |
| Histidine | —CH₂(4-imidazolyl) |
| Isoleucine | —CH(CH₃)CH₂CH₃ |
| Leucine | —CH₂CH(CH₃)₂ |
| Lysine | —(CH₂)₄NH₂ |
| Methionine | —(CH₂)₂SCH₃ |
| Phenylalanine | —CH₂Ph |
| Serine | —CH₂OH |
| Threonine | —CH(CH₃)OH |
| Tryptophan | —CH₂(3-indolyl) |
| Tyrosine | —CH₂(4- |
| | hydroxyphenyl) |
| Valine | —CH(CH₃)₂ |

Another naturally occurring amino acid is proline, in which the α-side chain terminates in a bond with the amino acid amine nitrogen atom.

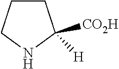

Some non-limiting examples of characterizing substituents of non-naturally occurring amino acids are shown in Table 2:

TABLE 2
Non-Natural Amino Acids

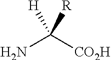

| Amino Acid | —R |
|---|---|
| α-Aminobutyric acid | —CH₂CH₃ |
| Ornithine | —(CH₂)₃NH₂ |
| Cyclohexylalanine | —CH₂C₆H₁₀ |
| Cyclopentylalanine | —CH₂C₅H₈ |
| Norvaline | —CH₂CH₂CH₃ |
| Norleucine | —(CH₂)₃CH₃ |

“Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; a “stable compound” or “stable structure” is meant to be a compound that is sufficiently robust to survive isolation to a useful degree of purity from a reaction mixture, and formulation into an efficacious therapeutic agent.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

Terminology related to “protecting”, “deprotecting,” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes described herein, the person of ordinary skill can readily envision those groups that would be suitable as “protecting groups.” Suitable groups for that purpose are discussed in standard textbooks in the field of chemistry, such as Greene, Protective Groups in Organic Synthesis, John Wiley & Sons, New York (1991), which is hereby incorporated by reference in its entirety.

The term “compounds of the invention”, and equivalent expressions, are meant to embrace compounds of general Formula (I), Formula (Ia), Formula (IV), Formula (IVa), Formula (V), and Formula (Va), as herein described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The compounds of the invention are nicotinate/nicotinamide riboside-based compounds and derivatives. Exemplary compounds include derivatives of nicotinamide (Nam), nicotinic acid (NA), nicotinamide ribose (NR), nicotinic mononucleotide (NMN), and nicotinic acid mononucleotide (NaMN).

In some embodiments, the compounds of the invention are zwitterions. The term “zwitterion” or “zwitterionic,” as used herein, refers to a neutral molecule with both positive and negative electrical charges. Zwitterions may also be called dipolar ions or inner salts, which are different from molecules that have dipoles at different locations within the molecule. Alternatively, the compounds of the invention may be ionic compounds possessing a counterion or counterions. Exemplary counter ions include, but are not limited to fluoride, chloride, bromide, iodide, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfonate, trifluoromethanesulfonate, trichloromethanesulfonate, tribromomethanesulfonate, and trifluoroacetate. The compounds of the invention may exist in a variety of different forms such as compounds associated with counterions (e.g., dry salts), but also in forms that are not associated with counterions (e.g., aqueous solution or organic solution).

The term “pharmaceutically acceptable salts” refers to the relatively non-toxic, inorganic, and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418, which are hereby incorporated by reference in their entirety). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. Suitable inorganic base addition salts are prepared from metal bases which include, for example, sodium hydride, sodium hydroxide, potassium hydroxide, calcium hydroxide, aluminium hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention.

The term “prodrug” refers to compounds that are rapidly transformed in vivo to yield the parent compound of the above formula, for example by hydrolysis in blood. Functional groups which may be rapidly transformed, by metabolic cleavage, in vivo form a class of groups reactive with the compounds of this application. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this application are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier (1985); Methods in Enzymology, K. Widder et al, Ed., Academic Press, 42, p. 309-396 (1985); A Textbook of Drug Design and Development, Krogsgaard-Larsen and H. Bundgaard, ed., Chapter 5; “Design and Applications of Prodrugs” p. 113-191 (1991); Advanced Drug Delivery Reviews, H. Bundgard, 8, p. 1-38 (1992); J. Pharm. Sci., 77:285 (1988); Nakeya et al, Chem. Pharm. Bull., 32:692 (1984); Higuchi et al., “Pro-drugs as Novel Delivery Systems,” Vol. 14 of the A.C.S. Symposium Series, and Bioreversible Carriers in Drug Design, Edward B. Roche, ed., American Pharmaceutical Association and Pergamon Press (1987), which are incorporated herein by reference in their entirety. Examples of prodrugs include, but are not limited to, acetate, formate, and benzoate derivatives of alcohol and amine functional groups in the compounds of the invention.

The term “solvate” refers to compounds of the present invention in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. Pharmaceutical acceptable solvates and hydrates may include a compound with one or more solvent or water molecules, or 1 to about 100, or 1 to about 10, or one to about 2, 3 or 4, solvent or water molecules. In general, solvates are formed by dissolving the compound in the appropriate solvent and isolating the solvate by cooling or using an antisolvent. The solvate is typically dried or azeotroped under ambient conditions.

Compounds described herein may contain one or more asymmetric centers and may thus give rise to enantiomers, diastereomers, and other stereoisomeric forms. Each chiral center may be defined, in terms of absolute stereochemistry, as (R)- or (S)—. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

This technology also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

In the characterization of some of the substituents, it is recited that certain substituents may combine to form rings. Unless stated otherwise, it is intended that such rings may exhibit various degrees of unsaturation (from fully saturated to fully unsaturated), may include heteroatoms and may be substituted with lower alkyl or alkoxy.

As used herein, the term “subject” includes any human or non-human animal. For example, the methods and compositions described herein can be used to treat a subject (e.g., a human patient) in need of treatment of skin affliction or skin condition. The subjects may be humans who are in need of treatment of inflammation, sun damage or natural aging.

A “therapeutically effective amount” means an amount of the compounds disclosed herein, or other active agent, set forth herein that, when administered to a subject, is effective in producing a therapeutic effect.

As used herein, “administering” refers to the physical introduction of a composition comprising a therapeutic agent to a subject, using any of the various methods and delivery systems known to those skilled in the art. Preferred routes of administration for the therapeutic agents described herein include topical, oral, intravenous, intraperitoneal, intramuscular, subcutaneous, spinal or other parenteral routes of administration. Administering can also be performed, for example, once, a plurality of times, and/or over one or more extended periods.

As used herein, the terms “treatment,” “treating”, “treat”, or the like, mean to alleviate or reduce the severity of at least one symptom or indication, to eliminate the causation of symptoms either on a temporary or permanent basis, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may result in a partial response (PR) or a complete response (CR).

The term “prevent,” as used herein, includes prophylactic treatment or treatment that prevents one or more symptoms or conditions of a disease, disorder, or conditions described herein (e.g., skin ageing). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “method of treating” means amelioration or relief from the symptoms and/or effects associated with the disorders described herein. As used herein, reference to “treatment” of a patient is intended to include prophylaxis.

Various aspects described herein are described in further detail in the following subsections.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation of Exemplary Compounds of the Disclosure

### Materials and Methods

All commercially available chemicals and reagent grade solvents were used without further purification unless otherwise specified. Thin-layer chromatography (TLC) was performed on silica gel plates using UV-light (254 and 365 nm) detection or visualizing agents. HPLC chromatography was carried out on a Porous Graphitic Carbon HPLC Column using an Agilent 1269 Infinity11 coupled with InfinityLab LC/MSD. NMR spectra were acquired at room temperature using a Bruker spectrometer at 400 MHz using the solvents individually identified. Chemical shifts (6) are given in parts per million (ppm) with reference to solvent signals [¹H-NMR: CDCl₃ (7.26 ppm), CD₃OD (3.30 ppm), DMSO-d₆ (2.49 ppm)]. Signal patterns are reported as s (singlet), d (doublet), t (triplet), q (quartet), quin (quintet), sex (sextet), sep (septet), m (multiplet), br (broad), dd (doublet of doublets), dt (doublet of triplets), td (triplet of doublets), and tt (triplet of triplets). Coupling constants (J) are given in Hz. LCMS analysis was conducted on an Agilent 1269 Infinity11 coupled with InfinityLab LC/MSD mass spectrometer. Samples were ionized by electrospray ionization (ESI) in positive mode and reported as m/z (relative intensity) for the molecular ion [M].

### Synthesis of Nicotinate/Nicotinamide Riboside Compounds and Derivatives

### ((2R,3S,4R,5R)-3,4-dihydroxy-5-(3-((phenylthio)carbonyl)pyridin-1-ium-1-yl)tetrahydrofuran-2-yl)methyl hydrogen phosphate (6)

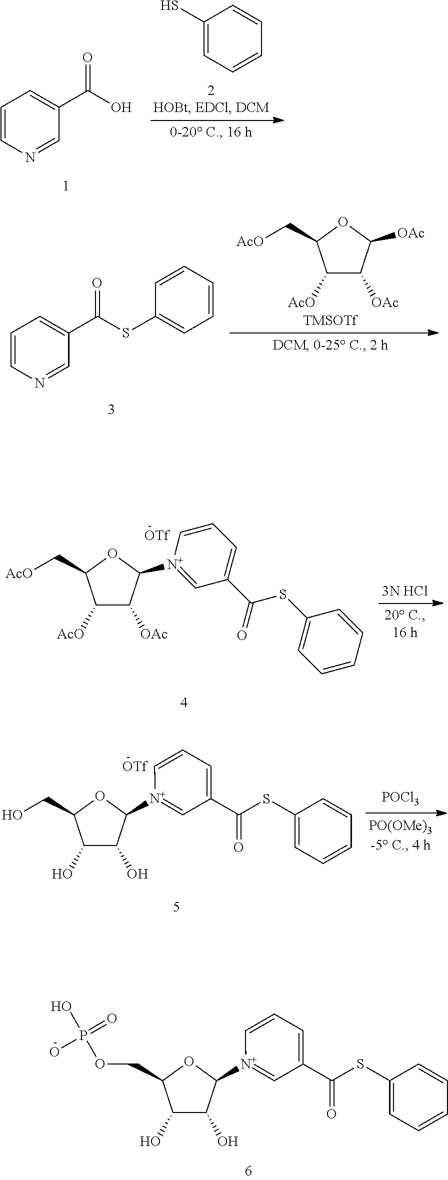

To a solution of nicotinic acid (1, 5 g, 40.61 mmol, 3.40 mL, 1 eq), and 1-Ethyl-3-(3-dimethylaminopropyl)carbodiimide (EDCI) (10.12 g, 52.80 mmol, 1.3 eq) in dichloromethane (DCM) (50 mL) was added dropwise hydroxybenzotriazole (HOBt) (7.13 g, 52.80 mmol, 1.3 eq) at 0° C. After addition, the mixture was stirred at 0° C. for 10 min, followed by dropwise addition of benzenethiol (2, 6.180 g, 56.09 mmol, 5.72 mL, 1.38 eq) at 0° C. The resulting mixture was stirred at 20° C. for 5 hours. LCMS (0-60AB/1.5 min, RT=1.033 min, 216.1 [M+H]⁺, ESI pos) showed the major peak with desired product was detected.

The mixture was diluted with ice water (150 mL), and extracted with DCM (150 mL). The combined organic layers were washed with brine (210 mL), dried over sodium sulfate (Na₂SO₄), filtered and concentrated under reduced pressure to give a residue. The aqueous phase was quenched with aq. sodium hypochlorite (NaClO). The residue was purified by column chromatography (SiO₂, Petroleum ether:Ethyl acetate=1:0 to 5:1) to afford S-phenyl pyridine-3-carbothioate (3, 7 g, 32.19 mmol, 79.26% yield) as colorless oil. LCMS: t_{R}=0.861 min, m/z=216.3 (M+H)+. ¹H NMR (400 MHz, CDCl₃) δ (ppm)=9.25 (d, J=2.1 Hz, 1H), 8.82 (d, J=4.8 Hz, 1H), 8.26 (br d, J=8.1 Hz, 1H), 7.54 (br s, 5H), 7.45-7.41 (m, 1H).

To a solution of (3R,4R,5R)-5-(acetoxymethyl)tetrahydrofuran-2,3,4-triyl triacetate (3, 4.69 g, 14.73 mmol, 1.2 eq), and tert-butyl nicotinate (2.2 g, 12.28 mmol, 1 eq) in DCM (30 mL) was added trimethylsilyl trifluoromethanesulfonate (TMSOTf) (1.36 g, 6.14 mmol, 1.11 mL, 0.5 eq) at 0° C. The mixture was stirred at 25° C. for 2 hours. LCMS (0-60AB/1.5 min, RT=0.915 min, 474.1 [M+H]+, ESI pos) showed the major peak with desired product was detected. The mixture was diluted with ice water (200 mL), then neutralized to pH 6-7 with saturated aqueous sodium bicarbonate (NaHCO₃). The residue was extracted with DCM (50 mL). The combined organic layers were washed with brine (250 mL), dried over Na₂SO₄, filtered and concentrated under reduced pressure to give a residue. The residue was purified by column chromatography (SiO₂, Petroleum ether:Ethyl acetate=1:0 to 0:1) to afford 1-((2R,3R,4R,5R)-3,4-diacetoxy-5-(acetoxymethyl)tetrahydrofuran-2-yl)-3-((phenylthio)carbonyl)pyridin-1-ium (4, 6 g, 12.64 mmol, 90.74% yield) as white solid. LCMS: t_{R}=0.915 min, m/z=474.1 (M+H)+. ¹H NMR (400 MHz, CDCl₃) δ 9.58-9.50 (m, 2H), 9.09 (d, J=8.2 Hz, 1H), 8.50-8.40 (m, 1H), 7.51 (s, 5H), 6.69 (d, J=3.8 Hz, 1H), 5.55-5.48 (m, 1H), 5.36 (t, J=5.6 Hz, 1H), 4.77-4.70 (m, 1H), 4.60-4.41 (m, 2H), 2.20-2.12 (m, 9H).

1-((2R,3R,4R,5R)-3,4-diacetoxy-5-(acetoxymethyl)tetrahydrofuran-2-yl)-3-((phenylthio)carbonyl)pyridin-1-ium (4, 1 g, 2.11 mmol, 1 eq) was suspended in hydrochloric acid (HClaq) (3 M, 10 mL, 14.24 eq) at 0° C. The mixture was stirred at 20° C. for 16 hours. LCMS (0-60AB/1.5 min, RT=0.741 min, 348.0[M+H]+, ESI pos) showed the major peak with desired product was detected. The reaction mixture was concentrated under reduced pressure to give a residue at 0° C. The crude product was purified by reversed-phase HPLC (MeCN/H₂O, neutral) to afford 1-((2R,3R,4S,5R)-3,4-dihydroxy-5-(hydroxymethyl)tetrahydrofuran-2-yl)-3-((phenylthio)carbonyl)pyridin-1-ium (5, 300 mg, 809.43 umol, 38.41% yield) as white solid. LCMS: t_{R}=0.727 min, m/z=348.0 (M+H)+. ¹H NMR (400 MHz, D₂O) δ 9.75 (s, 1H), 9.29 (d, J=6.2 Hz, 1H), 9.11 (br d, J=8.2 Hz, 1H), 8.35-8.23 (m, 1H), 7.63-7.50 (m, 5H), 6.25 (d, J=3.9 Hz, 1H), 4.51-4.41 (m, 2H), 4.36-4.29 (m, 1H), 4.08-3.99 (m, 1H), 3.90-3.82 (m, 1H).

To a solution of 1-((2R,3R,4S,5R)-3,4-dihydroxy-5-(hydroxymethyl)tetrahydrofuran-2-yl)-3-((phenylthio)carbonyl)pyridin-1-ium (5, 100 mg, 287.03 umol, 1 eq) in trimethyl phosphite (PO(OMe)₃) (1 mL) was added phosphoryl chloride (POCl₃) (0.6 mL) at 0° C. The mixture was stirred at 0° C. for 3 hours. LCMS (0-60CD/2 min, RT=0.412 min, 427.9[M+H]+, ESI pos) showed the major peak with desired product was detected. The mixture was adjusted to pH ˜7 with aq. NaHCO₃ at 0° C., then filtered to give a residue. The crude product was purified by reversed-phase HPLC (MeCN/H₂O, neutral) to afford ((2R,3S,4R,5R)-3,4-dihydroxy-5-(3-((phenylthio)carbonyl)pyridin-1-ium-1-yl)tetrahydrofuran-2-yl)methyl hydrogen phosphate (6) as white solid. LCMS: t_{R}=0.412 min, m/z=427.9 (M+H)⁺.

### 1-((2R,3R,4S,5R)-3,4-dihydroxy-5-((phosphonooxy)methyl)tetrahydrofuran-2-yl)-3-((6-(triphenylphosphonio)hexyl)carbamoyl)pyridin-1-ium (7)

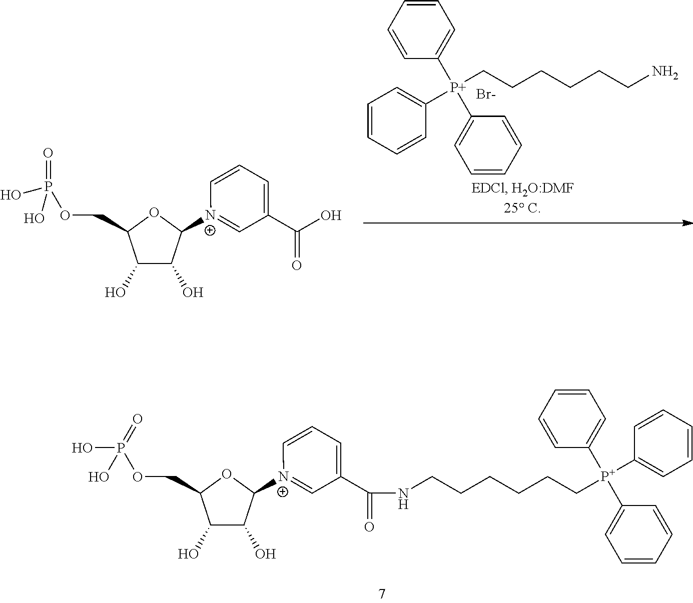 [SMILES: O=C(O)c1ccc[n+]([C@@H]2O[C@H](COP(=O)(O)O)[C@@H](O)[C@H]2O)c1 ; NCCCCCC[P+](c1ccccc1)(c1ccccc1)c1ccccc1 ; [Br-] ; O=C(NCCCCCC[P+](c1ccccc1)(c1ccccc1)c1ccccc1)c1ccc[n+]([C@@H]2O[C@H](COP(=O)(O)O)[C@@H](O)[C@H]2O)c1]

3-Carboxy-1-((2R,3R,4S,5R)-3,4-dihydroxy-5-((phosphonooxy)methyl)tetrahydrofuran-2-yl)pyridin-1-ium (1 mmol) was dissolved in a deionized water:DMF solution (1 M, 50:50) and sequentially added diisopropylethylamine (5 eq), and 1-ethyl-3-(3-dimethylaminopropyl)carbodiimide (1 eq). The mixture was stirred for 15 minutes at ambient temperature. To this solution (6-aminohexyl)triphenylphosphonium bromide hydrobromide (1 eq, 1M) in DMF was slowly added and stirred at 25° C. The reaction progression was monitored by LCMS. After completion of the reaction, the crude product was directly purified by reversed-phase HPLC (MeCN/H₂O). The purified fractions were lyophilized to afford 1-((2R,3R,4S,5R)-3,4-dihydroxy-5-((phosphonooxy)methyl)tetrahydrofuran-2-yl)-3-((6-(triphenylphosphonio)hexyl)carbamoyl)pyridin-1-ium (7) as a white solid (approximately 45% yield).

LCMS: LCMS (1-99AB mL/min, RT=5.757 min, 679.2 [M+H]⁺, ESI pos) showed the major peak with desired product was detected.

A general synthetic method for the formation of analogs of 7 is shown in Scheme 1 below.

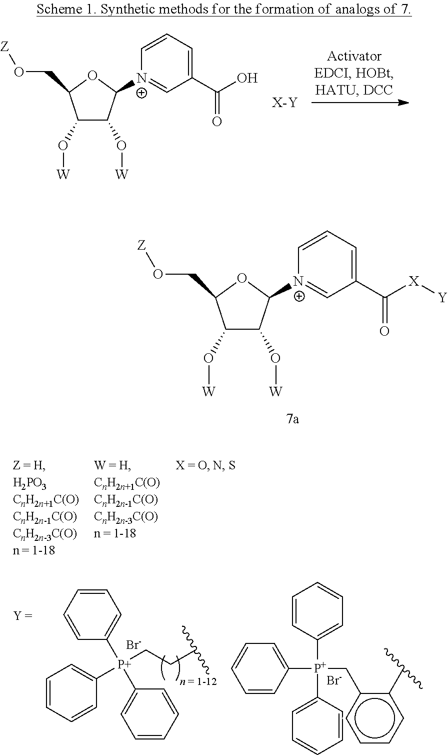

### ((2R,3S,4R,5R)-5-(3-(tert-butoxycarbonyl)pyridin-1-ium-1-yl)-3,4-dihydroxytetrahydrofuran-2-yl)methyl hydrogen phosphate (8)

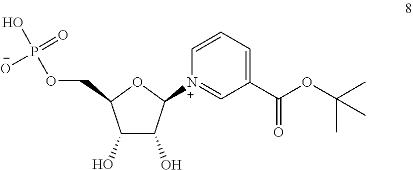 [SMILES: CC(C)(C)OC(=O)c1ccc[n+]([C@@H]2O[C@H](COP(=O)([O-])O)[C@@H](O)[C@H]2O)c1]

((2R,3S,4R,5R)-5-(3-(tert-butoxycarbonyl)pyridin-1-ium-1-yl)-3,4-dihydroxytetrahydrofuran-2-yl)methyl hydrogen phosphate (8) was formed using the method as disclosed for compound 7. LCMS: t_{R}=0.660 min, m/z=392.2 (M+H)⁺. ¹H NMR (400 MHz, D₂O) δ 9.41 (s, 1H), 9.33 (d, J=6.5 Hz, 1H), 9.05 (d, J=8.1 Hz, 1H), 8.31-8.24 (m, 1H), 6.19 (d, J=5.3 Hz, 1H), 4.63-4.59 (m, 1H), 4.52 (t, J=5.1 Hz, 1H), 4.44-4.39 (m, 1H), 4.31-4.24 (m, 1H), 4.17-4.10 (m, 1H), 1.61 (s, 9H).

### 3-((4-(4-aminobenzyl)phenyl)carbamoyl)-1-((2R,3R,4S,5R)-3,4-dihydroxy-5-((phosphonooxy)methyl)tetrahydrofuran-2-yl)pyridin-1-ium (9)

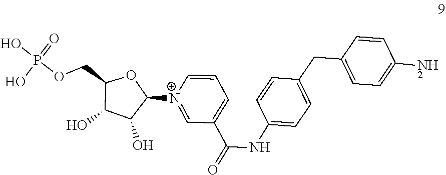 [SMILES: Nc1ccc(Cc2ccc(NC(=O)c3ccc[n+]([C@@H]4O[C@H](COP(=O)(O)O)[C@@H](O)[C@H]4O)c3)cc2)cc1]

3-((4-(4-aminobenzyl)phenyl)carbamoyl)-1-((2R,3R,4S,5R)-3,4-dihydroxy-5-((phosphonooxy)methyl)tetrahydrofuran-2-yl)pyridin-1-ium (9) was formed using the method as disclosed for compound 7. LCMS: t_{R}=16.34 min, m/z=516.15 (M+H)⁺.

### 6-(nicotinamido)hexyl)triphenylphosphonium (10)

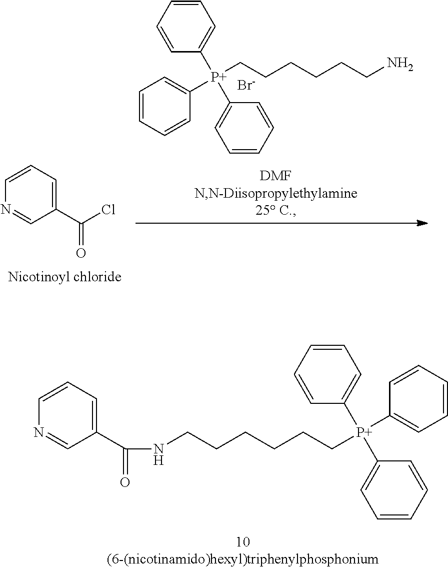 [SMILES: NCCCCCC[P+](c1ccccc1)(c1ccccc1)c1ccccc1 ; [Br-] ; O=C(Cl)c1cccnc1 ; O=C(NCCCCCC[P+](c1ccccc1)(c1ccccc1)c1ccccc1)c1cccnc1]

Nicotinoyl chloride (6-(nicotinamido)hexyl)triphenylphosphonium Nicotinoyl chloride (1 eq) was dissolved in DMF (1 M) and sequentially added diisopropylethylamine (5 eq) and (6-aminohexyl)triphenylphosphonium bromide hydrobromide (1 eq). The reaction progression was monitored by LCMS. After completion of the reaction, the reaction was quenched with 1M HCl and the concentrated under reduced pressure. This crude product was directly purified by reversed-phase HPLC (MeCN/H₂O) to afford 6-(nicotinamido)hexyl)triphenylphosphonium (10) as a white solid in an approximately 70% yield. LCMS: LCMS (1-99AB, 1.0 m/min, RT=5.902 min, 467.1 [M+H]⁺, ESI pos) showed the major peak with desired product was detected. ¹H NMR (400 MHz, METHANOL-d4) δ 8.93 (s, 1H), 8.69 (s, 1H), 8.46 (s, 1H), 8.22 (s, 1H), 7.81 (m, 15H), 7.54 (s, 1H), 3.39 (m, 4H), 1.65 (m, 6H), 1.43 (m, 2H).

A general synthetic method for the formation of analogs of 10 is shown in Scheme 2 below.

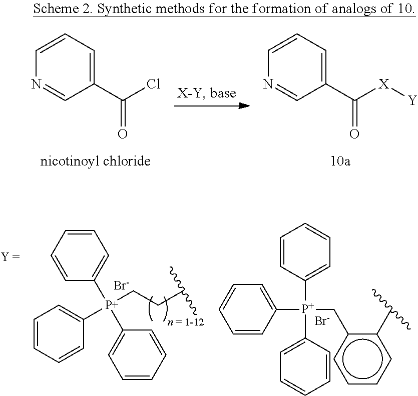

### 3-(tert-butoxycarbonyl)-1-((2R,3R,4R,5R)-3,4-diacetoxy-5-(acetoxymethyl)tetrahydrofuran-2-yl)pyridin-1-ium (11)

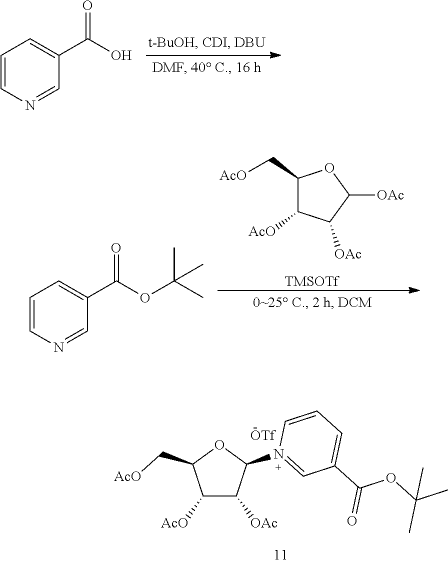

To a solution of nicotinic acid (1 eq) in DMF (50 mL) was added 1,1′-carbonyldiimidazole (CDI) (1 eq) under N₂. After addition, the mixture was stirred at 40° C. for 1 hour, followed by addition of t-BuOH (2 eq), and 1,8-Diazabicyclo [5.4.0]undec-7-ene (DBU) (61 eq). The resulting mixture was stirred at 40° C. for 16 hours. TLC (Petroleum ether:Ethyl acetate=0:1) showed a new spot was detected (R_{f}=0.67). EA (100 mL) was added to the mixture, the solution was washed with 10% acetic acid (20 mL), H₂O (50 mL), and aqueous 10% K₂CO₃ (50 mL), and dried over Na₂SO₄, filtered and concentrated under reduced pressure to afford tert-butyl nicotinate (5 g, 27.90 mmol, 68.69% yield) as a yellow oil. ¹H NMR (400 MHz, DMSO) δ 9.11-8.97 (m, 1H), 8.85-8.73 (m, 1H), 8.29-8.16 (m, 1H), 7.62-7.47 (m, 1H), 1.55 (s, 9H).

To a solution of (3R,4R,5R)-5-(acetoxymethyl)tetrahydrofuran-2,3,4-triyl triacetate (1.2 eq), was added tert-butyl nicotinate (1 eq) in DCM (30 mL) with TMSOTf (10.5 eq) at 0° C. The mixture was stirred at 25° C. for 16 hours. LCMS (0-60AB/1.5 min, RT=0.879 min, 438.2[M+H]⁺, ESI pos) showed the major peak with desired product was detected.

The residue was diluted with ice water (50 mL), and the mixture was neutralized to pH 6-7 with saturated aqueous NaHCO₃ (ca. 15 mL). The residue was extracted with DCM (50 mL). The combined organic layers were washed with brine (50 mL), dried over Na₂SO₄, filtered and concentrated under reduced pressure to give a residue. The residue was purified by column chromatography (SiO₂, Petroleum ether:Ethyl acetate=1:0 to 0:1) to afford 3-(tert-butoxycarbonyl)-1-((2R,3R,4R,5R)-3,4-diacetoxy-5-(acetoxymethyl)tetrahydrofuran-2-yl)pyridin-1-ium (11) (N-2-INT4, 3 g, 6.76 mmol, 40.38% yield) as a white solid. ¹H NMR (400 MHz, D2O) δ 9.44 (s, 1H), 9.30 (d, J=6.2 Hz, 1H), 9.05 (d, J=8.1 Hz, 1H), 8.42-8.33 (m, 1H), 6.70 (d, J=3.4 Hz, 1H), 5.66-5.57 (m, 1H), 5.41 (t, J=5.9 Hz, 1H), 4.77-4.70 (m, 1H), 4.52-4.40 (m, 2H), 2.15 (s, 3H), 2.10 (d, J=7.1 Hz, 6H), 1.61 (s, 9H).

A general synthetic method for the formation of analogs of 11 is shown in Scheme 3 below.

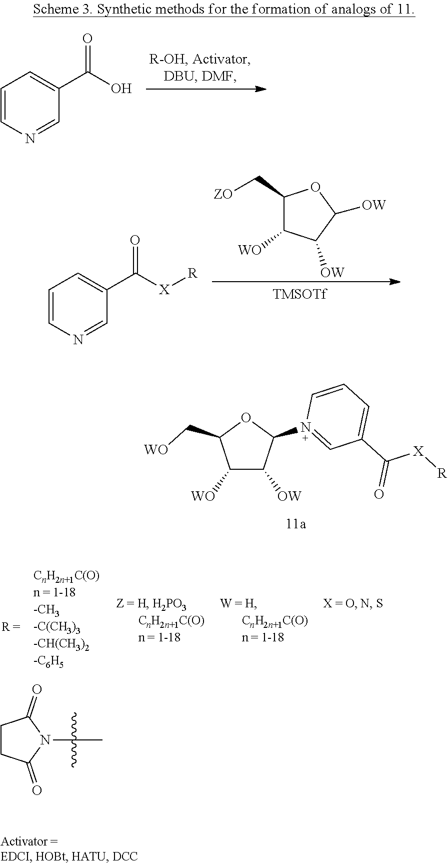

### Synthesis of Additional Nicotinate/Nicotinamide Riboside Compounds and Derivatives

### 3-(((S)-1-carboxy-2-(1H-indol-3-yl)ethyl)carbamoyl)-1-((2R,3R,4S,5R)-3,4-dihydroxy-5-((phosphonooxy)methyl)tetrahydrofuran-2-yl)pyridin-1-ium

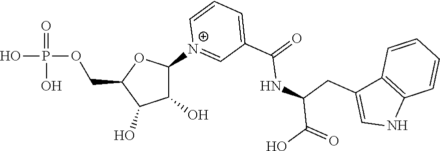 [SMILES: O=C(N[C@@H](Cc1c[nH]c2ccccc12)C(=O)O)c1ccc[n+]([C@@H]2O[C@H](COP(=O)(O)O)[C@@H](O)[C@H]2O)c1]

3-Carboxy-1-((2R,3R,4S,5R)-3,4-dihydroxy-5-((phosphonooxy)methyl)tetrahydrofuran-2-yl)pyridin-1-ium (1 mmol) is dissolved in a deionized water:DMF solution (1 M, 50:50) and diisopropylethylamine (5 eq), and 1-ethyl-3-(3-dimethylaminopropyl)carbodiimide (1 eq) are added. The mixture is stirred for 15 minutes at ambient temperature. L-Tryptophan (1 eq, 1M) in DMF is then slowly added to the solution and stirred at 25° C. The reaction progression is monitored by LCMS. After completion of the reaction, the crude product is directly purified by reversed-phase HPLC. The purified fractions are lyophilized to afford 3-(((S)-1-carboxy-2-(1H-indol-3-yl)ethyl)carbamoyl)-1-((2R,3R,4S,5R)-3,4-dihydroxy-5-((phosphonooxy)methyl)tetrahydrofuran-2-yl)pyridin-1-ium.

### 3-(((R)-1-amino-4-methyl-1-oxopentan-2-yl)carbamoyl)-1-((2R,3R,4S,5R)-3,4-dihydroxy-5-((phosphonooxy)methyl)tetrahydrofuran-2-yl)pyridin-1-ium

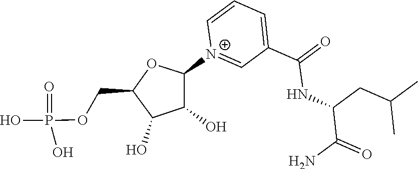 [SMILES: CC(C)C[C@@H](NC(=O)c1ccc[n+]([C@@H]2O[C@H](COP(=O)(O)O)[C@@H](O)[C@H]2O)c1)C(N)=O]

3-Carboxy-1-((2R,3R,4S,5R)-3,4-dihydroxy-5-((phosphonooxy)methyl)tetrahydrofuran-2-yl)pyridin-1-ium (1 mmol) is dissolved in a deionized water:DMF solution (1 M, 50:50) and diisopropylethylamine (5 eq), and 1-ethyl-3-(3-dimethylaminopropyl)carbodiimide (1 eq) are added. The mixture is stirred for 15 minutes at ambient temperature. (R)-2-amino-4-methylpentanamide (1 eq, 1M) in DMF is then slowly added to the solution and stirred at 25° C. The reaction progression is monitored by LCMS. After completion of the reaction, the crude product is directly purified by reversed-phase HPLC. The purified fractions are lyophilized to afford 3-(((R)-1-amino-4-methyl-1-oxopentan-2-yl)carbamoyl)-1-((2R,3R,4S,5R)-3,4-dihydroxy-5-((phosphonooxy)methyl)tetrahydrofuran-2-yl)pyridin-1-ium.

### 1-((2R,3R,4S,5R)-3,4-dihydroxy-5-((phosphonooxy)methyl)tetrahydrofuran-2-yl)-3-((((1R,2S,5R)-2-isopropyl-5-methylcyclohexyl)oxy)carbonyl)pyridin-1-ium

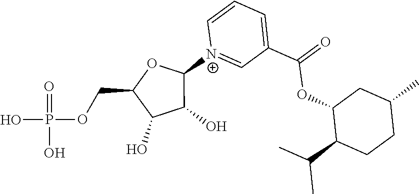 [SMILES: CC(C)[C@@H]1CC[C@@H](C)C[C@H]1OC(=O)c1ccc[n+]([C@@H]2O[C@H](COP(=O)(O)O)[C@@H](O)[C@H]2O)c1]

3-Carboxy-1-((2R,3R,4S,5R)-3,4-dihydroxy-5-((phosphonooxy)methyl)tetrahydrofuran-2-yl)pyridin-1-ium (1 mmol) is dissolved in a deionized water:DMF solution (1 M, 50:50) and diisopropylethylamine (5 eq), and 1-ethyl-3-(3-dimethylaminopropyl)carbodiimide (1 eq) are added. The mixture is stirred for 15 minutes at ambient temperature. Menthol (1 eq, 1M) in DMF is then slowly added to the solution and stirred at 25° C. The reaction progression is monitored by LCMS. After completion of the reaction, the crude product is directly purified by reversed-phase HPLC. The purified fractions are lyophilized to afford 1-((2R,3R,4S,5R)-3,4-dihydroxy-5-((phosphonooxy)methyl)tetrahydrofuran-2-yl)-3-((((1R,2S,5R)-2-isopropyl-5-methylcyclohexyl)oxy)carbonyl)pyridin-1-ium.

### Intermediate Formation

Not to be withheld to any theory, a proposed reaction mechanism of the novel procedure is outlined in Scheme 4. Transformation from NaMN to compound 9 is believed to undergo through the intermediate 9′. This is confirmed by treating NaMN with only EDCI and N,N-Diisopropylethylamine (DIPEA) (according to the novel procedure) which resulted in intermediate 9′. HPLC data show that the NAMN peak at t_{R}=14.8 min moved to t_{R}=18.5 min suggesting the formation of the intermediate 9′. By treating this mixture with 4,4′-methylenedianiline shifts the t_{R}=18.5 to the new amide (9) peak t_{R}=16.34 min.

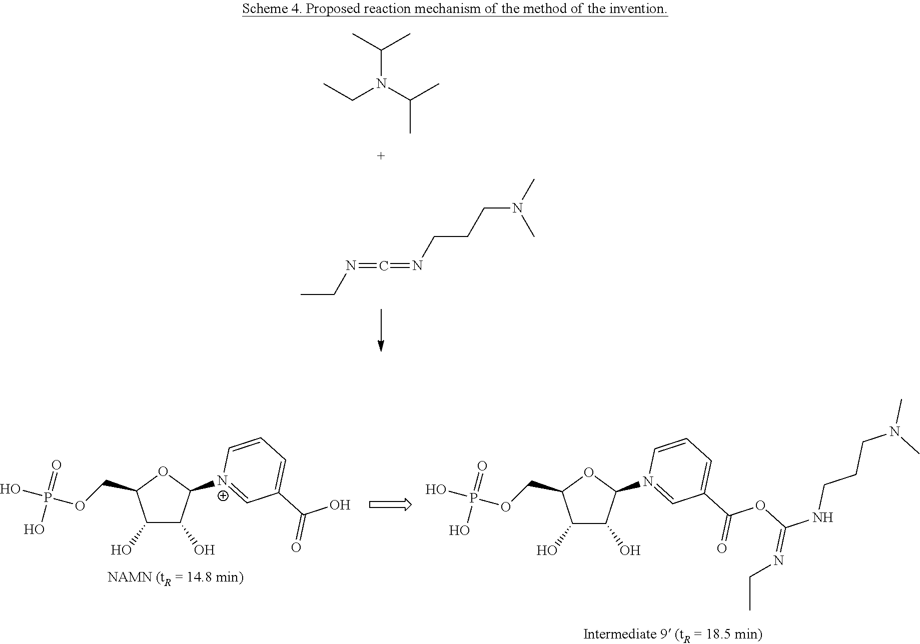

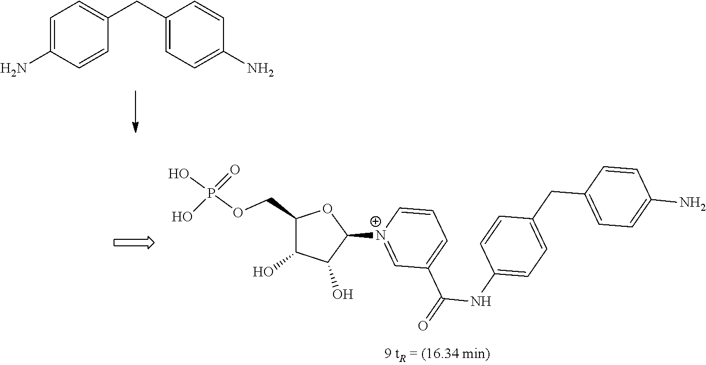 [SMILES: Nc1ccc(Cc2ccc(N)cc2)cc1 ; Nc1ccc(Cc2ccc(NC(=O)c3ccc[n+]([C@@H]4O[C@H](COP(=O)(O)O)[C@@H](O)[C@H]4O)c3)cc2)cc1]

### Example 2: Further Preparation of Exemplary Compounds of the Disclosure

### 2-isopropyl-5-methylcyclohexyl nicotinate

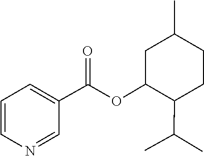 [SMILES: CC1CCC(C(C)C)C(OC(=O)c2cccnc2)C1]

1 eq of Nicotinoyl chloride hydrochloride was dissolved in 0.1 M DMF and treated with 1 eq of menthol at 0° C. The reaction was stirred for 24 hours. The crude product was purified by reversed-phase HPLC (MeCN/H2O, 0.1% TFA) to afford 2-isopropyl-5-methylcyclohexyl nicotinate in 94% yield as white solid. LCMS: t_{R}=0.727 min, m/z=262.0 (M+H)⁺.

### 8-hydroxy-6-oxo-6H-benzo[c]chromen-3-yl nicotinate

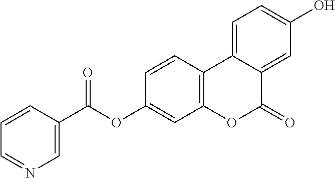 [SMILES: O=C(Oc1ccc2c(c1)oc(=O)c1cc(O)ccc12)c1cccnc1]

To a solution of 3,8-dihydroxy-6H-benzo[c]chromen-6-one (1, 330 mg, 1.45 mmol, 1 eq), nicotinoyl chloride (2, 1.29 g, 7.23 mmol, 73.40 uL, 5 eq, HCl) in MeCN (10 mL) was added DMAP (88.34 mg, 723.05 umol, 0.5 eq) and EDCI (554.43 mg, 2.89 mmol, 2 eq), TEA (585.32 mg, 5.78 mmol, 805.12 uL, 4 eq). The mixture was stirred at 25° C. for 48 hours. LCMS (5-95AB/1 min, RT=0.456 min, 334.1[M+H]⁺, ESI pos) showed the major peak with desired product was detected. The reaction mixture was filtered and concentrated under reduced pressure to give a residue. The residue was purified by prep-HPLC (column: Phenomenex Luna C8 250×50 mm×10 um; mobile phase: [water (HCl)-ACN]; B %: 20%-50%, 20 min) to afford 8-hydroxy-6-oxo-6H-benzo[c]chromen-3-yl nicotinate (N-16, 11.2 mg, 32.26 umol, 2.23% yield, 96% purity) as white solid. LCMS: t_{R}=0.461 min, m/z=334.1 (M+H)⁺.

### tert-butyl 18-((2-(nicotinamido)ethyl)amino)-18-oxooctadecanoate

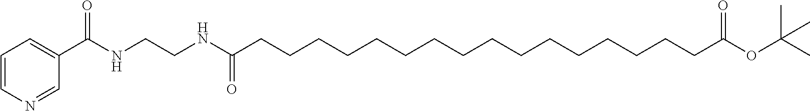 [SMILES: CC(C)(C)OC(=O)CCCCCCCCCCCCCCCCC(=O)NCCNC(=O)c1cccnc1]

To a solution of nicotinic acid (1, 10 g, 81.23 mmol, 6.80 mL, 1 eq) in DCM (50 mL), DMF (593.73 mg, 8.12 mmol, 624.98 uL, 0.1 eq) was added oxalyl dichloride (20.62 g, 162.46 mmol, 14.22 mL, 2 eq) at 0° C. The mixture was stirred at 25° C. for 2 hours. TLC (Petroleum ether:Ethyl acetate=0:1) showed the material was consumed (R_{f}=0.0), and a new spot was detected (R_{f}=0.26). The reaction mixture was filtered and concentrated under reduced pressure to afford nicotinoyl chloride (2, 10 g, 70.64 mmol, 86.97% yield) as a white solid. To a solution of nicotinoyl chloride (2, 1 g, 5.62 mmol, 6.80 mL, 1 eq, HCl), tert-butyl (2-aminoethyl)carbamate (3, 900.00 mg, 5.62 mmol, 882.35 uL, 1 eq) in DCM (10 mL) was added Et₃N (1.14 g, 11.23 mmol, 1.56 mL, 2 eq). The mixture was stirred at 25° C. for 2 hours. LCMS (5-95AB/1.5 min, RT=0.319 min, 266.2, [M+H]⁺, ESI pos) showed the major peak with desired product was detected. The reaction mixture was diluted with H₂O 80 mL and extracted with DCM 50 mL, dried over Na₂SO₄, filtered and concentrated under reduced pressure to afford tert-butyl (2-(nicotinamido)ethyl)carbamate (4, 1.5 g, crude) as a yellow oil. To this tert-butyl (2-(nicotinamido)ethyl)carbamate (4, 1.5 g, 5.65 mmol, 1 eq) in DCM (20 mL) was added TFA (3.22 g, 28.27 mmol, 2.09 mL, 5 eq). The mixture was stirred at 25° C. for 1 hour. LCMS (0-60CD/1.5 min, RT=0.338 min, 166.4, [M+H]⁺, ESI pos) showed the major peak with desired product was detected. The reaction mixture concentrated under reduced pressure to afford N-(2-aminoethyl)nicotinamide (5, 900 mg, 5.45 mmol, 96.36% yield) as a yellow oil. To this 18-(tert-butoxy)-18-oxooctadecanoic acid (6, 471.08 mg, 1.27 mmol, 1.05 eq) in DCM (5 mL) was added dropwise HATU (552.42 mg, 1.45 mmol, 1.2 eq), DIEA (469.43 mg, 3.63 mmol, 632.65 uL, 3 eq). After addition, the mixture was stirred at 25° C. for 30 min, and then N-(2-aminoethyl)nicotinamide (5, 200 mg, 1.21 mmol, 1 eq) in DCM (1 mL) was added. The resulting mixture was stirred at 25° C. for 16 hours. LCMS (5-95AB/1.5 min, RT=0.726 min, 518.4, [M+H]⁺, ESI pos) showed the major peak with desired product was detected. The residue was diluted with H₂O (60 mL) and extracted with DCM 100 mL (60 mL×3). The combined organic layers were dried over Na₂SO₄, filtered and concentrated under reduced pressure to give a residue. The residue was purified by prep-HPLC (column: Phenomenex luna C18 150×40 mm×15 um; mobile phase: [water(0.225% FA)-ACN]; B %: 80%-100%, 15 min) to afford tert-butyl 18-((2-(nicotinamido)ethyl)amino)-18-oxooctadecanoate (N-10, 185 mg, 352.03 umol, 29.08% yield, 98.52% purity) as a white solid. LCMS: t_{R}=0.724 min, m/z=518.5 (M+H)^{+ 1}H NMR (400 MHz, CDCl₃) δ 9.08 (s, 1H), 8.70 (br d, J=3.9 Hz, 1H), 8.16 (br d, J=8.1 Hz, 1H), 7.98 (br s, 1H), 7.42-7.35 (m, 1H), 6.51 (br t, J=5.3 Hz, 1H), 3.63-3.49 (m, 4H), 2.19 (t, J=7.5 Hz, 4H), 1.61-1.51 (m, 4H), 1.43 (s, 9H), 1.23 (br t, J=13.3 Hz, 24H).

### (E)-5-(4-(nicotinoyloxy)styryl)-1,3-phenylene diacetate

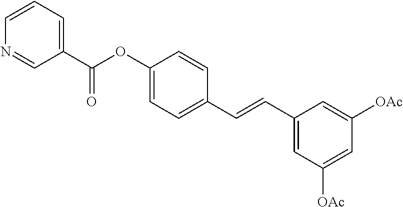 [SMILES: CC(=O)Oc1cc(/C=C/c2ccc(OC(=O)c3cccnc3)cc2)cc(OC(C)=O)c1]

To a solution of (E)-5-(4-hydroxystyryl)benzene-1,3-diol (1, 1 g, 4.38 mmol, 1 eq) in Py (5 mL) was added Ac2O (2.68 g, 26.29 mmol, 2.47 mL, 6 eq). The mixture was stirred at 80° C. for 1 hour. LCMS (5-95AB/1 min, RT=0.570 min, 355.0[M+H]⁺, ESI pos) showed the major peak with desired ms. The product was obtained after precipitation in 50 mL of water, filtration and two successive washings with water to afford (E)-5-(4-acetoxystyryl)-1,3-phenylene diacetate (2, 1.5 g, 4.23 mmol, 96.62% yield) as a yellow solid. To this solution of (E)-5-(4-acetoxystyryl)-1,3-phenylene diacetate (2, 3 g, 8.47 mmol, 1 eq), (E)-5-(4-hydroxystyryl)benzene-1,3-diol (1, 966.17 mg, 4.23 mmol, 0.5 eq) in DMSO (20 mL) was added K2CO3 (1.17 g, 8.47 mmol, 1 eq). The mixture was stirred at 25° C. for 16 hours. LCMS (5-95AB/1 min, RT=0.519 min, 312.2[M+H]⁺, ESI pos) showed the major peak with desired product was detected. The reaction mixture was diluted with H2O (150 mL) and extracted with EA (150 mL×3), dried over Na2SO4, filtered and concentrated under reduced pressure to give a residue. The residue was purified by column chromatography (SiO2, Petroleum ether:Ethyl acetate=1:0 to 0:1) to afford (E)-5-(4-hydroxystyryl)-1,3-phenylene diacetate (3, 2.6 g, 8.32 mmol, 98.33% yield) as a white solid.

LCMS: RT=0.519 min, m/z=312.2[M+H]+ To this solution of (E)-5-(4-hydroxystyryl)-1,3-phenylene diacetate (3, 3 g, 9.61 mmol, 1 eq), nicotinoyl chloride (4, 1.71 g, 9.61 mmol, 1 eq, HCl) in DCM (50 mL) was added TEA (2.92 g, 28.82 mmol, 4.01 mL, 3 eq). The mixture was stirred at 25° C. for 1 hour. LCMS (5-95AB/1 min, RT=0.548 min, 418.2[M+H]+, ESI pos) showed the major peak with desired ms. The reaction mixture was filtered and concentrated under reduced pressure to give a residue. The residue was purified by column chromatography (SiO2, Petroleum ether:Ethyl acetate=1:0 to 1:1) to afford (E)-5-(4-(nicotinoyloxy)styryl)-1,3-phenylene diacetate (N-39-INT4, 1 g, 2.34 mmol, 24.33% yield, 97.54% purity) as a white solid. QC of N-39-INT4. LCMS: RT=0.556 min, m/z=418.3 (M+H)+1H NMR (400 MHz, MeOD) δ 9.34-9.24 (m, 1H), 8.89-8.79 (m, 1H), 8.61-8.50 (m, 1H), 7.66-7.56 (m, 3H), 7.38 (s, 1H), 7.28-7.01 (m, 6H), 2.32-2.26 (m, 6H)

### (E)-5-(4-(nicotinoyloxy)styryl)-1,3-phenylene dinicotinate

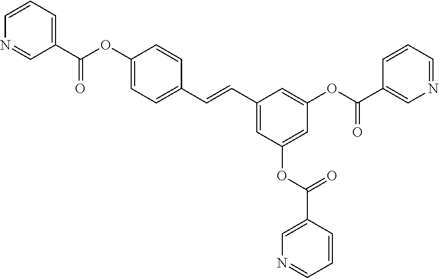 [SMILES: O=C(Oc1ccc(/C=C/c2cc(OC(=O)c3cccnc3)cc(OC(=O)c3cccnc3)c2)cc1)c1cccnc1]

3 eq of Nicotinoyl chloride hydrochloride was dissolved in 0.1 M DMF and treated with 1 eq of resveratol at 0° C. The reaction was stirred for 24 hours. The crude product was purified by reversed-phase HPLC (MeCN/H2O, 0.1% TFA) to afford (E)-5-(4-(nicotinoyloxy)styryl)-1,3-phenylene dinicotinate in 91% yield) as white solid. LCMS: t_{R}=0.9 min, m/z=544.0 (M+H)⁺.

### N-(2-aminoethyl)nicotinamide

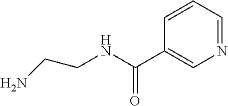 [SMILES: NCCNC(=O)c1cccnc1]

To a solution of nicotinoyl chloride (1, 5 g, 28.09 mmol, 6.80 mL, 1 eq, HCl), tert-butyl (2-aminoethyl)carbamate (2, 4.50 g, 28.09 mmol, 4.41 mL, 1 eq) in DCM (50 mL) was added Et3N (5.68 g, 56.17 mmol, 7.82 mL, 2 eq). The mixture was stirred at 25° C. for 2 hours. LCMS (5-95AB/1.5 min, RT=0.311 min, 266.1 [M+H]⁺, ESI pos) showed the major peak with desired product was detected. The reaction mixture was diluted with H2O 200 mL and extracted with DCM 150 mL, dried over Na2SO4, filtered and concentrated under reduced pressure to afford tert-butyl (2-(nicotinamido)ethyl)carbamate (3, 7 g, crude) as a yellow solid. To this solution of tert-butyl (2-(nicotinamido)ethyl)carbamate (3, 7 g, 26.38 mmol, 1 eq) in DCM (20 mL) was added TFA (15.04 g, 131.92 mmol, 9.77 mL, 5 eq). The mixture was stirred at 25° C. for 5 hour. LCMS (0-60CD/1.5 min, RT=0.309 min, 166.4[M+H]⁺, ESI pos) showed the major peak with desired product was detected. The reaction mixture was concentrated under reduced pressure. The crude product was purified by reversed-phase HPLC (0.1% TFA condition) to afford N-(2-aminoethyl)nicotinamide (N-13, 4 g, 14.30 mmol, 54.22% yield, 99.85% purity, TFA) as yellow solid. LCMS: tR=0.331 min, m/z=166.4 (M+H)^{+ 1}H NMR (400 MHz, DMSO) δ 9.07 (br s, 1H), 8.93 (br s, 1H), 8.78 (br d, J=4.4 Hz, 1H), 8.37-8.27 (m, 1H), 7.94 (br s, 2H), 7.68-7.58 (m, 1H), 3.59-3.49 (m, 2H), 3.07-2.98 (m, 2H)

### 2-aminoethyl nicotinate

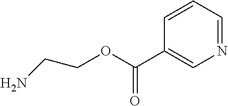 [SMILES: NCCOC(=O)c1cccnc1]

To a solution of nicotinoyl chloride (1, 2 g, 11.23 mmol, 1 eq, HCl) in DCM (50 mL) was added tert-butyl (2-hydroxyethyl)carbamate (1A, 1.81 g, 11.23 mmol, 1.74 mL, 1 eq) and TEA (2.27 g, 22.47 mmol, 3.13 mL, 2 eq) at 0° C. The mixture was stirred at 25° C. for 12 hours. LCMS (5-95AB/1.5 min, RT=0.448 min, 267.1[M+H]+, ESI pos) showed major peak with desired ms. The reaction mixture was filtered and concentrated under reduced pressure to afford 2-((tert-butoxycarbonyl)amino)ethyl nicotinate (2, 2.7 g, 10.14 mmol, 90.25% yield) as a yellow solid. To this solution of 2-((tert-butoxycarbonyl)amino)ethyl nicotinate (2, 2.7 g, 10.14 mmol, 1 eq) in dioxane (12 mL) was added HCl/dioxane (4 M, 12 mL, 4.73 eq) at 0° C. The mixture was stirred at 25° C. for 2 hours. LCMS (0-60CD/1.5 min, RT=0.436 min, 167.2[M+H]+, ESI pos) showed major peak with desired ms. The reaction mixture was filtered and concentrated under reduced pressure to give a residue. The residue was purified by reversed-phase HPLC (column: Phenomenex luna C18 (250×70 mm, 10 um); mobile phase: [water(HCl)-ACN]; gradient: 0%-20% B over 12 min) to afford 2-aminoethyl nicotinate (N-28-IN3, 2.8 g, 16.12 mmol, 95.7% purity) as a white solid. LCMS: RT=0.388 min, m/z=167.2 (M+H)⁺. ¹H NMR (400 MHz, MeOD) δ 9.46 (s, 1H), 9.07-8.95 (m, 2H), 8.07 (br s, 1H), 4.73-4.63 (m, 2H), 3.49-3.40 (m, 2H)

### 2-Hydroxyethyl Nicotinate

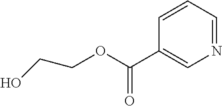 [SMILES: O=C(OCCO)c1cccnc1]

To a solution of nicotinic acid (5 g, 40.61 mmol, 3.40 mL, 1 eq), ethane-1,2-diol (2, 5.04 g, 81.23 mmol, 4.54 mL, 2 eq) in DCM (10 mL) were added EDCI (11.68 g, 60.92 mmol, 1.5 eq), DMAP (992.35 mg, 8.12 mmol, 0.2 eq), TEA (4.11 g, 40.61 mmol, 5.65 mL, 1 eq). The mixture was stirred at 25° C. for 16 hours. LCMS (0-60AB/1 min, RT=0.327 min, 168.1 [M+H]⁺, ESI pos) showed the major peak with desired product was detected. The reaction mixture was filtered and concentrated under reduced pressure to give a residue. The residue was purified by column chromatography (SiO2, Petroleum ether:Ethyl acetate=1:0 to 0:1). Then the residue was purified by prep-HPLC (column: Phenomenex luna C18 250×50 mm×10 um; mobile phase: [water(HCl)-ACN]; B %: 0%-25%, 13 min) to afford 2-hydroxyethyl nicotinate (N-20-INT3, 3.8 g, 22.41 mmol, 55.17% yield, 98.56% purity) as white solid. LCMS: t_{R}=0.326 min, m/z=168.1 (M+H)⁺; ¹H NMR (400 MHz, MeOD) 9.32 (d, J=0.9 Hz, 1H), 8.97-8.89 (m, 1H), 8.87-8.77 (m, 1H), 7.97-7.87 (m, 1H), 4.52-4.42 (m, 2H), 3.96-3.85 (m, 2H).

### 2-(2-(2-((2,6-Dichlorophenyl)Amino)Phenyl)Acetoxy)Ethyl Nicotinate

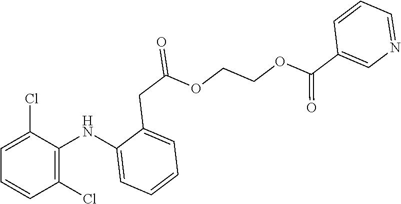 [SMILES: O=C(Cc1ccccc1Nc1c(Cl)cccc1Cl)OCCOC(=O)c1cccnc1]

To a solution of Diclofenac (1 eq), 2-hydroxyethyl nicotinate (2 eq) in DCM (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by column chromatography (SiO2, Petroleum ether:Ethyl acetate=1:0 to 0:1). Then the residue was purified by prep-HPLC (column: Phenomenex luna C18 250×50 mm×10 um; mobile phase: [water(HCl)-ACN]; B %: 0%-25%, 13 min) to 2-(2-(2-((2,6-dichlorophenyl)amino)phenyl)acetoxy)ethyl nicotinate (81% yield, 98% purity) as white solid. LCMS: t_{R}=0.82 min, m/z=467 (M+23)⁺.

### N-(2-(2-(2-((2,6-Dichlorophenyl)Amino)Phenyl)Acetamido)Ethyl)Nicotinamide

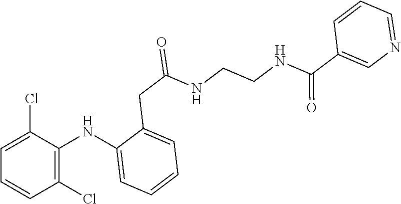 [SMILES: O=C(Cc1ccccc1Nc1c(Cl)cccc1Cl)NCCNC(=O)c1cccnc1]

To a solution of Diclofenac (1 eq), N-(2-aminoethyl)nicotinamide (2 eq) in DCM (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by column chromatography (SiO2, Petroleum ether:Ethyl acetate=1:0 to 0:1). Then the residue was purified by prep-HPLC (column: Phenomenex luna C18 250×50 mm×10 um; mobile phase: [water(HCl)-ACN]; B %: 0%-25%, 13 min) to get N-(2-(2-(2-((2,6-dichlorophenyl)amino)phenyl)acetamido)ethyl)nicotinamide (88% yield, 99% purity) as white solid. LCMS: t_{R}=8.1 min, m/z=444 (M+H)⁺.

### N-(2-(2-(4-isobutylphenyl)propanamido)ethyl)nicotinamide

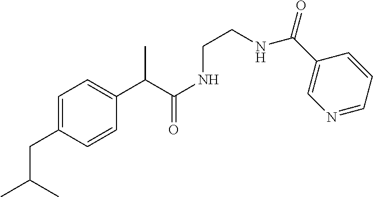 [SMILES: CC(C)Cc1ccc(C(C)C(=O)NCCNC(=O)c2cccnc2)cc1]

To a solution of Ibuprofen (1 eq), N-(2-aminoethyl)nicotinamide (2 eq) in DMF (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by prep-HPLC [water(HCl)-ACN]; B %: 0%-25%, 13 min) to get N-(2-(2-(4-isobutylphenyl)propanamido)ethyl)nicotinamide (82% yield, 98% purity) as white solid. LCMS: t_{R}=9.8 min, m/z=354 (M+H)⁺.

### 2-Methyl-2-(4-Methylpent-3-En-1-Yl)-7-Pentyl-2h-Chromen-5-Yl Nicotinate

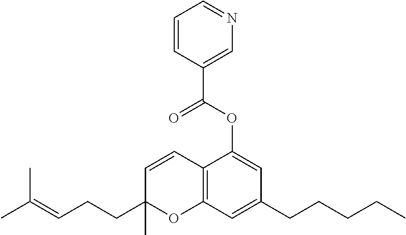 [SMILES: CCCCCc1cc(OC(=O)c2cccnc2)c2c(c1)OC(C)(CCC=C(C)C)C=C2]

To a solution of Cannabichromene (1 eq), nicotinic acid (2 eq) in DCM (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by column chromatography (SiO2, Petroleum ether:Ethyl acetate=1:0 to 0:1). Then the residue was purified by prep-HPLC (column: Phenomenex luna C18 250×50 mm×10 um; mobile phase: [water(HCl)-ACN]; B %: 0%-25%, 13 min) to get 2-methyl-2-(4-methylpent-3-en-1-yl)-7-pentyl-2H-chromen-5-yl nicotinate (84% yield, 99% purity) as white solid. LCMS: t_{R}=12.7 min, m/z=420 (M+H)⁺.

### N-(2-((2E,4E,6E,8E)-3,7-dimethyl-9-(2,6,6-trimethylcyclohex-1-en-1-yl)nona-2,4,6,8-tetraenamido)ethyl)nicotinamide

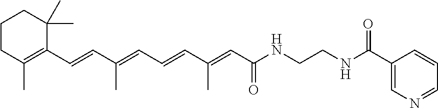 [SMILES: CC1=C(/C=C/C(C)=C/C=C/C(C)=C/C(=O)NCCNC(=O)c2cccnc2)C(C)(C)CCC1]

To a solution of retinoic acid (1 eq), N-(2-aminoethyl)nicotinamide (2 eq) in DMF (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by prep-HPLC [water(HCl)-ACN]; B %: 0%-25%, 13 min) to get N-(2-((2E,4E,6E,8E)-3,7-dimethyl-9-(2,6,6-trimethylcyclohex-1-en-1-yl)nona-2,4,6,8-tetraenamido)ethyl)nicotinamide (86% yield, 99% purity) as white solid. LCMS: t_{R}=7.1 min, m/z=450 (M+H)⁺.

### 2-(((2E,4E,6E,8E)-3,7-dimethyl-9-(2,6,6-trimethylcyclohex-1-en-1-yl)nona-2,4,6,8-tetraenoyl)oxy)ethyl nicotinate

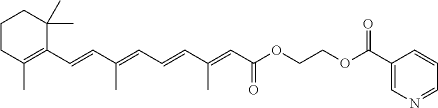 [SMILES: CC1=C(/C=C/C(C)=C/C=C/C(C)=C/C(=O)OCCOC(=O)c2cccnc2)C(C)(C)CCC1]

To a solution of retinoic acid (1 eq), 2-hydroxyethyl nicotinate (2 eq) in DMF (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by prep-HPLC [water(HCl)-ACN]; B %: 0%-25%, 13 min) to get N-(2-((2E,4E,6E,8E)-3,7-dimethyl-9-(2,6,6-trimethylcyclohex-1-en-1-yl)nona-2,4,6,8-tetraenamido)ethyl)nicotinamide (86% yield, 99% purity) as white solid. LCMS: t_{R}=12.7 min, m/z=449 (M+H)⁺.

### 2-(3,4-bis(nicotinoyloxy)phenyl)-3-hydroxy-4-oxo-4H-chromene-5,7-diyl dinicotinate

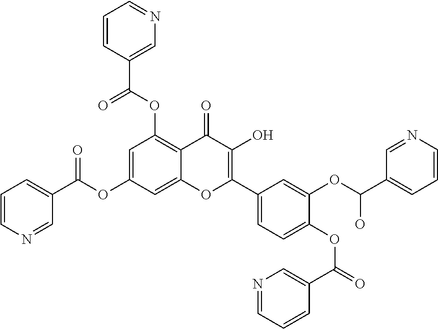 [SMILES: O=C(Oc1cc(OC(=O)c2cccnc2)c2c(=O)c(O)c(-c3ccc(OC(=O)c4cccnc4)c(OC(O)c4cccnc4)c3)oc2c1)c1cccnc1]

To a solution of Gallocatechol (1 eq), nicotinic acid (5 eq) in DMF (10 mL) were added EDCI (6 eq), DMAP (0.2 eq), TEA (10 eq). The mixture was stirred at 25° C. for 16 hours. LCMS showed the major peak with desired product was detected. The reaction mixture was filtered and concentrated under reduced pressure to give a residue. The residue was purified by prep-HPLC [water(HCl)-ACN]; B %: 0%-25%, 13 min) to get 2-(3,4-bis(nicotinoyloxy)phenyl)-3-hydroxy-4-oxo-4H-chromene-5,7-diyl dinicotinate (76% yield, 96% purity) as white solid. LCMS: t_{R}=0.5 min, m/z=723 (M+H)⁺.

### 2-((2-(4-Isobutylphenyl)Propanoyl)Oxy)Ethyl Nicotinate

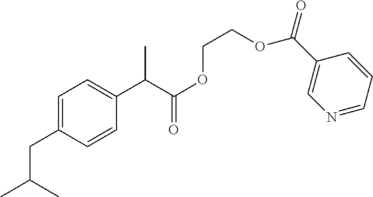 [SMILES: CC(C)Cc1ccc(C(C)C(=O)OCCOC(=O)c2cccnc2)cc1]

To a solution of Ibuprofen (1 eq), 2-hydroxyethyl nicotinate (2 eq) in DMF (10 mL) were added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a residue. The residue was purified by prep-HPLC [water(HCl)-ACN]; B %: 0%-25%, 13 min) to get 2-((2-(4-isobutylphenyl)propanoyl)oxy)ethyl nicotinate (86% yield, 99% purity) as white solid. LCMS: t_{R}=9.5 min, m/z=356 (M+H)⁺.

### (Nicotinamidomethyl)Triphenylphosphonium

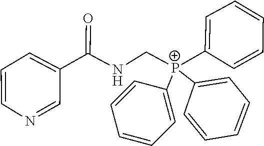 [SMILES: O=C(NC[P+](c1ccccc1)(c1ccccc1)c1ccccc1)c1cccnc1]

To a solution of N-(hydroxymethyl)nicotinamide (1, 200 mg, 1.31 mmol, 2 eq) in toluene (5 mL) and HCl (12 M, 54.77 μL, 1 eq) was added PPh₃ (189.62 mg, 722.97 μmol, 1.1 eq) in one portion. The mixture was stirred at 110° C. for 2 hours. LCMS (5-95AB/1 min, RT=0.402 min, 397.2[M]⁺, ESI pos) showed the major peak with desired product. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 10%-40%, 8 min) to afford (nicotinamidomethyl)triphenylphosphonium (N-22, 13.9 mg, 33.48 μmol, 5.09% yield, 95.72% purity) as a white solid. LCMS: t_{R}=0.402 min, m/z=397.2 (M)^{+ 1}H NMR (400 MHz, MeOD) 9.11 (d, J=1.9 Hz, 1H), 9.00 (d, J=5.6 Hz, 1H), 8.82-8.76 (m, 1H), 8.20-8.12 (m, 1H), 7.97-7.86 (m, 9H), 7.81-7.73 (m, 6H), 5.55 (d, J=4.6 Hz, 2H).

### ((Nicotinoyloxy)Methyl)Triphenylphosphonium

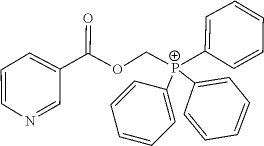 [SMILES: O=C(OC[P+](c1ccccc1)(c1ccccc1)c1ccccc1)c1cccnc1]

To a solution of nicotinoyl chloride (1, 200 mg, 1.41 mmol, 487.46 uL, 2 eq), (hydroxymethyl)triphenylphosphonium chloride (2, 232.26 mg, 706.44 umol, 1 eq) in MeCN (10 mL) was added Py (279.40 mg, 3.53 mmol, 285.10 uL, 5 eq). The mixture was stirred at 50° C. for 16 hours. LCMS (5-95AB/1 min, RT=0.427 min, 398.2[M]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 18%-48%, 8 min) to afford ((nicotinoyloxy)methyl)triphenylphosphonium (N-23, 38.10 mg, 89.54 μmol, 12.67% yield, 93.63% purity) as a colorless oil. LCMS: t_{R}=0.441 min, m/z=398.2 (M)^{+ 1}H NMR (400 MHz, MeOD) 9.24 (s, 1H), 9.12 (d, J=5.6 Hz, 1H), 8.95-8.88 (m, 1H), 8.25-8.19 (m, 1H), 8.02-7.91 (m, 9H), 7.87-7.81 (m, 6H), 6.49 (d, J=4.6 Hz, 2H).

### (2-(Nicotinamido)Benzyl)Triphenylphosphonium

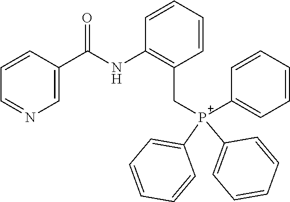 [SMILES: O=C(Nc1ccccc1C[P+](c1ccccc1)(c1ccccc1)c1ccccc1)c1cccnc1]

1 eq of Nicotinoyl chloride hydrochloride was dissolved in 0.1 M DMF and treated with 1 eq of (2-Aminobenzyl)triphenylphosphonium bromide at 0° C. The reaction was stirred for 24 hours. The crude product was purified by reversed-phase HPLC (MeCN/H2O, 0.1% TFA) to afford (2-(nicotinamido)benzyl)triphenylphosphonium in 94% yield as white solid. LCMS: t_{R}=5.0 min, m/z=473 (M+).

### (6-(Nicotinamido-2,4,5,6-D4)Hexyl)Triphenylphosphonium

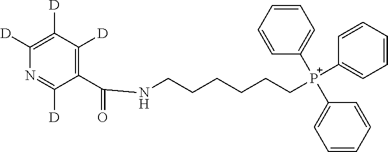 [SMILES: [2H]c1nc([2H])c(C(=O)NCCCCCC[P+](c2ccccc2)(c2ccccc2)c2ccccc2)c([2H])c1[2H]]

To a solution of 2,4,5,6-tetradeuteriopyridine-3-carboxylic acid (4, 200 mg, 1.57 mmol, 1 eq) in DCM (5 mL) was added DIEA (609.94 mg, 4.72 mmol, 822.02 uL, 3 eq) and HATU (717.79 mg, 1.89 mmol, 1.2 eq), the mixture was stirred at 25° C. for 30 min. Then (6-aminohexyl)triphenylphosphonium (3, 570.21 mg, 1.57 mmol, 1 eq) in DCM (1 mL) was added. The mixture was stirred at 25° C. for 1 hours. LCMS (5-95AB/1.5 min, RT=0.576 min, 471.3, [M+H]⁺, ESI pos) showed the major peak with desired product was detected. The reaction mixture was diluted with 1M HCl (100 mL) and extracted with DCM (100 mL), dried over Na₂SO₄, filtered and concentrated under reduced pressure to give a residue. The crude product was purified by prep-HPLC (column: Phenomenex luna C18 150×40 mm×15 um; mobile phase: [water(0.05% HCl)-ACN]; B %: 15%-45%, 10 min) to afford triphenyl-[6-[(2,4,5,6-tetradeuteriopyridine-3-carbonyl)amino]hexyl]phosphonium (420 mg, 890.61 umol, 56.62% yield) as a yellow solid. LCMS: t_{R}=0.433 min, m/z=471.2 (M+H)⁺.

### (2-(Nicotinamido)ethyl)triphenylphosphonium chloride

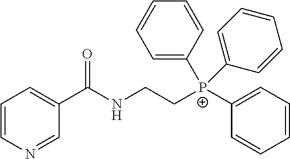 [SMILES: O=C(NCC[P+](c1ccccc1)(c1ccccc1)c1ccccc1)c1cccnc1]

To a solution of tert-butyl (2-bromoethyl)carbamate (1 g, 4.46 mmol, 1 eq) in MeCN (10 mL) was added PPh₃ (1.23 g, 4.69 mmol, 1.05 eq). The mixture was stirred at 85° C. for 16 hours. LCMS (5-95AB/1 min, RT=0.353 min, 306.3[M]⁺, ESI pos) showed the major peak with desired product was detected. The reaction mixture was filtered and concentrated under reduced pressure to give a residue. The crude product was purified by reversed-phase HPLC (0.1% FA condition, 100% water) to afford (2-aminoethyl)triphenylphosphonium (300 mg, 705.05 umol, 15.80% yield, 72% purity) as a yellow oil. To this solution of nicotinic acid (40.18 mg, 326.41 umol, 27.34 uL, 1 eq) in DCM (2 mL) was added HATU (148.93 mg, 391.70 umol, 1.2 eq), DIEA (126.56 mg, 979.24 umol, 170.57 uL, 3 eq). The mixture was stirred at 25° C. for 30 min. Then (2-aminoethyl)triphenylphosphonium (3, 100 mg, 326.41 umol, 1 eq) was added. The mixture was stirred at 25° C. for 2 hours. LCMS (5-95AB/1 min, RT=0.404 min, 411.2[M]⁺, ESI pos) showed the major peak with desired product. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 10%-40%, 8 min) to afford (2-(nicotinamido)ethyl)triphenylphosphonium chloride (22.8 mg, 54.86 μmol, 16.81% yield, 99% purity) as a yellow solid. LCMS: t_{R}=0397 min, m/z=411.3 (M)^{+ 1}H NMR (400 MHz, MeOD) 9.16 (br s, 1H), 8.98 (br d, J=4.8 Hz, 1H), 8.86-8.78 (m, 1H), 8.17-8.08 (m, 1H), 7.96-7.86 (m, 10H), 7.82-7.78 (m, 5H), 3.88-3.79 (m, 4H).

### (2-(Nicotinoyloxy)Ethyl)Triphenylphosphonium Trifluoroacetate

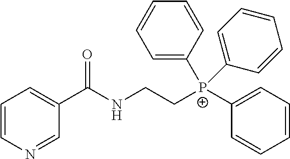 [SMILES: O=C(NCC[P+](c1ccccc1)(c1ccccc1)c1ccccc1)c1cccnc1]

1 eq of Nicotinoyl chloride hydrochloride was dissolved in 0.1 M DMF and treated with 1 eq of (2-Hydroxyethyl)triphenylphosphonium bromide at 0° C. The reaction was stirred for 24 hours. The crude product was purified by reversed-phase HPLC (MeCN/H2O, 0.1% TFA) to afford (2-(nicotinoyloxy)ethyl)triphenylphosphonium trifluoroacetate in 56% yield as white solid. LCMS: t_{R}=11.344 min, m/z=412 (M+).

### (5-((2-(Nicotinamido)Ethyl)Amino)-5-Oxopentyl)Triphenylphosphonium

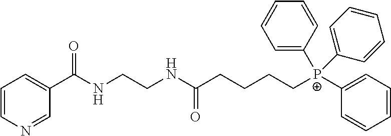 [SMILES: O=C(CCCC[P+](c1ccccc1)(c1ccccc1)c1ccccc1)NCCNC(=O)c1cccnc1]

To a solution of N-(2-aminoethyl)nicotinamide (1, 200 mg, 1.21 mmol, 1 eq) in MeCN (5 mL) was added EDCI (696.29 mg, 3.63 mmol, 3 eq), HOBt (490.79 mg, 3.63 mmol, 3 eq) and (4-carboxybutyl)triphenylphosphonium (2, 439.98 mg, 1.21 mmol, 1 eq). The mixture was stirred at 25° C. for 12 hours. LCMS (5-95AB/1 min, RT=0.408 min, 510.2[M+H]⁺, ESI pos) showed the major peak with desired MS. The reaction mixture was filtered and concentrated under reduced pressure to give a residue. The residue were purified by reversed-phase HPLC (column: Welch Xtimate C18 150×25 mm×5 um; mobile phase: [water(HCl)-ACN]; gradient:10%-40% B over 8 min) to afford (5-((2-(nicotinamido)ethyl)amino)-5-oxopentyl)triphenylphosphonium (N-40, 65.7 mg, 126.36 μmol, 10.44% yield, 98.2% purity) as a yellow solid. LCMS: RT=0.408 min, m/z=510.2 (M+H)^{+ 1}H NMR (400 MHz, CD₃OD) δ 9.26 (s, 1H), 9.01-8.99 (m, 2H), 8.22-8.20 (m, 1H), 7.89-7.81 (m, 3H), 7.77-7.75 (m, 12H), 3.51-3.31 (m, 6H), 2.29-2.25 (m, 2H), 1.86-1.83 (m, 2H), 1.71-1.69 (m, 2H)

### (5-((2-(Nicotinoyloxy)Ethyl)Amino)-5-Oxopentyl)Triphenylphosphonium

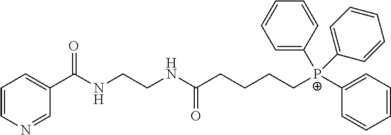 [SMILES: O=C(CCCC[P+](c1ccccc1)(c1ccccc1)c1ccccc1)NCCNC(=O)c1cccnc1]

To a solution of 2-aminoethyl nicotinate (200 mg, 1.20 mmol, 1 eq) in DCM (5 mL) were added EDCI (346.08 mg, 1.81 mmol, 1.5 eq), HOBt (243.94 mg, 1.81 mmol, 1.5 eq) and (4-carboxybutyl)triphenylphosphonium (437.38 mg, 1.20 mmol, 1 eq). The mixture was stirred at 25° C. for 12 hours. LCMS (5-95AB/1 min, RT=0.427 min, 511.4[M+H]⁺, ESI pos) showed the major peak with desired MS. The reaction mixture was filtered and concentrated under reduced pressure to give a residue. The residue was purified by reversed-phase HPLC (column: Welch Xtimate C18 150×25 mm×5 um; mobile phase: [water(HCl)-ACN]; gradient:15%-45% B over 8 min) to afford (5-((2-(nicotinoyloxy)ethyl)amino)-5-oxopentyl)triphenylphosphonium (123.1 mg, 217.41 μmol, 18.06% yield, 90.349% purity) as a yellow solid. LCMS: RT=0.427 min, m/z=511.4 (M+H)^{+ 1}H NMR (400 MHz, MeOD) δ 9.17 (s, 1H), 8.86-8.85 (m, 1H), 8.66-8.65 (m, 1H), 7.90-7.88 (m, 3H), 7.77-7.76 (m, 1H), 7.75-7.73 (m, 12H), 4.41-4.38 (m, 2H), 3.57-3.54 (m, 2H), 3.36-3.31 (m, 2H), 2.29-2.25 (m, 2H), 1.85-1.79 (m, 2H), 1.70-1.66 (m, 2H).

### (2-(Nicotinamido)Ethyl)Triphenylphosphonium Chloride

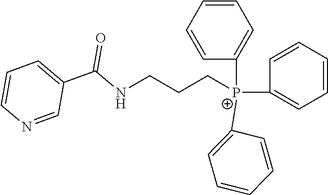 [SMILES: O=C(NCCC[P+](c1ccccc1)(c1ccccc1)c1ccccc1)c1cccnc1]

To a solution of nicotinic acid (4, 50 mg, 406.14 umol, 34.01 uL, 1 eq) in DCM (2 mL) was added HATU (185.31 mg, 487.37 umol, 1.2 eq), DIEA (209.96 mg, 1.62 mmol, 282.96 uL, 4 eq). The mixture was stirred at 25° C. for 30 min. Then (3-aminopropyl)triphenylphosphonium (3, 144.93 mg, 406.14 umol, 1 eq, HCl) was added. The mixture was stirred at 25° C. for 2 hours. LCMS (5-95AB/1 min, RT=0.401 min, 425.1[M]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 12%-42%, 8 min) to afford (2-(nicotinamido)ethyl)triphenylphosphonium chloride (N-26, 63.8 mg, 148.84 μmol, 36.65% yield, 99.26% purity) as a white solid. LCMS: t_{R}=0.401 min, m/z=425.3 (M)^{+ 1}H NMR (400 MHz, MeOD) 9.21 (s, 1H), 8.99-8.89 (m, 2H), 8.17-8.09 (m, 1H), 7.93-7.88 (m, 3H), 7.87-7.75 (m, 12H), 3.65 (t, J=6.8 Hz, 2H), 3.56-3.46 (m, 2H), 2.11-1.99 (m, 2H).

### (3-(Nicotinoyloxy)Propyl)Triphenylphosphonium Chloride

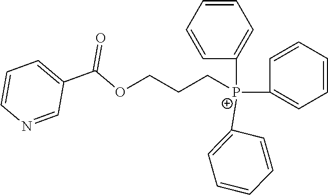 [SMILES: O=C(OCCC[P+](c1ccccc1)(c1ccccc1)c1ccccc1)c1cccnc1]

To a solution of nicotinic acid (1 g, 8.12 mmol, 680.27 uL, 1 eq), 3-bromopropan-1-ol (1.35 g, 9.75 mmol, 879.74 uL, 1.2 eq) in DCM (10 mL) were added EDCI (2.34 g, 12.18 mmol, 1.5 eq), DMAP (198.47 mg, 1.62 mmol, 0.2 eq), TEA (821.95 mg, 8.12 mmol, 1.13 mL, 1 eq). The mixture was stirred at 25° C. for 16 hours. LCMS (5-95AB/1 min, RT=0.442 min, 244.0[M+H]⁺, ESI pos) showed major peak with desired MS. The reaction mixture was filtered and concentrated under reduced pressure to give a residue. The residue was purified by column chromatography (SiO2, Petroleum ether:Ethyl acetate=1:0 to 3:1) to afford 3-bromopropyl nicotinate (1.1 g, 4.51 mmol, 55.48% yield) as white solid. To this solution of 3-bromopropyl nicotinate (500 mg, 2.05 mmol, 1 eq) in MeCN (10 mL) was added PPh₃ (564.15 mg, 2.15 mmol, 1.05 eq). The mixture was stirred at 80° C. for 16 hours. LCMS (5-95AB/1 min, RT=0.428 min, 426.1 [M+H]⁺, ESI pos) showed the major peak with desired product. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 18%-48%, 8 min) to afford (3-(nicotinoyloxy)propyl)triphenylphosphonium (116 mg, 259.46 umol, 12.67% yield, 95.39% purity) as yellow oil. LCMS: t_{R}=0.447 min, m/z=426.1 (M+H)^{+ 1}H NMR (400 MHz, MeOD) 9.48-9.43 (m, 1H), 9.19-9.07 (m, 2H), 8.29-8.22 (m, 1H), 7.94-7.77 (m, 15H), 4.62 (t, J=5.9 Hz, 2H), 3.74-3.62 (m, 2H), 2.29-2.17 (m, 2H).

### (6-((2-(Nicotinamido)Ethyl)Amino)-6-Oxohexyl)Triphenylphosphonium

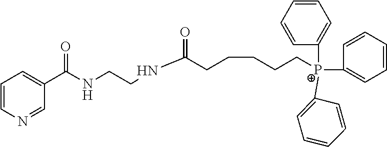 [SMILES: O=C(CCCCC[P+](c1ccccc1)(c1ccccc1)c1ccccc1)NCCNC(=O)c1cccnc1]

To a solution of N-(2-aminoethyl)nicotinamide (200 mg, 716.29 umol, 1 eq, TFA), (5-carboxypentyl)triphenylphosphonium (327.59 mg, 716.29 umol, 1 eq) in MeCN (5 mL) was added EDCI (411.94 mg, 2.15 mmol, 3 eq) and HOBt (290.36 mg, 2.15 mmol, 3 eq). The mixture was stirred at 25° C. for 1 hours. LCMS (5-95AB/1 min, RT=0.409 min, 524.3[M]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 15%-45%, 8 min) to afford (6-((2-(nicotinamido)ethyl)amino)-6-oxohexyl)triphenylphosphonium (111.5 mg, 208.14 μmol, 29.06% yield, 97.93% purity) as a colorless oil. LCMS: t_{R}=0.400 min, m/z=524.4 (M)^{+ 1}H NMR (400 MHz, MeOD) 9.28 (s, 1H), 9.06-8.99 (m, 2H), 8.26-8.20 (m, 1H), 7.94-7.76 (m, 15H), 3.59-3.40 (m, 6H), 2.21 (t, J=7.1 Hz, 2H), 1.77-1.54 (m, 6H).

### (6-((2-(Nicotinoyloxy)Ethyl)Amino)-6-Oxohexyl)Triphenylphosphonium

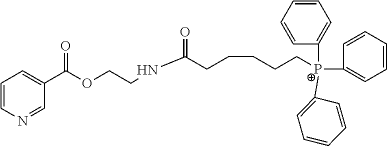 [SMILES: O=C(CCCCC[P+](c1ccccc1)(c1ccccc1)c1ccccc1)NCCOC(=O)c1cccnc1]

To a solution of 2-aminoethyl nicotinate (200 mg, 1.20 mmol, 1 eq) in DCM (10 mL) were added EDCI (346.08 mg, 1.81 mmol, 1.5 eq), (5-carboxypentyl)triphenylphosphonium (454.26 mg, 1.20 mmol, 1 eq) and HOBt (243.94 mg, 1.81 mmol, 1.5 eq). The mixture was stirred at 25° C. for 12 hours. LCMS (5-95AB/1.5 min, RT=0.412 min, 525.2[M+H]⁺, ESI pos) showed the major peak with desired product. The reaction mixture was filtered and concentrated under reduced pressure to give a residue. The crude product was purified by reversed-phase HPLC (column: Welch Xtimate C18 150×25 mm×5 um; mobile phase: [water(HCl)-ACN]; gradient:18%-48% B over 8 min) to afford (6-((2-(nicotinoyloxy)ethyl)amino)-6-oxohexyl)triphenylphosphonium (75.4 mg, 129.45 μmol, 10.76% yield, 90.23% purity) was obtained as a yellow solid. LCMS: t_{R}=0.412 min, m/z=525.2 (M+H)^{+ 1}H NMR (400 MHz, MeOD) δ 9.41 (s, 1H), 9.16-8.99 (m, 2H), 8.22 (dd, J=6.3, 8.1 Hz, 1H), 7.95-7.85 (m, 3H), 7.83-7.74 (m, 12H), 4.48 (t, J=5.4 Hz, 2H), 3.63-3.54 (m, 2H), 3.48-3.36 (m, 2H), 2.24-2.15 (m, 2H), 1.73-1.53 (m, 6H).

### (6-(Nicotinamido)Hexyl)Triphenylphosphonium Chloride

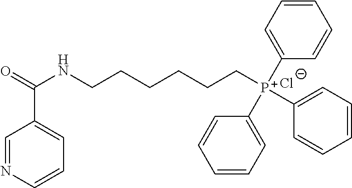 [SMILES: O=C(NCCCCCC[P+](c1ccccc1)(c1ccccc1)c1ccccc1)c1cccnc1 ; [Cl-]]

To a solution of nicotinic acid (2, 249.64 mg, 2.03 mmol, 169.82 uL, 1.05 eq) in THF (8 mL) was added DIEA (748.79 mg, 5.79 mmol, 1.01 mL, 3 eq) and HATU (881.17 mg, 2.32 mmol, 1.2 eq). Then (6-aminohexyl)triphenylphosphonium chloride (700 mg, 1.93 mmol, 1 eq) was added. The mixture was stirred at 20° C. for 16 hours. LCMS showed Reactant 1 was consumed completely and desired mass was detected. The reaction mixture was filtered to give a residue. The crude product was purified by reversed-phase HPLC (0.1% FA condition) to afford (6-(nicotinamido)hexyl)triphenylphosphonium chloride (100 mg, 213.88 umol, 42.66% yield) as a yellow oil. ¹H NMR (400 MHz, CD₃OD) δ 8.93 (d, J=1.7 Hz, 1H), 8.71-8.66 (m, 1H), 8.23-8.18 (m, 1H), 7.92-7.86 (m, 3H), 7.82-7.74 (m, 12H), 7.57-7.51 (m, 1H), 3.44-3.35 (m, 4H), 1.72-1.58 (m, 6H), 1.47-1.39 (m, 2H).

### (6-(Nicotinoyloxy)Hexyl)Triphenylphosphonium

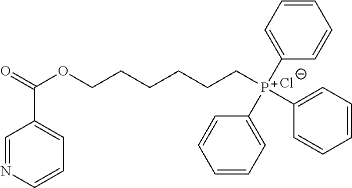 [SMILES: O=C(OCCCCCC[P+](c1ccccc1)(c1ccccc1)c1ccccc1)c1cccnc1 ; [Cl-]]

To a solution of nicotinic acid (173.25 mg, 1.41 mmol, 117.86 uL, 1.2 eq), K₂CO₃ (324.16 mg, 2.35 mmol, 2 eq) in MeCN (5 mL) was added (6-bromohexyl)triphenylphosphonium (500 mg, 1.17 mmol, 1 eq). The mixture was stirred at 25° C. for 16 hours. LCMS (5-95AB/1 min, RT=0.458 min, 468.4, [M+H]⁺, ESI pos) showed the major peak with desired MS. The reaction mixture was filtered, and concentrated under reduced pressure to give a residue. The residue was purified by prep-HPLC (column: Phenomenex luna C18 150×25 mm×10 um; mobile phase: [water(FA)-ACN]; B %: 22%-52%, 7 min) to afford (6-(nicotinoyloxy)hexyl)triphenylphosphonium (43.6 mg, 89.53 umol, 7.63% yield, 96.21% purity) as yellow oil. LCMS: t_{R}=0.468 min, m/z=468.3 (M+H)^{+ 1}H NMR (400 MHz, MeOD) δ 9.09 (d, J=1.6 Hz, 1H), 8.78-8.72 (m, 1H), 8.54 (s, 1H), 8.41-8.33 (m, 1H), 7.92-7.86 (m, 3H), 7.83-7.74 (m, 11H), 7.59-7.55 (m, 1H), 4.35 (t, J=6.6 Hz, 2H), 3.45-3.38 (m, 2H), 1.82-1.61 (m, 6H), 1.55-1.47 (m, 2H).

### (9-((2-(Nicotinamido)Ethyl)Amino)Nonyl)Triphenylphosphonium

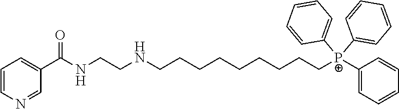 [SMILES: O=C(NCCNCCCCCCCCC[P+](c1ccccc1)(c1ccccc1)c1ccccc1)c1cccnc1]

A mixture of (9-bromononyl)triphenylphosphonium (HBr salt, 100 mg, 213.48 μmol, 1 eq), N-(2-aminoethyl)nicotinamide (298.03 mg, 1.07 mmol, 5 eq, TFA) in MeOH (2 mL), and then the mixture was stirred at 80° C. for 16 hours. LCMS (5-95AB/1 min, RT=0.395 min, 276.8[½M+H]⁺, ESI pos) showed the major peak with desired product was detected. The reaction mixture was filtered and concentrated under reduced pressure to give a residue. The crude product were purified by reversed-phase HPLC (column: Welch Xtimate C18 150×25 mm×5 um; mobile phase: [water(HCl)-ACN]; gradient:5%-35% B over 10 min) to afford (9-((2-(nicotinamido)ethyl)amino)nonyl)triphenylphosphonium (, HCl salt, 23.7 mg, 42.49 μmol, 19.90% yield, 99.09% purity) as a yellow gum. ¹H NMR (400 MHz, CD₃OD) 9.60 (s, 1H), 9.16 (d, J=6.3 Hz, 1H), 9.02 (d, J=8.1 Hz, 1H), 8.29-8.21 (m, 1H), 7.94-7.89 (m, 3H), 7.85-7.75 (m, 12H), 4.75-4.68 (m, 2H), 3.77 (t, J=5.9 Hz, 2H), 3.46-3.39 (m, 2H), 3.26 (t, J=5.9 Hz, 2H), 2.13-2.04 (m, 2H), 1.73-1.54 (m, 4H), 1.46-1.32 (m, 8H).

### (9-(2-(Nicotinoyloxy)Ethoxy)Nonyl)Triphenylphosphonium

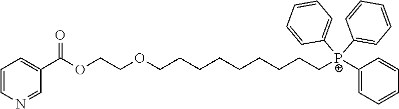 [SMILES: O=C(OCCOCCCCCCCCC[P+](c1ccccc1)(c1ccccc1)c1ccccc1)c1cccnc1]

To a solution of 2-((9-bromononyl)oxy)ethyl nicotinate (90 mg, 241.74 μmol, 1 eq) in MeCN (3 mL) was added PPh₃ (66.58 mg, 253.83 μmol, 1.05 eq). The mixture was stirred at 85° C. for 1 hour. LCMS (5-95AB/1.5 min, RT=0.518 min, 554.3[M+H]⁺, ESI pos) showed the major peak with desired product was detected. The reaction mixture was filtered and concentrated under reduced pressure to give a residue. The crude product was purified by reversed-phase HPLC (column: Welch Xtimate C18 150×25 mm×5 um; mobile phase: [water(HCl)-ACN]; gradient:32%-62% B over 9 min) to afford (9-(2-(nicotinoyloxy)ethoxy) nonyl)triphenylphosphonium (HCl salt, 12.8 mg, 22.37 μmol, 24.82% yield, 96.94% purity) as a yellow solid. QC of N-42 LCMS: RT=0.537 min, m/z=554.4 (M+H)^{+ 1}H NMR (400 MHz, CD₃OD) δ 9.39 (br s, 1H), 9.14-9.07 (m, 2H), 8.24 (br t, J=6.5 Hz, 1H), 7.93-7.89 (m, 3H), 7.85-7.77 (m, 12H), 4.65-4.57 (m, 2H), 3.88-3.79 (m, 2H), 3.54 (br t, J=6.5 Hz, 2H), 3.40 (br d, J=9.1 Hz, 2H), 1.74-1.56 (m, 6H), 1.32 (br d, J=2.9 Hz, 8H).

### (3-((2-(Nicotinamido)Ethyl)Amino)Propyl)Triphenyl Phosphonium Chloride

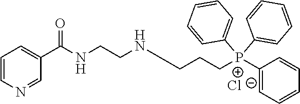 [SMILES: O=C(NCCNCCCC[P+](c1ccccc1)(c1ccccc1)c1ccccc1)c1cccnc1 ; Cl]

A mixture of (3-bromopropyl)triphenylphosphonium (100 mg, 260.23 umol, 1 eq), N-(2-aminoethyl)nicotinamide (363.31 mg, 1.30 mmol, 5 eq, TFA) in MeOH (2 mL) was degassed and purged with N₂ for 3 times, and then the mixture was stirred at 80° C. for 32 hours. LCMS (5-95AB/1.5 min, RT=0.301 min, 468.2[M+H]⁺, ESI pos) showed the major peak with desired product was detected. The reaction mixture concentrated under reduced pressure. The crude product was purified by prep-HPLC (column: YMC Triart C18 150×25 mm×5 um; mobile phase: [water(HCl)-ACN]; B %: 6%-36%, 10 min) to afford (3-((2 (nicotinamido)ethyl)amino)propyl)triphenylphosphonium chloride (25.21 mg, 53.55 umol, 20.58% yield, 99.53% purity) as white solid. LCMS: t_{R}=0.309 min, m/z=468.2 (M+H)^{+ 1}H NMR (400 MHz, MeOD) δ 9.72 (s, 1H), 9.19 (d, J=6.1 Hz, 1H), 9.00 (d, J=8.2 Hz, 1H), 8.27-8.18 (m, 1H), 7.94-7.75 (m, 15H), 4.98 (t, J=7.5 Hz, 2H), 3.78-3.67 (m, 4H), 3.24 (t, J=5.7 Hz, 2H), 2.56-2.44 (m, 2H).

### (3-(2-(Nicotinoyloxy)Ethoxy)Propyl)Triphenylphosphonium

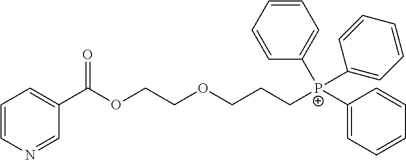 [SMILES: O=C(OCCOCCC[P+](c1ccccc1)(c1ccccc1)c1ccccc1)c1cccnc1]

To a solution of oxirane (950.83 mg, 21.58 mmol, 1.08 mL, 1 eq) in phosphoric acid (634.54 mg, 6.48 mmol, 377.70 μL, 0.3 eq) was added 3-bromopropan-1-ol (3 g, 21.58 mmol, 1.95 mL, 1 eq). The mixture was stirred at 5° C. for 16 hours. The reaction mixture was diluted with H₂O 150 mL and extracted with EA 150 mL, dried over Na₂SO₄, filtered and concentrated under reduced pressure to give a residue to afford 2-(3-bromopropoxy)ethan-1-ol (3.9 g, crude) as a colorless oil. To a solution of 2-(3-bromopropoxy)ethan-1-ol (646.54 mg, 3.53 mmol, 1 eq) in DCM (10 mL) was added dropwise TEA (714.84 mg, 7.06 mmol, 983.27 μL, 2 eq), and then nicotinoyl chloride (500 mg, 3.53 mmol, 1 eq) was added. The mixture was stirred at 25° C. for 2 hours. LCMS (5-95AB/1 min, RT=0.444 min, 290.1[M+H]⁺, ESI pos) showed the major peak with desired product was detected. The reaction mixture was filtered and concentrated under reduced pressure to give a residue. The residue was purified by column chromatography (SiO₂, Petroleum ether:Ethyl acetate=1:0 to 2:1), the residue was purified by prep-TLC (SiO₂, Petroleum ether:Ethyl acetate=2:1) to afford 2-(3-bromopropoxy)ethyl nicotinate (150 mg, 400.85 μmol, 11.35% yield, 77% purity) as a yellow oil. To this solution of 2-(3-bromopropoxy)ethyl nicotinate (5, 100 mg, 347.06 μmol, 1 eq) in MeCN (1 mL) was added PPh₃ (95.58 mg, 364.41 μmol, 1.05 eq). The mixture was stirred at 85° C. for 1 hour. LCMS (5-95AB/1 min, RT=0.452 min, 470.2[M]⁺, ESI pos) showed the major peak with desired product. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 18%-48%, 8 min) to afford (3-(2-(nicotinoyloxy)ethoxy)propyl)triphenylphosphonium (20.2 mg, 40.54 μmol, 11.68% yield, 94.42% purity) as a red gum. LCMS: t_{R}=0.450 min, m/z=470.3 (M+H)^{+ 1}H NMR (400 MHz, MeOD) 9.39 (s, 1H), 9.12-9.07 (m, 2H), 8.26-8.19 (m, 1H), 7.91-7.87 (m, 3H), 7.83-7.74 (m, 12H), 4.66-4.59 (m, 2H), 3.88-3.81 (m, 2H), 3.70 (t, J=5.3 Hz, 2H), 3.53-3.43 (m, 2H), 2.00-1.88 (m, 2H).

### (3-((2-(nicotinoyloxy)ethyl)amino)-3-oxopropyl)triphenylphosphonium

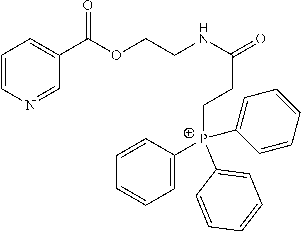 [SMILES: O=C(CC[P+](c1ccccc1)(c1ccccc1)c1ccccc1)NCCOC(=O)c1cccnc1]

To a solution of 2-aminoethyl nicotinate (200 mg, 986.98 μmol, 1 eq, HCl), (2-carboxyethyl)triphenylphosphonium (330.99 mg, 986.98 μmol, 1 eq) in DCM (10 mL) was added EDCI (283.81 mg, 1.48 mmol, 1.5 eq) and HOBt (200.05 mg, 1.48 mmol, 1.5 eq). The mixture was stirred at 25° C. for 16 hours. LCMS (5-95AB/1 min, RT=0.419 min, 438.3[M]⁺, ESI pos) showed the major peak with desired product. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 15%-45%, 8 min) to afford (3-((2-(nicotinoyloxy)ethyl)amino)-3-oxopropyl)triphenylphosphonium (87.1 mg, 173.94 μmol, 17.62% yield, 96.56% purity) as a colorless gum. LCMS: t_{R}=0.419 min, m/z=483.3 (M+H)^{+ 1}H NMR (400 MHz, MeOD) 9.48-9.43 (m, 1H), 9.18-9.07 (m, 2H), 8.29-8.23 (m, 1H), 7.92 (br s, 3H), 7.85-7.75 (m, 12H), 4.42 (t, J=5.3 Hz, 2H), 3.76-3.70 (m, 2H), 3.54 (t, J=5.3 Hz, 2H), 2.76-2.69 (m, 2H).

### (3-((2-(Nicotinamido)ethyl)amino)-3-oxopropyl)triphenylphosphonium

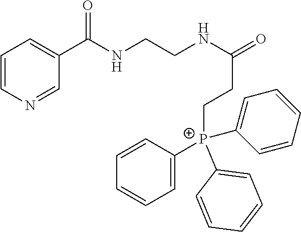 [SMILES: O=C(CC[P+](c1ccccc1)(c1ccccc1)c1ccccc1)NCCNC(=O)c1cccnc1]

To a solution of N-(2-aminoethyl)nicotinamide (200 mg, 716.29 umol, 1 eq, TFA), (2-carboxyethyl)triphenylphosphonium (240.21 mg, 716.29 umol, 1 eq) in MeCN (5 mL) was added EDCI (411.94 mg, 2.15 mmol, 3 eq) and HOBt (290.36 mg, 2.15 mmol, 3 eq). The mixture was stirred at 25° C. for 1 hours. LCMS (5-95AB/1 min, RT=0.395 min, 482.2[M]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 12%-42%, 8 min) to afford (3-((2-(nicotinamido)ethyl)amino)-3-oxopropyl)triphenylphosphonium (100.5 mg, 203.38 μmol, 28.39% yield, 97.65% purity) as a colorless oil. LCMS: t_{R}=0.383 min, m/z=482.3 (M)^{+ 1}H NMR (400 MHz, MeOD) 9.28 (br s, 1H), 9.05-8.93 (m, 2H), 8.19 (br d, J=4.4 Hz, 1H), 7.93-7.75 (m, 15H), 3.78-3.69 (m, 2H), 3.55-3.48 (m, 2H), 3.42-3.35 (m, 2H), 2.74-2.63 (m, 2H).

### (6-((2-(Nicotinamido)Ethyl)Amino)Hexyl)Triphenylphosphonium

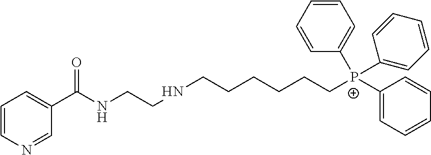 [SMILES: O=C(NCCNCCCCCC[P+](c1ccccc1)(c1ccccc1)c1ccccc1)c1cccnc1]

To a solution of N-(2-aminoethyl)nicotinamide (3.27 g, 11.73 mmol, 5 eq, TFA) in MeOH (25 mL) was added (6-bromohexyl)triphenylphosphonium (1 g, 2.35 mmol, 1 eq). The mixture was stirred at 80° C. for 16 hours. LCMS (5-95AB/1 min, RT=0.361 min, 510.3 [M+H]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 0%-30%, 8 min) to afford (6-((2-(nicotinamido)ethyl)amino)hexyl)triphenylphosphonium (8.2 mg, 15.90 umol, 6.78e-1% yield, 99% purity) as a yellow oil. LCMS: t_{R}=0.363 min, m/z=510.3 (M+H)^{+ 1}H NMR (400 MHz, MeOD) δ 9.63 (s, 1H), 9.16 (br d, J=5.3 Hz, 1H), 9.02 (br d, J=7.6 Hz, 1H), 8.24 (br t, J=6.3 Hz, 1H), 7.93-7.76 (m, 15H), 4.73 (br t, J=6.4 Hz, 2H), 3.77 (br t, J=5.5 Hz, 2H), 3.52-3.40 (m, 2H), 3.27 (br t, J=5.2 Hz, 2H), 2.10 (br s, 2H), 1.71 (br s, 4H), 1.50 (br s, 2H).

### (6-(2-(Nicotinoyloxy)ethoxy)hexyl)triphenylphosphonium

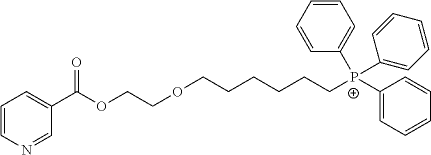 [SMILES: O=C(OCCOCCCCCC[P+](c1ccccc1)(c1ccccc1)c1ccccc1)c1cccnc1]

To a solution of oxirane (1.90 g, 43.17 mmol, 2.16 mL, 2 eq) in phosphoric acid (634.54 mg, 6.48 mmol, 377.70 μL, 0.3 eq) was added 6-bromohexan-1-ol (3.91 g, 21.58 mmol, 2.82 mL, 1 eq). The resulting mixture was stirred at 5° C. for 16 hours. The reaction mixture was diluted with H₂O (150 mL) and extracted with EA (150 mL), dried over Na₂SO₄, filtered and concentrated under reduced pressure to give a residue to afford 2-((6-bromohexyl)oxy)ethan-1-ol (4.8 g, crude) as a colorless oil. To this solution of 2-((6-bromohexyl)oxy)ethan-1-ol (795.18 mg, 3.53 mmol, 1 eq) in DCM (2 mL) was added dropwise TEA (714.85 mg, 7.06 mmol, 983.28 μL, 2 eq), and then nicotinoyl chloride (500 mg, 3.53 mmol, 1 eq) was added. The resulting mixture was stirred at 25° C. for 1 hour. LCMS (5-95AB/1 min, RT=0.541 min, 330.1 [M+H]⁺, ESI pos) showed the major peak with desired product. The reaction mixture was filtered and concentrated under reduced pressure to give a residue. The residue was purified by prep-TLC (SiO₂, Petroleum ether:Ethyl acetate=3:1) to afford 2-((6-bromohexyl)oxy)ethyl nicotinate (300 mg, 708.63 μmol, 20.06% yield, 78% purity) as a colorless oil. To this solution of 2-((6-bromohexyl)oxy)ethyl nicotinate (170 mg, 514.81 μmol, 1 eq) in MeCN (3 mL) was added dropwise PPh₃ (141.78 mg, 540.55 μmol, 1.05 eq). The resulting mixture was stirred at 85° C. for 1 hour. LCMS (5-95AB/1 min, RT=0.502 min, 512.4[M+H]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 24%-54%, 8 min) to afford (6-(2-(nicotinoyloxy)ethoxy)hexyl)triphenylphosphonium (21.2 mg, 39.29 μmol, 7.63% yield, 95% purity) as a colorless gum. LCMS: t_{R}=0.474 min, m/z=512.3 (M+H)^{+ 1}H NMR (400 MHz, MeOD) 9.41 (d, J=1.0 Hz, 1H), 9.15-9.07 (m, 2H), 8.27-8.21 (m, 1H), 7.93-7.86 (m, 3H), 7.83-7.73 (m, 12H), 4.60-4.55 (m, 2H), 3.83-3.77 (m, 2H), 3.51 (t, J=6.5 Hz, 2H), 3.45-3.36 (m, 2H), 1.69-1.53 (m, 6H), 1.45-1.38 (m, 2H).

### 1-((2r,3r,4s,5r)-3,4-Dihydroxy-5-(Hydroxymethyl)Tetrahydrofuran-2-Yl)-3-((Tetradecyloxy)Carbonyl)Pyridin-1-Ium

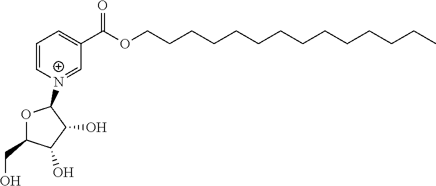 [SMILES: CCCCCCCCCCCCCCOC(=O)c1ccc[n+]([C@@H]2O[C@H](CO)[C@@H](O)[C@H]2O)c1]

To a solution of 1-((2R,3R,4R,5R)-3,4-diacetoxy-5-(acetoxymethyl)tetrahydrofuran-2-yl)-3-((tetradecyloxy)carbonyl)pyridin-1-ium (1, 100 mg, 172.80 μmol, 1 eq) was added HCl (3 M, 5 mL). The mixture was stirred at 25° C. for 16 hours. LCMS (5-95AB/1 min, RT=0.580 min, 452.2[M+H]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 45%-75%, 8 min) to afford 1-((2R,3R,4S,5R)-3,4-dihydroxy-5-(hydroxymethyl)tetrahydrofuran-2-yl)-3-((tetradecyloxy)carbonyl)pyridin-1-ium (14.1 mg, 29.23 μmol, 16.92% yield, 93.84% purity) as a white solid. LCMS: t_{R}=0.596 min, m/z=452.4 (M+H)⁺. ¹H NMR (400 MHz, MeOD) 9.85 (s, 1H), 9.45 (br d, J=6.3 Hz, 1H), 9.12 (br d, J=8.0 Hz, 1H), 8.29 (t, J=7.1 Hz, 1H), 6.20 (d, J=4.9 Hz, 1H), 4.50-4.39 (m, 4H), 4.31 (br d, J=3.1 Hz, 1H), 4.05-3.82 (m, 2H), 1.88-1.80 (m, 2H), 1.51-1.44 (m, 2H), 1.29 (s, 20H), 0.90 (br t, J=6.5 Hz, 3H).

### 1-((2r,3r,4s,5r)-3,4-Dihydroxy-5-(Hydroxymethyl)Tetrahydrofuran-2-Yl)-3-(Tetradecylcarbamoyl)Pyridin-1-Ium

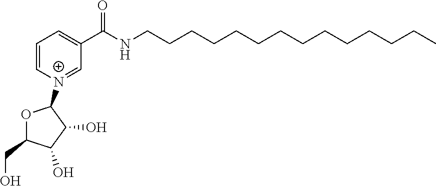 [SMILES: CCCCCCCCCCCCCCNC(=O)c1ccc[n+]([C@@H]2O[C@H](CO)[C@@H](O)[C@H]2O)c1]

To a solution of nicotinoyl chloride (3 g, 16.85 mmol, 1 eq, HCl) in DCM (50 mL) was added TEA (3.41 g, 33.70 mmol, 4.69 mL, 2 eq) and tetradecan-1-ol (2, 3.61 g, 16.85 mmol, 1 eq) at 0° C. The mixture was stirred at 25° C. for 16 hours. LCMS (5-95AB/1 min, RT=0.857 min, 320.3 [M+H]⁺, ESI pos) showed the major peak with desired product. The reaction mixture was filtered and concentrated under reduced pressure to give a residue. The residue was purified by column chromatography (SiO₂, Petroleum ether:Ethyl acetate=1:0 to 1:1) to afford tetradecyl nicotinate (5 g, 15.53 mmol, 92.17% yield, 99.25% purity) as a yellow solid. LCMS: t_{R}=0.867 min, m/z=320.4 (M+H)^{+ 1}H NMR (400 MHz, MeOD) 9.11 (d, J=1.9 Hz, 1H), 8.78-8.68 (m, 1H), 8.44-8.34 (m, 1H), 7.61-7.50 (m, 1H), 4.37 (br t, J=6.6 Hz, 2H), 1.85-1.74 (m, 2H), 1.45 (br d, J=7.8 Hz, 4H), 1.28 (br s, 18H), 0.89 (br t, J=6.4 Hz, 3H). To this solution of tetradecyl nicotinate (1 g, 3.13 mmol, 1 eq), (3R,4R,5R)-5-(acetoxymethyl)tetrahydrofuran-2,3,4-triyl triacetate (1.20 g, 3.76 mmol, 1.2 eq) in DCM (20 mL) was added TMSOTf (1.04 g, 4.70 mmol, 848.40 μL, 1.5 eq) at 0° C. The mixture was stirred at 25° C. for 16 hours. LCMS (5-95AB/1 min, RT=0.632 min, 578.5[M+H]⁺, ESI pos) showed the major peak with desired product. The reaction mixture was diluted with 1M NaHCO₃ 50 mL and extracted with DCM 50 mL, dried over Na₂SO₄, filtered and concentrated under reduced pressure to give a residue. The residue was purified by column chromatography (SiO₂, Petroleum ether:Ethyl acetate=1:0 to Ethyl acetate:MeOH=10:1), the residue was purified by prep-HPLC (column: Welch Xtimate C18 150×25 mm×5 um; mobile phase: [water(HCl)-ACN]; B %: 51%-81%, 8 min) to afford 1-((2R,3R,4R,5R)-3,4-diacetoxy-5-(acetoxymethyl)tetrahydrofuran-2-yl)-3-((tetradecyloxy)carbonyl)pyridin-1-ium (1 g, 1.71 mmol, 54.50% yield, 98.72% purity) as a colorless oil. LCMS: t_{R}=0.642 min, m/z=578.5 (M+H)^{+ 1}H NMR (400 MHz, MeOD) 9.66 (s, 1H), 9.36 (d, J=6.4 Hz, 1H), 9.22-9.16 (m, 1H), 8.41-8.34 (m, 1H), 6.62 (d, J=3.5 Hz, 1H), 5.63-5.54 (m, 1H), 5.43 (t, J=5.7 Hz, 1H), 4.85-4.82 (m, 1H), 4.64-4.48 (m, 4H), 2.21-2.14 (m, 9H), 1.88-1.80 (m, 2H), 1.52-1.45 (m, 2H), 1.29 (s, 20H), 0.93-0.88 (m, 3H). To this solution of 1-((2R,3R,4R,5R)-3,4-diacetoxy-5-(acetoxymethyl)tetrahydrofuran-2-yl)-3-((tetradecyloxy)carbonyl)pyridin-1-ium, 100 mg, 172.80 μmol, 1 eq) was added HCl (3 M, 5 mL). The mixture was stirred at 25° C. for 16 hours. LCMS (5-95AB/1 min, RT=0.580 min, 452.2[M+H]⁺, ESI pos) showed the major peak with desired product was detected. The reaction mixture was filtered and concentrated under reduced pressure to give a residue. The residue was purified by prep-HPLC (column: Welch Xtimate C18 150×25 mm×5 um; mobile phase: [water(HCl)-ACN]; B %: 45%-75%, 8 min) to afford 1-((2R,3R,4S,5R)-3,4-dihydroxy-5-(hydroxymethyl)tetrahydrofuran-2-yl)-3-((tetradecyloxy)carbonyl)pyridin-1-ium (14.1 mg, 29.23 μmol, 16.92% yield, 93.84% purity) as a white solid. LCMS: t_{R}=0.596 min, m/z=452.4 (M+H)⁺. ¹H NMR (400 MHz, MeOD) 9.85 (s, 1H), 9.45 (br d, J=6.3 Hz, 1H), 9.12 (br d, J=8.0 Hz, 1H), 8.29 (t, J=7.1 Hz, 1H), 6.20 (d, J=4.9 Hz, 1H), 4.50-4.39 (m, 4H), 4.31 (br d, J=3.1 Hz, 1H), 4.05-3.82 (m, 2H), 1.88-1.80 (m, 2H), 1.51-1.44 (m, 2H), 1.29 (s, 20H), 0.90 (br t, J=6.5 Hz, 3H).

### 1-((2R,3R,4S,5R)-3,4-Dihydroxy-5-(hydroxymethyl)tetrahydrofuran-2-yl)-3-((4-((E)-3,5-dihydroxystyryl)phenoxy)carbonyl)pyridin-1-ium

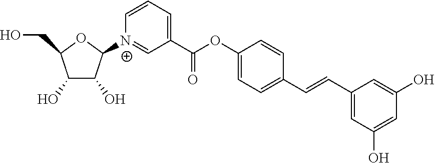 [SMILES: O=C(Oc1ccc(/C=C/c2cc(O)cc(O)c2)cc1)c1ccc[n+]([C@@H]2O[C@H](CO)[C@@H](O)[C@H]2O)c1]

To a solution of 1-((2R,3R,4R,5R)-3,4-diacetoxy-5-(acetoxymethyl)tetrahydrofuran-2-yl)-3-((4-((E)-3,5-diacetoxystyryl)phenoxy)carbonyl)pyridin-1-ium (400 mg, 591.15 μmol, 1 eq) in HCl (3 M, 5 mL, 25.37 eq) was stirred at 25° C. for 16 hours. LCMS (5-95AB/1 min, RT=0.382 min, 466.4[M+H]⁺, ESI pos) showed the major peak with desired ms. The reaction mixture was concentrated under reduced pressure to give a residue. The residue was purified by reversed-phase HPLC (0.1% HCl condition). Then the residue was purified by prep-HPLC (column: Phenomenex luna C18 150×25 mm×10 um; mobile phase: [water(HCl)-ACN]; gradient:5%-35% B over 10 min) to afford 1-((2R,3R,4S,5R)-3,4-dihydroxy-5-(hydroxymethyl)tetrahydrofuran-2-yl)-3-((4-((E)-3,5-dihydroxystyryl)phenoxy) carbonyl)pyridin-1-ium (13.1 mg, 26.83 μmol, 4.54% yield, 95.54% purity, HCl salt) as a yellow solid. LCMS: RT=0.378 min, m/z=466.1 (M+H)^{+ 1}H NMR (400 MHz, CD₃OD) δ 10.07 (s, 1H), 9.53 (d, J=6.3 Hz, 1H), 9.32-9.24 (m, 1H), 8.42-8.33 (m, 1H), 7.39 (d, J=8.6 Hz, 2H), 7.11-6.96 (m, 2H), 6.93 (d, J=8.6 Hz, 2H), 6.78 (d, J=8.6 Hz, 2H), 6.65 (t, J=1.8 Hz, 1H), 6.26 (d, J=4.8 Hz, 1H), 4.50-4.44 (m, 2H), 4.36-4.32 (m, 1H), 4.06 (s, 1H), 3.90-3.84 (m, 1H).

### 1-((2R,3R,4R,5R)-3,4-Diacetoxy-5-(acetoxymethyl)tetrahydrofuran-2-yl)-3-((4-((E)-3,5-diacetoxystyryl)phenoxy)carbonyl)pyridin-1-ium

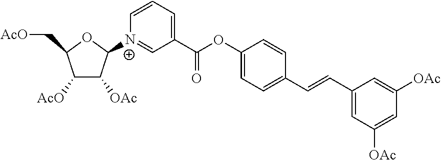 [SMILES: CC(=O)OC[C@H]1O[C@@H]([n+]2cccc(C(=O)Oc3ccc(/C=C/c4cc(OC(C)=O)cc(OC(C)=O)c4)cc3)c2)[C@H](OC(C)=O)[C@@H]1OC(C)=O]

To a solution of (E)-5-(4-(nicotinoyloxy)styryl)-1,3-phenylene diacetate (1 g, 2.40 mmol, 1 eq), (2S,3R,4R,5R)-5-(acetoxymethyl)tetrahydrofuran-2,3,4-triyl triacetate (915.00 mg, 2.87 mmol, 1.2 eq) in DCM (20 mL) was added TMSOTf (1.06 g, 4.79 mmol, 865.80 μL, 2 eq). The mixture was stirred at 25° C. for 2 hours. LCMS (5-95AB/1 min, RT=0.509 min, 676.3[M+H]⁺, ESI pos) showed the major peak with desired ms. The reaction mixture was diluted with 1M NaHCO₃ (150 mL) and extracted with DCM (150 mL), dried over Na₂SO₄, filtered and concentrated under reduced pressure to give a residue. The residue was purified by column chromatography (SiO₂, Petroleum ether:Ethyl acetate=1:0 to 0:1) to afford 1-((2R,3R,4R,5R)-3,4-diacetoxy-5-(acetoxymethyl)tetrahydrofuran-2-yl)-3-((4-((E)-3,5-diacetoxystyryl)phenoxy)carbonyl)pyridin-1-ium (1 g, 2.34 mmol, 24.33% yield, 97.54% purity, TfOH salt) as a yellow solid. LCMS: RT=0.509 min, m/z=676.3 (M+H)⁺. ¹H NMR (400 MHz, CD₃OD) δ 9.85 (s, 1H), 9.51-9.37 (m, 2H), 8.54-8.45 (m, 1H), 7.63 (d, J=8.5 Hz, 2H), 7.49 (t, J=1.6 Hz, 1H), 7.38-7.19 (m, 3H), 7.17-7.10 (m, 3H), 6.69 (d, J=3.5 Hz, 1H), 5.69-5.62 (m, 1H), 5.48 (t, J=5.8 Hz, 1H), 4.88-4.85 (m, 1H), 4.68-4.51 (m, 2H), 2.32 (d, J=15.8 Hz, 6H), 2.23-2.15 (m, 9H).

### 1-((2R,3R,4R,5R)-3,4-Diacetoxy-5-(acetoxymethyl)tetrahydrofuran-2-yl)-3-((tetradecyloxy)carbonyl)pyridin-1-ium

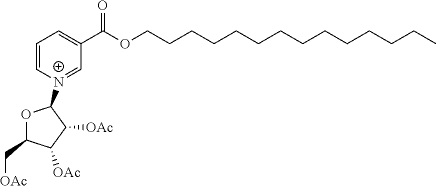 [SMILES: CCCCCCCCCCCCCCOC(=O)c1ccc[n+]([C@@H]2O[C@H](COC(C)=O)[C@@H](OC(C)=O)[C@H]2OC(C)=O)c1]

To a solution of tetradecyl nicotinate (1 g, 3.13 mmol, 1 eq), (3R,4R,5R)-5-(acetoxymethyl)tetrahydrofuran-2,3,4-triyl triacetate (2, 1.20 g, 3.76 mmol, 1.2 eq) in DCM (20 mL) was added TMSOTf (1.04 g, 4.70 mmol, 848.40 μL, 1.5 eq) at 0° C. The mixture was stirred at 25° C. for 16 hours. LCMS (5-95AB/1 min, RT=0.632 min, 578.5[M+H]⁺, ESI pos) showed the major peak with desired product. The reaction mixture was diluted with 1M NaHCO₃ 50 mL and extracted with DCM 50 mL, dried over Na₂SO₄, filtered and concentrated under reduced pressure to give a residue. The residue was purified by column chromatography (SiO₂, Petroleum ether:Ethyl acetate=1:0 to Ethyl acetate:MeOH=10:1), the residue was purified by prep-HPLC (column: Welch Xtimate C18 150×25 mm×5 um; mobile phase: [water(HCl)-ACN]; B %: 51%-81%, 8 min) to afford 1-((2R,3R,4R,5R)-3,4-diacetoxy-5-(acetoxymethyl)tetrahydrofuran-2-yl)-3-((tetradecyloxy)carbonyl)pyridin-1-ium (1 g, 1.71 mmol, 54.50% yield, 98.72% purity) as a colorless oil. LCMS: t_{R}=0.642 min, m/z=578.5 (M+H)⁺. ¹H NMR (400 MHz, MeOD) 9.66 (s, 1H), 9.36 (d, J=6.4 Hz, 1H), 9.22-9.16 (m, 1H), 8.41-8.34 (m, 1H), 6.62 (d, J=3.5 Hz, 1H), 5.63-5.54 (m, 1H), 5.43 (t, J=5.7 Hz, 1H), 4.85-4.82 (m, 1H), 4.64-4.48 (m, 4H), 2.21-2.14 (m, 9H), 1.88-1.80 (m, 2H), 1.52-1.45 (m, 2H), 1.29 (s, 20H), 0.93-0.88 (m, 3H).

### 1-((2R,3R,4R,5R)-3,4-Diacetoxy-5-(acetoxymethyl)tetrahydrofuran-2-yl)-3-(tetradecylcarbamoyl)pyridin-1-ium

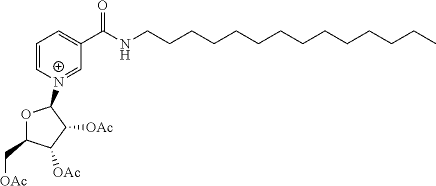 [SMILES: CCCCCCCCCCCCCCNC(=O)c1ccc[n+]([C@@H]2O[C@H](COC(C)=O)[C@@H](OC(C)=O)[C@H]2OC(C)=O)c1]

To a solution of N-tetradecylnicotinamide (3 g, 9.42 mmol, 1 eq), (3R,4R,5R)-5-(acetoxymethyl)tetrahydrofuran-2,3,4-triyl triacetate (3.00 g, 9.42 mmol, 1 eq) in DCM (50 mL) was added TMSOTf (4.19 g, 18.84 mmol, 3.40 mL, 2 eq). The mixture was stirred at 25° C. for 16 hour. LCMS (5-95AB/1 min, RT=0.611 min, 577.5[M+H]⁺, ESI pos) showed the major peak with desired product. The reaction mixture was diluted with 1M NaHCO₃ 50 mL and extracted with DCM 50 mL, dried over Na₂SO₄, filtered and concentrated under reduced pressure to give a residue. The residue was purified by column chromatography (SiO₂, Petroleum ether:Ethyl acetate=1:0 to Ethyl acetate:MeOH=10:1) to afford 1-((2R,3R,4R,5R)-3,4-diacetoxy-5-(acetoxymethyl)tetrahydrofuran-2-yl)-3-(tetradecylcarbamoyl)pyridin-1-ium (2.7 g, 4.51 mmol, 47.93% yield, 96.6% purity) as a colorless gum. LCMS: t_{R}=0.625 min, m/z=577.5 (M+H)⁺. ¹H NMR (400 MHz, MeOD) 9.59 (s, 1H), 9.29 (d, J=6.2 Hz, 1H), 9.06 (d, J=8.1 Hz, 1H), 8.39-8.31 (m, 1H), 6.59 (d, J=3.8 Hz, 1H), 5.63-5.58 (m, 1H), 5.45 (t, J=5.6 Hz, 1H), 4.85-4.81 (m, 1H), 4.66-4.48 (m, 2H), 3.51-3.44 (m, 2H), 2.23-2.15 (m, 9H), 1.74-1.64 (m, 2H), 1.31 (s, 22H), 0.92 (t, J=6.8 Hz, 3H).

### 1-((2R,3R,4R,5R)-3,4-Bis(propionyloxy)-5-((propionyloxy)methyl)tetrahydrofuran-2-yl)-3-carboxypyridin-1-ium

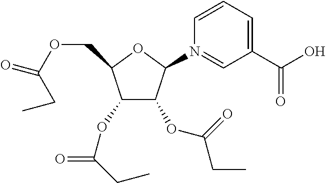

To a solution of nicotinic acid (10 g, 81.23 mmol, 6.80 mL, 1 eq) in HMDS (39.33 g, 243.69 mmol, 51.08 mL, 3 eq) was added (NH₄)₂SO₄ (536.68 mg, 4.06 mmol, 303.21 uL, 0.05 eq) in one portion. The mixture was stirred at 110° C. for 1 hour. TLC (Petroleum ether:Ethyl acetate=0:1) showed the material was consumed (R_{f}=0.01), and a new spot was detected (R_{f}=0.26). The reaction mixture was filtered and concentrated under reduced pressure to afford trimethylsilyl nicotinate (2, 14 g, 71.69 mmol, 88.25% yield) as colorless oil. ¹H NMR (400 MHz, DMSO) δ 9.07 (dd, J=0.6, 2.1 Hz, 1H), 8.79 (dd, J=1.7, 4.8 Hz, 1H), 8.26 (td, J=2.0, 7.9 Hz, 1H), 7.54 (ddd, J=0.6, 4.8, 7.9 Hz, 1H), 0.36 (s, 6H), 0.03 (s, 3H). To this solution of (3R,4R,5R)-5-(acetoxymethyl)tetrahydrofuran-2,3,4-triyl triacetate (23.31 g, 73.22 mmol, 1.1 eq), trimethylsilyl nicotinate (13 g, 66.57 mmol, 1 eq) in DCM (300 mL) was added TMSOTf (17.75 g, 79.88 mmol, 14.43 mL, 1.2 eq) at 0° C. The mixture was stirred at 25° C. for 1 hour. LCMS (0-60AB/1.5 min, RT=0.289 min, 382.1 [M+H]⁺, ESI pos) showed the major peak with desired product was detected. The product was dissolved in ca. 100 mL of DCM and the solution was poured into ice water. The mixture was neutralized to pH 6-7 with saturated aqueous NaHCO₃, colorless aqueous phase was separated from the yellowish organic phase.

The aqueous phase was evaporated under reduced pressure below 40° C. to give a white solid product. The crude product was purified by reversed-phase HPLC (MeCN/H₂O) to afford 1-((2R,3R,4R,5R)-3,4-diacetoxy-5-(acetoxymethyl)tetrahydrofuran-2-yl)pyridin-1-ium-3-carboxylate (13.5 g, 35.40 mmol, 53.18% yield) as white solid. ¹H NMR (400 MHz, D₂O) δ 9.38 (s, 1H), 9.07 (d, J=6.2 Hz, 1H), 8.93 (d, J=7.9 Hz, 1H), 8.24-8.09 (m, 1H), 6.55 (d, J=4.0 Hz, 1H), 5.61-5.43 (m, 2H), 4.93-4.82 (m, 1H), 4.52 (d, J=2.2 Hz, 2H), 2.20-2.04 (m, 9H). To this solution of 1-((2R,3R,4R,5R)-3,4-diacetoxy-5-(acetoxymethyl)tetrahydrofuran-2-yl) pyridin-1-ium-3-carboxylate (1 g, 2.62 mmol, 1 eq) in MeOH (10 mL) was added NH₃/MeOH (7 M, 10.00 mL, 26.69 eq) at −20° C. The mixture was stirred at 0° C. for 2 hours. LCMS (0-60AB/1.5 min, RT=0.127 min, 256.1 [M+H]⁺, ESI pos) showed the major peak with desired product was detected. The reaction mixture was concentrated under reduced pressure at 0° C. to afford 1-((2R,3R,4S,5R)-3,4-dihydroxy-5-(hydroxymethyl)tetrahydrofuran-2-yl) pyridine-1-ium-3-carboxylate (650 mg, 2.55 mmol, 97.12% yield) as white solid. ¹H NMR (400 MHz, D₂O) S 9.38 (s, 1H), 9.07 (d, J=6.2 Hz, 1H), 8.87 (d, J=8.1 Hz, 1H), 8.18-8.04 (m, 1H), 6.15 (d, J=4.6 Hz, 1H), 4.46-4.37 (m, 2H), 4.32-4.25 (m, 1H), 4.03-3.92 (m, 1H), 3.89-3.79 (m, 1H). To this solution of 1-((2R,3R,4S,5R)-3,4-dihydroxy-5-(hydroxymethyl)tetrahydrofuran-2-yl)pyridine-1-ium-3-carboxylate (200 mg, 783.63 umol, 1 eq) in Py (1.24 g, 15.67 mmol, 1.26 mL, 20 eq), H₂O (0.2 mL) was added dropwise propionic anhydride (1.53 g, 11.75 mmol, 1.51 mL, 15 eq) at 0° C. The resulting mixture was stirred at 25° C. for 2 hours. LCMS (0-60AB/1.5 min, RT=0.874 min, 424.2[M+H]⁺, ESI pos) showed the major peak with desired product was detected. The reaction mixture was filtered and concentrated under reduced pressure to give a residue. The residue was purified by prep-HPLC (column: Phenomenex C18 75×30 mm×3 um; mobile phase: [water(FA)-ACN]; B %: 12%-42%, 7 min) to afford 1-((2R,3R,4R,5R)-3,4-bis(propionyloxy)-5-((propionyloxy)methyl)tetrahydrofuran-2-yl) pyridin-1-ium-3-carboxylate (92.84 mg, 215.42 umol, 27.49% yield, 98.48% purity) as yellow solid. LCMS: t_{R}=0.726 min, m/z=424.2 (M+H)⁺

¹H NMR (400 MHz, D₂O) δ 9.38 (s, 1H), 9.09 (br d, J=6.1 Hz, 1H), 8.97 (br d, J=7.9 Hz, 1H), 8.20 (t, J=7.0 Hz, 1H), 6.58 (d, J=4.3 Hz, 1H), 5.61-5.50 (m, 2H), 4.92 (br d, J=1.5 Hz, 1H), 4.56 (br s, 2H), 2.53-2.39 (m, 6H), 1.13-1.01 (m, 9H).

### 1-((2R,3R,4R,5R)-3,4-Bis(butyryloxy)-5-((butyryloxy)methyl)tetrahydrofuran-2-yl)-3-carboxypyridin-1-ium

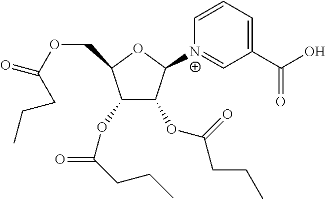 [SMILES: CCCC(=O)OC[C@H]1O[C@@H]([n+]2cccc(C(=O)O)c2)[C@H](OC(=O)CCC)[C@@H]1OC(=O)CCC]

To a solution of 3-carboxy-1-((2R,3R,4S,5R)-3,4-dihydroxy-5-(hydroxymethyl)tetrahydrofuran-2-yl)pyridin-1-ium (1, 240 mg, 940.35 μmol, 1 eq) in Py (30 mL) and H₂O (6 mL) was added butyric anhydride (2.23 g, 14.11 mmol, 2.31 mL, 15 eq) at 0° C. The mixture was stirred at 25° C. for 2 hours. LCMS (5-95AB/1.5 min, RT=0.467 min, 466.4[M+H]⁺, ESI pos) showed the major peak with desired ms. The reaction mixture was filtered and concentrated under reduced pressure to give a residue. The residue was purified by reversed-phase HPLC (column: Phenomenex luna C18 150×25 mm×10 um; mobile phase: [water(FA)-ACN]; gradient:20%-50% B over 10) to afford 1-((2R,3R,4R,5R)-3,4-bis(butyryloxy)-5-((butyryloxy)methyl)tetrahydrofuran-2-yl)-3-carboxypyridin-1-ium (14.3 mg, 28.72 μmol, 3.05% yield, 93.7% purity) as a yellow solid. LCMS: RT=0.468 min, m/z=466.0 (M+H)^{+ 1}H NMR (400 MHz, MeOD) δ 9.52 (s, 1H), 9.13 (d, J=6.4 Hz, 1H), 9.05 (d, J=7.8 Hz, 1H), 8.21 (dd, J=6.5, 7.6 Hz, 1H), 6.53 (d, J=4.4 Hz, 1H), 5.57 (t, J=4.9 Hz, 1H), 5.46 (t, J=5.3 Hz, 1H), 4.64-4.56 (m, 2H), 4.52-4.44 (m, 1H), 2.48-2.38 (m, 6H), 1.70-1.62 (m, 6H), 1.00-0.93 (m, 9H).

### 3-((Benzyloxy)carbonyl)-1-((2R,3R,4S,5R)-3,4-dihydroxy-5-(hydroxymethyl)tetrahydrofuran-2-yl)pyridin-1-ium

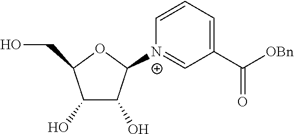 [SMILES: O=C(OCc1ccccc1)c1ccc[n+]([C@@H]2O[C@H](CO)[C@@H](O)[C@H]2O)c1]

To a solution of benzyl nicotinate (1, 1 g, 4.69 mmol, 1 eq) in DCM (20 mL) was added TMSOTf (1.25 g, 5.63 mmol, 1.02 mL, 1.2 eq) and (2S,3R,4R,5R)-5-(acetoxymethyl) tetrahydrofuran-2,3,4-triyl triacetate (1.64 g, 5.16 mmol, 1.1 eq) at 0° C. The mixture was stirred at 25° C. for 12 hours. LCMS (5-95AB/1.5 min, RT=0.402 min, 472.2[M+H]⁺, ESI pos) showed the major peak with desired ms. The reaction mixture was filtered and concentrated under reduced pressure to afford 3-((benzyloxy)carbonyl)-1-((2R,3R,4R,5R)-3,4-diacetoxy-5-(acetoxymethyl)tetrahydrofuran-2-yl)pyridin-1-ium (2, 3.5 g, 4.67 mmol, 99.50% yield, 62.993% purity) as a white solid. To a solution of 3-((benzyloxy)carbonyl)-1-((2R,3R,4R,5R)-3,4-diacetoxy-5-(acetoxymethyl)tetrahydrofuran-2-yl)pyridin-1-ium (2.5 g, 5.29 mmol, 1 eq) was added HCl (3 M, 25.00 mL). The mixture was stirred at 25° C. for 12 hours. LCMS (5-95AB/1 min, RT=0.352 min, 346.2[M+H]⁺, ESI pos) showed the major peak with desired ms. The reaction mixture was filtered and concentrated under reduced pressure to give a residue. The residue was purified by reversed-phase HPLC (column: Welch Xtimate C18 150×25 mm×5 um; mobile phase: [water(HCl)-ACN]; gradient:2%-32% B over 9 min) to afford 3-((benzyloxy)carbonyl)-1-((2R,3R,4S,5R)-3,4-dihydroxy-5-(hydroxymethyl)tetrahydrofuran-2-yl)pyridin-1-ium (N-53, 150 mg, 433.08 μmol, 8.18% yield, HCl salt) as a white solid. LCMS: Rt=0.369 min, m/z=346.2 (M+H)⁺. ¹H NMR (400 MHz, CD₃OD) δ 9.85 (s, 1H), 9.46 (d, J=6.4 Hz, 1H), 9.14 (d, J=8.0 Hz, 1H), 8.28 (dd, J=6.3, 7.8 Hz, 1H), 7.56-7.50 (m, 2H), 7.43-7.36 (m, 3H), 6.19 (d, J=5.0 Hz, 1H), 5.51 (s, 2H), 4.46-4.37 (m, 2H), 4.29 (dd, J=3.0, 4.8 Hz, 1H), 4.00 (dd, J=2.6, 12.3 Hz, 1H), 3.85 (dd, J=2.1, 12.3 Hz, 1H).

### 3-(benzylcarbamoyl)-1-((2R,3R,4S,5R)-3,4-dihydroxy-5-(hydroxymethyl)tetrahydrofuran-2-yl)pyridin-1-ium

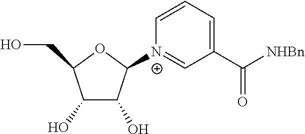

To a solution of 3-(benzylcarbamoyl)-1-((2R,3R,4R,5R)-3,4-diacetoxy-5-(acetoxymethyl)tetrahydrofuran-2-yl)pyridin-1-ium (100 mg, 212.10 μmol, 1 eq) in MeOH (3 mL) was added NH₃/MeOH (7 M, 3 mL, 99.01 eq) at 0° C. The mixture was stirred at 0° C. for 2 hours. LCMS (5-95AB/1 min, RT=0.349 min, 344.9[M+H]⁺, ESI pos) showed the major peak with desired ms. The reaction mixture was filtered and concentrated under reduced pressure to give a residue. The crude product were purified by reversed-phase HPLC (column: Welch Xtimate C18 150×25 mm×5 um; mobile phase: [water(HCl)-ACN]; gradient:0%-30% B over 8 min) to afford 3-(benzylcarbamoyl)-1-((2R,3R,4S,5R)-3,4-dihydroxy-5-(hydroxymethyl)tetrahydrofuran-2-yl)pyridin-1-ium (15.8 mg, 44.08 μmol, 20.79% yield, 96.364% purity) as a yellow solid. LCMS: RT=0.349 min, m/z=344.9 (M+H)⁺. ¹H NMR (400 MHz, MeOD) δ 9.69 (s, 1H), 9.42-9.40 (m, 1H), 9.02-9.00 (m, 1H), 8.29-8.27 (m, 1H), 7.42-7.29 (m, 5H), 6.17-6.16 (m, 1H), 4.66-4.65 (m, 2H), 4.44-4.41 (m, 2H), 4.31-4.30 (m, 1H), 4.00-3.87 (m, 1H), 3.31-3.30 (m, 1H).

### 3-((benzyloxy)carbonyl)-1-((2R,3R,4R,5R)-3,4-diacetoxy-5-(acetoxymethyl)tetrahydrofuran-2-yl)pyridin-1-ium

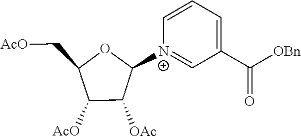 [SMILES: CC(=O)OC[C@H]1O[C@@H]([n+]2cccc(C(=O)OCc3ccccc3)c2)[C@H](OC(C)=O)[C@@H]1OC(C)=O]

To a solution of benzyl nicotinate (1 g, 4.69 mmol, 1 eq) in DCM (20 mL) was added TMSOTf (5 eq) and (2S,3R,4R,5R)-5-(acetoxymethyl) tetrahydrofuran-2,3,4-triyl triacetate (1.64 g, 5.16 mmol, 1.1 eq) at 0° C. The mixture was stirred at 50° C. for 12 hours. LCMS (. The reaction mixture was filtered and concentrated under reduced pressure and was purified by reversed-phase HPLC (0.1% FA condition) to afford 3-((benzyloxy)carbonyl)-1-((2R,3R,4R,5R)-3,4-diacetoxy-5-(acetoxymethyl)tetrahydrofuran-2-yl)pyridin-1-ium (3.5 g, 4.67 mmol, 60% yield, 99% purity) as a white solid. LCMS: Rt=5.14 min, m/z=472 M+.

### 3-(benzylcarbamoyl)-1-((2R,3R,4R,5R)-3,4-diacetoxy-5-(acetoxymethyl)tetrahydrofuran-2-yl)pyridin-1-ium

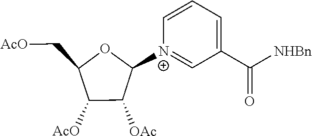

To a solution of nicotinoyl chloride (2 g, 11.23 mmol, 1 eq, HCl) in DCM (50 mL) was added BnNH₂ (1.20 g, 11.23 mmol, 1.22 mL, 1 eq) and TEA (2.27 g, 22.47 mmol, 3.13 mL, 2 eq) at 0° C. The mixture was stirred at 25° C. for 12 hours. LCMS (5-95AB/1.5 min, RT=0.376 min, 213.2[M+H]+, ESI pos) showed the major peak with desired ms. The reaction mixture was filtered and concentrated under reduced pressure to give a residue. The residue was purified by column chromatography (SiO₂, Petroleum ether/Ethyl acetate=1/0 to 0/1) to afford N-benzylnicotinamide (2 g, 9.32 mmol, 82.95% yield, 98.9% purity) as a white solid. LCMS: Rt=0.362 min, m/z=213.2 (M+H)⁺. To this solution of N-benzylnicotinamide (2, 500 mg, 2.36 mmol, 1 eq) in DCM (15 mL) was added TMSOTf (1.05 g, 4.71 mmol, 851.36 μL, 2 eq) and (2S,3R,4R,5R)-5-(acetoxymethyl)tetrahydrofuran-2,3,4-triyl triacetate (749.78 mg, 2.36 mmol, 1 eq) at 0° C. The mixture was stirred at 25° C. for 2 hours. LCMS (5-95AB/1.5 min, RT=0.410 min, 471.2[M+H]⁺, ESI pos) showed the major peak with desired ms. The reaction mixture was filtered and concentrated under reduced pressure to give a residue. The residue was purified by column chromatography (SiO₂, Petroleum ether/Ethyl acetate=1/0 to 0/1) to afford 3-(benzylcarbamoyl)-1-((2R,3R,4R,5R)-3,4-diacetoxy-5-(acetoxymethyl) tetrahydrofuran-2-yl)pyridin-1-ium (350 mg, 727.50 μmol, 30.88% yield, 98% purity) as a white solid. LCMS: RT=0.416 min, m/z=471.1 (M+H)⁺. ¹H NMR (400 MHz, CD₃OD) δ 9.60 (s, 1H), 9.28 (d, J=6.1 Hz, 1H), 9.07 (d, J=8.1 Hz, 1H), 8.37-8.28 (m, 1H), 7.41-7.27 (m, 5H), 6.57 (d, J=3.6 Hz, 1H), 5.61-5.55 (m, 1H), 5.42 (t, J=5.7 Hz, 1H), 4.83-4.79 (m, 1H), 4.65 (d, J=3.1 Hz, 2H), 4.62-4.46 (m, 2H), 2.19-2.09 (m, 9H)

### 1-((2R,3R,4R,5R)-3,4-bis(nicotinoyloxy)-5-((nicotinoyloxy)methyl)tetrahydrofuran-2-yl)-3-carboxypyridin-1-ium

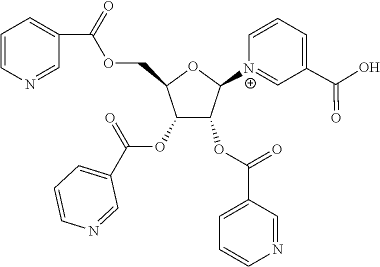 [SMILES: O=C(O)c1ccc[n+]([C@@H]2O[C@H](COC(=O)c3cccnc3)[C@@H](OC(=O)c3cccnc3)[C@H]2OC(=O)c2cccnc2)c1]

A mixture of (3R,4S,5R)-5-(hydroxymethyl)tetrahydrofuran-2,3,4-triol (1, 10.00 g, 66.61 mmol, 1 eq) in MeOH (80 mL) was added H₂SO₄ (1.96 g, 19.98 mmol, 1.07 mL, 0.3 eq) at 0° C., and then the mixture was stirred at 25° C. for 16 hours. TLC (Dichloromethane:Methanol=3:1) showed the material was consumed (R_{f}=0.5), and a new spot was detected (R_{f}=0.7). The mixture was neutralized to pH 9 with aq Na₂CO₃ at 0° C., then the mixture was filtered and then was concentrated under reduced pressure to afford (2R,3S,4R)-2-(hydroxymethyl)-5-methoxytetrahydrofuran-3,4-diol (8 g, 48.73 mmol, 73.16% yield) as a yellow oil. ¹H NMR (400 MHz, DMSO) δ 4.62 (s, 1H), 3.70 (d, J=4.8 Hz, 1H), 3.53-3.48 (m, 1H), 3.45-3.39 (m, 1H), 3.35-3.27 (m, 2H), 3.22 (s, 3H). To this solution of (2R,3S,4R)-2-(hydroxymethyl)-5-methoxytetrahydrofuran-3,4-diol (2, 1 g, 6.09 mmol, 1 eq) in DCM (30 mL) was added Py (4.82 g, 60.92 mmol, 4.92 mL, 10 eq) and nicotinoyl chloride (5.42 g, 30.46 mmol, 5 eq, HCl) at 0° C. The mixture was stirred at 25° C. for 16 hours. LCMS (5-95AB/1.5 min, RT=0.588 min, 480.2[M+H]⁺, ESI pos) showed the major peak with desired ms was detected. The residue was diluted with H₂O (100 mL) and extracted with DCM 100 mL (100 mL×3). The combined organic layers were dried over Na₂SO₄, filtered and concentrated under reduced pressure to give a residue. The crude product was purified by column chromatography (SiO₂, DCM:MeOH=1:0 to 10:1) to afford (3R,4R,5R)-2-methoxy-5-((nicotinoyloxy)methyl)tetrahydrofuran-3,4-diyl dinicotinate (2.5 g, 5.21 mmol, 85.60% yield) as a colorless oil. LCMS: Rt=0.588 min, m/z=480.2 (M+H⁺). ¹H NMR (400 MHz, DMSO) δ 9.18-8.90 (m, 3H), 8.86-8.74 (m, 3H), 8.39-8.13 (m, 3H), 7.62-7.47 (m, 3H), 5.84-5.74 (m, 1H), 5.61-5.50 (m, 1H), 5.42-5.25 (m, 1H), 4.89-4.62 (m, 2H), 4.59-4.48 (m, 1H), 3.32 (s, 3H). To this solution of (3R,4R,5R)-2-methoxy-5-((nicotinoyloxy)methyl)tetrahydrofuran-3,4-diyl dinicotinate (200 mg, 417.16 umol, 1 eq) in AcOH (2 mL) was added Ac₂O (127.76 mg, 1.25 mmol, 117.21 uL, 3 eq), H₂SO₄ (40.91 mg, 417.16 umol, 22.24 uL, 1 eq). The mixture was stirred at 25° C. for 1 hour. LCMS (5-95AB/1.5 min, RT=0.718 min, 508.2[M+H]⁺, ESI pos) showed the major peak with desired product was detected. The product was dissolved in ca. 50 mL of DCM and the solution was poured into ice. The mixture was neutralized to pH 6-7 with saturated aqueous NaHCO₃, dried over Na₂SO₄, filtered and concentrated under reduced pressure to give a residue. The crude product was purified by reversed-phase HPLC (0.1% FA condition) to afford (2S,3R,4R,5R)-2-acetoxy-5-((nicotinoyloxy)methyl)tetrahydrofuran-3,4-diyldinicotinate (100 mg, 197.06 umol, 47.24% yield) as a yellow oil. LCMS: Rt=0.718 min, m/z=508.2 (M+H+). ¹H NMR (400 MHz, CDCl₃) δ 9.34-9.05 (m, 3H), 8.88-8.75 (m, 3H), 8.40-8.13 (m, 3H), 7.51-7.33 (m, 3H), 6.78-6.40 (m, 1H), 5.95-5.65 (m, 2H), 4.88-4.71 (m, 2H), 4.69-4.57 (m, 1H), 2.20-2.08 (m, 3H). To this solution of nicotinic acid (5 g, 40.61 mmol, 3.40 mL, 1 eq) was added HMDS (19.66 g, 121.84 mmol, 25.54 mL, 3 eq) and (NH₄)₂SO₄ (268.34 mg, 2.03 mmol, 151.60 uL, 0.05 eq). The mixture was stirred at 110° C. for 1 hour. TLC (Petroleum ether:Ethyl acetate=0:1) indicated Reactant 1 was consumed completely and one new spot formed. The reaction mixture was concentrated under reduced pressure to give a residue to afford trimethylsilyl nicotinate (4 g, 20.48 mmol, 50.43% yield) as a colorless oil. To a solution of (2S,3R,4R,5R)-2-acetoxy-5-((nicotinoyloxy)methyl)tetrahydrofuran-3,4-diyldinicotinate (500 mg, 985.32 umol, 1 eq), trimethylsilyl nicotinate (230.91 mg, 1.18 mmol, 1.2 eq) in DCM (10 mL) was added TMSOTf (262.79 mg, 1.18 mmol, 213.65 uL, 1.2 eq). The mixture was stirred at 25° C. for 1 hour. LCMS (5-95AB/1.5 min, RT=0.363 min, 571.3, [M+H]⁺, ESI pos) showed the major peak with desired ms was detected. The reaction mixture was filtered and concentrated under reduced pressure to give a residue. The residue was purified by reversed-phase HPLC (0.1% FA condition). Then the residue was purified by prep-HPLC (column: Phenomenex Luna C18 150×25 mm×10 um; mobile phase: [water(FA)-ACN]; B %: 4%-34%, 10 min) to afford 1-((2R,3R,4R,5R)-3,4-bis(nicotinoyloxy)-5-((nicotinoyloxy)methyl) tetrahydrofuran-2-yl)-3-carboxypyridin-1-ium (20 mg, 34.99 umol, 3.55% yield) as a yellow solid. LCMS: t_{R}=0.363 min, m/z=571.2 (M+H)⁺. ¹H NMR (400 MHz, MeOD) δ 9.62 (s, 1H), 9.29 (d, J=6.1 Hz, 1H), 9.20-9.12 (m, 3H), 9.06 (br d, J=7.9 Hz, 1H), 8.80-8.73 (m, 3H), 8.53-8.39 (m, 3H), 8.25-8.16 (m, 1H), 7.63-7.52 (m, 3H), 6.94 (d, J=3.2 Hz, 1H), 6.12-6.04 (m, 2H), 5.33-5.26 (m, 1H), 5.05-4.97 (m, 2H)

### 3,3′-((ethane-1,2-diylbis(azanediyl))bis(carbonyl))bis(1-((2R,3R,4R,5R)-3,4-diacetoxy-5-(acetoxymethyl)tetrahydrofuran-2-yl)pyridin-1-ium)

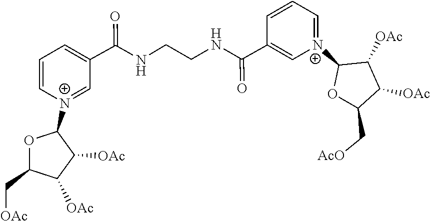 [SMILES: CC(=O)OC[C@H]1O[C@@H]([n+]2cccc(C(=O)NCCNC(=O)c3ccc[n+]([C@@H]4O[C@H](COC(C)=O)[C@@H](OC(C)=O)[C@H]4OC(C)=O)c3)c2)[C@H](OC(C)=O)[C@@H]1OC(C)=O]

To a solution of N,N′-(ethane-1,2-diyl)dinicotinamide (1 eq) in DMF (0.1 M) was added TMSOTf (5 eq) and (2S,3R,4R,5R)-5-(acetoxymethyl) tetrahydrofuran-2,3,4-triyl triacetate (1.64 g, 5.16 mmol, 4 eq) at 0° C. The mixture was stirred at 50° C. for 12 hours. LCMS (. The reaction mixture was filtered and concentrated under reduced pressure and was purified by reversed-phase HPLC (0.1% TFA condition) to afford 3,3′-((ethane-1,2-diylbis(azanediyl))bis(carbonyl))bis(1-((2R,3R,4R,5R)-3,4-diacetoxy-5 (acetoxymethyl)tetrahydrofuran-2-yl)pyridin-1-ium) (52% yield, 98% purity) as a white gummy solid. LCMS: Rt=10.35 min, m/z=788 (M+).

### 3,3′-((ethane-1,2-diylbis(oxy))bis(carbonyl))bis(1-((2R,3R,4R,5R)-3,4-diacetoxy-5-(acetoxymethyl)tetrahydrofuran-2-yl)pyridin-1-ium)

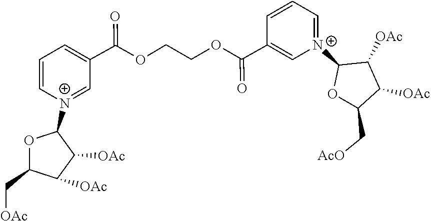 [SMILES: CC(=O)OC[C@H]1O[C@@H]([n+]2cccc(C(=O)OCCOC(=O)c3ccc[n+]([C@@H]4O[C@H](COC(C)=O)[C@@H](OC(C)=O)[C@H]4OC(C)=O)c3)c2)[C@H](OC(C)=O)[C@@H]1OC(C)=O]

To a solution of ethane-1,2-diyl dinicotinate (1 eq) in DMF (0.1 M) was added TMSOTf (5 eq) and (2S,3R,4R,5R)-5-(acetoxymethyl) tetrahydrofuran-2,3,4-triyl triacetate (1.64 g, 5.16 mmol, 4 eq) at 0° C. The mixture was stirred at 50° C. for 12 hours. LCMS (. The reaction mixture was filtered and concentrated under reduced pressure and was purified by reversed-phase HPLC (0.1% TFA condition) to afford 3,3′-((ethane-1,2-diylbis(oxy))bis(carbonyl))bis(1-((2R,3R,4R,5R)-3,4-diacetoxy-5-(acetoxymethyl)tetrahydrofuran-2-yl)pyridin-1-ium) (32% yield, 96% purity) as a white solid. LCMS: Rt=4.6 min, m/z=791 (M+H)⁺.

### 3-(tert-butoxycarbonyl)-1-((2R,3R,4R,5R)-3,4-diacetoxy-5-(acetoxymethyl)tetrahydrofuran-2-yl)pyridin-1-ium

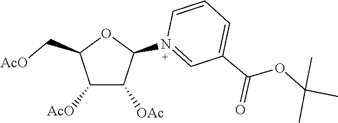

To a solution of nicotinic acid (1, 5 g, 40.61 mmol, 3.40 mL, 1 eq) in DMF (50 mL) was added with CDI (6.59 g, 40.61 mmol, 1 eq) under N₂. After addition, the mixture was stirred at 40° C. for 1 hour, and then t-BuOH (6.02 g, 81.23 mmol, 7.77 mL, 2 eq), DBU (6.18 g, 40.61 mmol, 6.12 mL, 1 eq) were added. The resulting mixture was stirred at 40° C. for 16 hours. TLC (Petroleum ether:Ethyl acetate=0:1) showed a new spot was detected (R_{f}=0.67). EA (100 mL) was added to the mixture, the solution was washed with 10% acetic acid (20 mL), H₂O (50 mL), and aqueous 10% K₂CO₃ (50 mL), and dried over Na₂SO₄, filtered and concentrated under reduced pressure to afford tert-butyl nicotinate (2, 5 g, 27.90 mmol, 68.69% yield) as a yellow oil. ¹H NMR (400 MHz, DMSO) δ 9.11-8.97 (m, 1H), 8.85-8.73 (m, 1H), 8.29-8.16 (m, 1H), 7.62-7.47 (m, 1H), 1.55 (s, 9H). To this solution of (3R,4R,5R)-5-(acetoxymethyl)tetrahydrofuran-2,3,4-triyl triacetate (3, 6.39 g, 20.09 mmol, 1.2 eq), tert-butyl nicotinate (2, 3 g, 16.74 mmol, 1 eq) in DCM (30 mL) was added with TMSOTf (1.86 g, 8.37 mmol, 1.51 mL, 0.5 eq) at 0° C. The mixture was stirred at 25° C. for 16 hours. LCMS (0-60AB/1.5 min, RT=0.879 min, 438.2[M+H]⁺, ESI pos) showed the major peak with desired product was detected. The residue was diluted with ice-H₂O (50 mL), and the mixture was neutralized to pH 6˜7 with saturated aqueous NaHCO₃ (ca. 15 mL). The residue was extracted with DCM (50 mL). The combined organic layers were washed with brine (50 mL), dried over Na₂SO₄, filtered and concentrated under reduced pressure to give a residue. The residue was purified by column chromatography (SiO₂, Petroleum ether:Ethyl acetate=1:0 to 0:1) to afford 3-(tert-butoxycarbonyl)-1-((2R,3R,4R,5R)-3,4-diacetoxy-5-(acetoxymethyl)tetrahydrofuran-2-yl)pyridin-1-ium (3 g, 6.76 mmol, 40.38% yield) as a white solid. LCMS: t_{R}=0.829 min, m/z=438.3 (M+H)^{+ 1}H NMR (400 MHz, D2O) δ 9.44 (s, 1H), 9.30 (d, J=6.2 Hz, 1H), 9.05 (d, J=8.1 Hz, 1H), 8.42-8.33 (m, 1H), 6.70 (d, J=3.4 Hz, 1H), 5.66-5.57 (m, 1H), 5.41 (t, J=5.9 Hz, 1H), 4.77-4.70 (m, 1H), 4.52-4.40 (m, 2H), 2.15 (s, 3H), 2.10 (d, J=7.1 Hz, 6H), 1.61 (s, 9H).

### 1-((2R,3R,4R,5R)-3,4-diacetoxy-5-(acetoxymethyl)tetrahydrofuran-2-yl)-3-(ethoxycarbonyl)pyridin-1-ium

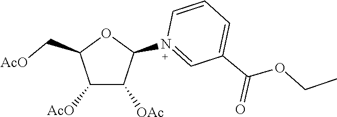

Ethyl nicotinate (1 eq) and β-D-ribofuranose tetraacetate was dissolved in 0.1 M DMF and treaded with 5 eq of TMSOTf. The mixture was stirred at 50° C. for 12 hours. LCMS (The reaction mixture was filtered and concentrated under reduced pressure and was purified by reversed-phase HPLC (0.1% TFA condition) to afford 1-((2R,3R,4R,5R)-3,4-diacetoxy-5-(acetoxymethyl)tetrahydrofuran-2-yl)-3-(ethoxycarbonyl)pyridin-1-ium (82% yield, 97% purity) as a white solid. LCMS: Rt=6.2 min, m/z=411.14 (M+H)⁺.

### 3-(tert-butoxycarbonyl)-1-((2R,3R,4S,5R)-3,4-dihydroxy-5-(hydroxymethyl)tetrahydrofuran-2-yl)pyridin-1-ium

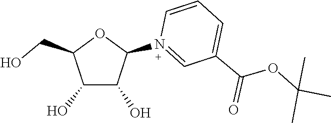

To a solution of 3-(tert-butoxycarbonyl)-1-((2R,3R,4R,5R)-3,4-diacetoxy-5-(acetoxymethyl)tetrahydrofuran-2-yl)pyridin-1-ium (1 g, 2.28 mmol, 1 eq) in MeOH (2 mL) was added NH₃/MeOH (7 M, 10.00 mL, 30.69 eq) at 0° C. The mixture was stirred at 0° C. for 1 hour. LCMS (0-60AB/1.5 min, RT=0.678 min, 312.2[M+H]⁺, ESI pos) showed the major peak with desired product was detected. The reaction mixture was concentrated under reduced pressure to give a residue at 0° C. The crude residue was purified by HPLC (reversed-phase eluting with MeCN/H₂O) to afford 3-(tert-butoxycarbonyl)-1-((2R,3R,4S,5R)-3,4-dihydroxy-5-(hydroxymethyl)tetrahydrofuran-2-yl)pyridin-1-ium (5, 200 mg, 601.91 umol, 26.39% yield) as a yellow oil. LCMS: t_{R}=0.616 min, m/z=312.2 (M+H)⁺. ¹H NMR (400 MHz, D₂O) δ 9.62 (s, 1H), 9.25 (d, J=6.4 Hz, 1H), 9.06 (d, J=8.2 Hz, 1H), 8.28-8.19 (m, 1H), 6.24 (d, J=4.2 Hz, 1H), 4.49-4.43 (m, 2H), 4.37-4.31 (m, 1H), 4.06-4.00 (m, 1H), 3.90-3.85 (m, 1H), 1.61 (s, 9H).

### 1-((2R,3R,4R,5R)-3,4-diacetoxy-5-(acetoxymethyl)tetrahydrofuran-2-yl)-3-(methoxycarbonyl)pyridin-1-ium

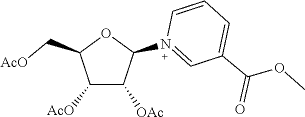

To a solution of (2S,3R,4R,5R)-5-(acetoxymethyl)tetrahydrofuran-2,3,4-triyl triacetate (1.39 g, 4.38 mmol, 1.2 eq) and methyl nicotinate (500 mg, 3.65 mmol, 1 eq) in DCM (10 mL) was added TMSOTf (810.37 mg, 3.65 mmol, 658.83 μL, 1 eq). The mixture was stirred at 25° C. for 16 hours. LCMS (5-95AB/1 min, RT=0.348 min, 396.2[M+H]⁺, ESI pos) showed the major peak with desired MS. The reaction mixture was diluted with ice H₂O (50 mL), the mixture was neutralized to pH 6˜7 with saturated aqueous NaHCO₃ (ca. 15 mL). The residue was extracted with DCM (50 mL). The combined organic layers were washed with brine (50 mL), dried over Na₂SO₄, filtered and concentrated under reduced pressure to give a residue. The residue was purified by column chromatography (SiO₂, Petroleum ether:Ethyl acetate=1:0 to 0:1) to afford 1-((2R,3R,4R,5R)-3,4-diacetoxy-5-(acetoxymethyl) tetrahydrofuran-2-yl)-3-(methoxycarbonyl)pyridin-1-ium (284.20 mg, 689.03 μmol, 27.31% yield, 96.097% purity, TfOH salt) as a yellow solid. LCMS: RT=0.358 min, m/z=396.3 (M+H)^{+ 1}H NMR (400 MHz, CD₃OD) δ 9.66 (s, 1H), 9.37 (d, J=6.4 Hz, 1H), 9.24-9.16 (m, 1H), 8.38 (dd, J=6.4, 7.9 Hz, 1H), 6.61 (d, J=3.6 Hz, 1H), 5.58 (dd, J=3.7, 5.6 Hz, 1H), 5.44 (t, J=5.8 Hz, 1H), 4.82 (td, J=3.0, 5.8 Hz, 1H), 4.63-4.48 (m, 2H), 4.08 (s, 3H), 2.18 (d, J=5.5 Hz, 6H), 2.15 (s, 3H).

### 3-carboxy-1-((2R,3R,4R,5R)-3,4-diacetoxy-5-(acetoxymethyl)tetrahydrofuran-2-yl)pyridin-1-ium

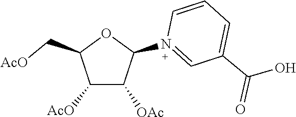

To a solution of nicotinic acid (10 g, 81.23 mmol, 6.80 mL, 1 eq) was added HMDS (39.33 g, 243.69 mmol, 51.08 mL, 3 eq) and (NH₄)₂SO₄ (536.68 mg, 4.06 mmol, 303.21 uL, 0.05 eq). The mixture was stirred at 110° C. for 1 hour. TLC (Petroleum ether:Ethyl acetate=0:1) showed the material was consumed (R_{f}=0.01), and a new spot was detected (R_{f}=0.26). The reaction mixture was filtered and concentrated under reduced pressure to afford trimethylsilyl nicotinate (2, 14 g, 71.69 mmol, 88.25% yield) as a colorless oil. ¹H NMR (400 MHz, DMSO) δ 9.07 (dd, J=0.6, 2.1 Hz, 1H), 8.79 (dd, J=1.7, 4.8 Hz, 1H), 8.26 (td, J=2.0, 7.9 Hz, 1H), 7.54 (ddd, J=0.6, 4.8, 7.9 Hz, 1H), 0.36 (s, 6H), 0.03 (s, 3H). To this solution of (3R,4R,5R)-5-(acetoxymethyl)tetrahydrofuran-2,3,4-triyl triacetate (23.31 g, 73.22 mmol, 1.1 eq), trimethylsilyl nicotinate (2, 13 g, 66.57 mmol, 1 eq) in DCM (300 mL) was added TMSOTf (17.75 g, 79.88 mmol, 14.43 mL, 1.2 eq) at 0° C. The mixture was stirred at 25° C. for 1 hour. LCMS (0-60AB/1.5 min, RT=0.289 min, 382.1 [M+H]⁺, ESI pos) showed the major peak with desired product was detected. The mixture was dissolved in ca. 100 mL of DCM, and the solution was poured into ice-water. The mixture was neutralized to pH 6-7 with saturated aqueous NaHCO₃, colorless aqueous phase was separated from the yellowish organic phase. The aqueous phase was evaporated under reduced pressure at less than 40° C. to give a white solid product. The crude product was purified by reversed-phase HPLC (MeCN/H₂O) to afford 3-carboxy-1-((2R,3R,4R,5R)-3,4-diacetoxy-5-(acetoxymethyl)tetrahydrofuran-2-yl)pyridin-1-ium (13.5 g, 35.40 mmol, 53.18% yield) as a white solid. LCMS: Rt=0.697 min, m/z=382.0 (M+H⁺). ¹H NMR (400 MHz, D₂O) δ 9.38 (s, 1H), 9.07 (d, J=6.2 Hz, 1H), 8.93 (d, J=7.9 Hz, 1H), 8.24-8.09 (m, 1H), 6.55 (d, J=4.0 Hz, 1H), 5.61-5.43 (m, 2H), 4.93-4.82 (m, 1H), 4.52 (d, J=2.2 Hz, 2H), 2.20-2.04 (m, 9H).

### 1-((2R,3R,4R,5R)-3,4-diacetoxy-5-(acetoxymethyl)tetrahydrofuran-2-yl)-3-(((2-isopropyl-5-methylcyclohexyl)oxy)carbonyl)pyridin-1-ium

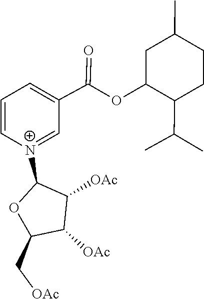 [SMILES: CC(=O)OC[C@H]1O[C@@H]([n+]2cccc(C(=O)OC3CC(C)CCC3C(C)C)c2)[C@H](OC(C)=O)[C@@H]1OC(C)=O]

To a solution of nicotinoyl chloride (2 g, 11.23 mmol, 1 eq, HCl) in DCM (50 mL) was added TEA (2.27 g, 22.47 mmol, 3.13 mL, 2 eq) and 2-isopropyl-5-methylcyclohexan-1-ol (2, 1.76 g, 11.23 mmol, 1.97 mL, 1 eq) at 0° C. The mixture was stirred at 25° C. for 12 hours. LCMS (5-95AB/1 min, RT=0.644 min, 261.9[M+H]⁺, ESI pos) showed the major peak with desired product was detected. The reaction mixture was filtered and concentrated under reduced pressure to give a residue. The residue was purified by column chromatography (SiO₂, Petroleum ether:Ethyl acetate=1:0 to 0:1) to afford 2-isopropyl-5-methylcyclohexyl nicotinate (3, 1 g, 3.82 mmol, 33.99% yield, 99.8% purity) as a yellow solid. LCMS: t_{R}=0.544 min, m/z=330.1 (M+H)⁺. To this solution of 2-isopropyl-5-methylcyclohexyl nicotinate (466 mg, 1.78 mmol, 1 eq), (2S,3R,4R,5R)-5-(acetoxymethyl)tetrahydrofuran-2,3,4-triyl triacetate (680.98 mg, 2.14 mmol, 1.2 eq) in MeCN (10 mL) was added TMSOTf (792.57 mg, 3.57 mmol, 644.36 μL, 2 eq). The mixture was stirred at 25° C. for 16 hours. LCMS (5-95AB/1 min, RT=0.536 min, 520.4[M]⁺, ESI pos) showed the major peak with desired product was detected. The reaction mixture was diluted with 1M NaHCO₃ aq. (50 mL) and extracted with DCM (50 mL), dried over Na₂SO₄, filtered and concentrated under reduced pressure to give a residue. The residue was purified by column chromatography (SiO₂, Petroleum ether:Ethyl acetate=1:0 to 0:1) to afford 1-((2R,3R,4R,5R)-3,4-diacetoxy-5-(acetoxymethyl)tetrahydrofuran-2-yl)-3-(((2-isopropyl-5 methylcyclohexyl)oxy)carbonyl)pyridin-1-ium (TfOH salt, 600 mg, 1.12 mmol, 62.86% yield, 97.25% purity) as a yellow solid. LCMS: t_{R}=0.536 min, m/z=520.4 (M+H)^{+ 1}H NMR (400 MHz, MeOD) 9.66 (br s, 1H), 9.36 (d, J=6.1 Hz, 1H), 9.23-9.16 (m, 1H), 8.43-8.33 (m, 1H), 6.62 (d, J=3.4 Hz, 1H), 5.62-5.55 (m, 1H), 5.47-5.40 (m, 1H), 5.15-5.06 (m, 1H), 4.85-4.81 (m, 1H), 4.63-4.48 (m, 2H), 2.20 (d, J=2.4 Hz, 3H), 2.16 (d, J=8.6 Hz, 6H), 2.09-2.06 (m, 1H), 2.03-1.92 (m, 2H), 1.84-1.76 (m, 2H), 1.71-1.56 (m, 2H), 1.30-1.19 (m, 2H), 0.99-0.94 (m, 6H), 0.84-0.79 (m, 3H).

### 1-((2R,3R,4S,5R)-3,4-dihydroxy-5-(hydroxymethyl)tetrahydrofuran-2-yl)-3-(((2-isopropyl-5-methylcyclohexyl)oxy)carbonyl)pyridin-1-ium

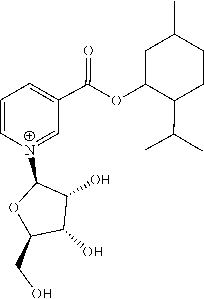 [SMILES: CC1CCC(C(C)C)C(OC(=O)c2ccc[n+]([C@@H]3O[C@H](CO)[C@@H](O)[C@H]3O)c2)C1]

To a solution of 1-((2R,3R,4R,5R)-3,4-diacetoxy-5-(acetoxymethyl)tetrahydrofuran-2-yl)-3-(((2-isopropyl-5-methylcyclohexyl)oxy)carbonyl)pyridin-1-ium (1, 430 mg, 825.99 μmol, 1 eq) was added aq. HCl (3 M, 18 mL, 65.38 eq). The mixture was stirred at 25° C. for 12 hours. LCMS (5-95AB/1 min, RT=0.456 min, 394.2[M+H]⁺, ESI pos) showed the major peak with desired ms. The reaction mixture was filtered and concentrated under reduced pressure to give a residue. The residue was purified by reversed-phase column (H₂O/MeCN) to afford 1-((2R,3R,4S,5R)-3,4-dihydroxy-5-(hydroxymethyl)tetrahydrofuran-2-yl)-3-(((2-isopropyl-5-methylcyclohexyl)oxy)carbonyl)pyridin-1-ium (105 mg, 257.87 μmol, 31.22% yield, 96.88% purity, HCl salt) as a yellow solid. LCMS: RT=0.477 min, m/z=394.3 (M+H)^{+ 1}H NMR (400 MHz, CD₃OD) δ 9.93-9.78 (m, 1H), 9.44 (t, J=6.6 Hz, 1H), 9.13 (d, J=8.1 Hz, 1H), 8.35-8.23 (m, 1H), 6.20 (t, J=4.3 Hz, 1H), 5.17-4.96 (m, 1H), 4.47-4.37 (m, 2H), 4.33-4.26 (m, 1H), 4.00 (dd, J=2.7, 12.3 Hz, 1H), 3.91-3.81 (m, 1H), 2.20-2.08 (m, 1H), 2.03-1.91 (m, 1H), 1.85-1.73 (m, 2H), 1.73-1.53 (m, 2H), 1.30-1.13 (m, 2H), 1.01-0.94 (m, 6H), 0.82 (dd, J=1.6, 6.9 Hz, 3H).

### 1-((2R,3R,4R,5R)-3,4-diacetoxy-5-(acetoxymethyl)tetrahydrofuran-2-yl)-3-((phenylthio)carbonyl)pyridin-1-ium

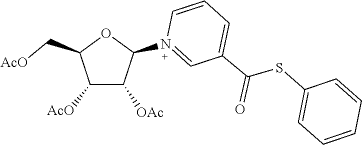

To a solution of nicotinic acid (5 g, 40.61 mmol, 3.40 mL, 1 eq), EDCI (10.12 g, 52.80 mmol, 1.3 eq) in DCM (50 mL) was added dropwise HOBt (7.13 g, 52.80 mmol, 1.3 eq) at 0° C. After addition, the mixture was stirred at 0° C. for 10 min, and then benzenethiol (6.180 g, 56.09 mmol, 5.72 mL, 1.38 eq) was added dropwise at 0° C. The resulting mixture was stirred at 20° C. for 5 hours. LCMS (0-60AB/1.5 min, RT=1.033 min, 216.1 [M+H]⁺, ESI pos) showed the major peak with desired product was detected. The residue was diluted with ice H₂O (150 mL), the residue was extracted with DCM (150 mL). The combined organic layers were washed with brine (210 mL), dried over Na₂SO₄, filtered and concentrated under reduced pressure to give a residue. The aqueous phase was quenched by aq. NaClO. The residue was purified by column chromatography (SiO₂, Petroleum ether:Ethyl acetate=1:0 to 5:1) to afford S-phenyl pyridine-3-carbothioate (7 g, 32.19 mmol, 79.26% yield) as colorless oil. LCMS: t_{R}=0.861 min, m/z=216.3 (M+H)^{+ 1}H NMR (400 MHz, CDCl₃) δ (ppm)=9.25 (d, J=2.1 Hz, 1H), 8.82 (d, J=4.8 Hz, 1H), 8.26 (br d, J=8.1 Hz, 1H), 7.54 (br s, 5H), 7.45-7.41 (m, 1H). To this solution of (3R,4R,5R)-5-(acetoxymethyl)tetrahydrofuran-2,3,4-triyl triacetate (4.69 g, 14.73 mmol, 1.2 eq), tert-butyl nicotinate (2, 2.2 g, 12.28 mmol, 1 eq) in DCM (30 mL) was added TMSOTf (1.36 g, 6.14 mmol, 1.11 mL, 0.5 eq) at 0° C. The mixture was stirred at 25° C. for 2 hours. LCMS (0-60AB/1.5 min, RT=0.915 min, 474.1[M+H]⁺, ESI pos) showed the major peak with desired product was detected. The residue was diluted with ice H₂O (200 mL), the mixture was neutralized to pH 6-7 with saturated aqueous NaHCO₃. The residue was extracted with DCM (50 mE). The combined organic layers were washed with brine (250 mL), dried over Na₂SO₄, filtered and concentrated under reduced pressure to give a residue. The residue was purified by column chromatography (SiO₂, Petroleum ether:Ethyl acetate=1:0 to 0:1) to afford 1-((2R,3R,4R,5R)-3,4-diacetoxy-5-(acetoxymethyl)tetrahydrofuran-2-yl)-3-((phenylthio)carbonyl)pyridin-1-ium (6 g, 12.64 mmol, 90.74% yield) as white solid. LCMS: t_{R}=0.915 min, m/z=474.1 (M+H)^{+ 1}H NMR (400 MHz, CDCl₃) δ 9.58-9.50 (m, 2H), 9.09 (d, J=8.2 Hz, 1H), 8.50-8.40 (m, 1H), 7.51 (s, 5H), 6.69 (d, J=3.8 Hz, 1H), 5.55-5.48 (m, 1H), 5.36 (t, J=5.6 Hz, 1H), 4.77-4.70 (m, 1H), 4.60-4.41 (m, 2H), 2.20-2.12 (m, 9H).

### 1-(3,4-diacetoxy-5-(acetoxymethyl)tetrahydrofuran-2-yl)-3-((((R)-2,5,7,8-tetramethyl-2-((4R,8R)-4,8,12-trimethyltridecyl)chroman-6-yl)oxy)carbonyl)pyridin-1-ium

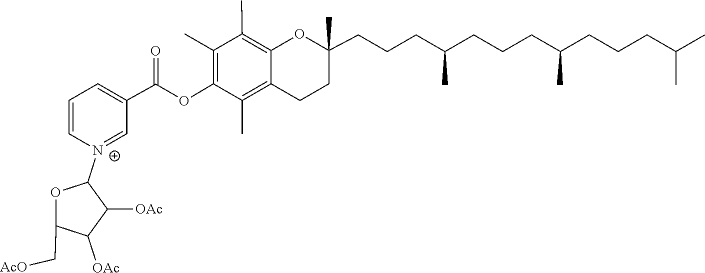 [SMILES: CC(=O)OCC1OC([n+]2cccc(C(=O)Oc3c(C)c(C)c4c(c3C)CC[C@@](C)(CCC[C@H](C)CCC[C@H](C)CCCC(C)C)O4)c2)C(OC(C)=O)C1OC(C)=O]

To a solution of ethane-(R)-2,5,7,8-tetramethyl-2-((4R,8R)-4,8,12-trimethyltridecyl)chroman-6-yl nicotinate (1 eq) in DMF (0.1 M) was added TMSOTf (5 eq) and (2S,3R,4R,5R)-5-(acetoxymethyl) tetrahydrofuran-2,3,4-triyl triacetate (1.64 g, 5.16 mmol, 3 eq) at 0° C. The mixture was stirred at 50° C. for 12 hours. LCMS (. The reaction mixture was filtered and concentrated under reduced pressure and was purified by reversed-phase HPLC (0.1% TFA condition) to afford 1-(3,4-diacetoxy-5-(acetoxymethyl)tetrahydrofuran-2-yl)-3-((((R)-2,5,7,8-tetramethyl-2-((4R,8R)-4,8,12-trimethyltridecyl)chroman-6-yl)oxy)carbonyl)pyridin-1-ium (22% yield, 96% purity) as a white solid. LCMS: Rt=6.9 min, m/z=794 (M)⁺.

### 1-((2R,3R,4S,5R)-3,4-dihydroxy-5-(hydroxymethyl)tetrahydrofuran-2-yl)-3-((phenylthio)carbonyl)pyridin-1-ium

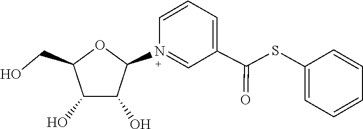

To a solution of 1-((2R,3R,4R,5R)-3,4-diacetoxy-5-(acetoxymethyl)tetrahydrofuran-2-yl)-3-((phenylthio)carbonyl)pyridin-1-ium (1 g, 2.11 mmol, 1 eq) was suspended in aq. HCl (3 M, 10 mL, 14.24 eq) at 0° C. The mixture was stirred at 20° C. for 16 hours. LCMS (0-60AB/1.5 min, RT=0.741 min, 348.0[M+H]⁺, ESI pos) showed the major peak with desired product was detected. The reaction mixture was concentrated under reduced pressure to give a residue at 0° C. The crude product was purified by reversed-phase HPLC (MeCN/H₂O, neutral) to afford 1-((2R,3R,4S,5R)-3,4-dihydroxy-5-(hydroxymethyl)tetrahydrofuran-2-yl)-3-((phenylthio)carbonyl)pyridin-1-ium (300 mg, 809.43 umol, 38.41% yield) as white solid. LCMS: t_{R}=0.727 min, m/z=348.0 (M+H)^{+ 1}H NMR (400 MHz, D₂O) δ 9.75 (s, 1H), 9.29 (d, J=6.2 Hz, 1H), 9.11 (br d, J=8.2 Hz, 1H), 8.35-8.23 (m, 1H), 7.63-7.50 (m, 5H), 6.25 (d, J=3.9 Hz, 1H), 4.51-4.41 (m, 2H), 4.36-4.29 (m, 1H), 4.08-3.99 (m, 1H), 3.90-3.82 (m, 1H).

### 1-((2R,3R,4R,5R)-3,4-diacetoxy-5-(acetoxymethyl)tetrahydrofuran-2-yl)-3-((6-(triphenylphosphonio)hexyl)carbamoyl)pyridin-1-ium

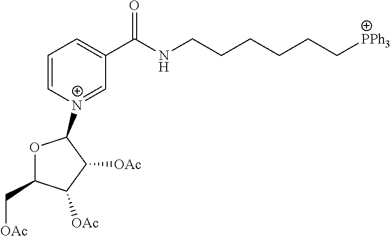

To a solution of (6-(nicotinamido)hexyl)triphenylphosphonium (1 eq) in DMF (0.1 M) was added TMSOTf (5 eq) and (2S,3R,4R,5R)-5-(acetoxymethyl) tetrahydrofuran-2,3,4-triyl triacetate (3 eq) at 0° C. The mixture was stirred at 50° C. for 12 hours. LCMS (. The reaction mixture was filtered and concentrated under reduced pressure and was purified by reversed-phase HPLC (0.1% TFA condition) to afford 1-((2R,3R,4R,5R)-3,4-diacetoxy-5-(acetoxymethyl)tetrahydrofuran-2-yl)-3-((6-(triphenylphosphonio)hexyl)carbamoyl)pyridin-1-ium (28% yield, 97% purity) as a white solid. LCMS: Rt=10.81 min, m/z=726 (M)⁺.

### 1-((2R,3R,4S,5R)-3,4-dihydroxy-5-(hydroxymethyl)tetrahydrofuran-2-yl)-3-((6-(triphenylphosphonio)hexyl)carbamoyl)pyridin-1-ium

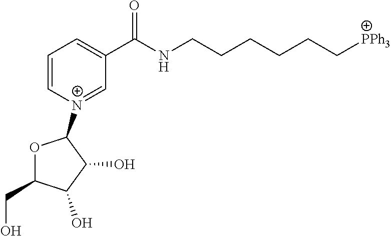

A solution of 1-((2R,3R,4R,5R)-3,4-diacetoxy-5-(acetoxymethyl)tetrahydrofuran-2-yl)-3-((6-(triphenylphosphonio)hexyl)carbamoyl)pyridin-1-ium (1 eq) was suspended in aq. HCl (3 M, 10 mL, 14.24 eq) at 0° C. The mixture was stirred at 20° C. for 16 hours. LCMS showed the major peak with desired product was detected. The reaction mixture was concentrated under reduced pressure to give a residue at 0° C. The crude product was purified by reversed-phase HPLC (MeCN/H₂O, neutral) to afford 1-((2R,3R,4S,5R)-3,4-dihydroxy-5-(hydroxymethyl)tetrahydrofuran-2-yl)-3-((6-(triphenylphosphonio)hexyl)carbamoyl)pyridin-1-ium (38% yield) as white solid. LCMS: t_{R}=4.7 min, m/z=601 (M+H)⁺.

### 1-((2R,3R,4R,5R)-3,4-diacetoxy-5-(acetoxymethyl)tetrahydrofuran-2-yl)-3-((3-(triphenylphosphonio)propyl)carbamoyl)pyridin-1-ium

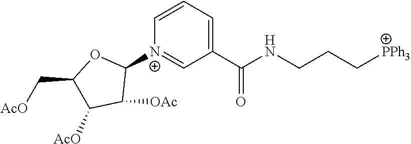

To a solution of (3-(nicotinamido)propyl)triphenylphosphonium (1 eq) in DMF (0.1 M) was added TMSOTf (5 eq) and (2S,3R,4R,5R)-5-(acetoxymethyl) tetrahydrofuran-2,3,4-triyl triacetate (3 eq) at 0° C. The mixture was stirred at 50° C. for 12 hours. LCMS (. The reaction mixture was filtered and concentrated under reduced pressure and was purified by reversed-phase HPLC (0.1% TFA condition) to afford 1-((2R,3R,4R,5R)-3,4-diacetoxy-5-(acetoxymethyl)tetrahydrofuran-2-yl)-3-((3-(triphenylphosphonio)propyl)carbamoyl)pyridin-1-ium (18% yield, 96% purity) as a white solid. LCMS: Rt=5.3 min, m/z=684 (M)⁺.

### 1-((2R,3R,4R,5R)-3,4-diacetoxy-5-(acetoxymethyl)tetrahydrofuran-2-yl)-3-((2-(6-(triphenylphosphonio)hexanamido)ethyl)carbamoyl)pyridin-1-ium

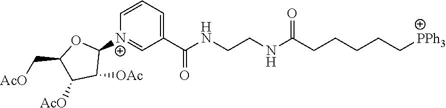

To a solution of (6-((2-(nicotinamido)ethyl)amino)-6-oxohexyl)triphenylphosphonium (1 eq) in DMF (0.1 M) was added TMSOTf (5 eq) and (2S,3R,4R,5R)-5-(acetoxymethyl) tetrahydrofuran-2,3,4-triyl triacetate (3 eq) at 0° C. The mixture was stirred at 50° C. for 12 hours. LCMS (. The reaction mixture was filtered and concentrated under reduced pressure and was purified by reversed-phase HPLC (0.1% TFA condition) to afford 1-((2R,3R,4R,5R)-3,4-diacetoxy-5-(acetoxymethyl)tetrahydrofuran-2-yl)-3-((2-(6-(triphenylphosphonio)hexanamido)ethyl)carbamoyl)pyridin-1-ium (55% yield, 97% purity) as a white solid. LCMS: Rt=6.503 min, m/z=783 (M)⁺.

### ((2R,3S,4R,5R)-3,4-dihydroxy-5-(3-(((2-isopropyl-5-methylcyclohexyl)oxy)carbonyl)pyridin-1-ium-1-yl)tetrahydrofuran-2-yl)methyl hydrogen phosphate

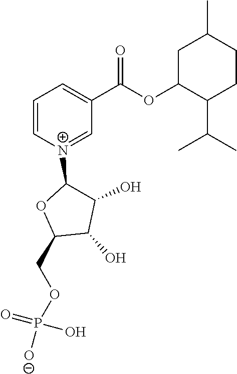 [SMILES: CC1CCC(C(C)C)C(OC(=O)c2ccc[n+]([C@@H]3O[C@H](COP(=O)([O-])O)[C@@H](O)[C@H]3O)c2)C1]

3-carboxy-1-((2R,3R,4S,5R)-3,4-dihydroxy-5-((phosphonooxy)methyl)tetrahydrofuran-2-yl) pyridin-1-ium (NAMN) (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution (2-isopropyl-5-methylcyclohexan-1-ol (1 eq) dissolved in 1 M solution of DMF at 25° C. was added and stirred vigorously. The reaction progression was monitored by LCMS. After completion of the reaction, the crude product was directly purified by reversed-phase HPLC (MeCN/H₂O, 0.01% TFA) to afford ((2R,3S,4R,5R)-3,4-dihydroxy-5-(3-(((2-isopropyl-5-methylcyclohexyl)oxy)carbonyl)pyridin-1-ium-1-yl)tetrahydrofuran-2-yl)methyl hydrogen phosphate as a white solid. LCMS: RT=0.338 min, m/z=474.1 (M+H)⁺. ¹H NMR (400 MHz, CD₃OD) δ 9.63-9.55 (m, 1H), 9.48 (d, J=5.4 Hz, 1H), 9.16-9.08 (m, 1H), 8.37-8.29 (m, 1H), 6.19 (t, J=5.0 Hz, 1H), 5.15-5.01 (m, 1H), 4.55-4.45 (m, 2H), 4.39-4.33 (m, 1H), 4.29-4.10 (m, 2H), 2.18-2.10 (m, 1H), 2.01-1.92 (m, 1H), 1.83-1.76 (m, 2H), 1.71-1.56 (m, 2H), 1.29-1.16 (m, 2H), 1.02-0.99 (m, 1H), 0.99-0.94 (m, 6H), 0.84-0.81 (m, 3H).

### ((2R,3S,4R,5R)-3,4-dihydroxy-5-(3-((tetradecyloxy)carbonyl)pyridin-1-ium-1-yl)tetrahydrofuran-2-yl)methyl hydrogen phosphate

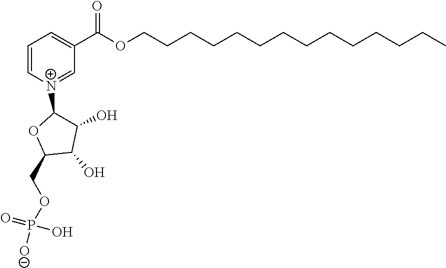 [SMILES: CCCCCCCCCCCCCCOC(=O)c1ccc[n+]([C@@H]2O[C@H](COP(=O)([O-])O)[C@@H](O)[C@H]2O)c1]

3-carboxy-1-((2R,3R,4S,5R)-3,4-dihydroxy-5-((phosphonooxy)methyl)tetrahydrofuran-2-yl) pyridin-1-ium (NAMN) (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution tetradecan-1-ol (1 eq) dissolved in 1 M solution of DMF at 25° C. was added and stirred vigorously. The reaction progression was monitored by LCMS. After completion of the reaction, the crude product was directly purified by reversed-phase HPLC (MeCN/H₂O, 0.01% TFA) to afford ((2R,3S,4R,5R)-3,4-dihydroxy-5-(3-((tetradecyloxy)carbonyl)pyridin-1-ium-1-yl)tetrahydrofuran-2-yl)methyl hydrogen phosphate as a white solid. LCMS: t_{R}=0.641 min, m/z=532.4 (M+H)⁺. ¹H NMR (400 MHz, MeOD) 9.58-9.50 (m, 2H), 9.13-9.08 (m, 1H), 8.37-8.29 (m, 1H), 6.18 (d, J=5.6 Hz, 1H), 4.54-4.46 (m, 4H), 4.38-4.35 (m, 1H), 4.29-4.10 (m, 2H), 1.88-1.81 (m, 2H), 1.50-1.45 (m, 2H), 1.29 (s, 20H), 0.92-0.88 (m, 3H).

### ((2R,3S,4R,5R)-3,4-dihydroxy-5-(3-(tetradecylcarbamoyl)pyridin-1-ium-1-yl)tetrahydrofuran-2-yl)methyl hydrogen phosphate

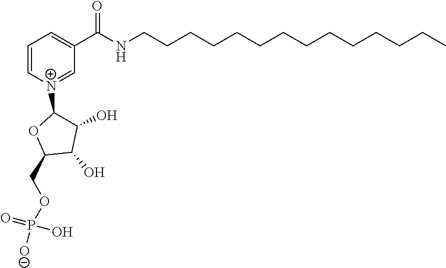 [SMILES: CCCCCCCCCCCCCCNC(=O)c1ccc[n+]([C@@H]2O[C@H](COP(=O)([O-])O)[C@@H](O)[C@H]2O)c1]

3-carboxy-1-((2R,3R,4S,5R)-3,4-dihydroxy-5-((phosphonooxy)methyl)tetrahydrofuran-2-yl) pyridin-1-ium (NAMN) (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution tetradecan-1-amine (1 eq) dissolved in 1 M solution of DMF at 25° C. was added and stirred vigorously. The reaction progression was monitored by LCMS. After completion of the reaction, the crude product was directly purified by reversed-phase HPLC (MeCN/H₂O, 0.01% TFA) to ((2R,3S,4R,5R)-3,4-dihydroxy-5-(3-(tetradecylcarbamoyl)pyridin-1-ium-1-yl)tetrahydrofuran-2-yl)methyl hydrogen phosphate as white solid. LCMS: t_{R}=0.566 min, m/z=531.5 (M+H)^{+ 1}H NMR (400 MHz, MeOD) 9.65 (s, 1H), 9.28 (d, J=6.3 Hz, 1H), 9.00 (d, J=8.0 Hz, 1H), 8.34-8.20 (m, 1H), 6.11 (d, J=6.1 Hz, 1H), 4.57-4.43 (m, 2H), 4.37-4.05 (m, 3H), 3.44 (t, J=7.3 Hz, 2H), 1.73-1.64 (m, 2H), 1.38 (br s, 2H), 1.29 (s, 20H), 0.90 (t, J=6.8 Hz, 3H).

### ((2R,3S,4R,5R)-5-(3-(tert-butoxycarbonyl)pyridin-1-ium-1-yl)-3,4-dihydroxytetrahydrofuran-2-yl)methyl hydrogen phosphate

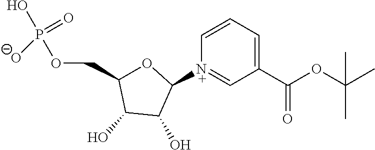 [SMILES: CC(C)(C)OC(=O)c1ccc[n+]([C@@H]2O[C@H](COP(=O)([O-])O)[C@@H](O)[C@H]2O)c1]

3-carboxy-1-((2R,3R,4S,5R)-3,4-dihydroxy-5-((phosphonooxy)methyl)tetrahydrofuran-2-yl) pyridin-1-ium (NAMN) (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution 2-methylpropan-2-ol (1 eq) dissolved in 1 M solution of DMF at 25° C. was added and stirred vigorously. The reaction progression was monitored by LCMS. After completion of the reaction, the crude product was directly purified by reversed-phase HPLC (MeCN/H₂O, 0.01% TFA) to afford ((2R,3S,4R,5R)-5-(3-(tert-butoxycarbonyl)pyridin-1-ium-1-yl)-3,4-dihydroxytetrahydrofuran-2-yl)methyl hydrogen phosphate as a white solid. LCMS: t_{R}=0.660 min, m/z=392.2 (M+H)⁺. ¹H NMR (400 MHz, D₂O) δ 9.41 (s, 1H), 9.33 (d, J=6.5 Hz, 1H), 9.05 (d, J=8.1 Hz, 1H), 8.31-8.24 (m, 1H), 6.19 (d, J=5.3 Hz, 1H), 4.63-4.59 (m, 1H), 4.52 (t, J=5.1 Hz, 1H), 4.44-4.39 (m, 1H), 4.31-4.24 (m, 1H), 4.17-4.10 (m, 1H), 1.61 (s, 9H).

### ((2R,3S,4R,5R)-5-(3-((6-bromohexyl)carbamoyl)pyridin-1-ium-1-yl)-3,4-dihydroxytetrahydrofuran-2-yl)methyl hydrogen phosphate

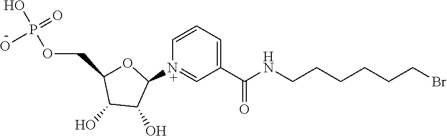 [SMILES: O=C(NCCCCCCBr)c1ccc[n+]([C@@H]2O[C@H](COP(=O)(O)O)[C@@H](O)[C@H]2O)c1]

3-carboxy-1-((2R,3R,4S,5R)-3,4-dihydroxy-5-((phosphonooxy)methyl)tetrahydrofuran-2-yl) pyridin-1-ium (NAMN) (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution 6-bromohexan-1-amine (1 eq) dissolved in 1 M solution of DMF at 25° C. was added and stirred vigorously. The reaction progression was monitored by LCMS. After completion of the reaction, the crude product was directly purified by reversed-phase HPLC (MeCN/H₂O, 0.01% TFA) to afford ((2R,3S,4R,5R)-5-(3-((6-bromohexyl)carbamoyl)pyridin-1-ium-1-yl)-3,4-dihydroxytetrahydrofuran-2-yl)methyl hydrogen phosphate as a white solid. LCMS: t_{R}=0.56 min, m/z=497.2 (M+H)⁺.

### ((2R,3S,4R,5R)-5-(3-((benzyloxy)carbonyl)pyridin-1-ium-1-yl)-3,4-dihydroxytetrahydrofuran-2-yl)methyl hydrogen phosphate

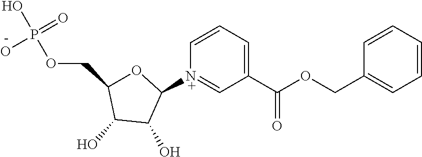 [SMILES: O=C(OCc1ccccc1)c1ccc[n+]([C@@H]2O[C@H](COP(=O)(O)O)[C@@H](O)[C@H]2O)c1]

3-carboxy-1-((2R,3R,4S,5R)-3,4-dihydroxy-5-((phosphonooxy)methyl)tetrahydrofuran-2-yl) pyridin-1-ium (NAMN) (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution phenylmethanol (1 eq) dissolved in 1 M solution of DMF at 25° C. was added and stirred vigorously. The reaction progression was monitored by LCMS. After completion of the reaction, the crude product was directly purified by reversed-phase HPLC (MeCN/H₂O, 0.01% TFA) to afford ((2R,3S,4R,5R)-5-(3-((benzyloxy)carbonyl)pyridin-1-ium-1-yl)-3,4-dihydroxytetrahydrofuran-2-yl)methyl hydrogen phosphate as a white solid. LCMS: Rt=0.181 min, m/z=426.1 (M+H⁺). ¹H NMR (400 MHz, D₂O) δ 9.40 (s, 1H), 9.26 (d, J=6.2 Hz, 1H), 8.97 (br d, J=8.1 Hz, 1H), 8.24-8.17 (m, 1H), 7.42-7.31 (m, 5H), 6.08 (d, J=5.3 Hz, 1H), 5.37 (s, 2H), 4.51 (br d, J=2.1 Hz, 1H), 4.44 (t, J=5.1 Hz, 1H), 4.35-4.30 (m, 1H), 4.20-3.99 (m, 2H).

### ((2R,3S,4R,5R)-3,4-dihydroxy-5-(3-((6-(triphenylphosphonio)hexyl)carbamoyl)pyridin-1-ium-1-yl)tetrahydrofuran-2-yl)methyl hydrogen phosphate

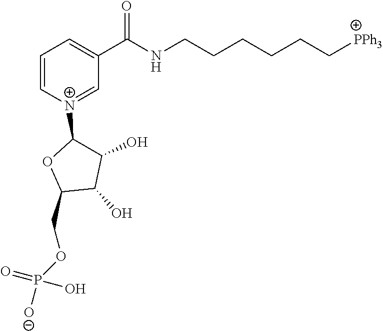

3-carboxy-1-((2R,3R,4S,5R)-3,4-dihydroxy-5-((phosphonooxy)methyl)tetrahydrofuran-2-yl)pyridin-1-ium (1 mmol) was dissolved in deionized water:DMF solution (5 M, 50:50) and sequentially added Diisopropylethylamine (5 eq), 1-Ethyl-3-(3-dimethylaminopropyl)carbodiimide (1 eq). To this solution (6-Aminohexyl)triphenylphosphonium bromide hydrobromide (1 eq) dissolved in 1 M solution of DMF at 25° C.). The reaction progression was monitored by LCMS. After completion of the reaction, the crude product was directly purified by reversed-phase HPLC (MeCN/H₂O) to afford 1-((2R,3R,4S,5R)-3,4-dihydroxy-5-((phosphonooxy)methyl)tetrahydrofuran-2-yl)-3-(tetradecylcarbamoyl)pyridin-1-ium as a white solid. LCMS: (1-99 H₂O/ACN m/min, RT=5.757 min, 679.2 [M+H]⁺, ESI pos) showed the major peak with desired product was detected.

### Example 3: Exemplary Biological Activity of Compounds of the Disclosure

### General NAD Assay Procedure

NHDF cells (Lonza Cat #CC-2511) were plated in 12-well tissue culture plates at a density of 200,000 cells per well in 0.75 mL growth medium (Lonza FGM-2 cat #CC-3132 containing growth factors; cat #CC-4126). The plates were then incubated overnight at 37° C. in a humidified atmosphere of 5% CO₂. The next day, dilutions of each test article were prepared in growth medium at 4× the desired final concentration. 250 μL of each dilution was added to respective wells to obtain desired concentration in each well. The cells were incubated for the desired length of time.

After the incubation, the cell monolayers in each well were washed with cold PBS. 400 μL of extraction buffer (provided in the NAD assay kit) was added and triturated 5-6 time. The cell lysate was collected in Eppendorf tubes and then flash frozen in a dry ice-methanol bath for 20 min then thawed at room temperature. The freeze-thaw cycle was repeated one additional time. The cell lysates were centrifuged and the supernatant collected stored at −80° C. until use.

An aliquot of extract was used to determine total protein concentration using Pierce BCA kit (Thermo Fisher Cat #23225). The total volumes of each sample were adjusted so that the total protein concentration was same in all samples.

50 μL of standards and each sample were added to appropriate wells of a 96-well plate, and the NAD assay according to the kit manufacturer's instructions (NAD/NADH Quantification Kit; Sigma-Aldrich; Catalog Number MAK037). The absorbance at 450 nm was measured using an Envision plate reader (Perkin Elmer) with default settings. The data was normalized against a blank. A standard curve was plotted and used to determine the total NAD level in each test sample.

### Nicotinic Acid Mononucleotide (NaMN)

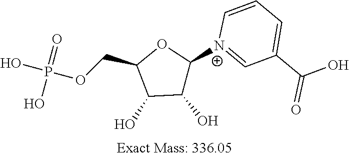 [SMILES: O=C(O)c1ccc[n+]([C@@H]2O[C@H](COP(=O)(O)O)[C@@H](O)[C@H]2O)c1]

Nicotinic acid mononucleotide (NaMN) was analyzed using the General NAD Assay Procedure described above. NaNM (“Sample 1”) was compared to nicotinamide mononucleotide (NMN) at varying concentration and times. The results are shown FIG. 1, as well as in Table 3 below.

TABLE 4
Average Total NAD (pmoles) Concentration from NAD Assay
Comparison of NaNM and NMN

| | | Conc. (μM) | | | |
|---|---|---|---|---|---|
| | | 20 | 10 | 5 | 0 |
| 3-Hour | Sample 1 | 59.0 | 55.0 | 38.1 | 28.2 |
| | NMN | 63.1 | 63.5 | 48.5 | 23.0 |
| 6-Hour | Sample 1 | 79.4 | 77.8 | 72.6 | 33.9 |
| | NMN | 58.6 | 54.9 | 50.9 | 31.1 |

### 1-((2R,3R,4S,5R)-3,4-dihydroxy-5-((phosphonooxy)methyl)tetrahydrofuran-2-yl)-3-((6-(triphenylphosphonio)hexyl)carbamoyl)pyridin-1-ium (7)

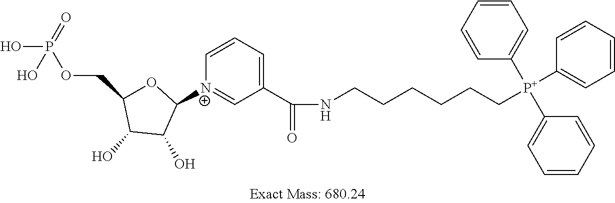 [SMILES: O=C(NCCCCCC[P+](c1ccccc1)(c1ccccc1)c1ccccc1)c1ccc[n+]([C@@H]2O[C@H](COP(=O)(O)O)[C@@H](O)[C@H]2O)c1]

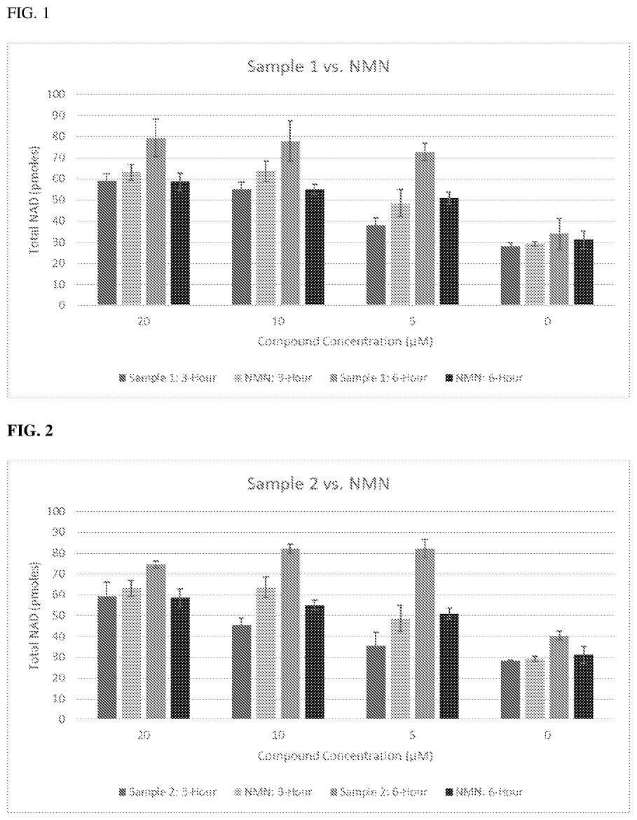

1-((2R,3R,4S,5R)-3,4-dihydroxy-5-((phosphonooxy)methyl)tetrahydrofuran-2-yl)-3-((6-(triphenylphosphonio)hexyl)carbamoyl)pyridin-1-ium (7) was analyzed using the General NAD Assay Procedure described above. 7 (“Sample 2”) was compared to nicotinamide mononucleotide (NMN) at varying concentration and times. The results are shown FIG. 2, as well as in Table 4 below. At the 6-hour time point, compound 7 causes a greater increase in NAD levels than NMN at all concentrations.

TABLE 3
Average Total NAD (pmoles) Concentration from NAD Assay
Comparison of Compound 7 and NMN

| | | Conc. (μM) | | | |
|---|---|---|---|---|---|
| | | 20 | 10 | 5 | 0 |
| 3-Hour | Sample 2 | 59.3 | 45.5 | 35.5 | 28.2 |
| | NMN | 63.1 | 63.5 | 48.5 | 23.0 |
| 6-Hour | Sample 2 | 74.5 | 82.0 | 82.2 | 40.5 |
| | NMN | 58.6 | 54.9 | 50.9 | 31.1 |

### 6-(nicotinamido)hexyl)triphenylphosphonium (10)

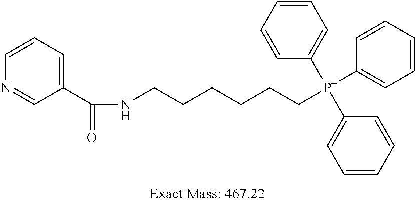 [SMILES: O=C(NCCCCCC[P+](c1ccccc1)(c1ccccc1)c1ccccc1)c1cccnc1]

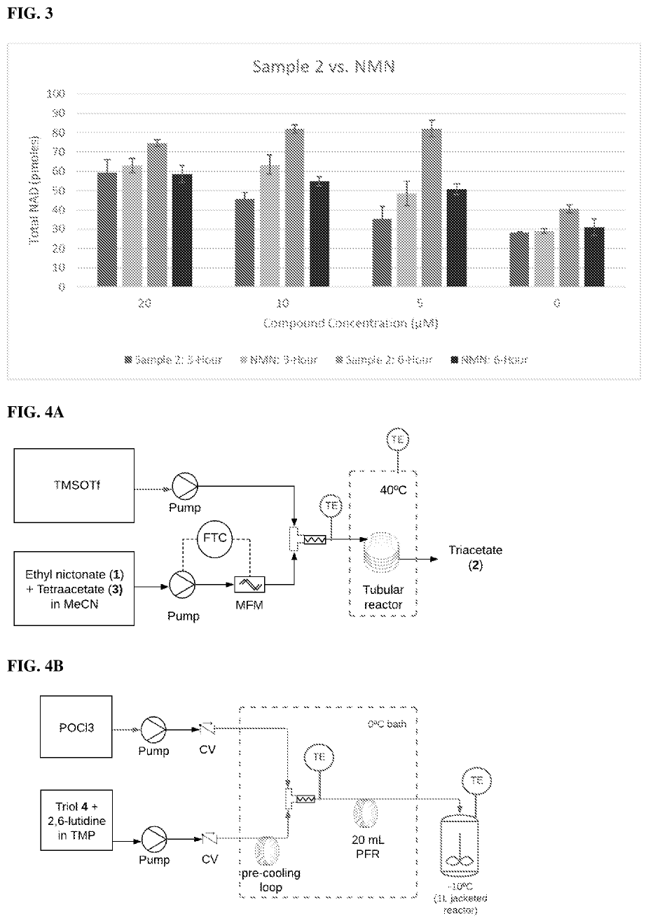

6-(nicotinamido)hexyl)triphenylphosphonium (10) was analyzed using the General NAD Assay Procedure described above. 10 (“Sample 5”) was compared to nicotinamide mononucleotide (NMN) at varying concentration and times. The results are shown FIG. 3, as well as in Table 5 below. At the 6-hour time point, compound 10 causes a greater increase in NAD levels than NMN at all concentrations.

TABLE 4
Average Total NAD (pmoles) Concentration from NAD Assay
Comparison of Compound 10 and NMN

| | | Conc. (μM) | | | |
|---|---|---|---|---|---|
| | | 20 | 10 | 5 | 0 |
| 3-Hour | Sample 5 | 60.0 | 57.5 | 41.6 | 30.1 |
| | NMN | 63.1 | 63.5 | 48.5 | 23.0 |
| 6-Hour | Sample 5 | 76.0 | 77.0 | 68.2 | 42.6 |
| | NMN | 58.6 | 54.9 | 50.9 | 31.1 |

### Example 4: Further Exemplary Biological Activity of Compounds of the Disclosure

The biological activity of certain compounds disclosed herein was determined using the following method:
1. Plate HaCaT cells in 12-well tissue culture plates at a density of 200,000 cells per well in 0.75 mL DMEM growth medium. Incubate overnight at 37° C. in a incubator at 5% CO₂.
2. Next day, prepare dilutions of each compound in growth medium at the desired final concentration.
3. Incubate cells as above for 4 hours.
4. After the incubation, wash cell monolayers in each well with 200 uL PBS and add 200 uL 0.25% Trypsin. Let cells lift for 15 minutes.
5. Once cells are lifted, collect cells into sterile 1.5 mL Eppendorf tube containing 400 uL of DMEM w/2% HI FBS.
6. Centrifuge tubes for 500×g for 5 min.
7. Aspirate supernatant and wash cells with 200 uL of cold PBS.
8. Centrifuge tubes for 500×g for 5 min.
9. Aspirate supernatant and add 400 μL of cold extraction buffer (provided in the NAD assay kit) and vortex each tube for 10 seconds.
10. Centrifuge samples at 13,000×g for 10 min to remove insoluble material.
11. Use a small aliquot of extract to determine total protein concentration by BCA protein assay kit.
12. Deproteinize samples by filtering them through a 10 kDa cut-off spin filter. Centrifuge at 13,000×g for 10 min.
13. After cell lysate is filtered, load 25 uL of samples to 96 well plate for NAD assay.
14. Add 25 uL of extraction buffer to each well with samples to dilute them 1:2. Add 50 uL of standards to appropriate wells.
15. Set up master reaction mix of cycling buffer and NAD cycling enzyme mix. Add 100 uL of mix to each well and incubate for 5 mpg.
16. Add 10 uL of developer into each well.
17. Let plate incubate at room temperature for 1 hr.
18. Stop the reaction by adding 10 uL of stop solution into each well and mix well.
19. Measure absorbance at 450 nm using plate reader.
20. Plot standard curve and normalize data to determine total NAD level.

| Compound | NAD+ level |
|---|---|
| 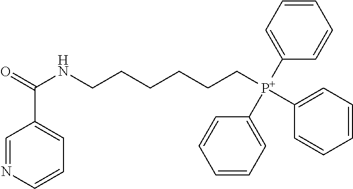 [SMILES: O=C(NCCCCCC[P+](c1ccccc1)(c1ccccc1)c1ccccc1)c1cccnc1] | 217 pmol/mg protein |
| 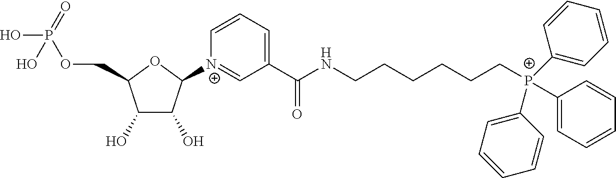 [SMILES: O=C(NCCCCCC[P+](c1ccccc1)(c1ccccc1)c1ccccc1)c1ccc[n+]([C@@H]2O[C@H](COP(=O)(O)O)[C@@H](O)[C@H]2O)c1] | 449 pmol/mg protein |
| 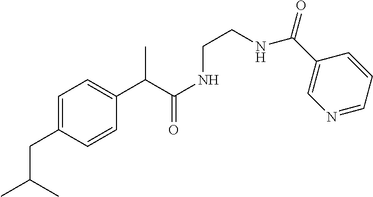 [SMILES: CC(C)Cc1ccc(C(C)C(=O)NCCNC(=O)c2cccnc2)cc1] | 697 pmol/mg protein |
| 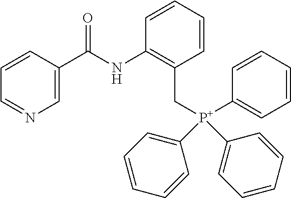 [SMILES: O=C(Nc1ccccc1C[P+](c1ccccc1)(c1ccccc1)c1ccccc1)c1cccnc1] | 666 pmol/mg protein |
| 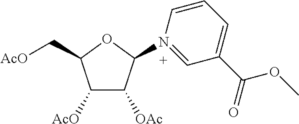 | 323 pmol/mg protein |
| 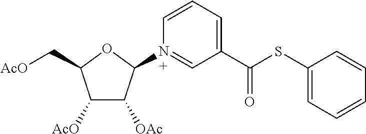 | <1 pmol/mg protein |
| 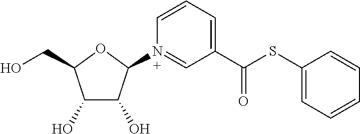 | <1 pmol/mg protein |
| 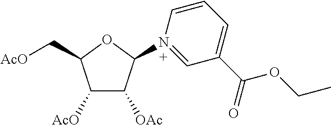 | 674 pmol/mg protein |
| 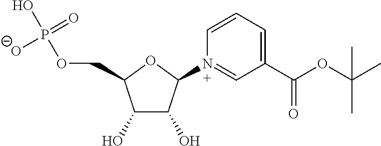 | <1 pmol/mg protein |
| 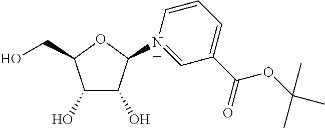 | 1162 pmol/mg protein |
| 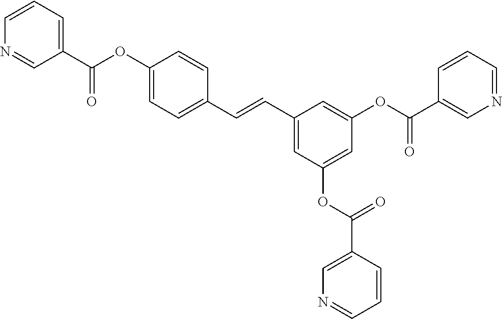 [SMILES: O=C(Oc1ccc(/C=C/c2cc(OC(=O)c3cccnc3)cc(OC(=O)c3cccnc3)c2)cc1)c1cccnc1] | 616 pmol/mg protein |
| 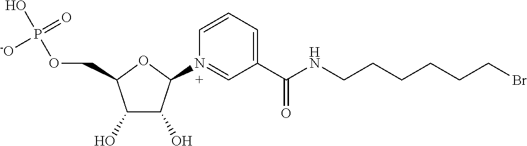 | 349 pmol/mg protein |
| 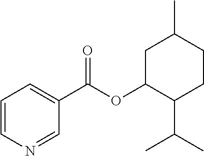 [SMILES: CC1CCC(C(C)C)C(OC(=O)c2cccnc2)C1] | <1 pmol/mg protein |
| 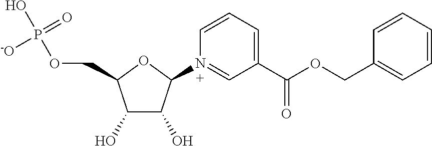 | <1 pmol/mg protein |
| 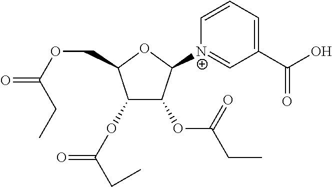 [SMILES: CCC(=O)OC[C@H]1O[C@@H]([n+]2cccc(C(=O)O)c2)[C@H](OC(=O)CC)[C@@H]1OC(=O)CC] | 667 pmol/mg protein |
| 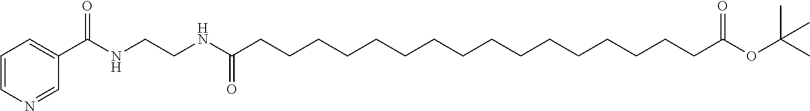 [SMILES: CC(C)(C)OC(=O)CCCCCCCCCCCCCCCCC(=O)NCCNC(=O)c1cccnc1] | 460 pmol/mg protein |
| 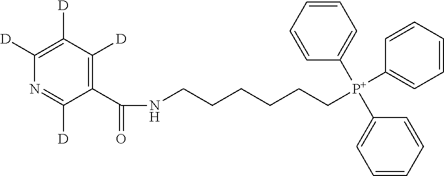 [SMILES: [2H]c1nc([2H])c(C(=O)NCCCCCC[P+](c2ccccc2)(c2ccccc2)c2ccccc2)c([2H])c1[2H]] | 435 pmol/mg protein |
| 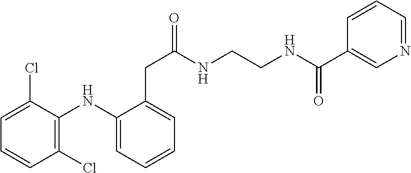 [SMILES: O=C(Cc1ccccc1Nc1c(Cl)cccc1Cl)NCCNC(=O)c1cccnc1] | 338 pmol/mg protein |
| 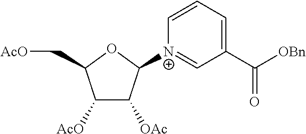 [SMILES: CC(=O)OC[C@H]1O[C@@H]([n+]2cccc(C(=O)OCc3ccccc3)c2)[C@H](OC(C)=O)[C@@H]1OC(C)=O] | 426 pmol/mg protein |
| 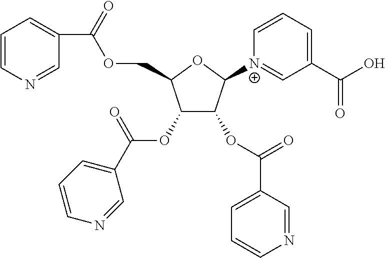 [SMILES: O=C(O)c1ccc[n+]([C@@H]2O[C@H](COC(=O)c3cccnc3)[C@@H](OC(=O)c3cccnc3)[C@H]2OC(=O)c2cccnc2)c1] | 656 pmol/mg protein |
| 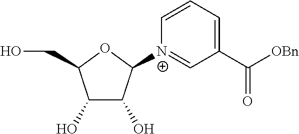 [SMILES: O=C(OCc1ccccc1)c1ccc[n+]([C@@H]2O[C@H](CO)[C@@H](O)[C@H]2O)c1] | 463 pmol/mg protein |
| 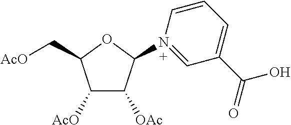 | 311 pmol/mg protein |
| 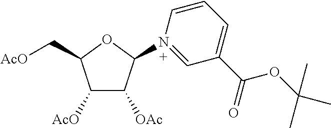 | <1 pmol/mg protein |
| 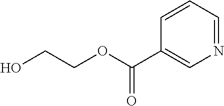 [SMILES: O=C(OCCO)c1cccnc1] | 501 pmol/mg protein |
| 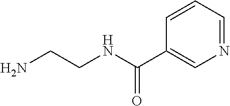 [SMILES: NCCNC(=O)c1cccnc1] | 587 pmol/mg protein |
| 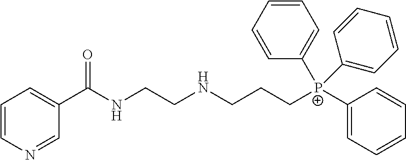 [SMILES: O=C(NCCNCCC[P+](c1ccccc1)(c1ccccc1)c1ccccc1)c1cccnc1] | 391 pmol/mg protein |
| 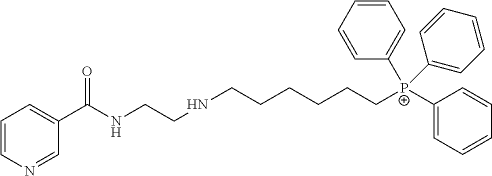 [SMILES: O=C(NCCNCCCCCC[P+](c1ccccc1)(c1ccccc1)c1ccccc1)c1cccnc1] | 378 pmol/mg protein |
| 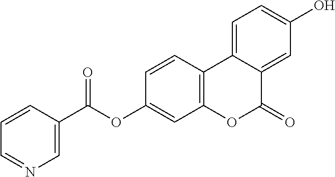 [SMILES: O=C(Oc1ccc2c(c1)oc(=O)c1cc(O)ccc12)c1cccnc1] | 681 pmol/mg protein |
| 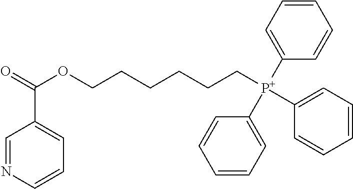 [SMILES: O=C(OCCCCCC[P+](c1ccccc1)(c1ccccc1)c1ccccc1)c1cccnc1] | <1 pmol/mg protein |
| 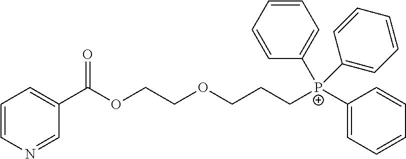 [SMILES: O=C(OCCOCCC[P+](c1ccccc1)(c1ccccc1)c1ccccc1)c1cccnc1] | <1 pmol/mg protein |
| 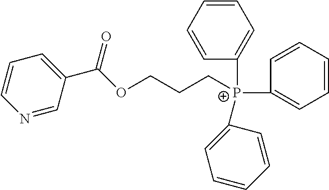 [SMILES: O=C(OCCC[P+](c1ccccc1)(c1ccccc1)c1ccccc1)c1cccnc1] | 576 pmol/mg protein |
| 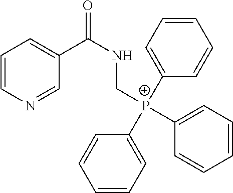 [SMILES: O=C(NC[P+](c1ccccc1)(c1ccccc1)c1ccccc1)c1cccnc1] | 521 pmol/mg protein |
| 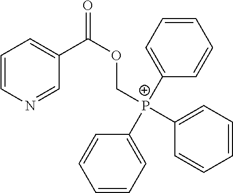 [SMILES: O=C(OC[P+](c1ccccc1)(c1ccccc1)c1ccccc1)c1cccnc1] | 319 pmol/mg protein |
| 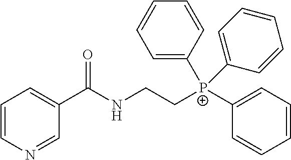 [SMILES: O=C(NCC[P+](c1ccccc1)(c1ccccc1)c1ccccc1)c1cccnc1] | 466 pmol/mg protein |
| 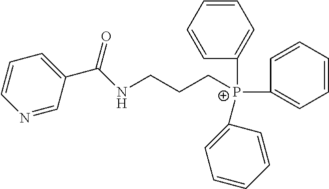 [SMILES: O=C(NCCC[P+](c1ccccc1)(c1ccccc1)c1ccccc1)c1cccnc1] | 560 pmol/mg protein |
| 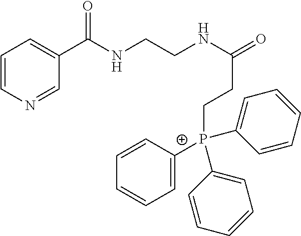 | 641 pmol/mg protein |
| 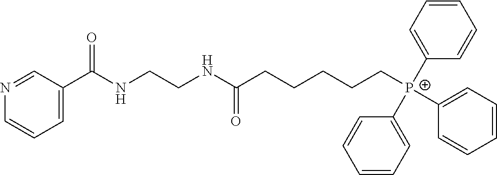 [SMILES: O=C(CCCCC[P+](c1ccccc1)(c1ccccc1)c1ccccc1)NCCNC(=O)c1cccnc1] | 607 pmol/mg protein |
| 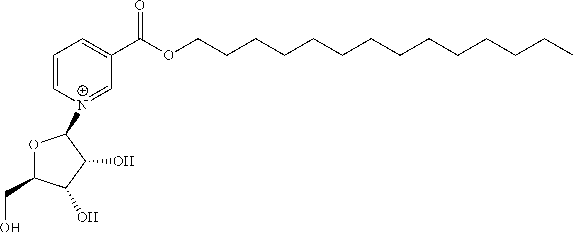 [SMILES: CCCCCCCCCCCCCCOC(=O)c1ccc[n+]([C@@H]2O[C@H](CO)[C@@H](O)[C@H]2O)c1] | <1 pmol/mg protein |
| 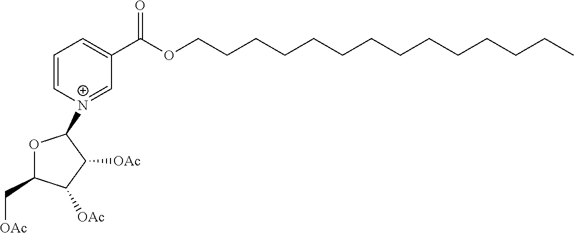 [SMILES: CCCCCCCCCCCCCCOC(=O)c1ccc[n+]([C@@H]2O[C@H](COC(C)=O)[C@@H](OC(C)=O)[C@H]2OC(C)=O)c1] | <1 pmol/mg protein |
| 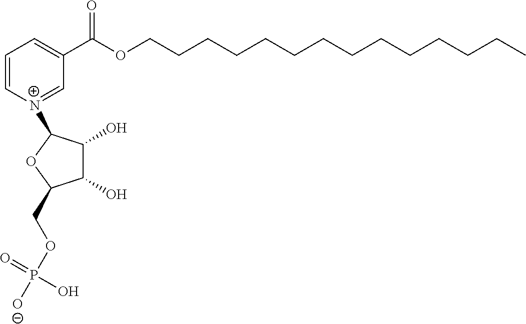 [SMILES: CCCCCCCCCCCCCCOC(=O)c1ccc[n+]([C@@H]2O[C@H](COP(=O)([O-])O)[C@@H](O)[C@H]2O)c1] | 475 pmol/mg protein |
| 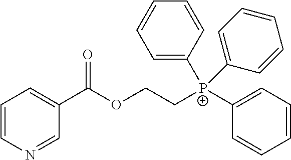 [SMILES: O=C(OCC[P+](c1ccccc1)(c1ccccc1)c1ccccc1)c1cccnc1] | <1 pmol/mg protein |
| 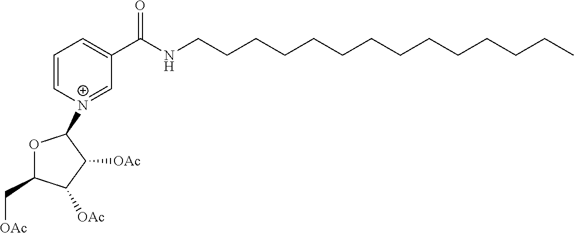 [SMILES: CCCCCCCCCCCCCCNC(=O)c1ccc[n+]([C@@H]2O[C@H](COC(C)=O)[C@@H](OC(C)=O)[C@H]2OC(C)=O)c1] | <1 pmol/mg protein |
| 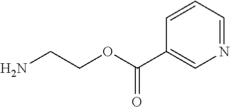 [SMILES: NCCOC(=O)c1cccnc1] | 562 pmol/mg protein |
| 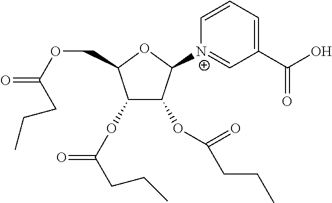 [SMILES: CCCC(=O)OC[C@H]1O[C@@H]([n+]2cccc(C(=O)O)c2)[C@H](OC(=O)CCC)[C@@H]1OC(=O)CCC] | 831 pmol/mg protein |
| 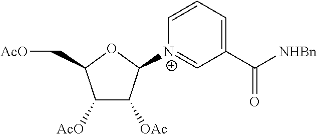 | 399 pmol/mg protein |
| 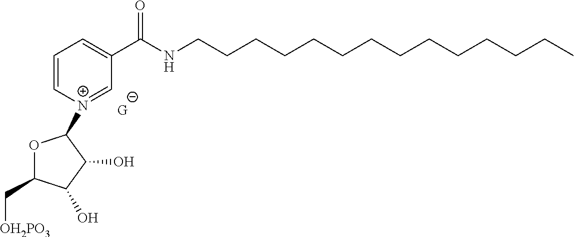 | <1 pmol/mg protein |
| 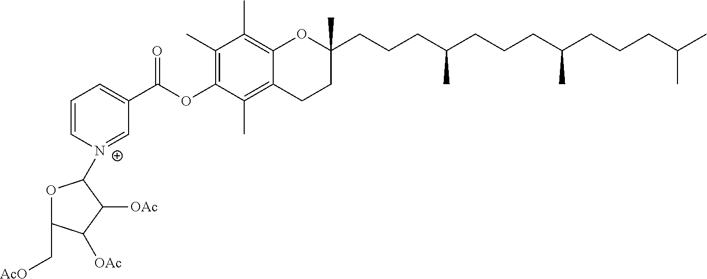 [SMILES: CC(=O)OCC1OC([n+]2cccc(C(=O)Oc3c(C)c(C)c4c(c3C)CC[C@@](C)(CCC[C@H](C)CCC[C@H](C)CCCC(C)C)O4)c2)C(OC(C)=O)C1OC(C)=O] | 301 pmol/mg protein |
| 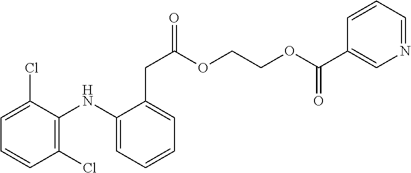 [SMILES: O=C(Cc1ccccc1Nc1c(Cl)cccc1Cl)OCCOC(=O)c1cccnc1] | <1 pmol/mg protein |
| 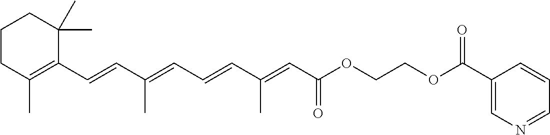 [SMILES: CC1=C(/C=C/C(C)=C/C=C/C(C)=C/C(=O)OCCOC(=O)c2cccnc2)C(C)(C)CCC1] | 788 pmol/mg protein |
| 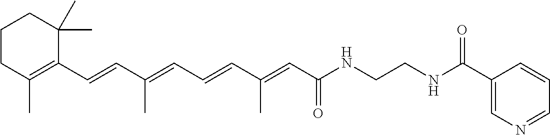 [SMILES: CC1=C(/C=C/C(C)=C/C=C/C(C)=C/C(=O)NCCNC(=O)c2cccnc2)C(C)(C)CCC1] | 815 pmol/mg protein |
| 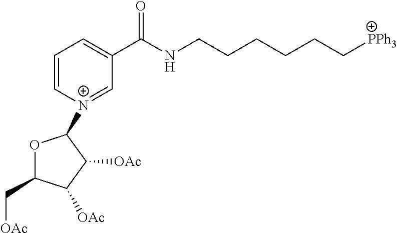 | 565 pmol/mg protein |
| 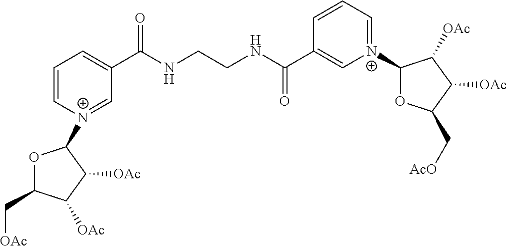 [SMILES: CC(=O)OC[C@H]1O[C@@H]([n+]2cccc(C(=O)NCCNC(=O)c3ccc[n+]([C@@H]4O[C@H](COC(C)=O)[C@@H](OC(C)=O)[C@H]4OC(C)=O)c3)c2)[C@H](OC(C)=O)[C@@H]1OC(C)=O] | 354 pmol/mg protein |
| 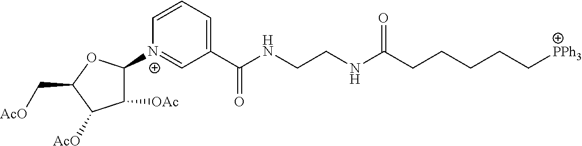 | 311 pmol/mg protein |
| 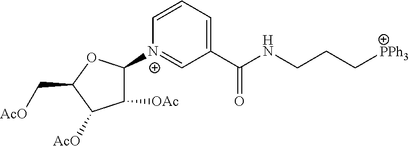 | 292 pmol/mg protein |
| 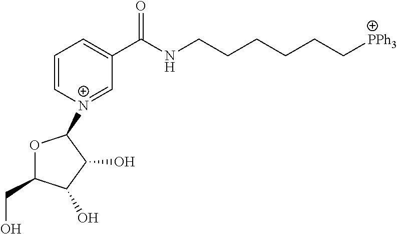 | 285 pmol/mg protein |
| 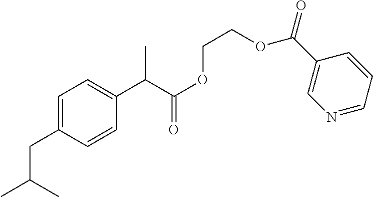 [SMILES: CC(C)Cc1ccc(C(C)C(=O)OCCOC(=O)c2cccnc2)cc1] | 539 pmol/mg protein |
| 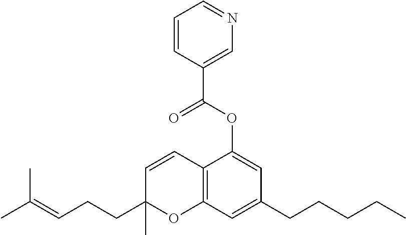 [SMILES: CCCCCc1cc(OC(=O)c2cccnc2)c2c(c1)OC(C)(CCC=C(C)C)C=C2] | 375 pmol/mg protein |
| 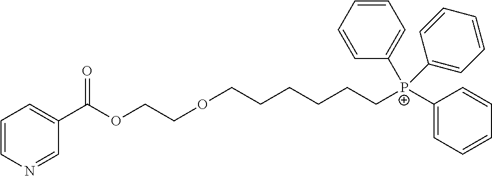 [SMILES: O=C(OCCOCCCCCC[P+](c1ccccc1)(c1ccccc1)c1ccccc1)c1cccnc1] | 230 pmol/mg protein |
| 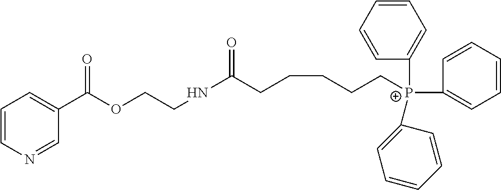 [SMILES: O=C(CCCCC[P+](c1ccccc1)(c1ccccc1)c1ccccc1)NCCOC(=O)c1cccnc1] | <1 pmol/mg protein |
| 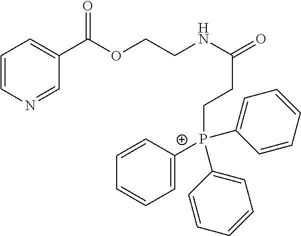 | 478 pmol/mg protein |
| 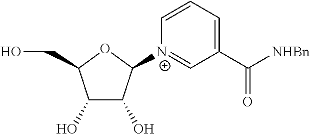 | 274 pmol/mg protein |
| 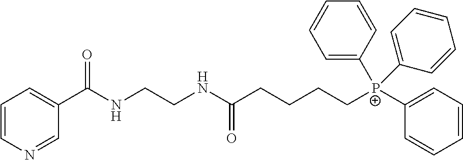 [SMILES: O=C(CCCC[P+](c1ccccc1)(c1ccccc1)c1ccccc1)NCCNC(=O)c1cccnc1] | 495 pmol/mg protein |
| 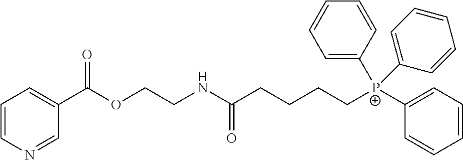 [SMILES: O=C(CCCC[P+](c1ccccc1)(c1ccccc1)c1ccccc1)NCCOC(=O)c1cccnc1] | 508 pmol/mg protein |
| 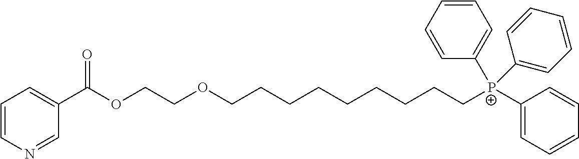 [SMILES: O=C(OCCOCCCCCCCCC[P+](c1ccccc1)(c1ccccc1)c1ccccc1)c1cccnc1] | <1 pmol/mg protein |
| 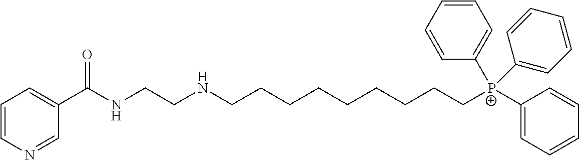 [SMILES: O=C(NCCNCCCCCCCCC[P+](c1ccccc1)(c1ccccc1)c1ccccc1)c1cccnc1] | 395 pmol/mg protein |
| 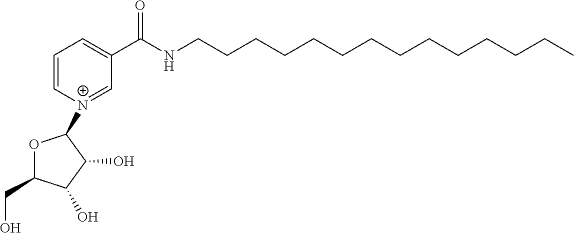 [SMILES: CCCCCCCCCCCCCCNC(=O)c1ccc[n+]([C@@H]2O[C@H](CO)[C@@H](O)[C@H]2O)c1] | <1 pmol/mg protein |
| 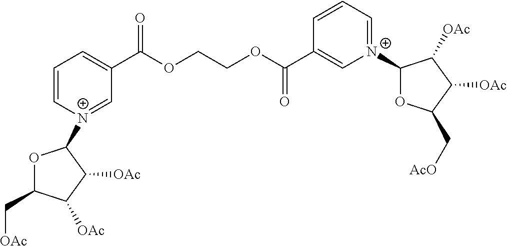 [SMILES: CC(=O)OC[C@H]1O[C@@H]([n+]2cccc(C(=O)OCCOC(=O)c3ccc[n+]([C@@H]4O[C@H](COC(C)=O)[C@@H](OC(C)=O)[C@H]4OC(C)=O)c3)c2)[C@H](OC(C)=O)[C@@H]1OC(C)=O] | 655 pmol/mg protein |
| 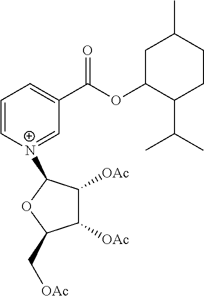 [SMILES: CC(=O)OC[C@H]1O[C@@H]([n+]2cccc(C(=O)OC3CC(C)CCC3C(C)C)c2)[C@H](OC(C)=O)[C@@H]1OC(C)=O] | 258 pmol/mg protein |
| 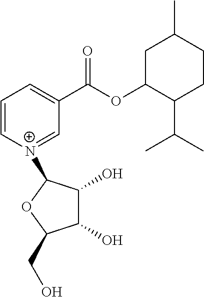 [SMILES: CC1CCC(C(C)C)C(OC(=O)c2ccc[n+]([C@@H]3O[C@H](CO)[C@@H](O)[C@H]3O)c2)C1] | 464 pmol/mg protein |
| 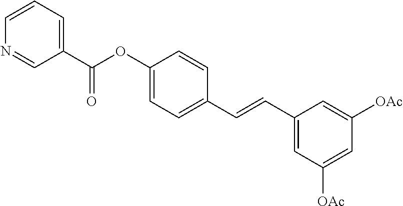 [SMILES: CC(=O)Oc1cc(/C=C/c2ccc(OC(=O)c3cccnc3)cc2)cc(OC(C)=O)c1] | 122 pmol/mg protein |
| 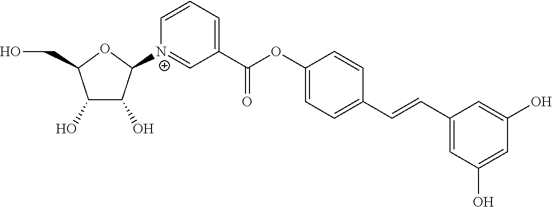 [SMILES: O=C(Oc1ccc(/C=C/c2cc(O)cc(O)c2)cc1)c1ccc[n+]([C@@H]2O[C@H](CO)[C@@H](O)[C@H]2O)c1] | 94 pmol/mg protein |
| 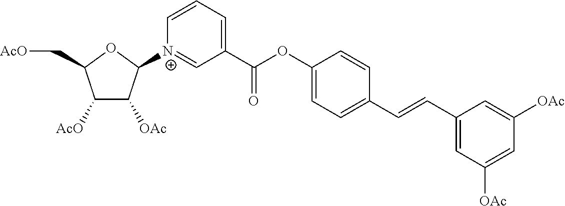 [SMILES: CC(=O)OC[C@H]1O[C@@H]([n+]2cccc(C(=O)Oc3ccc(/C=C/c4cc(OC(C)=O)cc(OC(C)=O)c4)cc3)c2)[C@H](OC(C)=O)[C@@H]1OC(C)=O] | <1 pmol/mg protein |
| 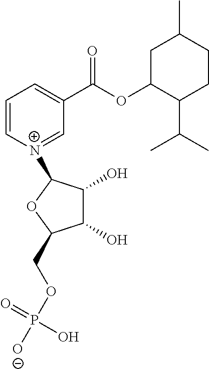 [SMILES: CC1CCC(C(C)C)C(OC(=O)c2ccc[n+]([C@@H]3O[C@H](COP(=O)([O-])O)[C@@H](O)[C@H]3O)c2)C1] | 472 pmol/mg protein |
| 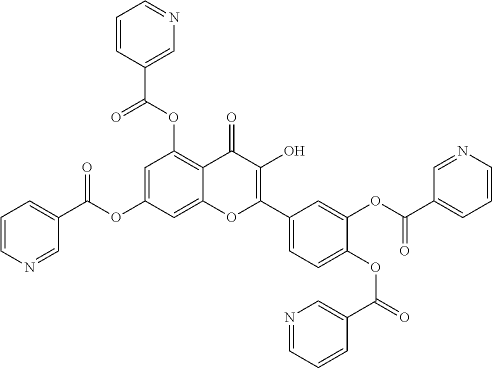 [SMILES: O=C(Oc1cc(OC(=O)c2cccnc2)c2c(=O)c(O)c(-c3ccc(OC(=O)c4cccnc4)c(OC(=O)c4cccnc4)c3)oc2c1)c1cccnc1] | 328 pmol/mg protein |

### Example 5: Exemplary Preparation of Nicotinic Acid Mononucleoside

NAMN was prepared under flow chemistry conditions using the route exemplified in Scheme 5. Firstly, the ribose material was alkylated using ethyl nicotinate in the presence of trimethylsilyl triflate (TMSOTf) in acetonitrile. The resulting triacetate product was then deacetylated using sodium ethoxide in ethanol with subsequent reprotonation using sulfuric acid before being purified via liquid-liquid extraction. Water was then removed from the resulting triol via lyophilization, and the resulting purified triol is then phosphorylated using phosphoryl chloride to give the ethyl ester form of NAMN. The ester group is then saponifled using aqueous sodium hydroxide to give the final product.

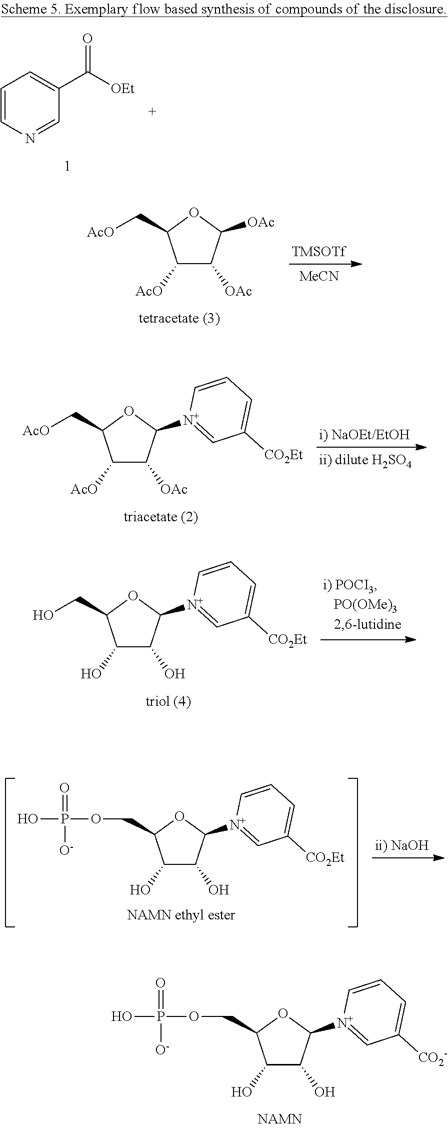

Exemplary flow setups are depicted in FIG. 4.

Briefly β-D-ribofuranose 3 (105.3 g, 330.8 mmol) was dissolved in 5 volumes of acetonitrile (413.8 g, 526.4 mL). Next, ethyl nicotinate (75 g, 496.2 mmol) was added to the solution. The KF of acetonitrile was kept low (below 300 ppm). The density of the solution was measured to be 0.883 g/mL.

The starting material 3 solution was pumped at a flow rate of 4.4 g/min using a diaphragm pump and a mass flow meter. TMSOTf was pumped from a stainless steel syringe using a syringe pump at a flow rate of 0.667 mL/min (or 0.817 g/min). The two solutions were mixed using a static mixer and the thermocouple was used to measure the heat of reaction. Next, the crude solution entered the PFR (30 mL) inside the CASCADE reactor at 40° C. for a residence time of 5 minutes. After exiting the CASCADE reactor, the resulting reaction was collected in a collection flask and stored under nitrogen until further use.

The oil containing triacetate 2 (67.9 w/w %) was used in its concentrated form from the alkylation step.

A solution of sodium ethoxide in ethanol (21 w/w %; Sigma Aldrich) (332.1 g, 382.6 mL) was added to 81.1 g of ethanol to make a 16.9 w/w % NaOEt solution. A solution of sulfuric acid (98 w/w %) (202.4 g, 2.02 mol, 110 mL) was slowly dissolved into water (540 g) to make a 3 M H₂SO₄ solution with stoichiometric water. The crude solution was concentrated at 20-25° C. by rotovap. A quantitative ¹H-NMR was taken to obtain potency by using dimethyl sulfone as internal standard in D₂O. The total mass of oil was 222.4 g (67.9 w/w % 2), giving a 99.9% yield. This step was performed in batch.

A portion of the oil (151.3 g, 67.9 w/w % 2) was dissolved in ethanol (400.4 g, 507.5 mL, 5 vol) and added to a cooled (−5° C.) solution of NaOEt in EtOH (16.9 w/w %) (410.9 g, 480.6 mL, 5.55 equiv) to give a homogeneous dark brown solution. The deprotection was complete within 7 minutes. Dilute sulfuric acid (261.1 g, 142.7 mL, 2.3 equiv wrt 2) was slowly added, maintaining a temperature under 0° C., until pH 7 was achieved. Sodium bicarbonate could be added if the pH was lower than 7. The quench is performed in the minimal amount of time to prevent nicotinic acid from forming.

The heterogeneous solution was then filtered. Ethanol (4 vol with respect to 4) was used to wash the solids. The filtrate was then concentrated by rotovap at 20-30° C. to a crude weight of 139.7 g (48.3 w/w % 4) (×1.75 the theoretical mass of triol 4). Quantitative ¹H-NMR was taken to obtain potency by using dimethyl sulfone as internal standard in D₂O, indicating 85% yield before lyophilization.

The oil was dissolved in water (279.4 mL) and washed with toluene (×3, 116 mL). The resulting aqueous solution containing triol 4 was lyophilized in portions over 2-4 days.

After lyophilization, quantitative ¹H-NMR was taken to obtain potency by using dimethyl sulfone as internal standard in D₂O. The potency of each lyophilized LOT of triol 4 can be seen in the table below. The yield after lyophilization was 72.3%.

The lyophilized triol 4 solids (5.11 g, 7.2 mmol, 61.0 w/w % 4 LOT #JS17-47-6-B-lyo) were dissolved in trimethyl phosphate (18.7 g, 15.6 mL, 5 vol) to give a homogeneous brown solution. The triol 4 solution (23.2 g, 13.5 w/w %) was assayed against an HPLC calibration curve to get potency. 2,6-lutidine (0.78 g, 7.23 mmol, 1 equiv) was added to the solution to bring the total concentration to 13.0 w/w %. The density of the solution was measured to be 1.27 g/mL. The solution was charged into a plastic syringe. POCl₃ was used neat from the bottle and charged into an air-tight glass syringe.

The triol 4 solution was pumped at a flow rate of 0.079 mL/min using a syringe pump. POCl₃ was pumped from a glass syringe using a syringe pump at a flow rate of 0.023 g/min. Check valves were included to ensure there was no back-flow. As both streams entered the dry ice/IPA bath controlled at 0° C., the triol 4 solution was pre-cooled through a 3 mL pre-cooling PFR before meeting the POCl₃ feed.

The two solutions were mixed using a static mixer and the thermocouple was used to measure the heat of reaction. The reaction had a residence time of 60 minutes in a 20 mL PFR at 0° C. to give 95% conversion to NAMN ethyl ester. After exiting the cold bath, the crude product stream was collected in a collection flask held at −10° C. The product was collected for 63 minutes.

The crude phosphorylation containing NAMN ethyl ester was used directly from phosphorylation step. Sodium hydroxide (40 g, 1 mol) was dissolved in water (960 g) to make a 1 M NaOH solution. This step was performed in batch.

The crude NAMN ethyl ester solution was saponified at 18° C. by adding 1 M NaOH aq solution (107 mL, 107 mmol, 59.5 equiv). The saponification occurred over 24 hours, with reaction monitoring by HPLC and pH monitoring by a digital pH probe. The final pH of the solution was 9. Once the saponification was complete as indicated by HPLC, a portion of the material was lyophilized down and assayed by quantitative ¹H-NMR. The overall yield for the phosphorylation and saponification is 66%.

A comparison of different routes to prepare NAMN is set forth in Table 3.

TABLE 5
Comparison of Exemplrary Methods of Preparing NAMN

| Step | | Route A | Route B |
|---|---|---|---|
| 1 (Alkylation) | Description | 5 min residence time, 5 vol MeCN, 1.1 equiv | |
| | | EtNic, 1.1 equiv TMSOTf | |
| | Physical | Homogeneous | |
| | properties | | |
| | Mode | Flow - PFR | |
| 2 (Deprotection) | Description | Concentrate from step | Telescope steps 1 and |
| | | 1, solvent swap to | 2 in flow, deprotect |
| | | EtOH, deprotect with | with 5.75 equiv NaOEt |
| | | 5.75 equiv NaOEt in | in EtOH, quench with |
| | | EtOH, quench with | dilute H₂SO₄ |
| | | dilute H₂SO₄ | |
| | Physical | Deprotection is | Heterogeneous during |
| | properties | homogeneous; filtration | deprotection; filtration |
| | | after quench is quite | after quench is |
| | | slow | moderate |
| | Mode | Flow - PFR | Flow - CSTR |
| 3 | Description | 5 vol TMP, 8 equiv POCl₃, 0 to −5° C., 1 equiv | |
| (Phosphorylation) | | 2,6-lutidine | |
| | Physical | Transient solids at | Heterogeneous |
| | properties | beginning of reaction - | |
| | | homogeneous within | |
| | | minutes | |
| | Mode | Flow - PFR | Flow - CSTR or batch |

| | | |
|---|---|---|
| 4 (Saponification) | Description | Slow addition of NaOF aq solution up to pH 9 |
| | | (typically 37 equiv), held at 18° C. |
| | Physical | Heterogeneous at beginning - homogeneous after |
| | properties | all NaOH aq solution added |
| | Mode | Flow - CSTR (controlled by pH monitoring) |

To summarize, disclosed herein is an alternative route to synthesizing NAMN ethyl nicotinate and β-D-ribofuranose 1,2,3,5-tetraacetate by forming an ethyl ester intermediate. Additionally, analytical methods were carefully constructed in order to efficiently monitor reaction progress and product purity throughout the synthesis. Process development and intensification in batch significantly improved reaction cost and performance at each step. Reported reaction times for each step were reduced from days to hours/minutes. Screening studies were performed to assess the optimal reagents/solvents required to increase kinetics, solubility, and concentration of each step. Each step was evaluated for continuous flow and a proof-of-concept run was demonstrated when appropriate. Furthermore, a cost model for the process demonstrated herein was developed with a feasible path towards a raw material cost being significantly less than $800/kg.

### Example 6: Further Exemplary Preparation of Nicotinic Acid Mononucleoside

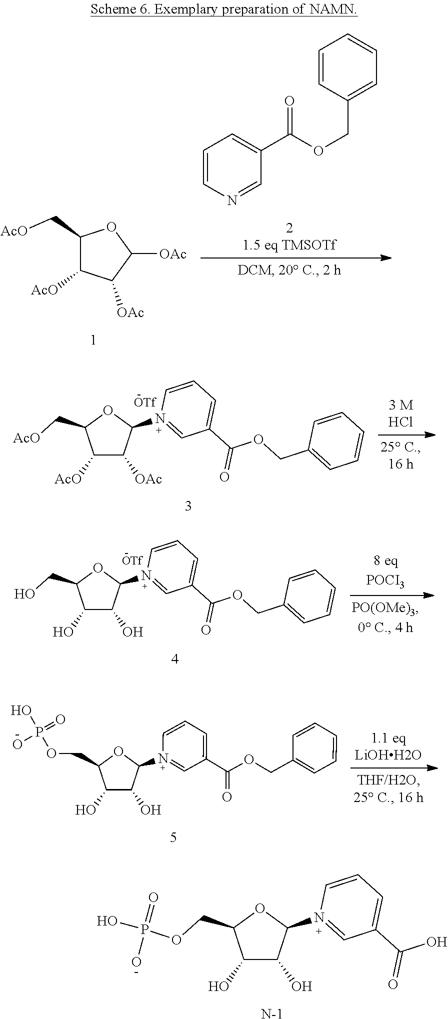

### General Procedure for Preparation of Compound 3.

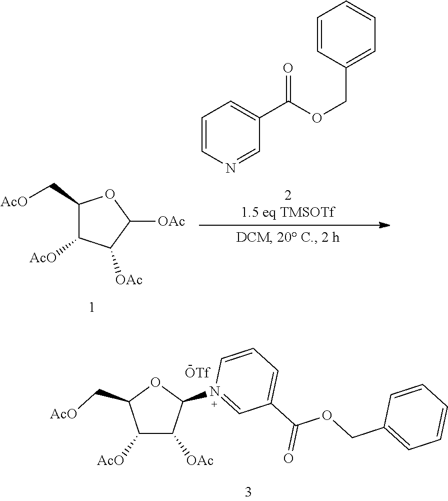

A mixture of (3R,4R,5R)-5-(acetoxymethyl)tetrahydrofuran-2,3,4-triyl triacetate (1, 50 g, 157.10 mmol, 1 eq), benzyl nicotinate (2, 35.17 g, 164.95 mmol, 1.05 eq) in DCM (500 mL) was added TMSOTf (52.37 g, 235.64 mmol, 42.58 mL, 1.5 eq), and then the mixture was stirred at 20° C. for 2 hr under N₂ atmosphere. LCMS (5-95AB/1.5 min, RT=0.653 min, 472.3[M+H]⁺, ESI pos) showed the major peak with desired product was detected. The residue was diluted with ice H₂O 400 mL, the mixture was neutralized to pH 6-7 with saturated aqueous NaHCO₃, The residue was extracted with DCM 100 mL. The combined organic layers were washed with brine 350 mL, dried over Na₂SO₄, filtered and concentrated under reduced pressure to give a residue. The residue was purified by column chromatography (SiO₂, Petroleum ether:Ethyl acetate=1:0 to 0:1) to afford 3-((benzyloxy)carbonyl)-1-((2R,3R,4R,5R)-3,4-diacetoxy-5-(acetoxymethyl)tetrahydrofuran-2-yl)pyridin-1-ium (3, 70 g, 148.16 mmol, 94.31% yield) as a yellow oil. LCMS: Rt=0.653 min, m/z=472.3 (M+H⁺); ¹H NMR (400 MHz) δ 9.58 (s, 1H), 9.46 (br d, J=6.2 Hz, 1H), 9.03 (d, J=8.1 Hz, 1H), 8.37-8.30 (m, 1H), 7.47-7.37 (m, 5H), 6.63 (d, J=3.8 Hz, 1H), 5.50-5.44 (m, 3H), 5.33 (t, J=5.6 Hz, 1H), 4.76-4.67 (m, 1H), 4.57-4.41 (m, 2H), 2.14-2.07 (m, 9H).

### General Procedure for Preparation of Compound 4.

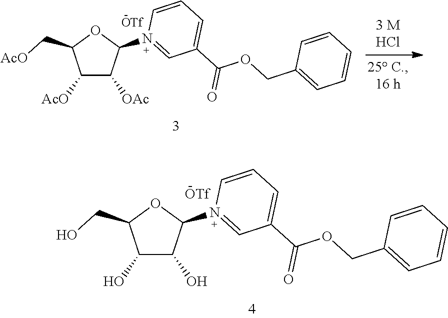

A mixture of 3-((benzyloxy)carbonyl)-1-((2R,3R,4R,5R)-3,4-diacetoxy-5-(acetoxymethyl) tetrahydrofuran-2-yl)pyridin-1-ium (3, 70 g, 148.16 mmol, 1 eq), HCl (3 M, 700.00 mL, 14.17 eq), and then the mixture was stirred at 25° C. for 16 hours. LCMS (0-60AB/1.5 min, RT=0.689 min, 346.0[M+H]⁺, ESI pos) showed the major peak with desired product was detected. The reaction mixture was concentrated under reduced pressure to remove HCl at 25° C. The residue was diluted with H₂O (500 mL) and extracted with DCM (300 mL). The aqueous phase was freeze-dried to afford 3-((benzyloxy)carbonyl)-1-((2R,3R,4S,5R)-3,4-dihydroxy-5-(hydroxymethyl)tetrahydrofuran-2-yl)pyridin-1-ium (4, 50 g, crude, HCl) as a yellow solid. LCMS: Rt=0.689 min, m/z=346.0 (M+H⁺).

### General Procedure for Preparation of Compound 5.

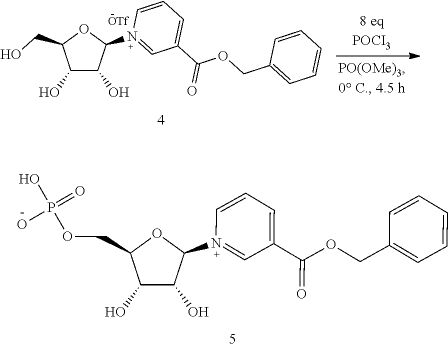

To a solution of 3-((benzyloxy)carbonyl)-1-((2R,3R,4S,5R)-3,4-dihydroxy-5-(hydroxymethyl) tetrahydrofuran-2-yl)pyridin-1-ium (4, 40 g, 115.49 mmol, 1 eq) in PO(OMe)₃ (200 mL) was added dropwise POCl₃ (80 mL) at 0° C. for 0.5 h. The mixture was stirred at 0° C. for 4 hours. LCMS (quenched by ice H₂O) (0-60AB/1.5 min, RT=0.256 min, 426.1[M+H]⁺, ESI pos) showed the major peak with desired MS was detected. The reaction mixture was added slowly to H₂O (200 mL) at 0° C., then the mixture was stirred at 0° C. for 1.5 h. The solution was purified by reversed-phase HPLC (neutral condition, keep H₂O washed 0.5 hours, mobile phase: [water-ACN]; B %: 0%-30%) to afford ((2R,3S,4R,5R)-5-(3-((benzyloxy) carbonyl)pyridine-1-ium-1-yl)-3,4-dihydroxytetrahydrofuran-2-yl)methyl hydrogen phosphate (5, 15 g, 28.56 mmol, 30.53% yield, 81% purity) as a yellow solid. LCMS: Rt=0.181 min, m/z=426.1 (M+H⁺); ¹H NMR (400 MHz, D₂O) δ 9.40 (s, 1H), 9.26 (d, J=6.2 Hz, 1H), 8.97 (br d, J=8.1 Hz, 1H), 8.24-8.17 (m, 1H), 7.42-7.31 (m, 5H), 6.08 (d, J=5.3 Hz, 1H), 5.37 (s, 2H), 4.51 (br d, J=2.1 Hz, 1H), 4.44 (t, J=5.1 Hz, 1H), 4.35-4.30 (m, 1H), 4.20-3.99 (m, 2H).

### General Procedure for Preparation of N-1.

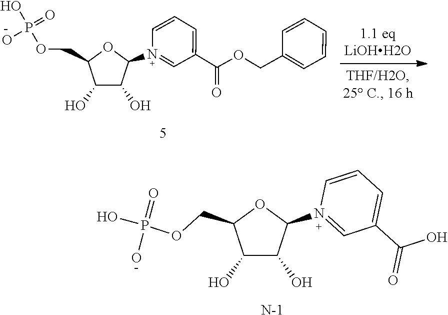

To a solution of ((2R,3S,4R,5R)-5-(3-((benzyloxy)carbonyl)pyridine-1-ium-1-yl)-3,4-dihydroxytetrahydrofuran-2-yl)methyl hydrogen phosphate (5, 8 g, 18.81 mmol, 1 eq) in THF (60 mL), H₂O (60 mL) was added LiOH·H₂O (955.05 mg, 22.76 mmol, 1.2 eq). The mixture was stirred at 25° C. for 16 hours. Special LCMS (0-30AB_7MIN_T3_5CM, RT=0.790 min, 336.0[M+H]⁺, ESI pos) showed the major peak with desired product was detected. The reaction mixture was purified by ion-exchange resin(H) form to afford ((2R,3S,4R,5R)-5-(3-carboxypyridin-1-ium-1-yl)-3,4-dihydroxytetrahydrofuran-2-yl)methyl hydrogen phosphate (N-1, 2 g, 5.55 mmol, 29.50% yield, 93% purity) as a yellow solid. LCMS: t_{R}=0.814 min, m/z=336.0 (M+H)⁺; ¹H NMR (400 MHz, D₂O) δ 9.45 (s, 1H), 9.28 (d, J=6.2 Hz, 1H), 9.03 (br d, J=8.1 Hz, 1H), 8.30-8.19 (m, 1H), 6.18 (d, J=5.3 Hz, 1H), 4.62-4.57 (m, 1H), 4.51 (t, J=5.1 Hz, 1H), 4.43-4.37 (m, 1H), 4.29-4.10 (m, 2H).

### INCORPORATION BY REFERENCE

All U.S. patents and U.S. and PCT patent application publications mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by formula (VI) or a pharmaceutically acceptable salt thereof: 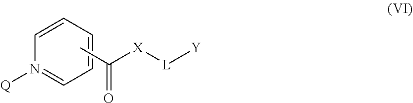
wherein:
Q is absent, 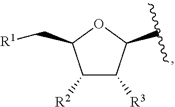
or H;
R¹ is HPO₄, H₂PO₄, —OH, —OAcyl, or —OC(O)R⁴;
R² and R³ are independently —OH, —C(O)R⁴, —C(O)OR⁴, —C(O)NHR⁴ or halogen;
R⁴ is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
L is a bond, C₁₋₂₀ alkyl, aryl, heteroaryl, arylalkylaryl, arylalkyl, alkoxy, or —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
Y is —C(O)NH₂, —C(O)OH, —R⁵, —P(R⁷)₃, —NH₂, —NHR⁵, 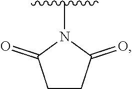 [SMILES: *N1C(=O)CCC1=O]
—SH, or —OH;
R⁵ is —C(O)R⁴, 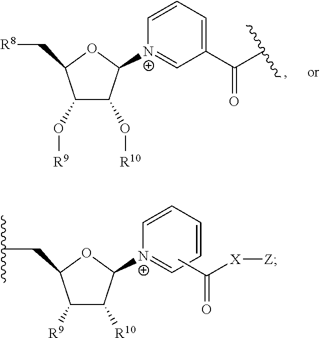
R⁷ is individually selected at each occurrence from the group consisting of substituted or unsubstituted alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —H, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z is H, or C₁₋₂₀ alkyl; or Z and R¹ are optionally taken together as a bond, forming a macrocycle,
with the proviso that if X is O, NH, or NR⁷, and L is C₁₋₂₀ alkyl, aryl, heteroaryl, or alkoxy, then Y is not —C(O)NH₂, —C(O)OH, —R⁵, —NH₂, —NHR^{S}, —SH, or —OH.

2. The compound of claim 1, wherein the compound has a structure represented by formula VIa: 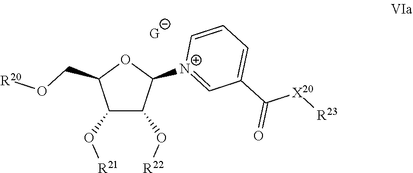
wherein,
R²⁰ is H, P(O)₂OH, P(O)(OH)₂, or acyl;
R²¹ and R²² are each independently H or acyl;
R²³ is H, alkyl, cycloalkyl, aralkyl, or aryl;
R²⁴ is H or alkyl;
X²⁰ is O, N(R²⁴), or S; and
G is an anion.

3. The compound of claim 2, wherein R² is H.

4. The compound of claim 2, wherein R² is P(O)(OH)₂.

5. The compound of claim 2, wherein R² is acyl (e.g., alkylacyl or heteroarylacyl).

6. The compound of any one of claims 2-5, wherein R²¹ is H.

7. The compound of any one of claims 2-5, wherein R²¹ is acyl (e.g., alkylacyl or heteroarylacyl).

8. The compound of any one of claims 2-7, wherein R²² is H.

9. The compound of any one of claims 2-7, wherein R²² is acyl (e.g., alkylacyl or heteroarylacyl).

10. The compound of any one of claims 2-9, wherein X²⁰ is O.

11. The compound of any one of claims 2-9, wherein X²⁰ is NH.

12. The compound of any one of claims 2-9, wherein X²⁰ is S.

13. The compound of any one of claims 2-12, wherein R²³ is H.

14. The compound of any one of claims 2-12, wherein R²³ is alkyl.

15. The compound of claim 14, wherein R²³ is alkylaminoalkyl.

16. The compound of claim 14, wherein R²³ is alkylamidoalkyl.

17. The compound of any one of claims 2-12, wherein R²³ is aralkyl (e.g., benzyl).

18. The compound of any one of claims 2-12, wherein R²³ is aryl (e.g., phenyl).

19. The compound of any one of claims 2-12, wherein R²³ is cycloalkyl (e.g., cyclohexyl).

20. The compound of any one of claims 2-19, wherein R²³ is substituted with triarylphosphonium (e.g., P⁺(Ph)₃).

21. The compound of any one of claims 2-20, wherein R²³ is substituted with vinyl (e.g., phenylvinyl, such as dihydroxyphenylvinyl or diacetylphenylvinyl).

22. The compound of any one of claims 2-21, wherein R²³ is substituted with amido.

23. The compound of claim 22, wherein R²³ is substituted with

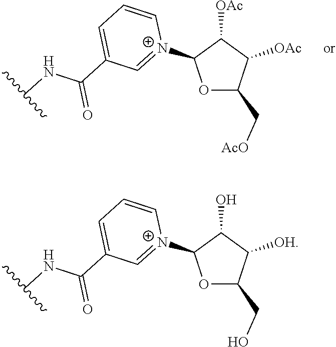 [SMILES: *NC(=O)c1ccc[n+]([C@@H]2O[C@H](COC(C)=O)[C@@H](OC(C)=O)[C@H]2OC(C)=O)c1 ; "or" ; *NC(=O)c1ccc[n+]([C@@H]2O[C@H](CO)[C@@H](O)[C@H]2O)c1]

24. The compound of any one of claims 2-23, wherein R²³ is substituted with ester.

25. The compound of claim 24, wherein R²³ is substituted with

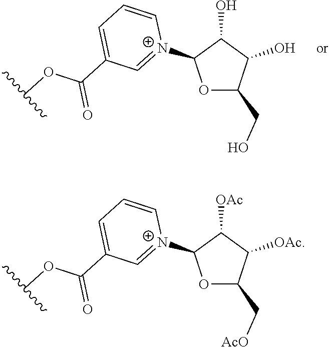 [SMILES: *OC(=O)c1ccc[n+]([C@@H]2O[C@H](CO)[C@@H](O)[C@H]2O)c1 ; "or" ; *OC(=O)c1ccc[n+]([C@@H]2O[C@H](COC(C)=O)[C@@H](OC(C)=O)[C@H]2OC(C)=O)c1]

26. The compound of any one of claims 2-25, wherein R²³ is substituted with halo (e.g., bromo).

27. The compound of any one of claims 2-26, wherein R²³ is substituted with alkyl.

28. The compound of any one of claims 2-27, wherein G is a pharmaceutically acceptable anion.

29. The compound of claim 1, wherein the compound is selected from the group consisting of: 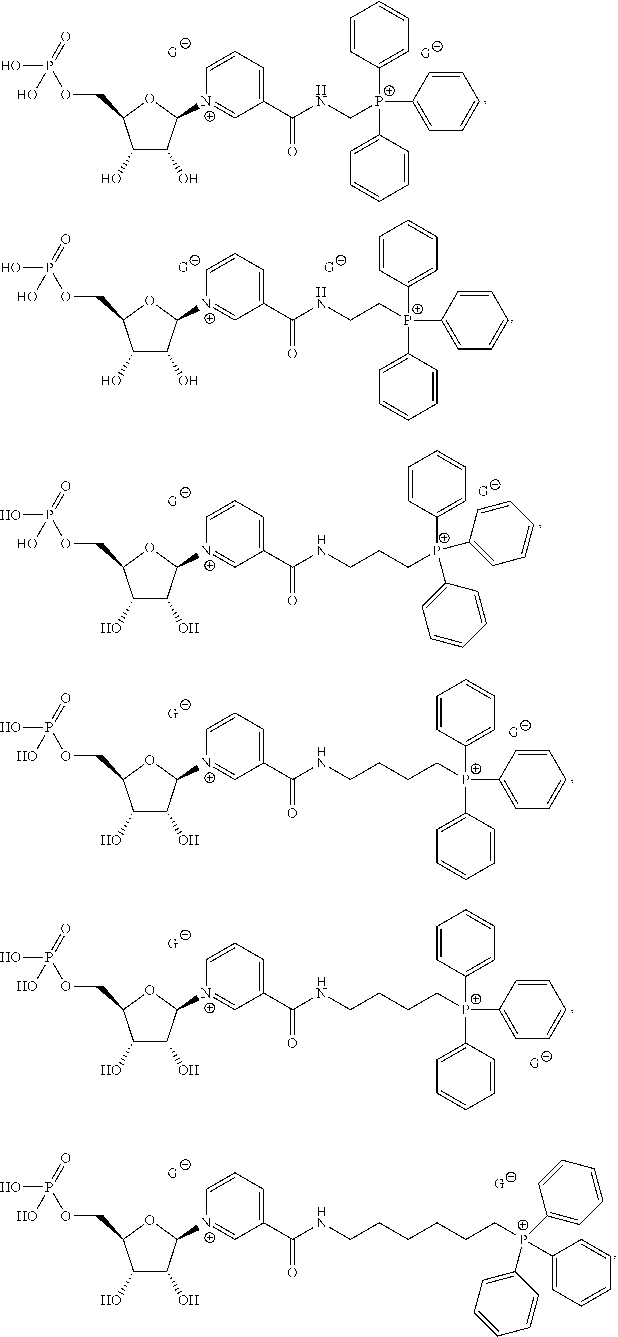 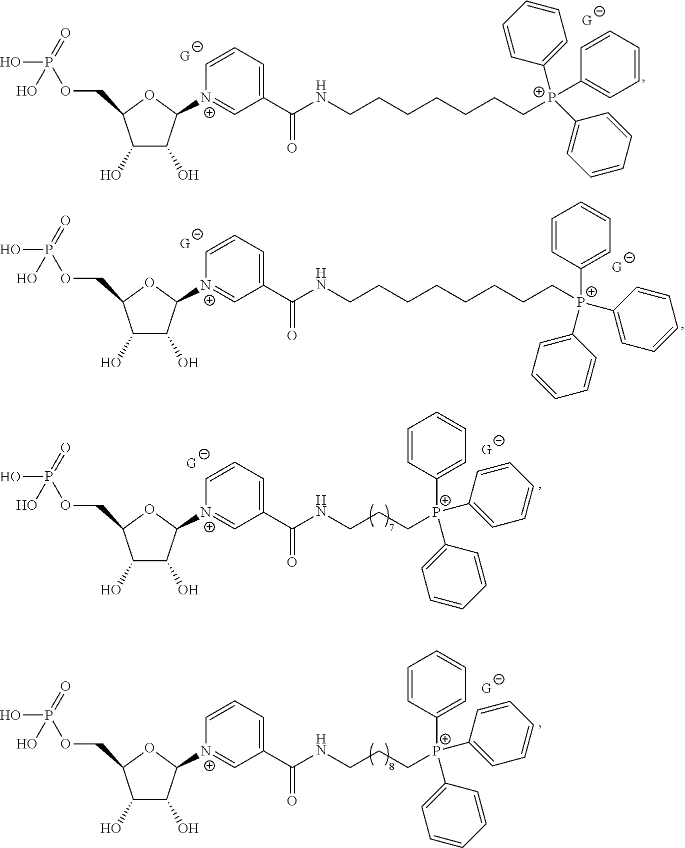 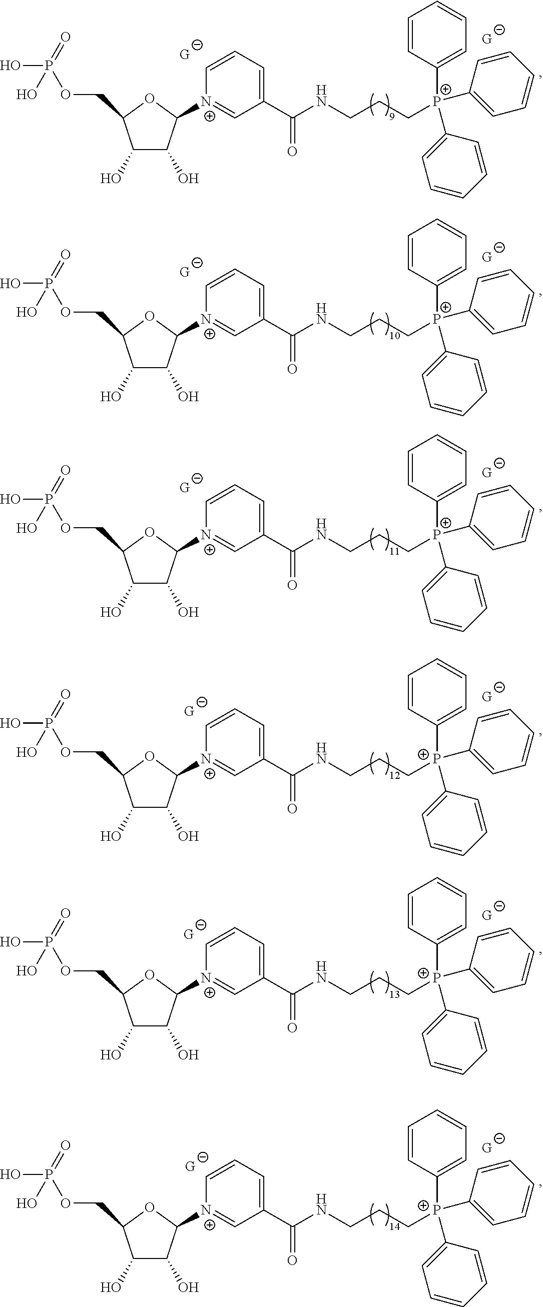 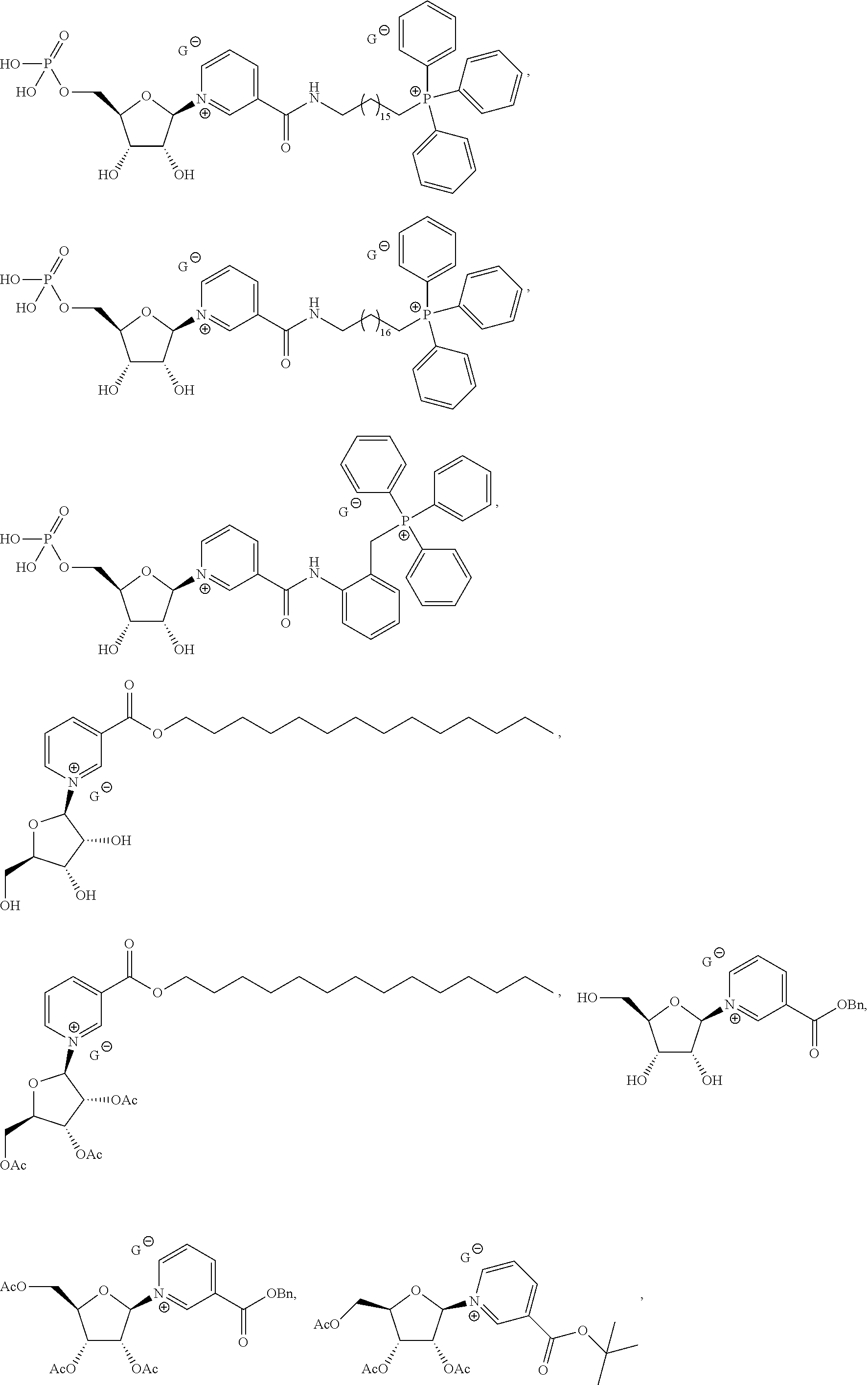 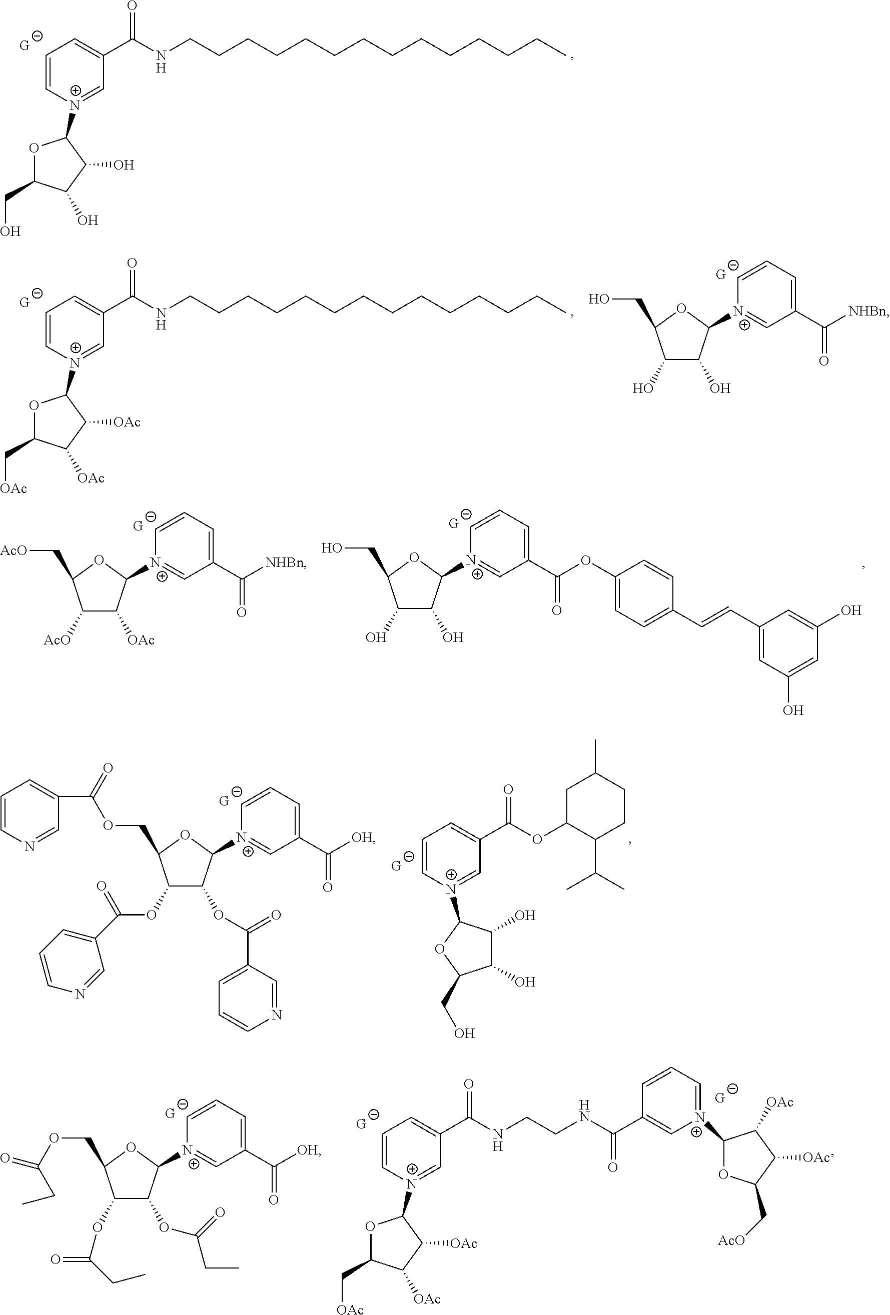 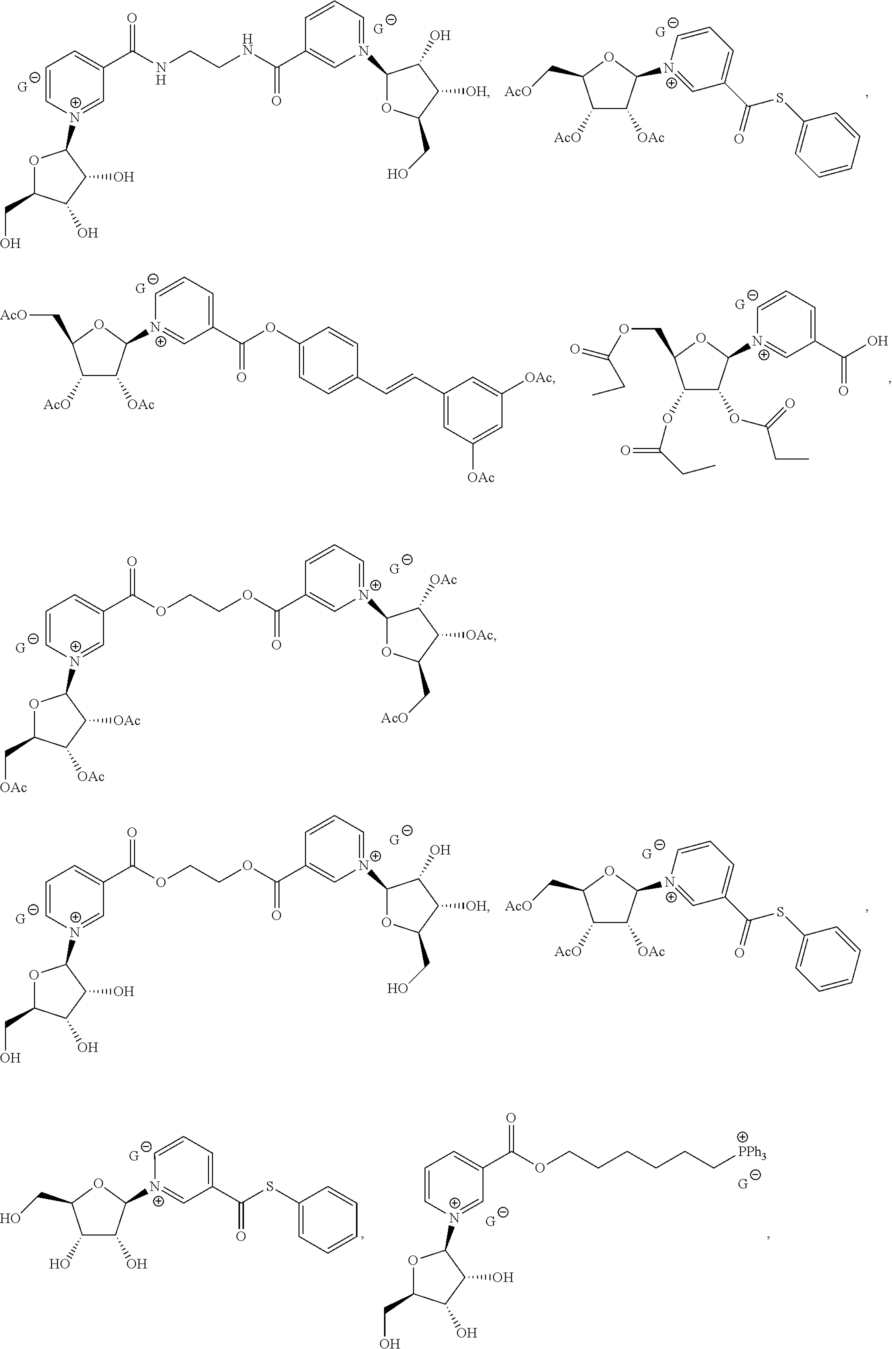 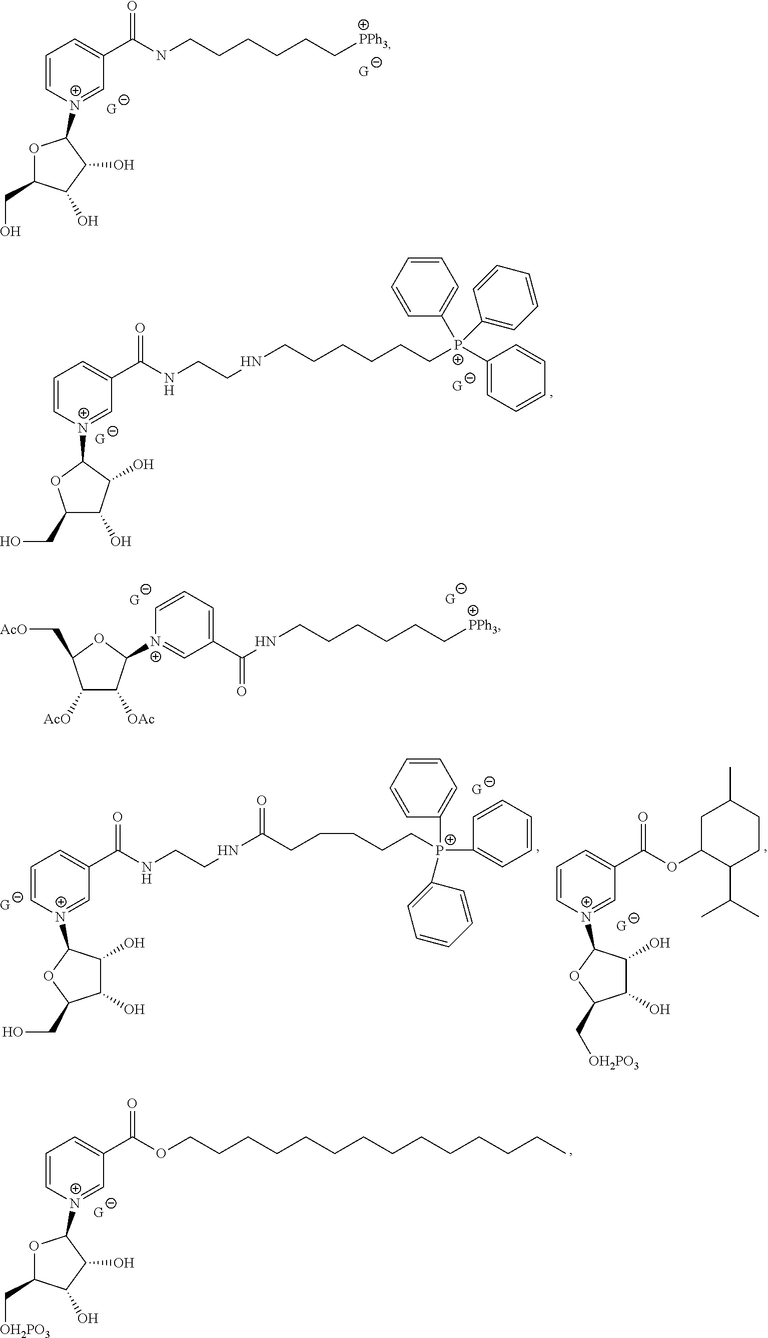 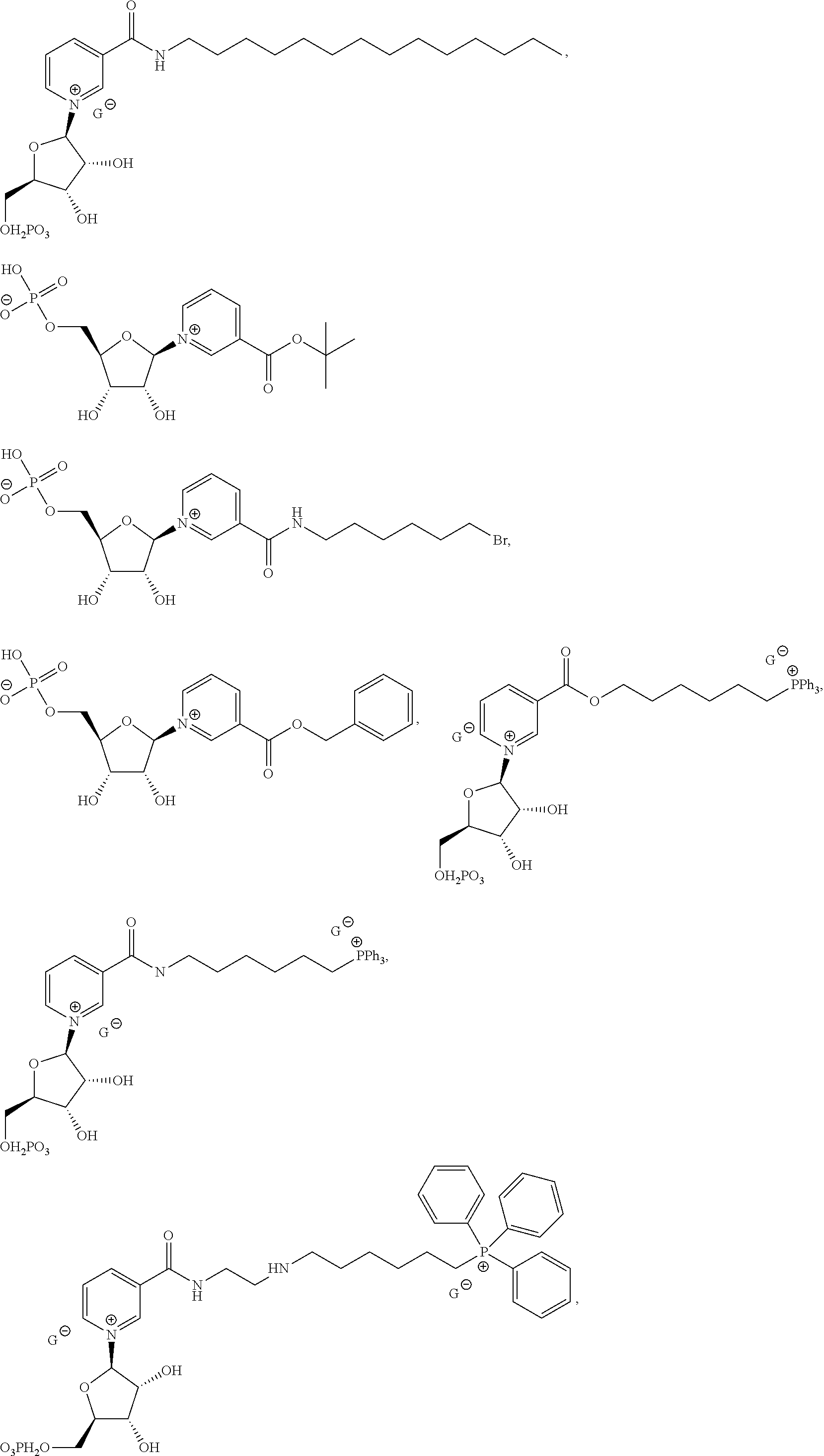 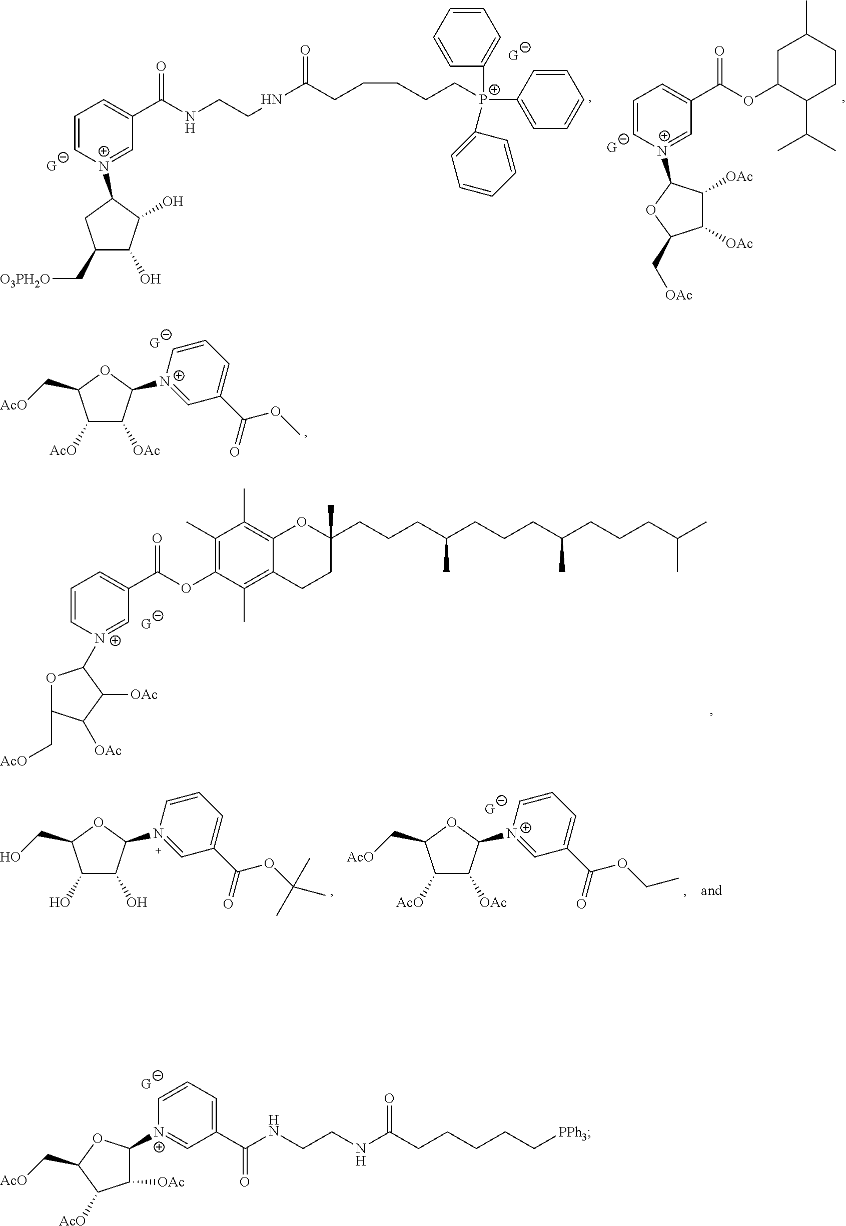
and
wherein G is a pharmaceutically acceptable anion.

30. The compound of claim 1, wherein the compound has a structure represented by formula VIb: 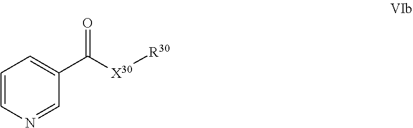
wherein,
R³⁰ is alkyl, aryl, heteroaryl, or cycloalkyl;
X³⁰ is O, N(R³⁴), or S; and
R³⁴ is H or alkyl.

31. The compound of claim 30, wherein X³⁰ is NH.

32. The compound of claim 30, wherein X³⁰ is O.

33. The compound of any one of claims 30-32, wherein R³⁰ is alkyl.

34. The compound of any one of claims 30-32, wherein R³⁰ is cycloalkyl.

35. The compound of any one of claims 30-32, wherein R³⁰ is aryl.

36. The compound of any one of claims 30-32, wherein R³⁰ is heteroaryl.

37. The compound of any one of claims 30-36, wherein R³⁰ is substituted with triarylphosphonium (e.g., P⁺(Ph)₃).

38. The compound of any one of claims 30-37, wherein R³⁰ is substituted with alkyl.

39. The compound of any one of claims 30-38, wherein R³⁰ is substituted with hydroxyl.

40. The compound of any one of claims 30-39, wherein R³⁰ is substituted with amido (e.g., alkylamido, esteralkylamido, alkylarylalkylamido, arylaminoaralkylamido, retionylamido, or triarylphosphoniumalkylamido).

41. The compound of any one of claims 30-40, wherein R³⁰ is substituted with amino (e.g., triarylphosphoniumalkylamino).

42. The compound of any one of claims 30-41, wherein R³⁰ is substituted with alkoxy (e.g., triarylphosphoniumalkoxy).

43. The compound of any one of claims 30-42, wherein R³⁰ is substituted with alkenyl (e.g., arylvinyl).

44. The compound of any one of claims 30-43, wherein R³⁰ is substituted with ester (e.g., alkylarylester, arylaminoaralkylester, retionylester, or triarylphosphoniumalkylester).

45. The compound of claim 1, wherein the compound is selected from the group consisting of: 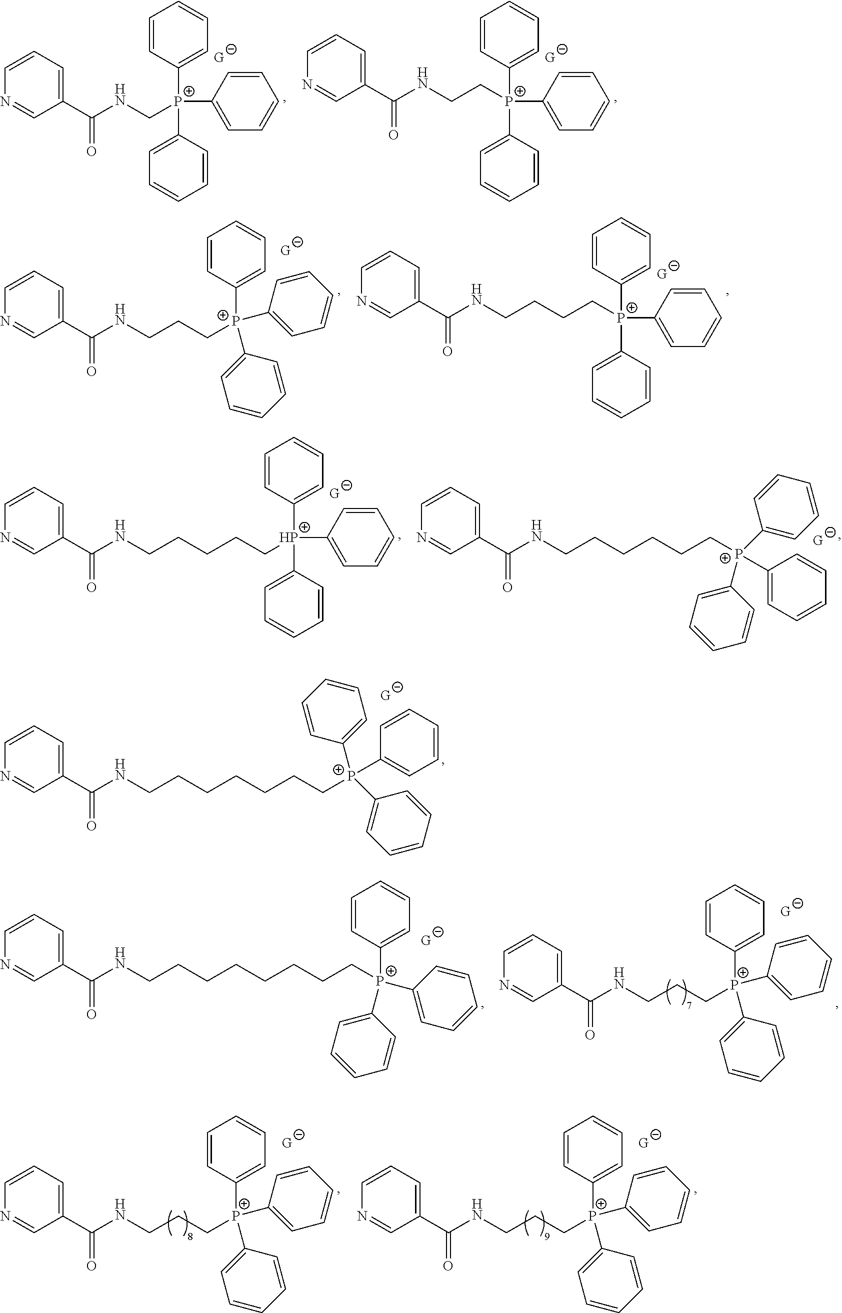 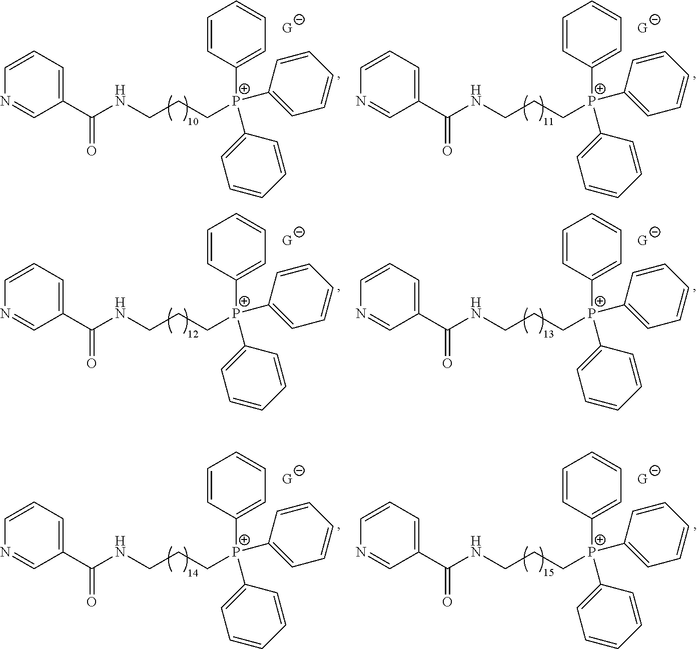 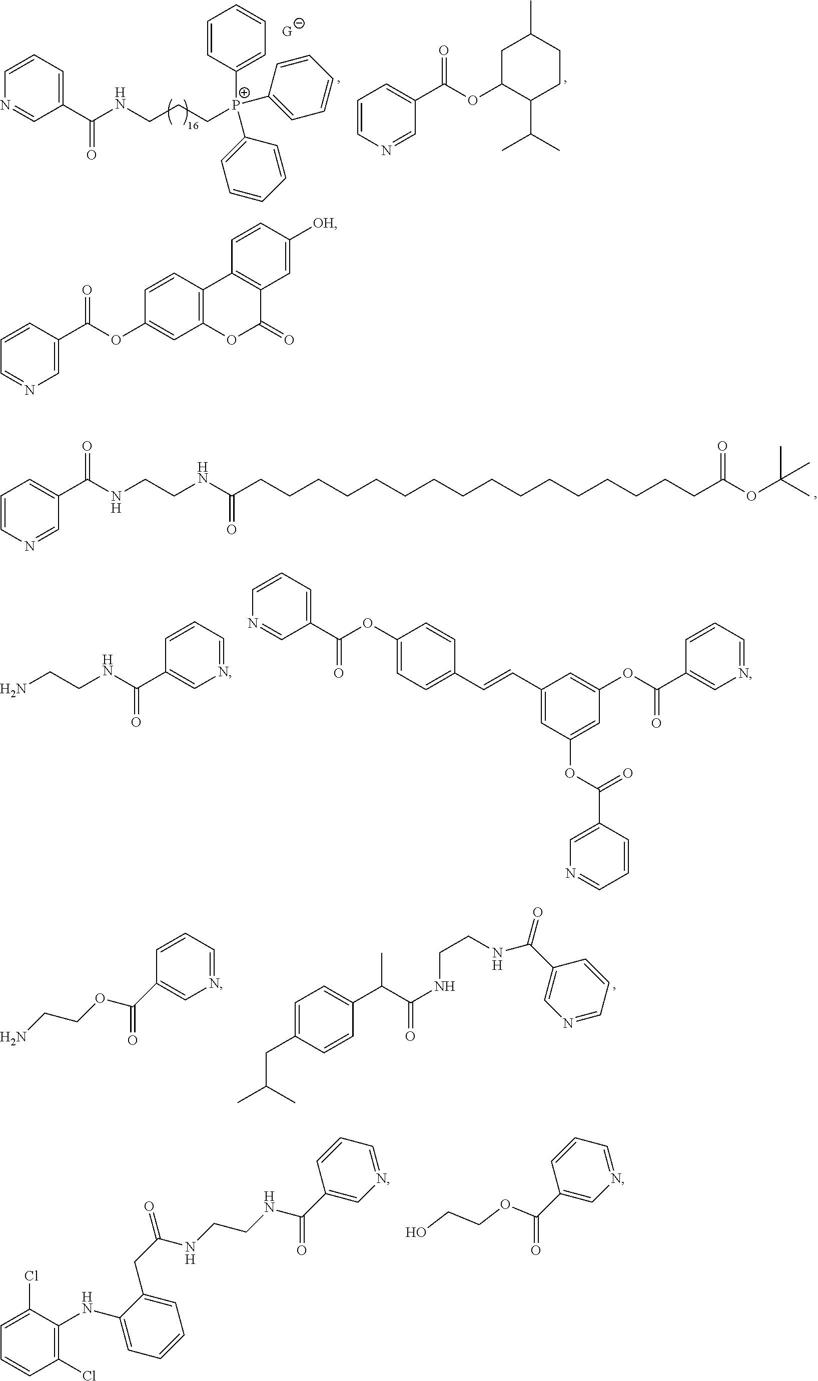 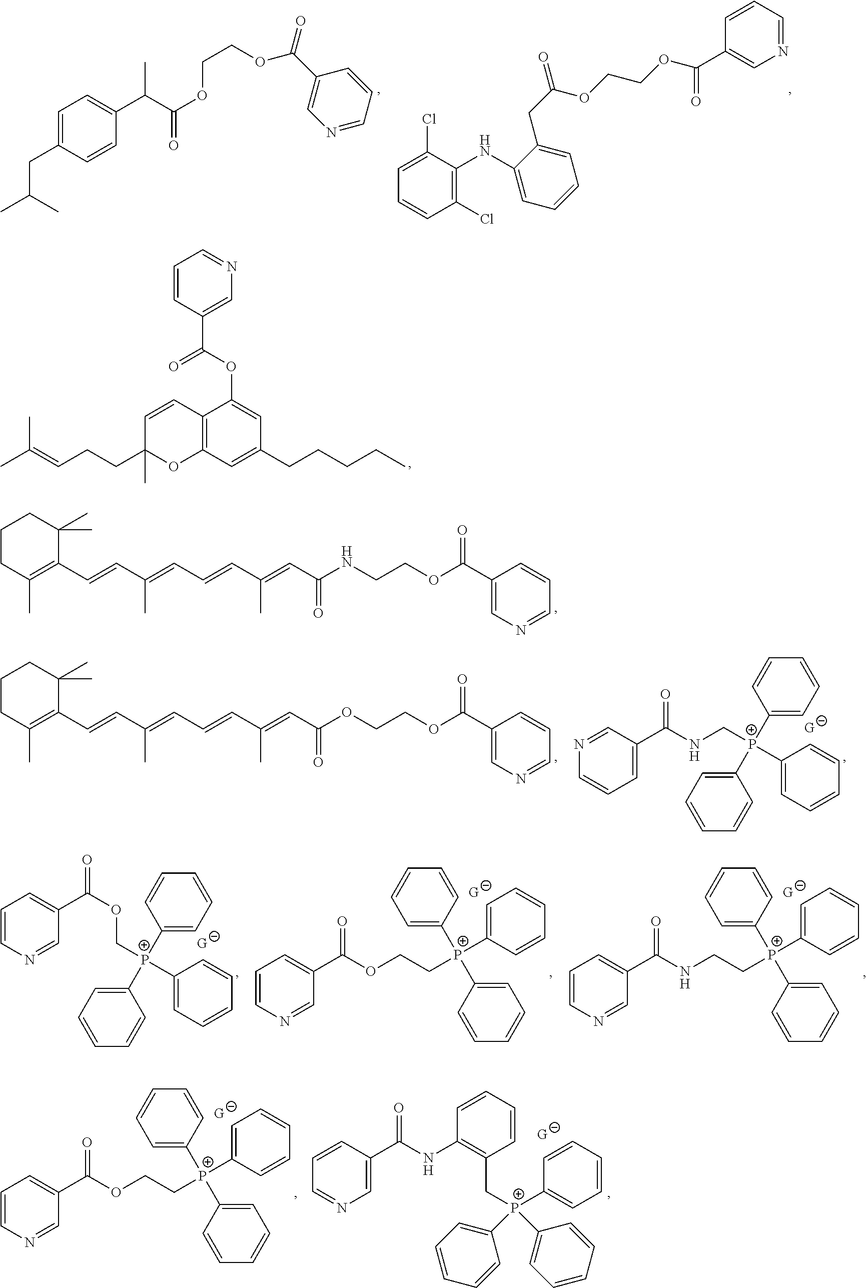 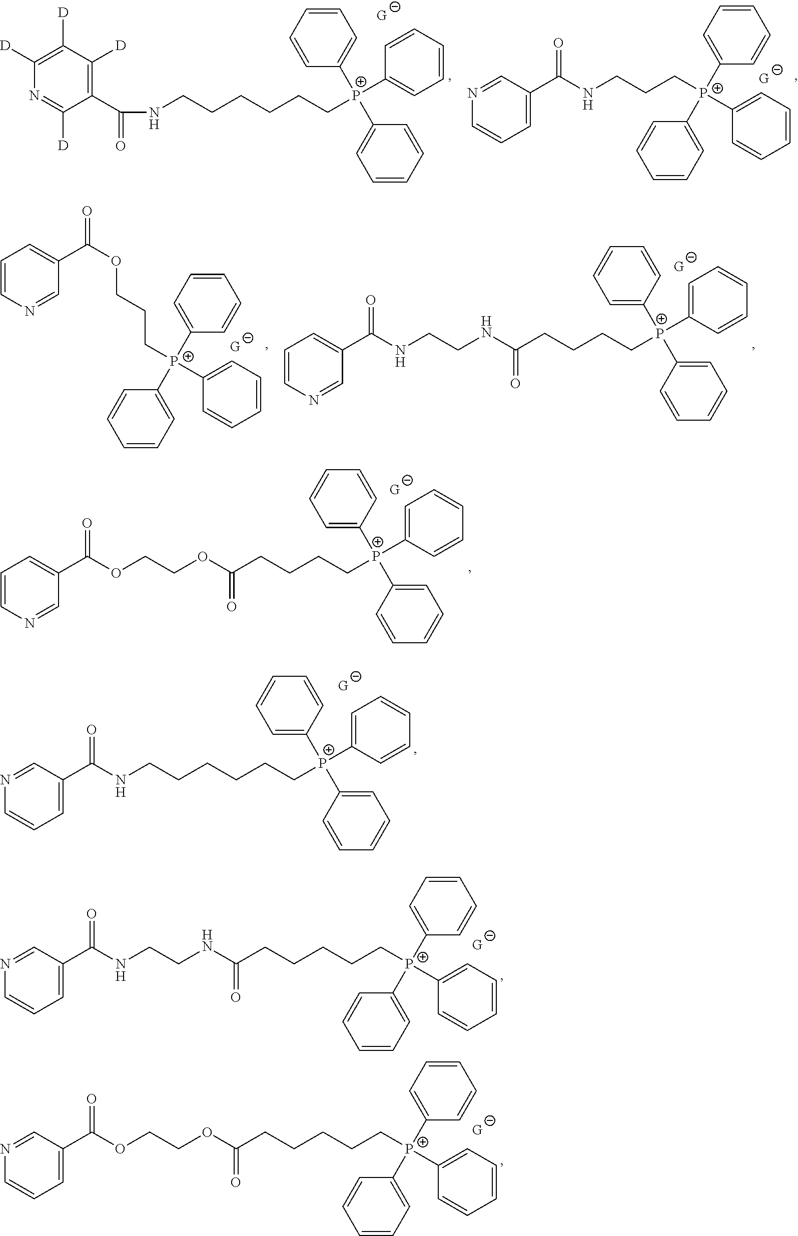 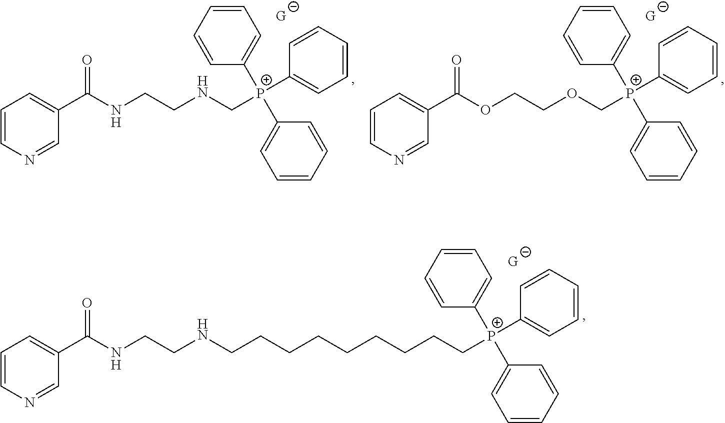 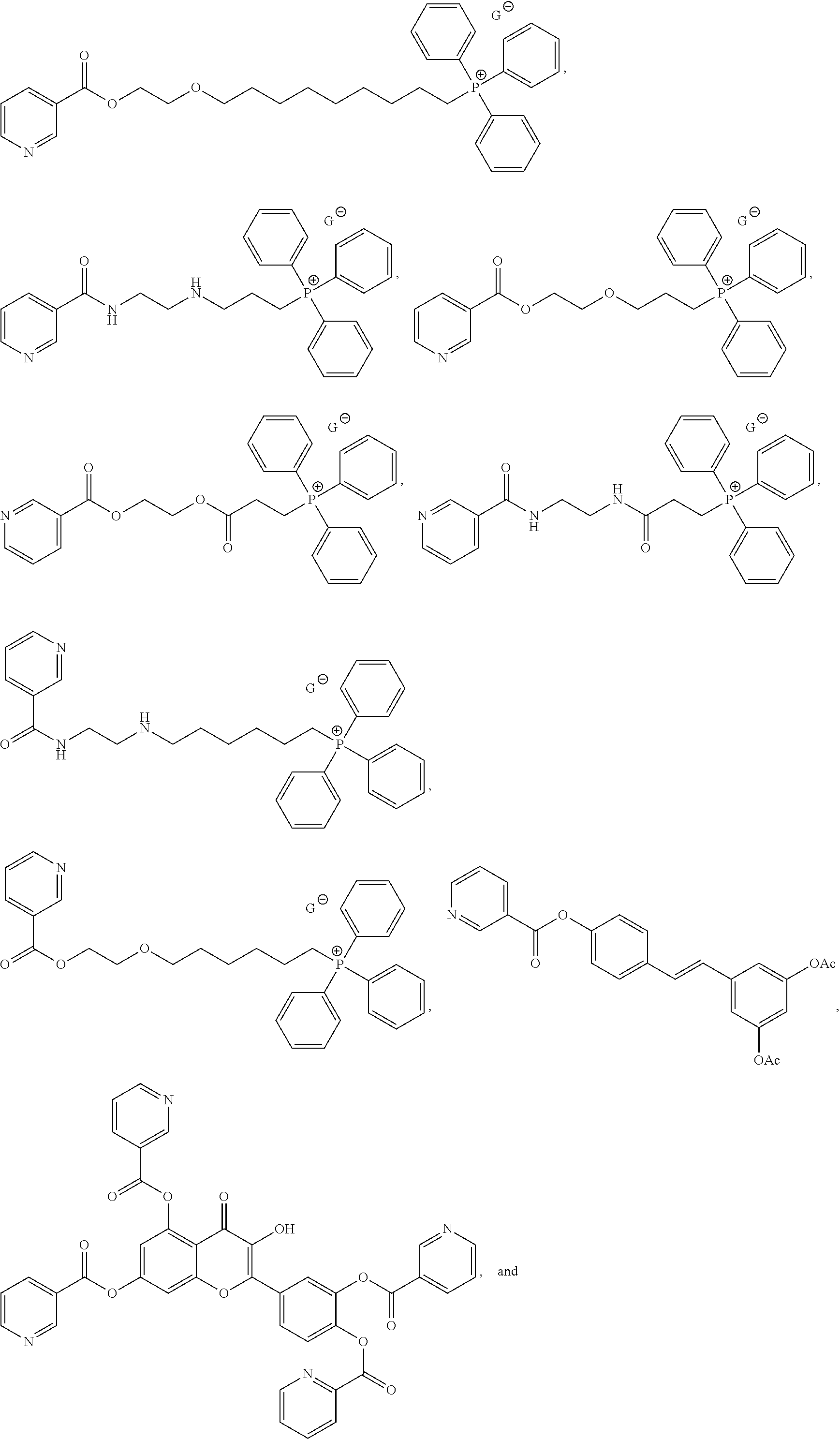 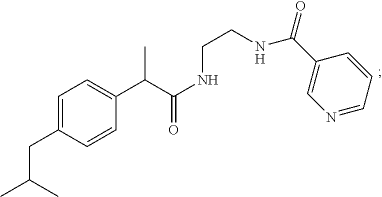 [SMILES: CC(C)Cc1ccc(C(C)C(=O)NCCNC(=O)c2cccnc2)cc1]
and
wherein G is a pharmaceutically acceptable anion.

46. A nicotinate/nicotinamide riboside compound or derivative of formula (V), or a salt, hydrate, or solvate thereof: 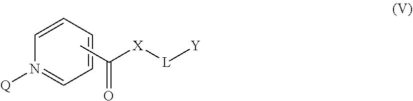
wherein:
Q is absent, 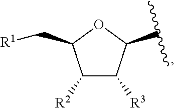
or H;
R¹ is HPO₄, H₂PO₄, —OH, or —OC(O)R⁴;
R² and R³ are independently —OH, —C(O)R⁴, —C(O)OR⁴, —C(O)NHR⁴ or halogen;
R⁴ is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
L is a bond, C₁₋₂₀ alkyl, aryl, heteroaryl, arylalkylaryl, arylalkyl, alkoxy, or —R¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
Y is —C(O)NH₂, —C(O)OH, —R⁵, —P(R⁷)₃, —NH₂, —NHR⁵, 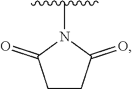 [SMILES: *N1C(=O)CCC1=O]
—SH, or —OH;
R⁵ is —C(O)R⁴, 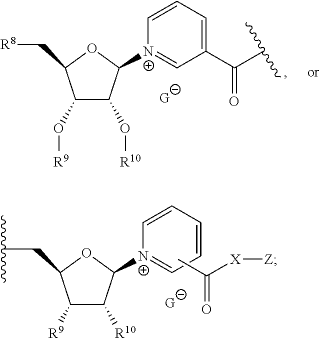
R⁷ is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —H, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
G is an anion (e.g., a pharmaceutically acceptable anion); and
Z is H, or C₁₋₂₀ alkyl; or Z and R¹ are optionally taken together as a bond, forming a macrocycle,
with the proviso that if X is O, NH, or NR⁷, and L is C₁₋₂₀ alkyl, aryl, heteroaryl, or alkoxy, then Y is not —C(O)NH₂, —C(O)OH, —R⁵, —NH₂, —NHR⁵, —SH, or —OH.

47. The compound of claim 46, wherein
Q is 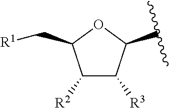
R¹ is H₂PO₄;
R² is —OH;
R³ is —OH;
X is NH; and
Y is —P(R⁷)₃.

48. The compound of claim 47, selected from the group consisting of 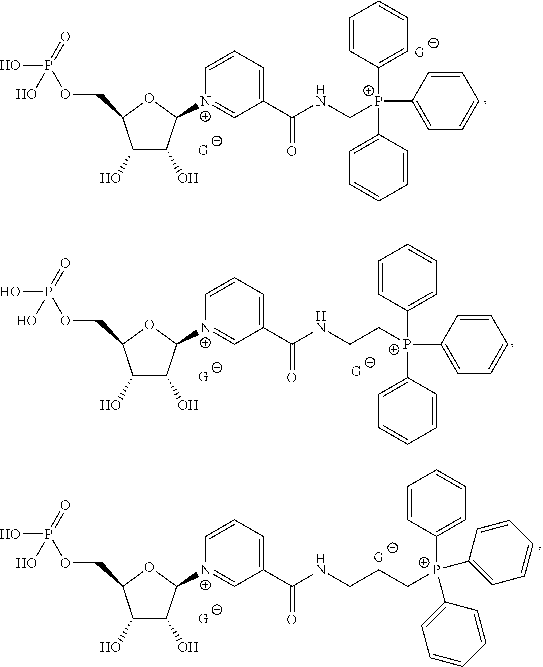 [SMILES: O=C(NC[P+](c1ccccc1)(c1ccccc1)c1ccccc1)c1ccc[n+]([C@@H]2O[C@H](COP(=O)(O)O)[C@@H](O)[C@H]2O)c1 ; O=C(NCC[P+](c1ccccc1)(c1ccccc1)c1ccccc1)c1ccc[n+]([C@@H]2O[C@H](COP(=O)(O)O)[C@@H](O)[C@H]2O)c1 ; O=C(NCCC[P+](c1ccccc1)(c1ccccc1)c1ccccc1)c1ccc[n+]([C@@H]2O[C@H](COP(=O)(O)O)[C@@H](O)[C@H]2O)c1] 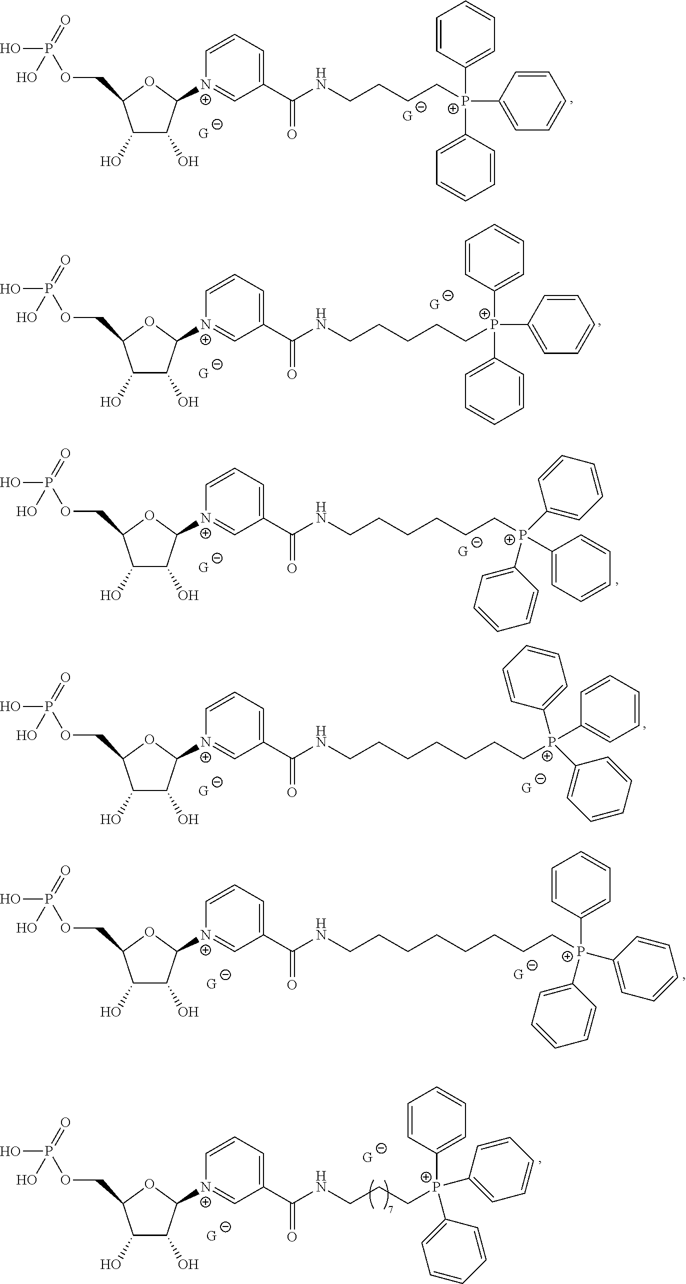 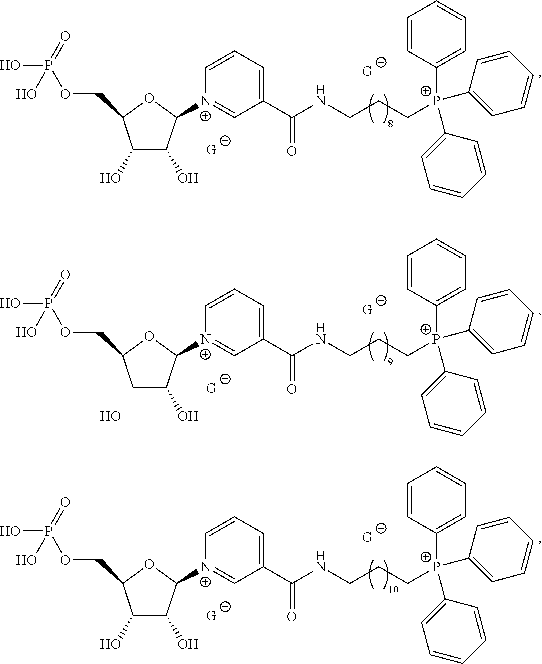 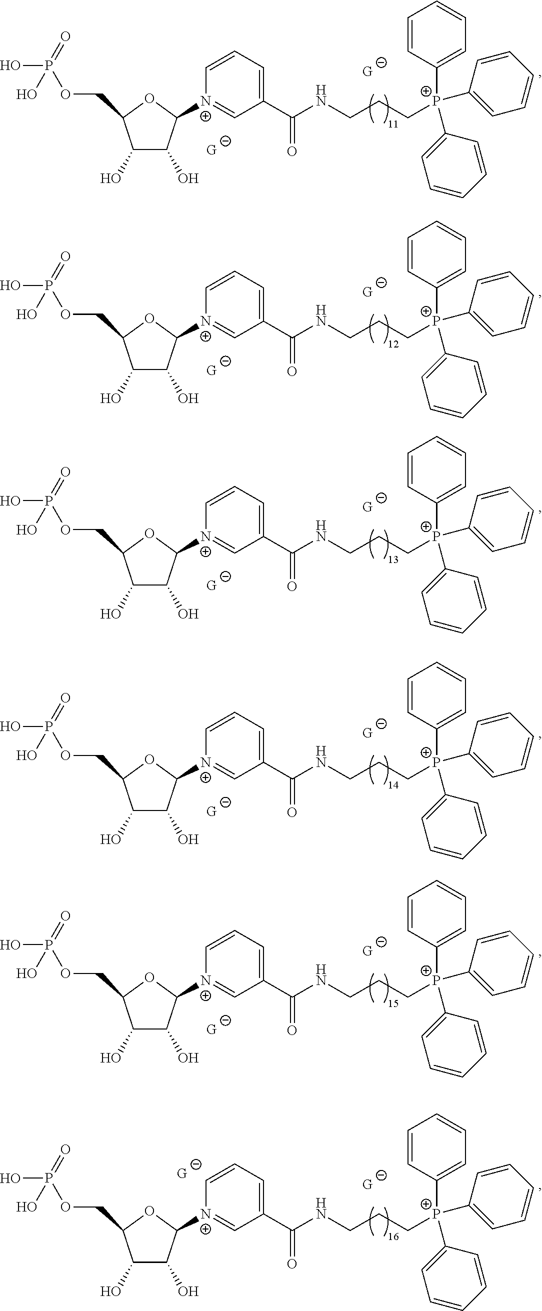 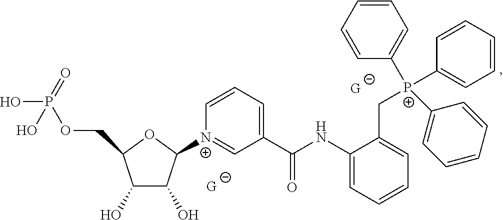 [SMILES: O=C(Nc1ccccc1C[P+](c1ccccc1)(c1ccccc1)c1ccccc1)c1ccc[n+]([C@@H]2O[C@H](COP(=O)(O)O)[C@@H](O)[C@H]2O)c1]
and combinations thereof.

49. The compound of claim 48, wherein
Q is absent;
R² is —OH;
R³ is —OH;
X is NH; and
Y is —P(R′)₃.

50. The compound of claim 48, selected from the group consisting of: 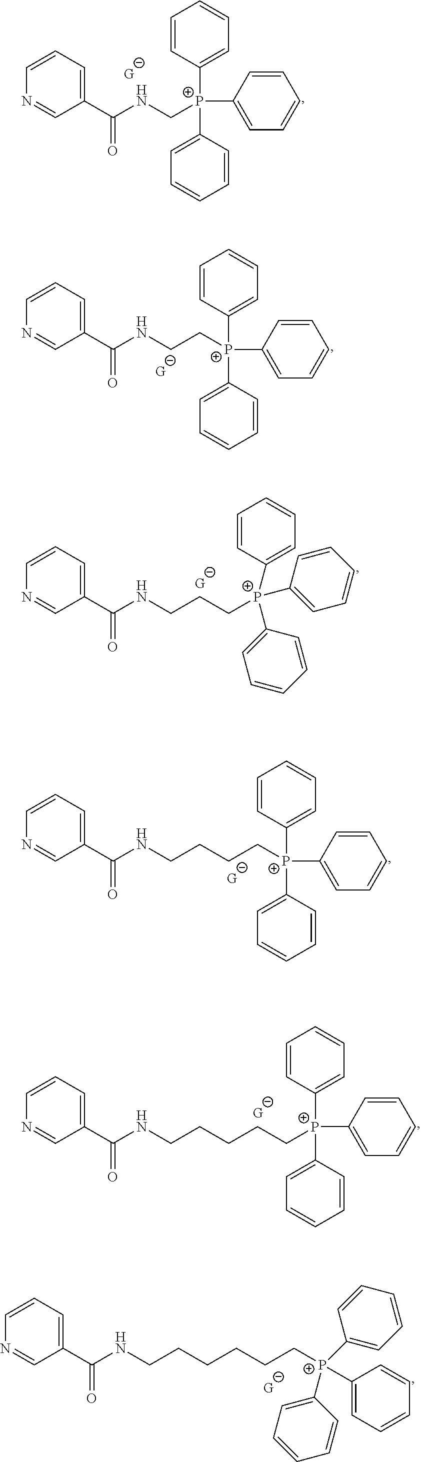 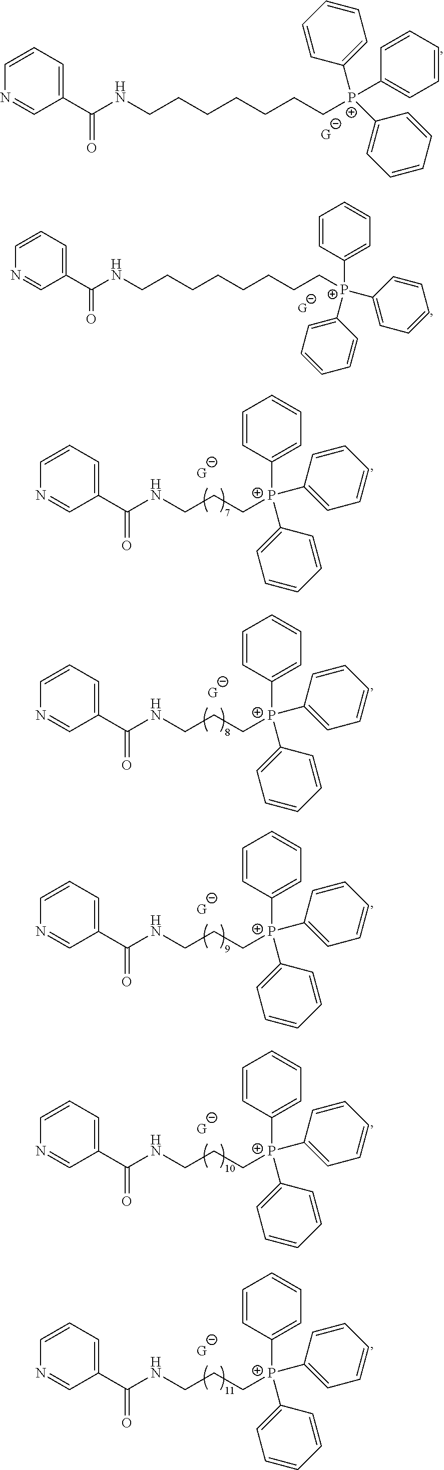 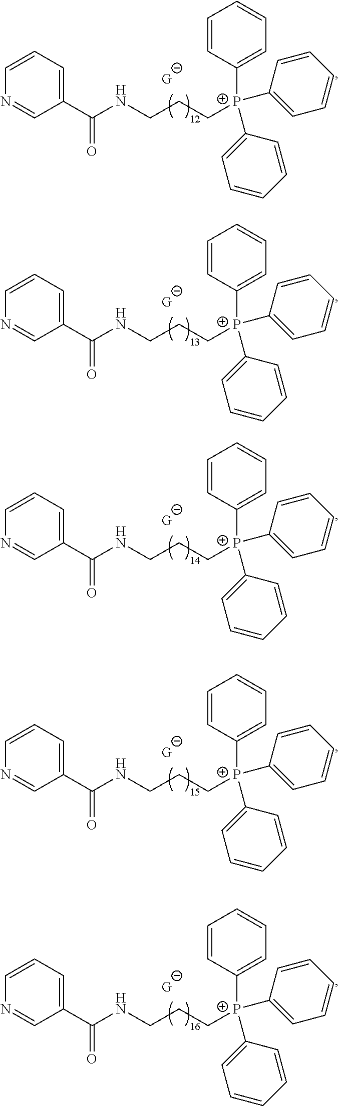
and combinations thereof.

51. A compound or derivative of formula (IV), or a salt, hydrate, or solvate thereof: 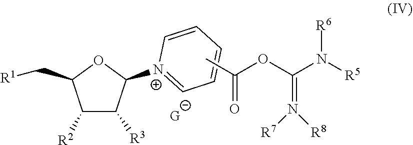
wherein:
R¹ is HPO₄, H₂PO₄, —OH, or —OC(O)R⁴;
R² and R³ are independently —OH, —C(O)R⁴, —C(O)OR⁴, —C(O)NHR⁴ or a halogen;
R⁴ is —H, C₁₋₂₀ alkyl, C₃₋₁₀ cycloalkyl, heterocyclyl, aryl, or heteroaryl, wherein the C₁₋₂₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
R⁵, R⁶, R⁷, R⁸ are independently a lone pair, H, a C₁₋₁₀ alkyl, C₃₋₁₀ cycloalkyl, heterocyclyl, aryl, or heteroaryl, wherein the C₁₋₁₀ alkyl and C₃₋₁₀ cycloalkyl are optionally substituted with -alkyl, —O-alkyl, —N(R⁹)₂;
R⁹ is —H, or a C₁₋₁₀ alkyl; and
G is an anion (e.g., a pharmaceutically acceptable anion).

52. The compound of claim 51, wherein
R¹ is H₂PO₄;
R² is —OH; and
R³ is —OH.

53. The compound of claim 51, wherein the compound is selected from the group consisting of: 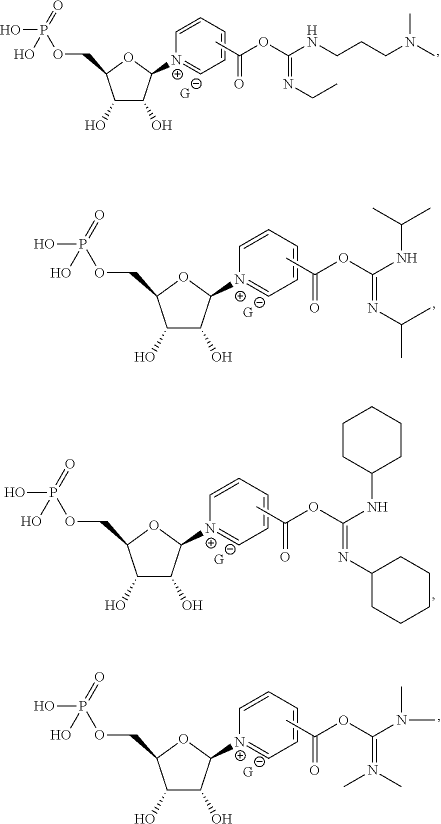
and combinations thereof.

54. A nicotinate/nicotinamide riboside compound or derivative of formula (V), or a salt, hydrate, or solvate thereof: 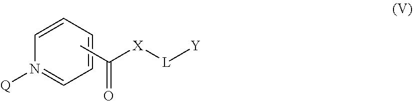
wherein:
Q is absent, 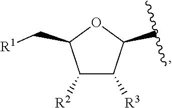
or H;
R¹ is HPO₄, H₂PO₄, —OH, or —OC(O)R⁴;
R² and R³ are independently —OH, —C(O)R⁴, —C(O)OR⁴, —C(O)NHR⁴ or halogen;
R⁴ is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
L is a bond, C₁₋₂₀ alkyl, aryl, heteroaryl, arylalkylaryl, arylalkyl, alkoxy, or —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
Y is —C(O)NH₂, —C(O)OH, —R⁵, —P(R⁷)₃, —NH₂, —NHR⁵, 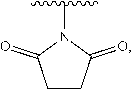 [SMILES: *N1C(=O)CCC1=O]
—SH, or —OH;
R⁵ is —C(O)R⁴, 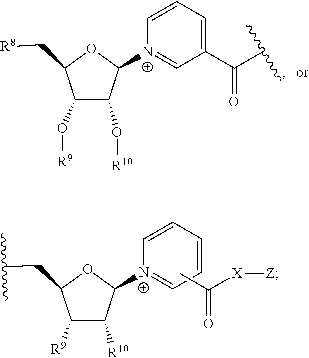
R⁷ is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —H, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z is H, or C₁₋₂₀ alkyl; or Z and R¹ are optionally taken together as a bond, forming a macrocycle,
with the proviso that if X is O, NH, or NR⁷, and L is C₁₋₂₀ alkyl, aryl, heteroaryl, or alkoxy, then Y is not —C(O)NH₂, —C(O)OH, —R⁵, —NH₂, —NHR⁵, —SH, or —OH.

55. The compound of claim 54, wherein
Q is 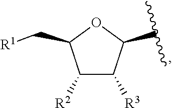
R¹ is H₂PO₄;
R² is —OH;
R³ is —OH;
X is NH; and
Y is —P(R⁷)₃.

56. The compound of claim 55, selected from the group consisting of 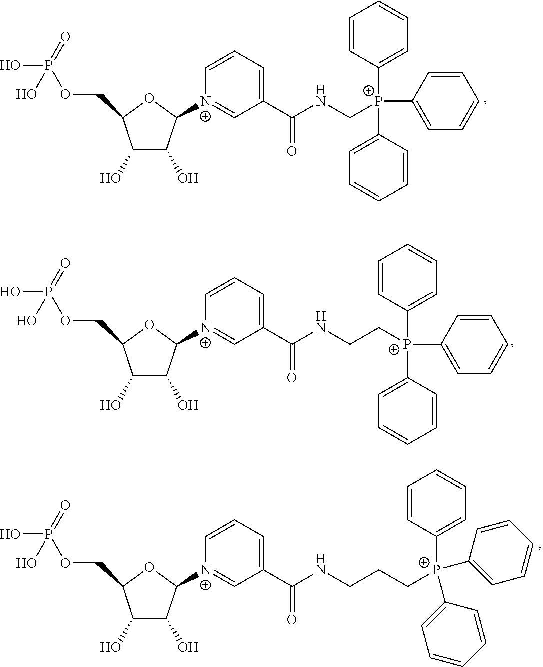 [SMILES: O=C(NC[P+](c1ccccc1)(c1ccccc1)c1ccccc1)c1ccc[n+]([C@@H]2O[C@H](COP(=O)(O)O)[C@@H](O)[C@H]2O)c1 ; O=C(NCC[P+](c1ccccc1)(c1ccccc1)c1ccccc1)c1ccc[n+]([C@@H]2O[C@H](COP(=O)(O)O)[C@@H](O)[C@H]2O)c1 ; O=C(NCCC[P+](c1ccccc1)(c1ccccc1)c1ccccc1)c1ccc[n+]([C@@H]2O[C@H](COP(=O)(O)O)[C@@H](O)[C@H]2O)c1] 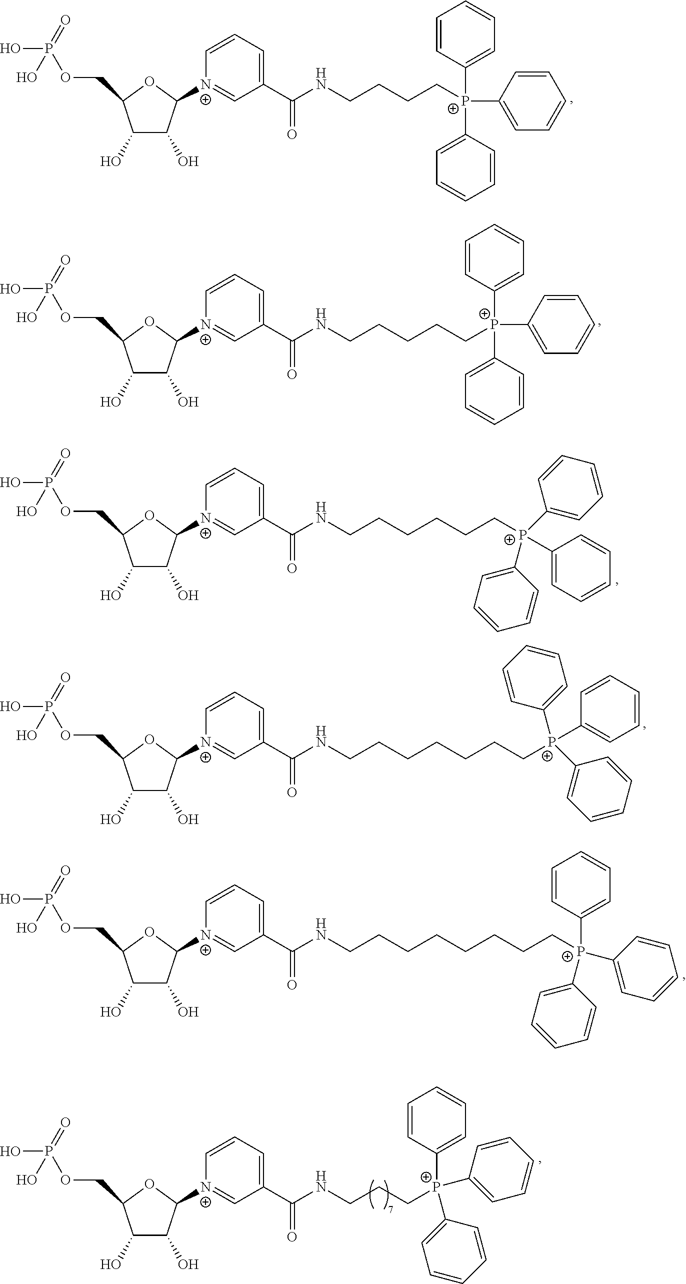 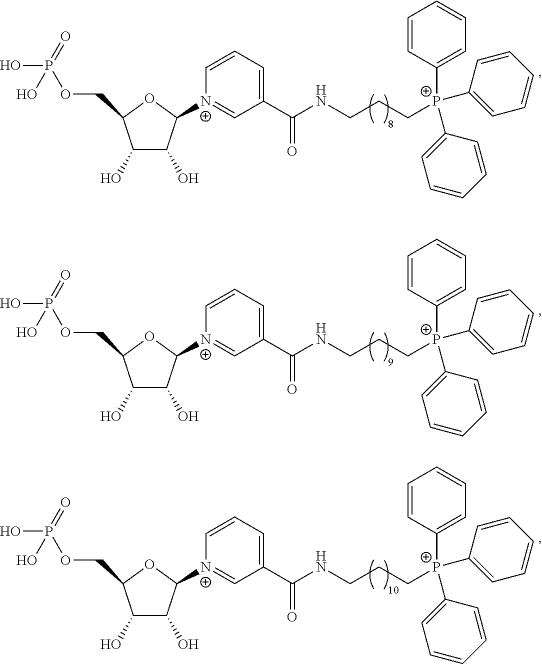 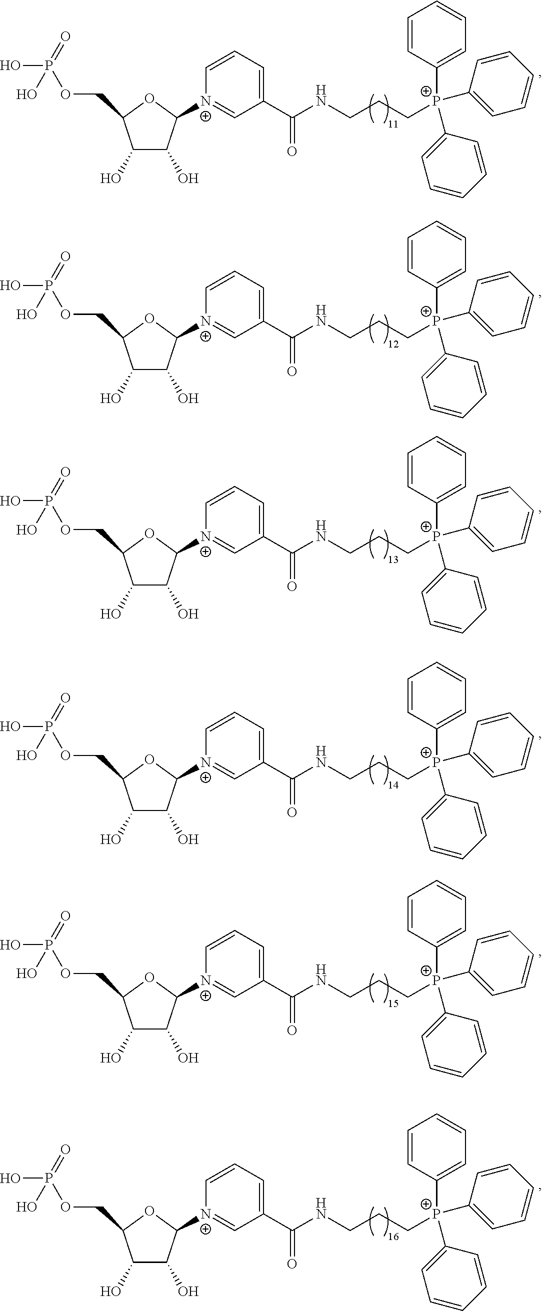 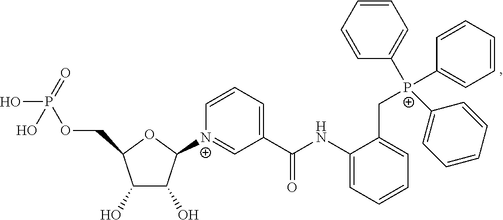 [SMILES: O=C(Nc1ccccc1C[P+](c1ccccc1)(c1ccccc1)c1ccccc1)c1ccc[n+]([C@@H]2O[C@H](COP(=O)(O)O)[C@@H](O)[C@H]2O)c1]
and combinations thereof.

57. The compound of claim 54, wherein
Q is absent;
R² is —OH;
R³ is —OH;
X is NH; and
Y is —P(R⁷)₃.

58. The compound of claim 54, selected from the group consisting of: 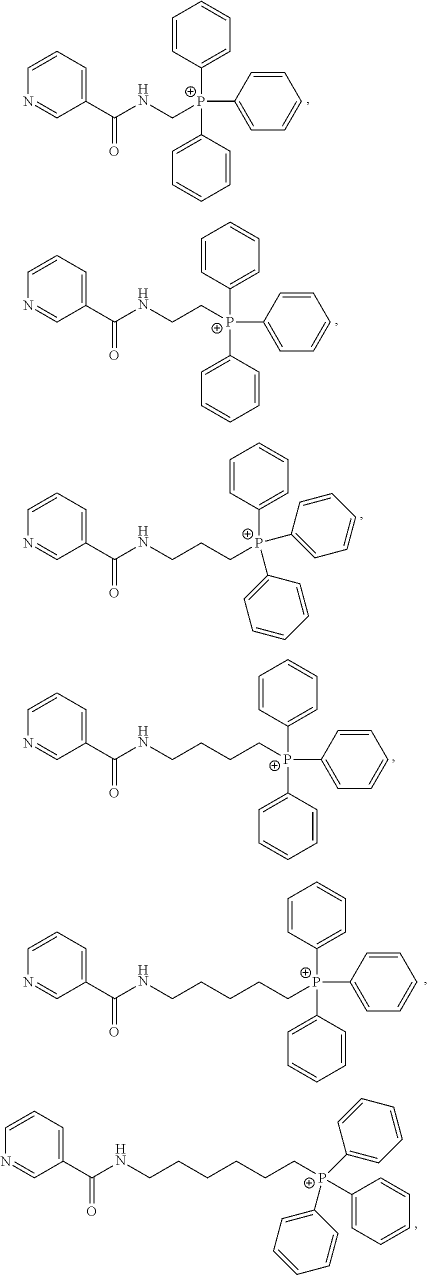 [SMILES: O=C(NC[P+](c1ccccc1)(c1ccccc1)c1ccccc1)c1cccnc1 ; O=C(NCC[P+](c1ccccc1)(c1ccccc1)c1ccccc1)c1cccnc1 ; O=C(NCCC[P+](c1ccccc1)(c1ccccc1)c1ccccc1)c1cccnc1 ; O=C(NCCCC[P+](c1ccccc1)(c1ccccc1)c1ccccc1)c1cccnc1 ; O=C(NCCCCC[P+](c1ccccc1)(c1ccccc1)c1ccccc1)c1cccnc1 ; O=C(NCCCCCC[P+](c1ccccc1)(c1ccccc1)c1ccccc1)c1cccnc1] 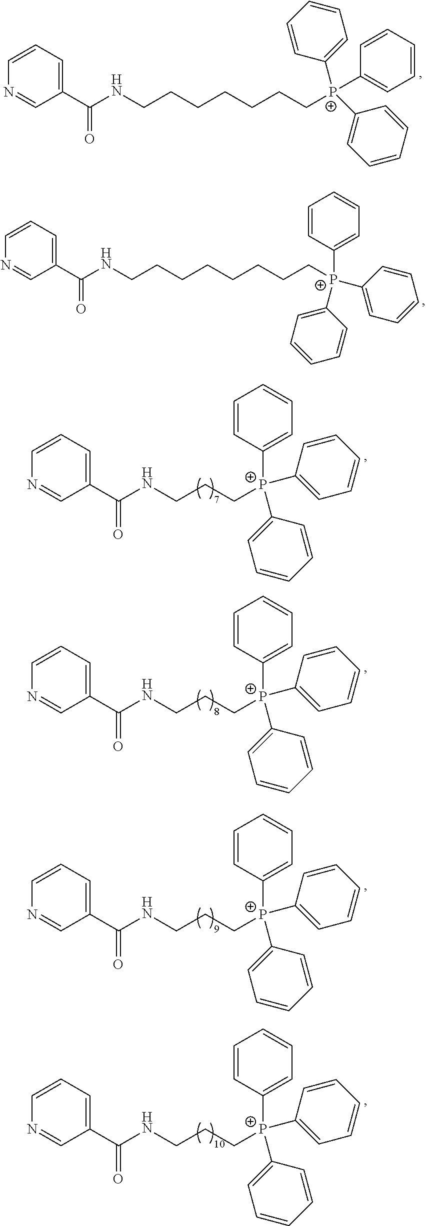 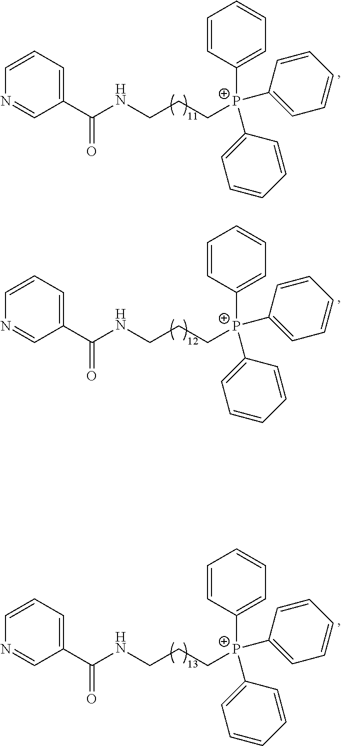 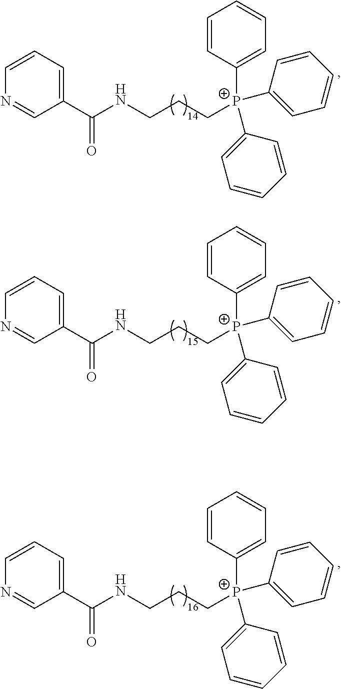
and combinations thereof.

59. A compound or derivative of formula (IV), or a salt, hydrate, or solvate thereof: 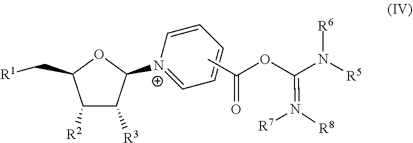
wherein:
R¹ is HPO₄, H₂PO₄, —OH, or —OC(O)R⁴;
R² and R³ are independently —OH, —C(O)R⁴, —C(O)OR⁴, —C(O)NHR⁴ or a halogen;
R⁴ is —H, C₁₋₂₀ alkyl, C₃₋₁₀ cycloalkyl, heterocyclyl, aryl, or heteroaryl, wherein the C₁₋₂₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
R⁵, R⁶, R⁷, R⁸ are independently a lone pair, H, a C₁₋₁₀ alkyl, C₃₋₁₀ cycloalkyl, heterocyclyl, aryl, or heteroaryl, wherein the C₁₋₁₀alkyl and C₃₋₁₀ cycloalkyl are optionally substituted with -alkyl, —O-alkyl, —N(R⁹)₂; and
R⁹ is —H, or a C₁₋₁₀ alkyl.

60. The compound of claim 59, wherein
R¹ is H₂PO₄;
R² is —OH; and
R³ is —OH.

61. The compound of claim 59, wherein the compound is selected from the group consisting of: 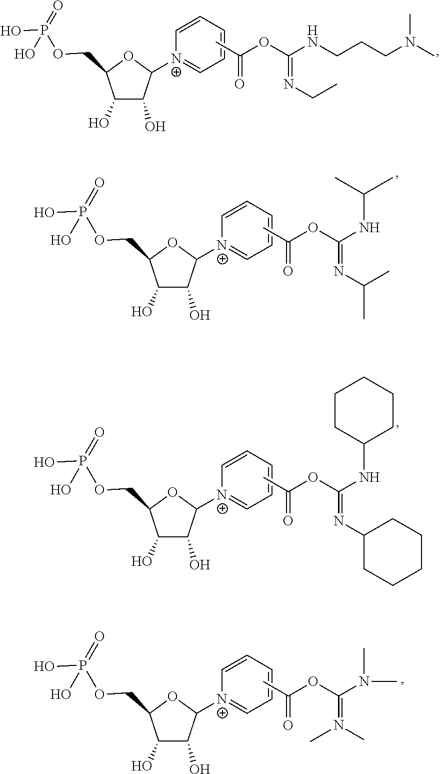
and combinations thereof.

62. A pharmaceutical composition comprising the compound of any one of claims 1-61 and a pharmaceutically acceptable excipient.

63. A composition comprising:
a compound according to any one of claims 1-61; and an acceptable carrier.

64. The composition of claim 63, wherein the carrier is a cosmetically acceptable carrier.

65. The composition of claim 64, wherein the cosmetically acceptable carrier comprises at least one of the group consisting of an additive, a colorant, an emulsifier, a fragrance, a humectant, a polymerizable monomer, a stabilizer, a solvent, and a surfactant.

66. The composition of claim 63, wherein the carrier is a pharmaceutically acceptable carrier.

67. The composition of claim 66, wherein the pharmaceutically acceptable carrier is selected from the group consisting of binders, disintegrating agents, lubricants, corrigents, solubilizing agents, suspension aids, emulsifying agents, coating agents, cyclodextrins, and/or buffers.

68. A method of making a compound of any one of claims 1-61, comprising:
providing a nicotinate/nicotinamide riboside compound or derivative of formula (II), or a salt, hydrate, or solvate thereof 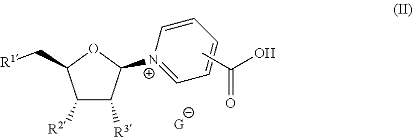
wherein:
R^{1′} is HPO₄, H₂PO₄, —OH, or —OC(O)R^{4′};
R^{2′} and R^{3′} are independently —OH, —C(O)R^{4′}, —C(O)OR^{4′}, —C(O)NHR^{4′} or halogen; and
R^{4′} is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
contacting the compound or derivative of formula (II), or a salt, hydrate, or solvate thereof, with a coupling agent and a compound of formula (III)
H—X′-L′-Y′ (III)
wherein:
X′ is O, NH, NR^{7′} or S;
L′ is a bond, C₁₋₂₀ alkyl, aryl, heteroaryl, arylalkylaryl, arylalkyl, alkoxy, —R^{11′}—S—S—R^{11′}—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
R^{4″} is a C₁₋₂₀ alkyl optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a′}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
Y′ is C₁₋₂₀ alkyl; perfluoroalkyl, —C(O)NH₂, —C(O)OH, —R^{5′}, —C(R^{6′})₃, —P(R^{7′})₃, —NH₂, —NHR^{5′}, 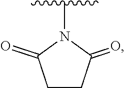 [SMILES: *N1C(=O)CCC1=O]
—SH, —OH;
R^{5′} is —C(O)R^{4″}, 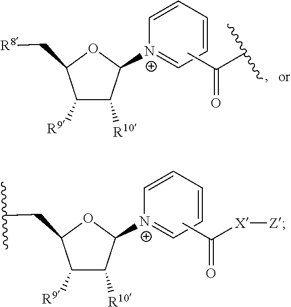
R^{6′} is individually selected at each occurrence from the group consisting of C₁₋₆ alkyl, cycloalkyl, heterocyclyl, heteroaryl, aryl, —H, -halogen, —OH, and —NH₂;
R^{7′} is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R^{8′} is HPO₄, H₂PO₄, —OH or —OC(O)R^{4″};
R^{9′} and R^{10′} are independently —OH, —C(O)R^{4″}, —C(O)OR^{4″}, —C(O)NHR^{4″} or halogen;
R^{11′} is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
Z′ is H, or C₁₋₂₀ alkyl; and
G is an anion

69. A method of making a compound of any one of claims 1-61 or derivative of formula (I), or a salt, hydrate, or solvate thereof: 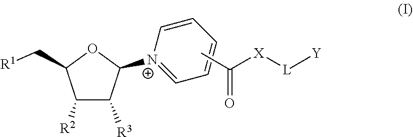
wherein:
R¹ is HPO₄, H₂PO₄, —OH, or —OC(O)R⁴;
R² and R³ are independently —OH, —C(O)R⁴, —C(O)OR⁴, —C(O)NHR⁴ or a halogen;
R⁴ is-H, C₁₋₂₀ alkyl, C₃₋₁₀ cycloalkyl, heterocyclyl, aryl, or heteroaryl, wherein the C₁₋₂₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
X is O, NH, NR⁷, or S;
L is a bond, C₁₋₂₀ alkyl, aryl, heteroaryl, arylalkylaryl, arylalkyl, alkoxy, —R¹¹—S—S—R¹¹—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
Y is C₁₋₂₀ alkyl, perfluoroalkyl, —C(O)NH₂, —C(O)OH, —R⁵, —C(R⁶)₃, —P(R⁷)₃, —NH₂, —NHR⁵, 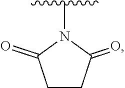 [SMILES: *N1C(=O)CCC1=O]
—SH, or —OH;
R⁵ is —C(O)R⁴, 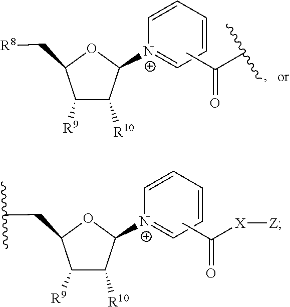
R⁶ is individually selected at each occurrence from the group consisting of C₁₋₆ alkyl, cycloalkyl, heterocyclyl, heteroaryl, aryl, —H, -halogen, —OH, and —NH₂;
R⁷ is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R⁸ is HPO₄, H₂PO₄, —OH or —OC(O)R⁴;
R⁹ and R¹⁰ are independently —H, —C(O)R⁴, —C(O)OR⁴, —C(O)NHR⁴ or halogen;
R¹¹ is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z is —H, or C₁₋₂₀ alkyl; or Z and R¹ are optionally taken together as a bond, forming a macrocycle;
comprising the steps of:
providing a nicotinate/nicotinamide riboside compound or derivative of formula (II), or a salt, hydrate, or solvate thereof 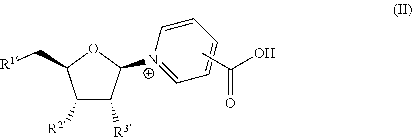
wherein:
R^{1′} is HPO₄, H₂PO₄, —OH, or —OC(O)R^{4′};
R^{2′} and R^{3′} are independently —OH, —C(O)R^{4′}, —C(O)OR^{4′}, —C(O)NHR^{4′} or halogen; and R^{4′} is —H, C₁₋₂₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
contacting the compound or derivative of formula (II), or a salt, hydrate, or solvate thereof, with a coupling agent and a compound of formula (III)
H—X′-L′-Y′ (III)
wherein:
X′ is O, NH, NR^{7′} or S;
L′ is a bond, C₁₋₂₀ alkyl, aryl, heteroaryl, arylalkylaryl, arylalkyl, alkoxy, —R^{11′}—S—S—R^{11′}—, wherein the C₁₋₂₀ alkyl is optionally substituted with one or more groups selected from an amino acid side chain, —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, and the aryl, heteroaryl, arylalkylaryl, arylalkyl, and alkoxy, are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{b}, —CO₂R^{b}, —O—C(O)R^{b}, —NHC(O)R^{b}, —NR^{b}C(O)R^{b}, —NO₂, —CN, and —SO₂R^{b}, wherein each R^{b} is independently Ar, C₁₋₆ alkyl, or CH₂Ar; and Ar is an aryl or heteroaryl;
R^{4″} is a C₁₋₂₀ alkyl optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a′}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl;
Y′ is C₁₋₂₀ alkyl; perfluoroalkyl, —C(O)NH₂, —C(O)OH, —R^{5′}, —C(R^{6′})₃, —P(R^{7′})₃, —NH₂, —NHR^{5′}, 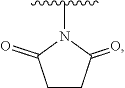 [SMILES: *N1C(=O)CCC1=O]
—SH, —OH;
R^{5′} is —C(O)R^{4″}, 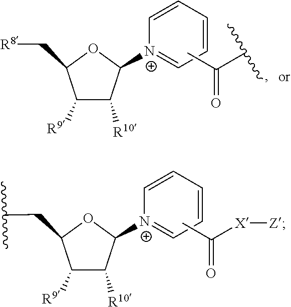
R^{6′} is individually selected at each occurrence from the group consisting of C₁₋₆ alkyl, cycloalkyl, heterocyclyl, heteroaryl, aryl, —H, -halogen, —OH, and —NH₂;
R^{7′} is individually selected at each occurrence from the group consisting of substituted or unsubstituted C₁₋₆ alkyl, substituted or unsubstituted cycloalkyl, substituted or unsubstituted heterocyclyl, substituted or unsubstituted heteroaryl, and substituted or unsubstituted aryl;
R^{8′} is HPO₄, H₂PO₄, —OH or —OC(O)R^{4″};
R^{9′} and R^{10′} are independently —OH, —C(O)R^{4″}, —C(O)OR^{4″}, —C(O)NHR^{4″} or halogen;
R^{11′} is C₁₋₁₀ alkyl, C₃₋₁₀ cycloalkyl, heterocyclyl, aryl, or heteroaryl, wherein the C₁₋₁₀ alkyl, C₃₋₁₀ cycloalkyl, heterocyclyl, aryl, heteroaryl are optionally substituted with one or more groups selected from —OH, halogen, -alkyl, —O-alkyl, —N-alkyl, -alkenyl, -alkynyl, —O-aryl, —O-heteroaryl, —N-aryl, —N-heteroaryl, -aryl, —C(O)R^{a}, —CO₂R^{a}, —O—C(O)R^{a}, —NHC(O)R^{a}, —NR^{a}C(O)R^{a}, —NO₂, —CN, and —SO₂R^{a}, wherein each R^{a} is independently Ar, C₁₋₆ alkyl, or CH₂Ar; and Ar is an aryl or heteroaryl; and
Z′ is H, or C₁₋₂₀ alkyl;
under conditions to produce the compound or derivative of formula (I), or salt, hydrate, or solvate thereof; and
isolating the compound or derivative of formula (I), or salt, hydrate, or solvate thereof.

70. The method of claim 68 or 69 wherein the coupling agent is selected from the group consisting of 1-ethyl-3-(3-dimethylaminopropyl)carbodiimide (EDC); dicyclohexylcarbodiimide (DCC); diisopropylcarbodiimide (DIC); (Benzotriazol-1-yloxy)tris(dimethylamino)phosphonium hexafluorophosphate (BOP); (Benzotriazol-1-yloxy)tripyrrolidinophosphonium hexafluorophosphate (PyBOP); (7-Azabenzotriazol-1-yloxy)tripyrrolidinophosphonium hexafluorophosphate (PyAOP); Bromotripyrrolidinophosphonium hexafluorophosphate (PyBrOP); O-(Benzotriazol-1-yl)-N,N,N′,N′-tetramethyluronium tetrafluoroborate (TBTU); O-(6-Chlorobenzotriazol-1-yl)-N,N,N′,N′-tetramethyluronium tetrafluoroborate (TCTU); O—(N-succinimidyl)-1,1,3,3-tetramethyl-uronium tetrafluoroborate (TSTU); O-(5-Norbornene-2,3-dicarboximido)-N,N,N′,N′-tetramethyluronium tetrafluoroborate (TNTU); (2-(1H-benzotriazol-1-yl)-1,1,3,3-tetramethyluronium hexafluorophosphate) (HBTU); O-(7-Azabenzotriazol-1-yl)-N,N,N′,N′-tetramethyl uronium hexafluorophosphate (HATU); O-(6-Chlorobenzotriazol-1-yl)-N,N,N′,N′-tetramethyluronium hexafluorophosphate (HCTU); 3-(Diethylphosphoryloxy)-1,2,3-benzotriazin-4(3H)-one (DEPBT); 1,1′-carbonyldiimidazole (CDI), and combinations thereof.

71. The method of claim 68 or 69, wherein the coupling agent is 1-ethyl-3-(3-dimethylaminopropyl)carbodiimide (EDC).

72. The method of any one of claims 68-71, wherein the conditions to produce the compound of any one of claims 1-61 comprises reacting the compound of formula (III) with the compound of formula (II) in the presence of base and a coupling agent.

73. The method of claim 72, wherein the base is an amine.

74. The method of claim 72 or 73, wherein the base is selected from the group consisting of triethylamine; diisopropylethylamine; tributylamine; N-methylmorpholine; pyridine; 2,6-lutidine; and N-methylimidazole, and combinations thereof.

75. The method of claim 72, wherein the base is diisopropylethylamine.

76. The method of any one of claims 72-75, wherein the base is present in about 1.1 molar equivalents or greater of the coupling agent, the compound of formula (II) and/or the compound of formula (III).

77. The method of any one of claims 72-76, wherein the said reacting comprises:
(i) dissolving the compound of formula (II) in a solvent, or solvent mixture, to form a first solution;
(ii) adding the base and coupling agent to the first solution to form a basic solution;
(iii) adding the compound of formula (III) to the basic solution; and
(iv) isolating the compound of any one of claims 1-61.

78. The method of claim 77, wherein the solvent or solvent mixture is selected from the group consisting of water, dimethylformamide (DMF), chloroform, dichloromethane, dichloroethane, acetonitrile, dimethyl sulfoxide (DMSO), benzene, toluene, xylenes, chlorobenzene, tetrahydrofuran, methanol, ethanol, isopropanol, 1-butanol, 2-butanol, t-butyl alcohol, 2-butanone, hexane, hexane isomers, cyclohexane, ethers, diethylene glycol, acetone, ethyl acetate, butanone, 1,4-dioxane, and combinations thereof.

79. The method of any one of claims 72-78, wherein the reacting is carried out in air.

80. The method of any one of claims 72-79, wherein the reacting is performed at a temperature of about 0° C. to about 100° C.

81. The method of claim 80, wherein the temperature is about 25° C.

82. A method of increasing the level of NAD+ in a cell, comprising:
contacting a cell with a compound according to any one of claims 1-61 under conditions effective to increase the level of NAD+ in the cell.

83. The method of claim 81, wherein the cell is a skin cell.

84. A method of increasing intercellular NAD+ in a subject, comprising:
administering to a subject in need thereof a compound according to anyone of claims 1-61 in an amount effective to increase the intercellular NAD+ in the subject.

85. The method of claim 84, wherein the subject is a human subject.

86. A method of treating a skin affliction or skin condition comprising:
administering to a subject in need thereof, a therapeutically effective amount of a composition of any one of claims 1-61.

87. The method of claim 86, wherein the skin affliction or skin condition are disorders or diseases associated with or caused by inflammation, sun damage or natural aging.

88. The method of claim 89, wherein the skin affliction or skin condition is selected from the group consisting of contact dermatitis, irritant contact dermatitis, allergic contact dermatitis, atopic dermatitis, actinic keratosis, keratinization disorders, eczema, epidermolysis bullosa diseases, exfoliative dermatitis, seborrheic dermatitis, erythema multiformed, erythema nodosum, damage caused by the sun or other light sources, discoid lupus erythematosus, dermatomyositis, psoriasis, skin cancer and the effects of natural aging.

89. The method of any one of claims 86-89, wherein the composition is administered topically, to the skin as an ointment, lotion, cream, microemulsion, gel, or solution.

90. A method of treating a disease or disorder associate with cell death, or to protect cells from cell death, the method comprising administering to a subject in need thereof, the composition of any one of claims 1-61.

91. The method of claim 90, wherein the disease or disorder is associated with neural cell death, neuronal dysfunction, or muscular cell death or dysfunction.

92. The method of claim 91, wherein the disease or disorder is selected from the group consisting of Parkinson's disease; Alzheimer's disease; multiple sclerosis; amyotropic lateral sclerosis; muscular dystrophy; AIDS; fulminant hepatitis; Creutzfeld-Jakob disease; retinitis pigmentosa; cerebellar degeneration; myelodysplasis; aplastic anemia; ischemic diseases; myocardial infarction; stroke; hepatic diseases; alcoholic hepatitis; hepatitis B; hepatitis C; osteoarthritis; atherosclerosis; alopecia; damage to the skin due to UV light; lichen planus; atrophy of the skin; cataract; graft rejections; and cell death caused by surgery, drug therapy, chemical exposure or radiation exposure.

93. A method of treating a disease or disorder in a subject in need thereof, comprising administering a therapeutically amount of a compound of any one of claims 1-61 or a pharmaceutically acceptable salt thereof to the subject.

94. The method of claim 93, wherein the disease or disorder is selected from the group consisting of Parkinson's disease; Alzheimer's disease; multiple sclerosis; amyotropic lateral sclerosis; muscular dystrophy; AIDS; fulminant hepatitis; Creutzfeld-Jakob disease; retinitis pigmentosa; cerebellar degeneration; myelodysplasis; aplastic anemia; ischemic diseases; myocardial infarction; stroke; hepatic diseases; alcoholic hepatitis; hepatitis B; hepatitis C; osteoarthritis; atherosclerosis; alopecia; damage to the skin due to UV light; lichen planus; atrophy of the skin; cataract; graft rejections; and cell death caused by surgery, drug therapy, chemical exposure or radiation exposure.

95. A method of treating a skin condition in a subject in need thereof, comprising administering a therapeutically amount of a compound of any one of claims 1-61 or a pharmaceutically acceptable salt thereof to the subject.

96. The method of claim 95, wherein the skin condition is selected from the group consisting of contact dermatitis, irritant contact dermatitis, allergic contact dermatitis, atopic dermatitis, actinic keratosis, keratinization disorders, eczema, epidermolysis bullosa diseases, exfoliative dermatitis, seborrheic dermatitis, erythema multiformed, erythema nodosum, damage caused by the sun or other light sources, discoid lupus erythematosus, dermatomyositis, psoriasis, skin cancer and the effects of natural aging.

97. A method of making Compound 1, wherein the method is performed as depicted in Scheme I: 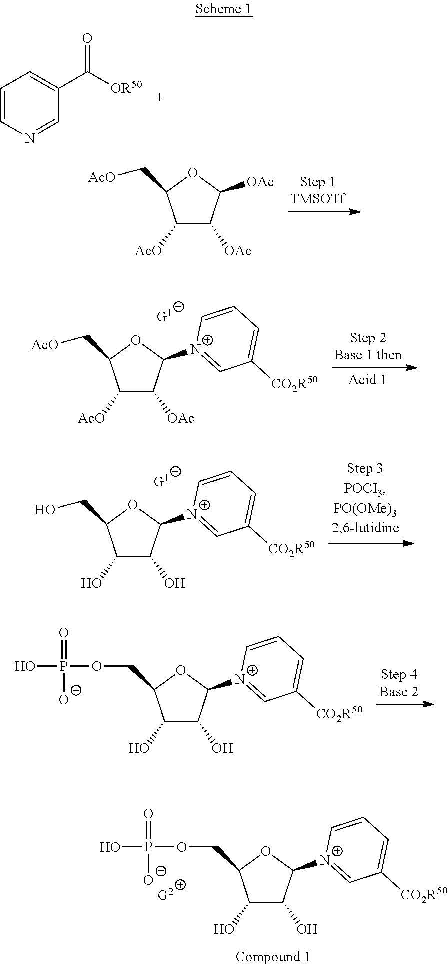
wherein
R⁵⁰ is alkyl;
G¹ is an anion; and
G² is a cation.

98. The method of claim 97, wherein Step 1 is performed under flow conditions.

99. The method of claim 97 or 98, wherein Step 2 is performed under flow conditions.

100. The method of any one of claims 97-99, wherein Step 3 is performed under flow conditions.

101. The method of any one of claims 97-100, wherein Step 4 is performed under flow conditions.

102. The method of any one of claims 97-101, wherein Base 1 is a hydroxide (e.g., aqueous sodium hydroxide).

103. The method of any one of claims 97-101, wherein Acid 1 is a mineral acid (e.g., aqueous sulfuric acid).

104. The method of any one of claims 97-101, wherein Base 2 is a hydroxide (e.g., aqueous sodium hydroxide).

105. The method of any one of claims 97-104, wherein the method is performed in acetonitrile.

106. The method of any one of claims 97-104, wherein the method is performed in a mixture of acetonitrile and ethanol.

107. A method of making Compound 1, wherein the method is performed as depicted in Scheme II: 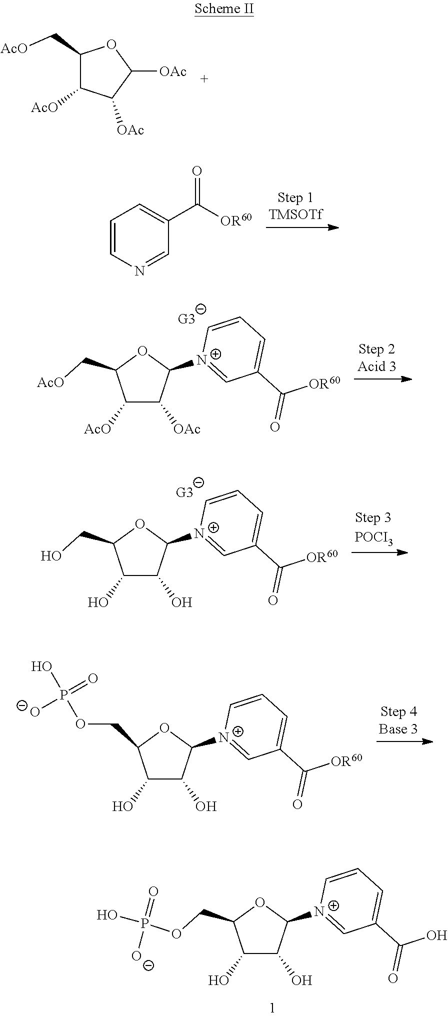
wherein
R⁵¹ is alkyl or aralkyl; and
G³ is an anion.

108. The method of claim 107, wherein Step 1 is performed in a halogenated hydrocarbon solvent (e.g., dichloromethane).

109. The method of claim 107 or 108, wherein Acid 3 is a mineral acid (e.g., aqueous hydrochloric acid).

110. The method of claim 109, wherein the mineral acid is the solvent.

111. The method of any one of claims 107-110, wherein Base 3 is a hydroxide (e.g., aqueous lithium hydroxide).

112. The method of any one of claims 107-111, wherein Step 4 is performed in a mixture of an organic solvent and water (e.g., tetrahydrofuran and water).

113. The method of any one of claims 107-112, wherein R⁵⁰ is aralkyl (e.g., benzyl).
